(19) United States
(12) Patent Application Publication
(10) Pub. No.: US 2016/0040149 A1
(43) Pub. Date: Feb. 11, 2016

(54) Compositions Having Dicamba Decarboxylase Activity and Methods of Use

(71) Applicants: PIONEER HI-BRED INTERNATIONAL INC., Johnston, IA (US); ARZEDA CORP., Seatteal, WA (US)

(72) Inventors: Eric Althoff, Seattle, WA (US); Yi-En Andrew Ban, Seattle, WA (US); Linda A. Castle, Mountain View, CA (US); Daniela Grabs, Seattle, WA (US); Jian Lu, Union City, CA (US); Phillip A. Patten, Portola Valley, CA (US); Yumin Tao, Fremont, CA (US); Alexandre Zanghellini, Seattle, WA (US)

(21) Appl. No.: 14/776,321

(22) PCT Filed: Mar. 14, 2014
(86) PCT No.: PCT/US14/29769

§ 371 Date: Sep. 14, 2015

Related U.S. Application Data
(60) Provisional application No. 61/782,586, filed on Mar. 14, 2013.

(51) Int. Cl. C12N 9/88; C12P 7/22; C12Q 1/68; C12P 7/00; G01N 33/573

(52) U.S. Cl. CPC C12N 9/88; C12P 7/00; G01N 33/573; C12Q 1/6876; C12P 7/22; C12Y 401/01; G01N 2333/988; C12Q 2600/158

(57) ABSTRACT

Compositions and methods comprising polynucleotides and polypeptides having dicamba decarboxylase activity are provided. Further provided are nucleic acid constructs, host cells, plants, plant cells, explants, seeds and grain having the dicamba decarboxylase sequences. Various methods of employing the dicamba decarboxylase sequences are provided. Such methods include, for example, methods for decarboxylating an auxin-analog, method for producing an auxin-analog tolerant plant, plant cell, explant or seed and methods of controlling weeds in a field containing a crop employing the plants and/or seeds disclosed herein. Methods are also provided to identify additional dicamba decarboxylase variants.

### CROSS-REFERENCE TO RELATED APPLICATIONS

This application claims the benefit of U.S. Provisional Application No. 61/782,586, filed on Mar. 14, 2013, which is incorporated herein by reference in its entirety.

### FIELD OF THE INVENTION

This invention is in the field of molecular biology. More specifically, this invention pertains to method and compositions comprising polypeptides having dicamba decarboxylase activity and methods of their use.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 36446_{—}0076P1_Sequence_Listing.txt, created on Mar. 14, 2013, and having a size of 2,416,640 bytes and is filed concurrently with the specification. The sequence listing contained in this ASCII formatted document is part of the specification and is herein incorporated by reference in its entirety.

### BACKGROUND OF THE INVENTION

In the commercial production of crops, it is desirable to easily and quickly eliminate unwanted plants (i.e., “weeds”) from a field of crop plants. An ideal treatment would be one which could be applied to an entire field but which would eliminate only the unwanted plants while leaving the crop plants unharmed. One such treatment system would involve the use of crop plants which are tolerant to a herbicide so that when the herbicide was sprayed on a field of herbicide-tolerant crop plants or an area of cultivation containing the crop, the crop plants would continue to thrive while non-herbicide-tolerant weeds were killed or severely damaged. Ideally, such treatment systems would take advantage of varying herbicide properties so that weed control could provide the best possible combination of flexibility and economy. For example, individual herbicides have different longevities in the field, and some herbicides persist and are effective for a relatively long time after they are applied to a field while other herbicides are quickly broken down into other and/or non-active compounds.

Crop tolerance to specific herbicides can be conferred by engineering genes into crops which encode appropriate herbicide metabolizing enzymes and/or insensitive herbicide targets. In some cases these enzymes, and the nucleic acids that encode them, originate in a plant. In other cases, they are derived from other organisms, such as microbes. See, e.g., Padgette et al. (1996) “New weed control opportunities: Development of soybeans with a Roundup Ready® gene” and Vasil (1996) “Phosphinothricin-resistant crops,” both in Herbicide-Resistant Crops, ed. Duke (CRC Press, Boca Raton, Fla.) pp. 54-84 and pp. 85-91. Indeed, transgenic plants have been engineered to express a variety of herbicide tolerance genes from a variety of organisms.

While a number of herbicide-tolerant crop plants are presently commercially available, improvements in every aspect of crop production, weed control options, extension of residual weed control, and improvement in crop yield are continuously in demand. Particularly, due to local and regional variation in dominant weed species, as well as, preferred crop species, a continuing need exists for customized systems of crop protection and weed management which can be adapted to the needs of a particular region, geography, and/or locality. A continuing need therefore exists for compositions and methods of crop protection and weed management.

### BRIEF SUMMARY OF THE INVENTION

Compositions and methods comprising polynucleotides and polypeptides having dicamba decarboxylase activity are provided. Further provided are nucleic acid constructs, host cells, plants, plant cells, explants, seeds and grain having the dicamba decarboxylase sequences. Various methods of employing the dicamba decarboxylase sequences are provided. Such methods include, for example, methods for decarboxylating an auxin-analog, method for producing an auxin-analog tolerant plant, plant cell, explant or seed and methods of controlling weeds in a field containing a crop employing the plants and/or seeds disclosed herein. Methods are also provided to identify additional dicamba decarboxylase variants.

### BRIEF DESCRIPTION OF THE DRAWINGS

FIG. 1 provides a schematic showing chemical structures of substrate dicamba (A) and of products including (B) carbon dioxide (C) 2,5-dichloro anisole (D) 4-chloro-3-methoxy phenol and (E) 2,5-dichloro phenol formed from reactions catalyzed by dicamba decarboxylases.

FIG. 2 shows that soybean germination is not affected by the dicamba decarboxylation product 2,5-dichloro anisole.

FIG. 3 shows that Arabidopsis root growth on MS medium (A). The root growth is inhibited by dicamba (B, luM; C, 10 uM) but not affected by 4-chloro-3-methoxy phenol (D, luM; E, 10 uM) or 2,5-dichloro phenol (F, luM; G, 10 uM).

FIG. 4 provides the phylogenic relationship of 108 decarboxylase homologs using CLUSTAL W. The phylogenetic tree was inferred using the Neighbor-Joining method (Saitou and Nei (1987) Molecular Biology and Evolution 4:406-425). The bootstrap consensus tree inferred from 1000 replicates is taken to represent the evolutionary history of the taxa analyzed (Felsenstein (1985) Evolution 39:783-791). Branches corresponding to partitions reproduced in less than 50% bootstrap replicates are collapsed. The evolutionary distances were computed using the Poisson correction method (Zuckerkandl and Pauling (1965) In Evolving Genes and Proteins by Bryson and Vogel, pp. 97-166. Academic Press, New York) and are in the units of the number of amino acid substitutions per site. The analysis involved 108 amino acid sequences. All positions containing gaps and missing data were eliminated. There were a total of 85 positions in the final dataset. Evolutionary analyses were conducted in MEGA5 (Tamura et al. (2011) Molecular Biology and Evolution 28: 2731-2739). Filled circle: Proteins with dicamba decarboxylase activity. Open circle: Proteins with no detected dicamba decarboxylase activity. Open diamond: Proteins with low, but detectable dicamba decarboxylase activity. See Table 1 for sequence sources.

FIG. 5 shows dicamba decarboxylation activity of SEQ ID NO:1 and SEQ ID NO:109 in a ¹⁴C assay using E. coli recombinant strains. 90 ul of IPTG-induced E. coli cells was incubated with 2 mM [¹⁴C]-carboxyl-labeled dicamba in ¹⁴C assay as described in Example 1. Panel A, reaction at time 0; Panel B, reaction was carried out for one hour; Panel C, reaction was carried out for four hours; Panel D, reaction was carried out for twelve hours. Sample 1 and 2 are two E. coli BL21 cell lines expressing SEQ ID NO:1. Sample 3 and 4 are two E. coli BL21 cell lines expressing SEQ ID NO:109. Sample 5 is a control E. coli BL21 cell line. Darker signal indicates higher dicamba decarboxylase activity.

FIG. 6 is a substrate concentration versus reaction velocity graph depicting protein kinetic activity improvement of SEQ ID NO:123 over SEQ ID NO:109.

FIG. 7 shows the distribution of neutral or beneficial amino acid changes respective to position in SEQ ID NO:109 from the N-terminus to the C-terminus of the protein.

FIG. 8 shows structural locations of amino acid positions of SEQ ID NO:109 where at least one point mutation led to greater than 1.6-fold higher dicamba decarboxylase activity. These positions are mapped with amino acid side chains shown. Arrows: Conserved regions.

FIG. 9 shows variants with improved activity based from a ¹⁴C-assay screening of the first round of a recombinatorial library in 384-well format. Each square represents ¹⁴CO₂ generated from cells expressing one shuffled protein variant. Darker signal indicates higher dicamba decarboxylase activity. Each marked rectangle has 8 controls including 4 positive proteins (backbone for the library) and 4 negative controls. Reactions were carried out for 2 hours and filters were exposed for 3 days.

FIG. 10 provides the active site model and reaction mechanism for decarboxylation.

FIG. 11 provides a three-dimensional representation of the catalytic residues and metal for a decarboxylation reaction in a protein scaffold.

FIG. 12 provides the constraints for the distances between the key atoms of each sidechain, metal, and dicamba transition state.

FIG. 13 provides possible loop structures used in computational design of dicamba decarboxylase.

FIG. 14 provides the structures of various auxin-analog herbicides.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Enzymatic decarboxylation reactions, with the exception of orotidine decarboxylase have not been studied or researched in detail. There is little information about their mechanism or enzymatic rates and no significant work done to improve their catalytic efficiency nor their substrate specificity. Decarboxylation reactions catalyze the release of CO₂ from their substrates which is quite remarkable given the energy requirements to break a carbon-carbon sigma bond, one of the strongest known in nature.

In examining the structure of the auxin-analog, dicamba, the importance of the carboxylate (—CO₂— or —CO₂H) to its function was identified and enzymes were successfully identified and designed that would remove the carboxylate moiety efficiently rendering a significantly different product than dicamba. Such work is of particular interest for the auxin-analog herbicides, such as dicamba (3,6-dichloro-2-methoxy benzoic acid) and 2,4-D or derivatives or metabolic products thereof. These compounds have been used in agriculture to effectively control broadleaf weeds in crop fields including corn and wheat for many years. They have also been shown to be effective in controlling recently emerged weed species that have gained resistance to the widely-used herbicide glyphosate. However, crops of dicot species including soybean are extremely sensitive to dicamba. To enable the application of auxin-analog herbicides in these crop fields, an auxin-analog herbicide tolerance trait is needed.

Methods and compositions are provided which allow for the decarboxylation of auxin-analogs. Specifically, polypeptides having dicamba decarboxylase activity are provided. As demonstrated herein, dicamba decarboxylase polypeptides can decarboxylate auxin-analogs, including auxin-analog herbicides, such as dicamba, or derivatives or metabolic products thereof, and thereby reduce the herbicidal toxicity of the auxin-analog to plants.

### II. Compositions

A. Dicamba Decarboxylase Polypeptides and Polynucleotides Encoding the Same

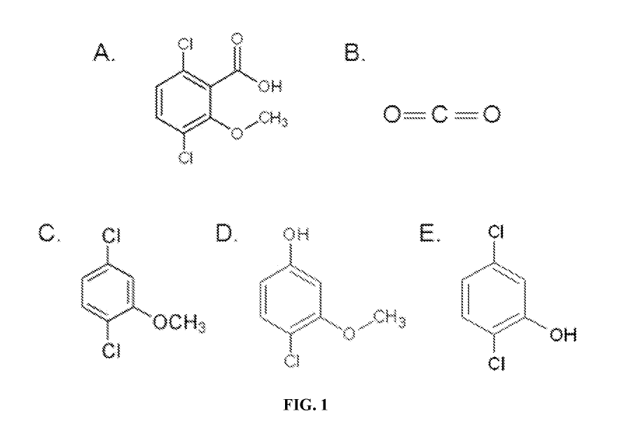

As used herein, a “dicamba decarboxylase polypeptide” or a polypeptide having “dicamba decarboxylase activity” refers to a polypeptide having the ability to decarboxylate dicamba. “Decarboxylate” or “decarboxylation” refers to the removal of a COOH (carboxyl group), releasing CO₂ and replacing the carboxyl group with a proton. FIG. 1 provides a schematic showing chemical structures of dicamba and products that can result following decarboxylation of dicamba. As shown in FIG. 1, along with a simple decarboxylation to produce CO₂, a variety of factors during the reaction can influence which additional biproducts are formed. With regard to FIG. 1, C is the simplest decarboxylation where the CO₂ is replaced by a proton, D is the product after decarboxylation and chlorohydrolase activity, and E is the product after decarboxylation and demethylase or methoxyhydrolase activity.

A variety of dicamba decarboxylases are provided, including but not limited to, the sequences set forth in SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 or 129 or active variant or fragments thereof and the polynucleotides encoding the same.

In further embodiments, a variety of dicamba decarboxylases are provided, including but not limited to, the sequences set forth in SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, and 1042, or active variant or fragments thereof and the polynucleotides encoding the same.

Further provided herein are a variety of dicamba decarboxylases are provided, including but not limited to, a polypeptide having dicamba decarboxylase activity; wherein the polypeptide having dicamba decarboxylase activity further comprises:

```
(SEQ ID NO: 1041)
                                5 10
Met Ala Xaa Gly Lys Val Xaa Leu Glu Glu His Xaa
                15 20
Ala Ile Xaa Xaa Thr Leu Xaa Xaa Xaa Ala Xaa Phe
25 30 35
Val Pro Xaa Xaa Tyr Xaa Lys Xaa Leu Xaa His Arg
                        40 45
Leu Xaa Asp Xaa Gln Xaa Xaa Arg Leu Xaa Xaa Met
        50 55 60
Asp Xaa His Xaa Ile Xaa Xaa Met Xaa Leu Ser Leu
                                65 70
Xaa Ala Xaa Xaa Xaa Gln Xaa Xaa Xaa Xaa Arg Xaa
                75 80
Xaa Ala Xaa Xaa Xaa Ala Xaa Arg Xaa Asn Asp Xaa
85 90 95
Xaa Ala Glu Xaa Xaa Ala Xaa Xaa Xaa Xaa Arg Phe
                        100 105
Xaa Ala Phe Xaa Xaa Xaa Pro Xaa Xaa Asp Xaa Xaa
        110 115 120
Xaa Ala Xaa Xaa Glu Leu Gln Arg Xaa Val Xaa Xaa
                                125 130
Leu Gly Xaa Val Gly Ala Xaa Val Asn Gly Phe Ser
                135 140
Xaa Glu Gly Asp Xaa Xaa Thr Pro Leu Tyr Tyr Asp
145 150 155
Leu Pro Xaa Tyr Arg Pro Phe Trp Xaa Glu Val Glu
                        160 165
Lys Leu Asp Val Pro Phe Tyr Leu His Pro Xaa Asn
        170 175 180
Pro Leu Pro Gln Asp Xaa Arg Ile Tyr Xaa Gly His
                                185 190
Pro Trp Leu Leu Gly Pro Thr Trp Ala Phe Ala Gln
                195 200
Glu Thr Xaa Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Xaa
        230 235 240
Xaa Arg Ile Asp His Arg Xaa Xaa Xaa Xaa Xaa Xaa
                                245 250
Pro Pro Xaa Tyr Xaa Ala Lys Xaa Xaa Phe Xaa Asp
                255 260
Tyr Phe Xaa Glu Asn Phe Xaa Xaa Thr Thr Ser Gly
265 270 275
Asn Phe Arg Thr Gln Thr Leu Ile Asp Ala Ile Leu
                        280 285
Glu Xaa Gly Ala Asp Arg Ile Leu Phe Ser Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Xaa Thr Asn Ala Xaa Xaa Leu Phe Lys
325
Leu Asp Xaa Xaa,
```

wherein

Xaa at position 3 is Gln, Gly, Met or Pro; Xaa at position 7 is Ala or Cys; Xaa at position 12 is Phe, Met, Val or Trp; Xaa at position 15 is Pro or Thr; Xaa at position 16 is Glu or Ala; Xaa at position 19 is Gln, Glu or Asn; Xaa at position 20 is Asp, Cys, Phe, Met or Trp; Xaa at position 21 is Ser, Ala, Gly or Val; Xaa at position 23 is Gly or Asp; Xaa at position 27 is Gly, Ala, Asp, Glu, Pro, Arg, Ser, Thr or Tyr; Xaa at position 28 is Asp, Cys, Glu, Phe or Gly; Xaa at position 30 is Trp, Leu or Val; Xaa at position 32 is Glu or Val; Xaa at position 34 is Gln, Ala or Trp; Xaa at position 38 is Leu, Ile, Met, Arg, Thr or Val; Xaa at position 40 is Ile, Met, Ser or Val; Xaa at position 42 is Asp, Ala, Gly, Lys, Met, Ser or Thr; Xaa at position 43 is Thr, Cys, Asp, Glu, Gly, Met, Gln, Arg or Tyr; Xaa at position 46 is Lys, Gly, Asn or Arg; Xaa at position 47 is Leu, Cys, Glu, Lys or Ser; Xaa at position 50 is Ala, Lys, Arg, Ser, Thr or Val; Xaa at position 52 is Gly, Glu, Leu, Asn or Gln; Xaa at position 54 is Glu or Gly; Xaa at position 55 is Thr or Leu; Xaa at position 57 is Ile, Ala or Val; Xaa at position 61 is Asn, Ala, Gly, Leu or Ser; Xaa at position 63 is Pro or Val; Xaa at position 64 is Ala, Gly, His or Ser; Xaa at position 65 is Val or Cys; Xaa at position 67 is Ala or Ser; Xaa at position 68 is Ile or Gln; Xaa at position 69 is Pro, Gly, Arg, Ser or Val; Xaa at position 70 is Asp or His; Xaa at position 72 is Arg, Lys or Val; Xaa at position 73 is Lys, Glu, Gln or Arg; Xaa at position 75 is Ile or Arg; Xaa at position 76 is Glu or Gly; Xaa at position 77 is Ile, Met, Arg, Ser or Val; Xaa at position 79 is Arg or Gln; Xaa at position 81 is Ala or Ser; Xaa at position 84 is Val, Cys, Phe or Met; Xaa at position 85 is Leu or Ala; Xaa at position 88 is Glu or Lys; Xaa at position 89 is Cys, Ile or Val; Xaa at position 91 is Lys or Arg; Xaa at position 92 is Arg or Lys; Xaa at position 93 is Pro, Ala or Arg; Xaa at position 94 is Asp, Cys, Gly, Gln or Ser; Xaa at position 97 is Leu, Lys or Arg; Xaa at position 100 is Ala, Gly or Ser; Xaa at position 101 is Ala or Gly; Xaa at position 102 is Leu or Val; Xaa at position 104 is Leu or Met; Xaa at position 105 is Gln or Gly; Xaa at position 107 is Pro or Val; Xaa at position 108 is Asp or Glu; Xaa at position 109 is Ala, Gly, Met or Val; Xaa at position 111 is Thr, Ala, Cys, Gly, Ser or Val; Xaa at position 112 is Glu, Gly, Arg or Ser; Xaa at position 117 is Cys, Ala or Thr; Xaa at position 119 is Asn, Ala, Cys, Arg or Ser; Xaa at position 120 is Asp or Thr; Xaa at position 123 is Phe or Leu; Xaa at position 127 is Leu or Met; Xaa at position 133 is Gln or Val; Xaa at position 137 is Gly, Ala or Glu; Xaa at position 138 is Gln or Gly; Xaa at position 147 is Gln or Ile; Xaa at position 153 is Gly or Lys; Xaa at position 167 is Arg or Glu; Xaa at position 174 is Ser or Ala; Xaa at position 178 is Asp or Glu; Xaa at position 195 is Ala or Gly; Xaa at position 212 is Arg, Gly or Gln; Xaa at position 214 is Asn or Gln; Xaa at position 220 is Met or Leu; Xaa at position 228 is Met or Leu; Xaa at position 229 is Trp or Tyr; Xaa at position 235 is Val or Ile; Xaa at position 236 is Ala, Gly, Gln or Trp; Xaa at position 237 is Trp or Leu; Xaa at position 238 is Val, Gly or Pro; Xaa at position 239 is Lys, Ala, Asp, Glu, Gly or His; Xaa at position 240 is Leu, Ala, Asp, Glu, Gly or Val; Xaa at position 243 is Arg, Ala, Asp, Lys, Ser or Val; Xaa at position 245 is Pro or Ala; Xaa at position 248 is Arg or Lys; Xaa at position 249 is Arg or Pro; Xaa at position 251 is Met or Val; Xaa at position 255 is Asn, Ala, Leu, Met, Gln, Arg or Ser; Xaa at position 259 is His or Trp; Xaa at position 260 is Ile or Leu; Xaa at position 278 is Ile or Leu; Xaa at position 298 is Ser, Ala or Thr; Xaa at position 299 is Asp or Ala; Xaa at position 302 is Asn or Ala; Xaa at position 303 is Ala, Cys, Asp, Glu or Ser; Xaa at position 304 is Thr or Val; Xaa at position 312 is Val or Leu; Xaa at position 316 is Arg or Ser; Xaa at position 320 is Arg or Leu; Xaa at position 321 is Arg or Asn; Xaa at position 327 is Gly, Leu, Gln or Val; Xaa at position 328 is Ala, Cys, Asp, Arg, Ser, Thr or Val; wherein one or more amino acid(s) designated by Xaa in SEQ ID NO: 1041 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

Further provided herein are a variety of dicamba decarboxylases are provided, including but not limited to, a polypeptide having dicamba decarboxylase activity; wherein the polypeptide having dicamba decarboxylase activity further comprises:

```
(SEQ ID NO: 1042)
                                5 10
Met Ala Gln Gly Xaa Val Ala Leu Glu Glu His Phe
                15 20
Ala Ile Pro Xaa Thr Leu Xaa Asp Xaa Ala Xaa Phe
25 30 35
Val Pro Xaa Xaa Tyr Xaa Lys Glu Leu Gln His Arg
                        40 45
Leu Xaa Asp Xaa Gln Asp Xaa Arg Leu Xaa Xaa Met
        50 55 60
Asp Xaa His Xaa Ile Xaa Thr Met Xaa Leu Ser Leu
                                65 70
Xaa Ala Xaa Xaa Val Gln Xaa Ile Xaa Asp Arg Xaa
                75 80
Xaa Ala Ile Glu Xaa Ala Xaa Arg Ala Asn Asp Xaa
85 90 95
Leu Ala Glu Glu Xaa Ala Lys Arg Pro Xaa Arg Phe
                        100 105
Leu Ala Phe Ala Ala Leu Pro Xaa Gln Asp Xaa Xaa
        110 115 120
Ala Ala Xaa Xaa Glu Leu Gln Arg Xaa Val Xaa Xaa
                                125 130
Leu Gly Phe Val Gly Ala Xaa Val Asn Gly Phe Ser
                135 140
Xaa Glu Gly Asp Gly Gln Thr Pro Leu Tyr Tyr Asp
145 150 155
Leu Pro Gln Tyr Arg Pro Phe Trp Xaa Glu Val Glu
                        160 165
Lys Leu Asp Val Pro Phe Tyr Leu His Pro Arg Asn
        170 175 180
Pro Leu Pro Gln Asp Xaa Arg Ile Tyr Asp Gly His
                                185 190
Pro Trp Leu Leu Gly Pro Thr Trp Ala Phe Ala Gln
                195 200
Glu Thr Ala Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Met
        230 235 240
Xaa Arg Ile Asp His Arg Xaa Xaa Trp Val Xaa Xaa
                                245 250
Pro Pro Xaa Tyr Xaa Ala Lys Arg Arg Phe Met Asp
                255 260
Tyr Phe Xaa Glu Asn Phe Xaa Ile Thr Thr Ser Gly
265 270 275
Asn Phe Arg Thr Gln Thr Leu Ile Asp Ala Ile Leu
                        280 285
Glu Ile Gly Ala Asp Arg Ile Leu Phe Xaa Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Arg Thr Asn Ala Xaa Xaa Leu Phe Lys
325
Leu Asp Xaa Xaa
```

wherein
Xaa at position 5 is Lys or Leu; Xaa at position 16 is Glu or Ala; Xaa at position 19 is Gln or Asn; Xaa at position 21 is Ser or Ala; Xaa at position 23 is Gly or Asp; Xaa at position 27 is Gly or Ser; Xaa at position 28 is Asp, Cys or Glu; Xaa at position 30 is Trp or Leu; Xaa at position 38 is Leu or Met; Xaa at position 40 is Ile or Met; Xaa at position 43 is Thr, Glu or Gln; Xaa at position 46 is Lys, Asn or Arg; Xaa at position 47 is Leu or Glu; Xaa at position 50 is Ala, Lys or Arg; Xaa at position 52 is Gly, Glu or Gin; Xaa at position 54 is Glu or Gly; Xaa at position 57 is Ile or Val; Xaa at position 61 is Asn or Ala; Xaa at position 63 is Pro or Val; Xaa at position 64 is Ala or Gly; Xaa at position 67 is Ala, Gly or Ser; Xaa at position 69 is Pro, Gly or Val; Xaa at position 72 is Arg or Val; Xaa at position 73 is Lys, Glu or Gln; Xaa at position 77 is Ile or Leu; Xaa at position 79 is Arg or Lys; Xaa at position 84 is Val, Phe or Met; Xaa at position 89 is Cys or Val; Xaa at position 94 is Asp or Gly; Xaa at position 104 is Leu or Met; Xaa at position 107 is Pro or Val; Xaa at position 108 is Asp or Glu; Xaa at position 111 is Thr or Ser; Xaa at position 112 is Glu or Ser; Xaa at position 117 is Cys or Thr; Xaa at position 119 is Asn, Ala or Arg; Xaa at position 120 is Asp or Thr; Xaa at position 127 is Leu or Met; Xaa at position 133 is Gin or Val; Xaa at position 153 is Gly or Lys; Xaa at position 174 is Ser or Ala; Xaa at position 212 is Arg or Gly; Xaa at position 214 is Asn or Gln; Xaa at position 220 is Met or Leu; Xaa at position 229 is Trp or Tyr; Xaa at position 235 is Val or Ile; Xaa at position 236 is Ala or Gly; Xaa at position 239 is Lys, Glu or His; Xaa at position 240 is Leu, Ala or Glu; Xaa at position 243 is Arg or Asp; Xaa at position 245 is Pro or Ala; Xaa at position 255 is Asn or Leu; Xaa at position 259 is His or Trp; Xaa at position 286 is Ser or Ala; Xaa at position 298 is Ser, Ala or Thr; Xaa at position 299 is Asp or Ala; Xaa at position 302 is Asn or Ala; Xaa at position 303 is Ala or Glu; Xaa at position 304 is Thr or Ala; Xaa at position 312 is Val or Leu; Xaa at position 320 is Arg or Leu; Xaa at position 321 is Arg or Asn; Xaa at position 327 is Gly, Leu or Val; Xaa at position 328 is Ala, Asp, Arg, Ser or Thr; wherein one or more amino acid(s) designated by Xaa in SEQ ID NO: 1042 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

Further provided herein are a variety of dicamba decarboxylases are provided, including but not limited to, a polypeptide having dicamba decarboxylase activity; wherein the polypeptide having dicamba decarboxylase activity further comprises:

```
(SEQ ID NO: 1043)
                                5 10
Met Ala Xaa Gly Lys Val Xaa Leu Glu Glu His Xaa
                15 20
Ala Ile Xaa Xaa Thr Leu Xaa Xaa Xaa Ala Xaa Phe
25 30 35
Val Pro Xaa Xaa Tyr Xaa Lys Xaa Leu Xaa His Arg
                        40 45
Leu Xaa Asp Xaa Gln Xaa Xaa Arg Leu Xaa Xaa Met
        50 55 60
Asp Xaa His Xaa Ile Xaa Xaa Met Xaa Leu Ser Leu
                                65 70
Xaa Ala Xaa Xaa Xaa Gln Xaa Xaa Xaa Xaa Arg Xaa
                75 80
Xaa Ala Xaa Xaa Xaa Ala Xaa Arg Xaa Asn Asp Xaa
85 90 95
Xaa Ala Glu Xaa Xaa Ala Xaa Xaa Xaa Xaa Arg Phe
                        100 105
Xaa Ala Phe Xaa Xaa Xaa Pro Xaa Xaa Asp Xaa Xaa
        110 115 120
Xaa Ala Xaa Xaa Glu Leu Gln Arg Xaa Val Xaa Xaa
                                125 130
Leu Gly Xaa Val Gly Ala Xaa Val Asn Gly Phe Ser
                135 140
Xaa Glu Gly Asp Xaa Xaa Thr Pro Leu Tyr Tyr Asp
145 150 155
Leu Pro Xaa Tyr Arg Pro Phe Trp Xaa Glu Val Glu
                        160 165
Lys Leu Asp Val Pro Phe Tyr Leu His Pro Xaa Asn
        170 175 180
Pro Leu Pro Gln Asp Xaa Arg Ile Tyr Xaa Gly His
                                185 190
Pro Trp Leu Leu Gly Pro Thr Trp Ala Phe Ala Gln
                195 200
Glu Thr Xaa Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Xaa
        230 235 240
Xaa Arg Ile Asp His Arg Xaa Xaa Xaa Xaa Xaa Xaa
                                245 250
Pro Pro Xaa Tyr Xaa Ala Lys Xaa Xaa Phe Xaa Asp
                255 260
Tyr Phe Xaa Glu Asn Phe Xaa Xaa Thr Thr Ser Gly
265 270 275
Asn Phe Arg Thr Gln Thr Leu Ile Asp Ala Ile Leu
                        280 285
Glu Xaa Gly Ala Asp Arg Ile Leu Phe Ser Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Xaa Thr Asn Ala Xaa Xaa Leu Phe Lys
325
Leu Asp Xaa Xaa,
```

wherein

Xaa at position 3 is Gin, Gly, Met or Pro; Xaa at position 7 is Ala or Cys; Xaa at position 12 is Phe, Met, Val or Trp; Xaa at position 15 is Pro or Thr; Xaa at position 16 is Glu or Ala; Xaa at position 19 is Gln, Glu or Asn; Xaa at position 20 is Asp, Cys, Phe, Met or Trp; Xaa at position 21 is Ser, Ala, Gly or Val; Xaa at position 23 is Gly or Asp; Xaa at position 27 is Gly, Ala, Asp, Glu, Pro, Arg, Ser, Thr or Tyr; Xaa at position 28 is Asp, Cys, Glu, Phe or Gly; Xaa at position 30 is Trp, Leu or Val; Xaa at position 32 is Glu or Val; Xaa at position 34 is Gln, Ala or Trp; Xaa at position 38 is Leu, Ile, Met, Arg, Thr or Val; Xaa at position 40 is Ile, Met, Ser or Val; Xaa at position 42 is Asp, Ala, Gly, Lys, Met, Ser or Thr; Xaa at position 43 is Thr, Cys, Asp, Glu, Gly, Met, Gin, Arg or Tyr; Xaa at position 46 is Lys, Gly, Asn or Arg; Xaa at position 47 is Leu, Cys, Glu, Lys or Ser; Xaa at position 50 is Ala, Lys, Arg, Ser, Thr or Val; Xaa at position 52 is Gly, Glu, Leu, Asn or Gln; Xaa at position 54 is Glu or Gly; Xaa at position 55 is Thr or Leu; Xaa at position 57 is Ile, Ala or Val; Xaa at position 61 is Asn, Ala, Gly, Leu or Ser; Xaa at position 63 is Pro or Val; Xaa at position 64 is Ala, Gly, His or Ser; Xaa at position 65 is Val or Cys; Xaa at position 67 is Ala or Ser; Xaa at position 68 is Ile or Gln; Xaa at position 69 is Pro, Gly, Arg, Ser or Val; Xaa at position 70 is Asp or His; Xaa at position 72 is Arg, Lys or Val; Xaa at position 73 is Lys, Glu, Gln or Arg; Xaa at position 75 is Ile or Arg; Xaa at position 76 is Glu or Gly; Xaa at position 77 is Ile, Met, Arg, Ser or Val; Xaa at position 79 is Arg or Gln; Xaa at position 81 is Ala or Ser; Xaa at position 84 is Val, Cys, Phe or Met; Xaa at position 85 is Leu or Ala; Xaa at position 88 is Glu or Lys; Xaa at position 89 is Cys, Ile or Val; Xaa at position 91 is Lys or Arg; Xaa at position 92 is Arg or Lys; Xaa at position 93 is Pro, Ala or Arg; Xaa at position 94 is Asp, Cys, Gly, Gln or Ser; Xaa at position 97 is Leu, Lys or Arg; Xaa at position 100 is Ala, Gly or Ser; Xaa at position 101 is Ala or Gly; Xaa at position 102 is Leu or Val; Xaa at position 104 is Leu or Met; Xaa at position 105 is Gln or Gly; Xaa at position 107 is Pro or Val; Xaa at position 108 is Asp or Glu; Xaa at position 109 is Ala, Gly, Met or Val; Xaa at position 111 is Thr, Ala, Cys, Gly, Ser or Val; Xaa at position 112 is Glu, Gly, Arg or Ser; Xaa at position 117 is Cys, Ala or Thr; Xaa at position 119 is Asn, Ala, Cys, Arg or Ser; Xaa at position 120 is Asp or Thr; Xaa at position 123 is Phe or Leu; Xaa at position 127 is Leu or Met; Xaa at position 133 is Gln or Val; Xaa at position 137 is Gly, Ala or Glu; Xaa at position 138 is Gln or Gly; Xaa at position 147 is Gln or Ile; Xaa at position 153 is Gly or Lys; Xaa at position 167 is Arg or Glu; Xaa at position 174 is Ser or Ala; Xaa at position 178 is Asp or Glu; Xaa at position 195 is Ala or Gly; Xaa at position 212 is Arg, Gly or Gln; Xaa at position 214 is Asn or Gln; Xaa at position 220 is Met or Leu; Xaa at position 228 is Met or Leu; Xaa at position 229 is Trp or Tyr; Xaa at position 235 is Asn, Val or Ile; Xaa at position 236 is Ala, Gly, Gln or Trp; Xaa at position 237 is Trp or Leu; Xaa at position 238 is Val, Gly or Pro; Xaa at position 239 is Lys, Ala, Asp, Glu, Gly or His; Xaa at position 240 is Leu, Ala, Asp, Glu, Gly or Val; Xaa at position 243 is Arg, Ala, Asp, Lys, Ser or Val; Xaa at position 245 is Pro or Ala; Xaa at position 248 is Arg or Lys; Xaa at position 249 is Arg or Pro; Xaa at position 251 is Met or Val; Xaa at position 255 is Asn, Ala, Leu, Met, Gln, Arg or Ser; Xaa at position 259 is His or Trp; Xaa at position 260 is Ile or Leu; Xaa at position 278 is Ile or Leu; Xaa at position 298 is Ser, Ala or Thr; Xaa at position 299 is Asp or Ala; Xaa at position 302 is Asn or Ala; Xaa at position 303 is Ala, Cys, Asp, Glu or Ser; Xaa at position 304 is Thr or Val; Xaa at position 312 is Val or Leu; Xaa at position 316 is Arg or Ser; Xaa at position 320 is Arg or Leu; Xaa at position 321 is Arg or Asn; Xaa at position 327 is Gly, Leu, Gln or Val; Xaa at position 328 is Ala, Cys, Asp, Arg, Ser, Thr or Val; wherein one or more amino acid(s) designated by Xaa in SEQ ID NO: 1043 is an amino acid different from the corresponding amino acid of SEQ ID NO: 1; and wherein the polypeptide having dicamba decarboxylase activity has increased dicamba decarboxylase activity compared to the polypeptide of SEQ ID NO: 1.

Further provided herein are a variety of dicamba decarboxylases are provided, including but not limited to, a polypeptide having dicamba decarboxylase activity; wherein the polypeptide having dicamba decarboxylase activity further comprises:

```
(SEQ ID NO: 1044)
                                5 10
Met Ala Gln Gly Xaa Val Ala Leu Glu Glu His Phe
                15 20
Ala Ile Pro Xaa Thr Leu Xaa Asp Xaa Ala Xaa Phe
25 30 35
Val Pro Xaa Xaa Tyr Xaa Lys Glu Leu Gln His Arg
                        40 45
Leu Xaa Asp Xaa Gln Asp Xaa Arg Leu Xaa Xaa Met
        50 55 60
Asp Xaa His Xaa Ile Xaa Thr Met Xaa Leu Ser Leu
                                65 70
Xaa Ala Xaa Xaa Val Gln Xaa Ile Xaa Asp Arg Xaa
                75 80
Xaa Ala Ile Glu Xaa Ala Xaa Arg Ala Asn Asp Xaa
85 90 95
Leu Ala Glu Glu Xaa Ala Lys Arg Pro Xaa Arg Phe
                        100 105
Leu Ala Phe Ala Ala Leu Pro Xaa Gln Asp Xaa Xaa
        110 115 120
Ala Ala Xaa Xaa Glu Leu Gln Arg Xaa Val Xaa Xaa
                                125 130
Leu Gly Phe Val Gly Ala Xaa Val Asn Gly Phe Ser
                135 140
Xaa Glu Gly Asp Gly Gln Thr Pro Leu Tyr Tyr Asp
145 150 155
Leu Pro Gln Tyr Arg Pro Phe Trp Xaa Glu Val Glu
                        160 165
Lys Leu Asp Val Pro Phe Tyr Leu His Pro Arg Asn
        170 175 180
Pro Leu Pro Gln Asp Xaa Arg Ile Tyr Asp Gly His
                                185 190
Pro Trp Leu Leu Gly Pro Thr Trp Ala Phe Ala Gln
                195 200
Glu Thr Ala Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Met
        230 235 240
Xaa Arg Ile Asp His Arg Xaa Xaa Trp Val Xaa Xaa
                                245 250
Pro Pro Xaa Tyr Xaa Ala Lys Arg Arg Phe Met Asp
                255 260
Tyr Phe Xaa Glu Asn Phe Xaa Ile Thr Thr Ser Gly
265 270 275
Asn Phe Arg Thr Gln Thr Leu Ile Asp Ala Ile Leu
                        280 285
Glu Ile Gly Ala Asp Arg Ile Leu Phe Xaa Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Arg Thr Asn Ala Xaa Xaa Leu Phe Lys
325
Leu Asp Xaa Xaa
```

wherein
Xaa at position 5 is Lys or Leu; Xaa at position 16 is Glu or Ala; Xaa at position 19 is Gln or Asn; Xaa at position 21 is Ser or Ala; Xaa at position 23 is Gly or Asp; Xaa at position 27 is Gly or Ser; Xaa at position 28 is Asp, Cys or Glu; Xaa at position 30 is Trp or Leu; Xaa at position 38 is Leu or Met; Xaa at position 40 is Ile or Met; Xaa at position 43 is Thr, Glu or Gln; Xaa at position 46 is Lys, Asn or Arg; Xaa at position 47 is Leu or Glu; Xaa at position 50 is Ala, Lys or Arg; Xaa at position 52 is Gly, Glu or Gln; Xaa at position 54 is Glu or Gly; Xaa at position 57 is Ile or Val; Xaa at position 61 is Asn or Ala; Xaa at position 63 is Pro or Val; Xaa at position 64 is Ala or Gly; Xaa at position 67 is Ala, Gly or Ser; Xaa at position 69 is Pro, Gly or Val; Xaa at position 72 is Arg or Val; Xaa at position 73 is Lys, Glu or Gin; Xaa at position 77 is Ile or Leu; Xaa at position 79 is Arg or Lys; Xaa at position 84 is Val, Phe or Met; Xaa at position 89 is Cys or Val; Xaa at position 94 is Asp or Gly; Xaa at position 104 is Leu or Met; Xaa at position 107 is Pro or Val; Xaa at position 108 is Asp or Glu; Xaa at position 111 is Thr or Ser; Xaa at position 112 is Glu or Ser; Xaa at position 117 is Cys or Thr; Xaa at position 119 is Asn, Ala or Arg; Xaa at position 120 is Asp or Thr; Xaa at position 127 is Leu or Met; Xaa at position 133 is Gln or Val; Xaa at position 153 is Gly or Lys; Xaa at position 174 is Ser or Ala; Xaa at position 212 is Arg or Gly; Xaa at position 214 is Asn or Gln; Xaa at position 220 is Met or Leu; Xaa at position 229 is Trp or Tyr; Xaa at position 235 is Asn, Val or Ile; Xaa at position 236 is Ala or Gly; Xaa at position 239 is Lys, Glu or His; Xaa at position 240 is Leu, Ala or Glu; Xaa at position 243 is Arg or Asp; Xaa at position 245 is Pro or Ala; Xaa at position 255 is Asn or Leu; Xaa at position 259 is His or Trp; Xaa at position 286 is Ser or Ala; Xaa at position 298 is Ser, Ala or Thr; Xaa at position 299 is Asp or Ala; Xaa at position 302 is Asn or Ala; Xaa at position 303 is Ala or Glu; Xaa at position 304 is Thr or Ala; Xaa at position 312 is Val or Leu; Xaa at position 320 is Arg or Leu; Xaa at position 321 is Arg or Asn; Xaa at position 327 is Gly, Leu or Val; Xaa at position 328 is Ala, Asp, Arg, Ser or Thr; wherein one or more amino acid(s) designated by Xaa in SEQ ID NO: 1044 is an amino acid different from the corresponding amino acid of SEQ ID NO: 1; and wherein the polypeptide having dicamba decarboxylase activity has increased dicamba decarboxylase activity compared to the polypeptide of SEQ ID NO: 1.

Further provided herein is the geometry of the active site of the dicamba decarboxylase enzymes. See Example 5. Thus, in other embodiments, dicamba decarboxylases are provided which comprise a catalytic residue geometry as set forth in Table 3 or a substantially similar geometry. As demonstrated herein, computational methods were performed to develop the minimal requirements and constraints for a dicamba decarboxylase active site. See Example 5 and Table 3 which provide the catalytic residue geometry for a dicamba decarboxylase polypeptide. Briefly, as summarized in both Table 3 and Table 6, catalytic residues #1-4 serve primarily to coordinate the metal within the active site. Most frequently they are histidine, aspartic acid, and glutamic acid. Catalytic residue #5 serves as the proton donor which adds the proton to the aromatic ring displacing the carboxylate. These five catalytic residues are critical to the dicamba decarboxylase activity. Thus, in specific embodiments, the dicamba decarboxylase comprises an active site having a catalytic residue geometry as set forth in Table 3 or having a substantially similar catalytic residue geometry.

As used herein, “a substantially similar catalytic residue geometry” is intended to describe a metal cation chelated directly by four catalytic residues composed of histidine, aspartic acid, and/or glutamic acid (but can also have tyrosine, asparagine, glutamine cysteine at at least one position) in a trigonal bipyramidal or other three-dimensional metal-coordination arrangements as allowed by the coordinated metal and its oxidative state. In specific embodiments, the four catalytic residues are composed of histidine, aspartic acid, and/or glutamic acid. Metal cations can include, zinc, cobalt, iron, nickel, copper, or manganese. (See, Huo, et al. Biochemistry. 2012 51:5811-21; Glueck, et al, Chem. Soc. Rev., 2010, 39, 313-328; Liu, et al, Biochemistry. 2006 45:10407-10411; Li, et al, Biochemistry 2006, 45:6628-6634, each of which is herein incorporated by reference). In one specific embodiment, the metal ion comprises zinc. Additionally a histidine residue (or other similarly polar side chain) is located near the 5^{th} ligand position of the metal and is positioned so as to donate a proton during the carboxylation step along the enzyme's mechanistic pathway. Substantially similar catalytic geometry is further meant to comprise of this constellation of 5 catalytic residues all within at least 1.5, 1, 0.9, 0.8, 0.7, 0.6, 0.5, 0.4, 0.3, 0.2, or 0.1 Angstroms of their ideal median value as shown in Table 3. In other embodiments, the substantially similar catalytic geometry comprises this constellation of 5 catalytic residues all within at least 0.5 Angstroms of their ideal or median value as shown in Table 3. It is recognized that a substantially similar catalytic residue geometry can comprise any combination of catalytic residues, metals and median distance to the metal atom disclosed above or in Table 3.

As demonstrated herein, the dicamba decarboxylase catalytic residue geometry set forth in Table 3 was present in natural protein structures or by homology modeling of the protein sequences. Additional active site residues were computationally designed in order to introduce dicamba binding and dicamba decarboxylation activity into an alpha-amino-beta-carboxymuconate-epsilon-semialdehyde-decarboxylase (SEQ ID NO:95) and a 4-oxalomesaconate hydratase (SEQ ID NO:100) by these methods. Neither of the native proteins have dicamba decarboxylase activity. Variants of the carboxymuconate-epsilon-semialdehyde-decarboxylase (SEQ ID NO:95) having the dicamba decarboxylase catalytic residue geometry set forth in Table 3 were generated and are set forth in SEQ ID NOS: 117, 118, and 119. Each of these sequences are shown herein to have dicamba decarboxylase activity. Likewise, variants of the oxalomesaconate hydratase (SEQ ID NO:100) having the dicamba decarboxylase catalytic residue geometry set forth in Table 3 were generated and are set forth in SEQ ID NOS: 120, 121 and 122. Each of these sequences are shown herein to have dicamba decarboxylase activity. In addition, polypeptides with native dicamba decarboxylase activity such as the amidohydrolase set forth in SEQ ID NO: 41 and the 2,6-dihydroxybenzoate decarboxylase set forth in SEQ ID NO:1 already possessed the dicamba decarboxylase catalytic residue geometry set forth in Table 3. The active site around the catalytic residues was computationally designed to recognize, bind, and be more catalytically efficient towards dicamba. The variants of these sequences having the catalytic residue geometry set forth in Table 3 are found in SEQ ID NOS; 109, 110, 111, 112, 113, 114, 115, and 116. Each of these variant sequences having the dicamba decarboxylase catalytic residue geometry set forth in Table 3 displays an increase in dicamba decarboxylase activity. Thus, dicamba decarboxylases are provided which have a catalytic residue geometry as set forth in Table 3 or having a substantially similar catalytic residue geometry.

i. Active Fragments of Dicamba Decarboxylase Sequences

Fragments and variants of dicamba decarboxylase polynucleotides and polypeptides can be employed in the methods and compositions disclosed herein. By “fragment” is intended a portion of the polynucleotide or a portion of the amino acid sequence and hence protein encoded thereby. Fragments of a polynucleotide may encode protein fragments that retain dicamba decarboxylase activity. Thus, fragments of a nucleotide sequence may range from at least about 20 nucleotides, about 50 nucleotides, about 100 nucleotides, and up to the full-length polynucleotide encoding the dicamba decarboxylase polypeptides.

A fragment of a dicamba decarboxylase polynucleotide that encodes a biologically active portion of a dicamba decarboxylase polypeptide will encode at least 50, 75, 100, 150, 175, 200, 225, 250, 275, 300, 325, 350, 375, 400, 410, 415, 420, 425, 430, 435, 440, 480, 500, 550, 600, 620 contiguous amino acids, or up to the total number of amino acids present in a full-length dicamba decarboxylase polypeptide as set forth in, for example, SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 or 129 or an active variant or fragment thereof. A fragment of a dicamba decarboxylase polynucleotide that encodes a biologically active portion of a dicamba decarboxylase polypeptide will comprise the total number of amino acids present in a full-length dicamba decarboxylase polypeptide as set forth in, for example, SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, and 1042.

In other embodiments, a fragment of a dicamba decarboxylase polynucleotide that encodes a biologically active portion of a dicamba decarboxylase polypeptide will encode at least 50, 75, 100, 150, 175, 200, 225, 250, 275, 300, 325, 328 contiguous amino acids, or up to the total number of amino acids present in a full-length dicamba decarboxylase polypeptide as set forth in, for example, a polypeptide having dicamba decarboxylase activity; wherein the polypeptide having dicamba decarboxylase activity further comprises:

```
(SEQ ID NO: 1041)
                                5 10
Met Ala Xaa Gly Lys Val Xaa Leu Glu Glu His Xaa
                15 20
Ala Ile Xaa Xaa Thr Leu Xaa Xaa Xaa Ala Xaa Phe
25 30 35
Val Pro Xaa Xaa Tyr Xaa Lys Xaa Leu Xaa His Arg
                        40 45
Leu Xaa Asp Xaa Gln Xaa Xaa Arg Leu Xaa Xaa Met
        50 55 60
Asp Xaa His Xaa Ile Xaa Xaa Met Xaa Leu Ser Leu
                                65 70
Xaa Ala Xaa Xaa Xaa Gln Xaa Xaa Xaa Xaa Arg Xaa
                75 80
Xaa Ala Xaa Xaa Xaa Ala Xaa Arg Xaa Asn Asp Xaa
85 90 95
Xaa Ala Glu Xaa Xaa Ala Xaa Xaa Xaa Xaa Arg Phe
                        100 105
Xaa Ala Phe Xaa Xaa Xaa Pro Xaa Xaa Asp Xaa Xaa
        110 115 120
Xaa Ala Xaa Xaa Glu Leu Gln Arg Xaa Val Xaa Xaa
                                125 130
Leu Gly Xaa Val Gly Ala Xaa Val Asn Gly Phe Ser
                135 140
Xaa Glu Gly Asp Xaa Xaa Thr Pro Leu Tyr Tyr Asp
145 150 155
Leu Pro Xaa Tyr Arg Pro Phe Trp Xaa Glu Val Glu
                        160 165
Lys Leu Asp Val Pro Phe Tyr Leu His Pro Xaa Asn
        170 175 180
Pro Leu Pro Gln Asp Xaa Arg Ile Tyr Xaa Gly His
                                185 190
Pro Trp Leu Leu Gly Pro Thr Trp Ala Phe Ala Gln
                195 200
Glu Thr Xaa Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Xaa
        230 235 240
Xaa Arg Ile Asp His Arg Xaa Xaa Xaa Xaa Xaa Xaa
                                245 250
Pro Pro Xaa Tyr Xaa Ala Lys Xaa Xaa Phe Xaa Asp
                255 260
Tyr Phe Xaa Glu Asn Phe Xaa Xaa Thr Thr Ser Gly
265 270 275
Asn Phe Arg Thr Gln Thr Leu Ile Asp Ala Ile Leu
                        280 285
Glu Xaa Gly Ala Asp Arg Ile Leu Phe Ser Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Xaa Thr Asn Ala Xaa Xaa Leu Phe Lys
325
Leu Asp Xaa Xaa,
```

wherein
Xaa at position 3 is Gln, Gly, Met or Pro; Xaa at position 7 is Ala or Cys; Xaa at position 12 is Phe, Met, Val or Trp; Xaa at position 15 is Pro or Thr; Xaa at position 16 is Glu or Ala; Xaa at position 19 is Gln, Glu or Asn; Xaa at position 20 is Asp, Cys, Phe, Met or Trp; Xaa at position 21 is Ser, Ala, Gly or Val; Xaa at position 23 is Gly or Asp; Xaa at position 27 is Gly, Ala, Asp, Glu, Pro, Arg, Ser, Thr or Tyr; Xaa at position 28 is Asp, Cys, Glu, Phe or Gly; Xaa at position 30 is Trp, Leu or Val; Xaa at position 32 is Glu or Val; Xaa at position 34 is Gln, Ala or Trp; Xaa at position 38 is Leu, Ile, Met, Arg, Thr or Val; Xaa at position 40 is Ile, Met, Ser or Val; Xaa at position 42 is Asp, Ala, Gly, Lys, Met, Ser or Thr; Xaa at position 43 is Thr, Cys, Asp, Glu, Gly, Met, Gin, Arg or Tyr; Xaa at position 46 is Lys, Gly, Asn or Arg; Xaa at position 47 is Leu, Cys, Glu, Lys or Ser; Xaa at position 50 is Ala, Lys, Arg, Ser, Thr or Val; Xaa at position 52 is Gly, Glu, Leu, Asn or Gln; Xaa at position 54 is Glu or Gly; Xaa at position 55 is Thr or Leu; Xaa at position 57 is Ile, Ala or Val; Xaa at position 61 is Asn, Ala, Gly, Leu or Ser; Xaa at position 63 is Pro or Val; Xaa at position 64 is Ala, Gly, His or Ser; Xaa at position 65 is Val or Cys; Xaa at position 67 is Ala or Ser; Xaa at position 68 is Ile or Gln; Xaa at position 69 is Pro, Gly, Arg, Ser or Val; Xaa at position 70 is Asp or His; Xaa at position 72 is Arg, Lys or Val; Xaa at position 73 is Lys, Glu, Gln or Arg; Xaa at position 75 is Ile or Arg; Xaa at position 76 is Glu or Gly; Xaa at position 77 is Ile, Met, Arg, Ser or Val; Xaa at position 79 is Arg or Gln; Xaa at position 81 is Ala or Ser; Xaa at position 84 is Val, Cys, Phe or Met; Xaa at position 85 is Leu or Ala; Xaa at position 88 is Glu or Lys; Xaa at position 89 is Cys, Ile or Val; Xaa at position 91 is Lys or Arg; Xaa at position 92 is Arg or Lys; Xaa at position 93 is Pro, Ala or Arg; Xaa at position 94 is Asp, Cys, Gly, Gln or Ser; Xaa at position 97 is Leu, Lys or Arg; Xaa at position 100 is Ala, Gly or Ser; Xaa at position 101 is Ala or Gly; Xaa at position 102 is Leu or Val; Xaa at position 104 is Leu or Met; Xaa at position 105 is Gln or Gly; Xaa at position 107 is Pro or Val; Xaa at position 108 is Asp or Glu; Xaa at position 109 is Ala, Gly, Met or Val; Xaa at position 111 is Thr, Ala, Cys, Gly, Ser or Val; Xaa at position 112 is Glu, Gly, Arg or Ser; Xaa at position 117 is Cys, Ala or Thr; Xaa at position 119 is Asn, Ala, Cys, Arg or Ser; Xaa at position 120 is Asp or Thr; Xaa at position 123 is Phe or Leu; Xaa at position 127 is Leu or Met; Xaa at position 133 is Gln or Val; Xaa at position 137 is Gly, Ala or Glu; Xaa at position 138 is Gin or Gly; Xaa at position 147 is Gln or Ile; Xaa at position 153 is Gly or Lys; Xaa at position 167 is Arg or Glu; Xaa at position 174 is Ser or Ala; Xaa at position 178 is Asp or Glu; Xaa at position 195 is Ala or Gly; Xaa at position 212 is Arg, Gly or Gln; Xaa at position 214 is Asn or Gln; Xaa at position 220 is Met or Leu; Xaa at position 228 is Met or Leu; Xaa at position 229 is Trp or Tyr; Xaa at position 235 is Val or Ile; Xaa at position 236 is Ala, Gly, Gln or Trp; Xaa at position 237 is Trp or Leu; Xaa at position 238 is Val, Gly or Pro; Xaa at position 239 is Lys, Ala, Asp, Glu, Gly or His; Xaa at position 240 is Leu, Ala, Asp, Glu, Gly or Val; Xaa at position 243 is Arg, Ala, Asp, Lys, Ser or Val; Xaa at position 245 is Pro or Ala; Xaa at position 248 is Arg or Lys; Xaa at position 249 is Arg or Pro; Xaa at position 251 is Met or Val; Xaa at position 255 is Asn, Ala, Leu, Met, Gln, Arg or Ser; Xaa at position 259 is His or Trp; Xaa at position 260 is Ile or Leu; Xaa at position 278 is Ile or Leu; Xaa at position 298 is Ser, Ala or Thr; Xaa at position 299 is Asp or Ala; Xaa at position 302 is Asn or Ala; Xaa at position 303 is Ala, Cys, Asp, Glu or Ser; Xaa at position 304 is Thr or Val; Xaa at position 312 is Val or Leu; Xaa at position 316 is Arg or Ser; Xaa at position 320 is Arg or Leu; Xaa at position 321 is Arg or Asn; Xaa at position 327 is Gly, Leu, Gln or Val; Xaa at position 328 is Ala, Cys, Asp, Arg, Ser, Thr or Val; wherein one or more amino acid(s) designated by Xaa in SEQ ID NO: 1041 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

In other embodiments, a fragment of a dicamba decarboxylase polynucleotide that encodes a biologically active portion of a dicamba decarboxylase polypeptide will encode at least 50, 75, 100, 150, 175, 200, 225, 250, 275, 300, 325, 328 contiguous amino acids, or up to the total number of amino acids present in a full-length dicamba decarboxylase polypeptide as set forth in, for example, a polypeptide having dicamba decarboxylase activity; wherein the polypeptide having dicamba decarboxylase activity further comprises:

```
(SEQ ID NO: 1042)
                                5 10
Met Ala Gln Gly Xaa Val Ala Leu Glu Glu His Phe
                15 20
Ala Ile Pro Xaa Thr Leu Xaa Asp Xaa Ala Xaa Phe
25 30 35
Val Pro Xaa Xaa Tyr Xaa Lys Glu Leu Gln His Arg
                        40 45
Leu Xaa Asp Xaa Gln Asp Xaa Arg Leu Xaa Xaa Met
        50 55 60
Asp Xaa His Xaa Ile Xaa Thr Met Xaa Leu Ser Leu
                                65 70
Xaa Ala Xaa Xaa Val Gln Xaa Ile Xaa Asp Arg Xaa
                75 80
Xaa Ala Ile Glu Xaa Ala Xaa Arg Ala Asn Asp Xaa
85 90 95
Leu Ala Glu Glu Xaa Ala Lys Arg Pro Xaa Arg Phe
                        100 105
Leu Ala Phe Ala Ala Leu Pro Xaa Gln Asp Xaa Xaa
        110 115 120
Ala Ala Xaa Xaa Glu Leu Gln Arg Xaa Val Xaa Xaa
                                125 130
Leu Gly Phe Val Gly Ala Xaa Val Asn Gly Phe Ser
                135 140
Xaa Glu Gly Asp Gly Gln Thr Pro Leu Tyr Tyr Asp
145 150 155
Leu Pro Gln Tyr Arg Pro Phe Trp Xaa Glu Val Glu
                        160 165
Lys Leu Asp Val Pro Phe Tyr Leu His Pro Arg Asn
        170 175 180
Pro Leu Pro Gln Asp Xaa Arg Ile Tyr Asp Gly His
                                185 190
Pro Trp Leu Leu Gly Pro Thr Trp Ala Phe Ala Gln
                195 200
Glu Thr Ala Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Met
        230 235 240
Xaa Arg Ile Asp His Arg Xaa Xaa Trp Val Xaa Xaa
                                245 250
Pro Pro Xaa Tyr Xaa Ala Lys Arg Arg Phe Met Asp
                255 260
Tyr Phe Xaa Glu Asn Phe Xaa Ile Thr Thr Ser Gly
265 270 275
Asn Phe Arg Thr Gln Thr Leu Ile Asp Ala Ile Leu
                        280 285
Glu Ile Gly Ala Asp Arg Ile Leu Phe Xaa Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Arg Thr Asn Ala Xaa Xaa Leu Phe Lys
325
Leu Asp Xaa Xaa
```

wherein
Xaa at position 5 is Lys or Leu; Xaa at position 16 is Glu or Ala; Xaa at position 19 is Gln or Asn; Xaa at position 21 is Ser or Ala; Xaa at position 23 is Gly or Asp; Xaa at position 27 is Gly or Ser; Xaa at position 28 is Asp, Cys or Glu; Xaa at position 30 is Trp or Leu; Xaa at position 38 is Leu or Met; Xaa at position 40 is Ile or Met; Xaa at position 43 is Thr, Glu or Gin; Xaa at position 46 is Lys, Asn or Arg; Xaa at position 47 is Leu or Glu; Xaa at position 50 is Ala, Lys or Arg; Xaa at position 52 is Gly, Glu or Gin; Xaa at position 54 is Glu or Gly; Xaa at position 57 is Ile or Val; Xaa at position 61 is Asn or Ala; Xaa at position 63 is Pro or Val; Xaa at position 64 is Ala or Gly; Xaa at position 67 is Ala, Gly or Ser; Xaa at position 69 is Pro, Gly or Val; Xaa at position 72 is Arg or Val; Xaa at position 73 is Lys, Glu or Gln; Xaa at position 77 is Ile or Leu; Xaa at position 79 is Arg or Lys; Xaa at position 84 is Val, Phe or Met; Xaa at position 89 is Cys or Val; Xaa at position 94 is Asp or Gly; Xaa at position 104 is Leu or Met; Xaa at position 107 is Pro or Val; Xaa at position 108 is Asp or Glu; Xaa at position 111 is Thr or Ser; Xaa at position 112 is Glu or Ser; Xaa at position 117 is Cys or Thr; Xaa at position 119 is Asn, Ala or Arg; Xaa at position 120 is Asp or Thr; Xaa at position 127 is Leu or Met; Xaa at position 133 is Gln or Val; Xaa at position 153 is Gly or Lys; Xaa at position 174 is Ser or Ala; Xaa at position 212 is Arg or Gly; Xaa at position 214 is Asn or Gln; Xaa at position 220 is Met or Leu; Xaa at position 229 is Trp or Tyr; Xaa at position 235 is Val or Ile; Xaa at position 236 is Ala or Gly; Xaa at position 239 is Lys, Glu or His; Xaa at position 240 is Leu, Ala or Glu; Xaa at position 243 is Arg or Asp; Xaa at position 245 is Pro or Ala; Xaa at position 255 is Asn or Leu; Xaa at position 259 is His or Trp; Xaa at position 286 is Ser or Ala; Xaa at position 298 is Ser, Ala or Thr; Xaa at position 299 is Asp or Ala; Xaa at position 302 is Asn or Ala; Xaa at position 303 is Ala or Glu; Xaa at position 304 is Thr or Ala; Xaa at position 312 is Val or Leu; Xaa at position 320 is Arg or Leu; Xaa at position 321 is Arg or Asn; Xaa at position 327 is Gly, Leu or Val; Xaa at position 328 is Ala, Asp, Arg, Ser or Thr; wherein one or more amino acid(s) designated by Xaa in SEQ ID NO: 1042 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

In other embodiments, a fragment of a dicamba decarboxylase polynucleotide that encodes a biologically active portion of a dicamba decarboxylase polypeptide will encode a region of the polypeptide that is sufficient to form the dicamba decarboxylase catalytic residue geometry as set forth in Table 3 or having a substantially similar catalytic residue geometry.

Thus, a fragment of a dicamba decarboxylase polynucleotide encodes a biologically active portion of a dicamba decarboxylase polypeptide. A biologically active portion of a dicamba decarboxylase polypeptide can be prepared by isolating a portion of one of the polynucleotides encoding a dicamba decarboxylase polypeptide, expressing the encoded portion of the dicamba decarboxylase polypeptides (e.g., by recombinant expression in vitro), and assaying for dicamba decarboxylase activity. Polynucleotides that are fragments of a dicamba decarboxylase nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length polynucleotide encoding a dicamba decarboxylase polypeptide disclosed herein.

ii. Active Variants of Dicamba Decarboxylase Sequences

“Variant” protein is intended to mean a protein derived from the protein by deletion (i.e., truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity, that is, dicamba decarboxylases activity.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide. For polynucleotides, conservative variants include those sequences that, because of the degeneracy of the genetic code, encode the amino acid sequence of one of the dicamba decarboxylase polypeptides. Naturally occurring variants such as these can be identified with the use of well-known molecular biology techniques, as, for example, with polymerase chain reaction (PCR) and hybridization techniques, and sequencing techniques as outlined below. Variant polynucleotides also include synthetically derived polynucleotides, such as those generated, for example, by using site-directed mutagenesis or gene synthesis but which still encode a dicamba decarboxylase polypeptide or through computation modeling.

In other embodiments, biologically active variants of a dicamba decarboxylase polypeptide (and the polynucleotide encoding the same) will have a percent identity across their full length of at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polypeptide of any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 or 129 as determined by sequence alignment programs and parameters described elsewhere herein.

In other embodiments, biologically active variants of a dicamba decarboxylase polypeptide (and the polynucleotide encoding the same) will have a percent identity across their full length of at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polypeptide of any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, and 1042, as determined by sequence alignment programs and parameters described elsewhere herein.

In other embodiments, biologically active variants of a dicamba decarboxylase polypeptide (and the polynucleotide encoding the same) will have a percent identity across their full length of at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polypeptide comprising:

```
(SEQ ID NO: 1041)
                                5 10
Met Ala Xaa Gly Lys Val Xaa Leu Glu Glu His Xaa
                15 20
Ala Ile Xaa Xaa Thr Leu Xaa Xaa Xaa Ala Xaa Phe
25 30 35
Val Pro Xaa Xaa Tyr Xaa Lys Xaa Leu Xaa His Arg
                        40 45
Leu Xaa Asp Xaa Gln Xaa Xaa Arg Leu Xaa Xaa Met
        50 55 60
Asp Xaa His Xaa Ile Xaa Xaa Met Xaa Leu Ser Leu
                                65 70
Xaa Ala Xaa Xaa Xaa Gln Xaa Xaa Xaa Xaa Arg Xaa
                75 80
Xaa Ala Xaa Xaa Xaa Ala Xaa Arg Xaa Asn Asp Xaa
85 90 95
Xaa Ala Glu Xaa Xaa Ala Xaa Xaa Xaa Xaa Arg Phe
                        100 105
Xaa Ala Phe Xaa Xaa Xaa Pro Xaa Xaa Asp Xaa Xaa
        110 115 120
Xaa Ala Xaa Xaa Glu Leu Gln Arg Xaa Val Xaa Xaa
                                125 130
Leu Gly Xaa Val Gly Ala Xaa Val Asn Gly Phe Ser
                135 140
Xaa Glu Gly Asp Xaa Xaa Thr Pro Leu Tyr Tyr Asp
145 150 155
Leu Pro Xaa Tyr Arg Pro Phe Trp Xaa Glu Val Glu
                        160 165
Lys Leu Asp Val Pro Phe Tyr Leu His Pro Xaa Asn
        170 175 180
Pro Leu Pro Gln Asp Xaa Arg Ile Tyr Xaa Gly His
                                185 190
Pro Trp Leu Leu Gly Pro Thr Trp Ala Phe Ala Gln
                195 200
Glu Thr Xaa Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Xaa
        230 235 240
Xaa Arg Ile Asp His Arg Xaa Xaa Xaa Xaa Xaa Xaa
                                245 250
Pro Pro Xaa Tyr Xaa Ala Lys Xaa Xaa Phe Xaa Asp
                255 260
Tyr Phe Xaa Glu Asn Phe Xaa Xaa Thr Thr Ser Gly
265 270 275
Asn Phe Arg Thr Gln Thr Leu Ile Asp Ala Ile Leu
                        280 285
Glu Xaa Gly Ala Asp Arg Ile Leu Phe Ser Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Xaa Thr Asn Ala Xaa Xaa Leu Phe Lys
325
Leu Asp Xaa Xaa,
```

wherein

Xaa at position 3 is Gln, Gly, Met or Pro; Xaa at position 7 is Ala or Cys; Xaa at position 12 is Phe, Met, Val or Trp; Xaa at position 15 is Pro or Thr; Xaa at position 16 is Glu or Ala; Xaa at position 19 is Gln, Glu or Asn; Xaa at position 20 is Asp, Cys, Phe, Met or Trp; Xaa at position 21 is Ser, Ala, Gly or Val; Xaa at position 23 is Gly or Asp; Xaa at position 27 is Gly, Ala, Asp, Glu, Pro, Arg, Ser, Thr or Tyr; Xaa at position 28 is Asp, Cys, Glu, Phe or Gly; Xaa at position 30 is Trp, Leu or Val; Xaa at position 32 is Glu or Val; Xaa at position 34 is Gln, Ala or Trp; Xaa at position 38 is Leu, Ile, Met, Arg, Thr or Val; Xaa at position 40 is Ile, Met, Ser or Val; Xaa at position 42 is Asp, Ala, Gly, Lys, Met, Ser or Thr; Xaa at position 43 is Thr, Cys, Asp, Glu, Gly, Met, Gln, Arg or Tyr; Xaa at position 46 is Lys, Gly, Asn or Arg; Xaa at position 47 is Leu, Cys, Glu, Lys or Ser; Xaa at position 50 is Ala, Lys, Arg, Ser, Thr or Val; Xaa at position 52 is Gly, Glu, Leu, Asn or Gln; Xaa at position 54 is Glu or Gly; Xaa at position 55 is Thr or Leu; Xaa at position 57 is Ile, Ala or Val; Xaa at position 61 is Asn, Ala, Gly, Leu or Ser; Xaa at position 63 is Pro or Val; Xaa at position 64 is Ala, Gly, His or Ser; Xaa at position 65 is Val or Cys; Xaa at position 67 is Ala or Ser; Xaa at position 68 is Ile or Gln; Xaa at position 69 is Pro, Gly, Arg, Ser or Val; Xaa at position 70 is Asp or His; Xaa at position 72 is Arg, Lys or Val; Xaa at position 73 is Lys, Glu, Gln or Arg; Xaa at position 75 is Ile or Arg; Xaa at position 76 is Glu or Gly; Xaa at position 77 is Ile, Met, Arg, Ser or Val; Xaa at position 79 is Arg or Gln; Xaa at position 81 is Ala or Ser; Xaa at position 84 is Val, Cys, Phe or Met; Xaa at position 85 is Leu or Ala; Xaa at position 88 is Glu or Lys; Xaa at position 89 is Cys, Ile or Val; Xaa at position 91 is Lys or Arg; Xaa at position 92 is Arg or Lys; Xaa at position 93 is Pro, Ala or Arg; Xaa at position 94 is Asp, Cys, Gly, Gln or Ser; Xaa at position 97 is Leu, Lys or Arg; Xaa at position 100 is Ala, Gly or Ser; Xaa at position 101 is Ala or Gly; Xaa at position 102 is Leu or Val; Xaa at position 104 is Leu or Met; Xaa at position 105 is Gln or Gly; Xaa at position 107 is Pro or Val; Xaa at position 108 is Asp or Glu; Xaa at position 109 is Ala, Gly, Met or Val; Xaa at position 111 is Thr, Ala, Cys, Gly, Ser or Val; Xaa at position 112 is Glu, Gly, Arg or Ser; Xaa at position 117 is Cys, Ala or Thr; Xaa at position 119 is Asn, Ala, Cys, Arg or Ser; Xaa at position 120 is Asp or Thr; Xaa at position 123 is Phe or Leu; Xaa at position 127 is Leu or Met; Xaa at position 133 is Gln or Val; Xaa at position 137 is Gly, Ala or Glu; Xaa at position 138 is Gln or Gly; Xaa at position 147 is Gln or Ile; Xaa at position 153 is Gly or Lys; Xaa at position 167 is Arg or Glu; Xaa at position 174 is Ser or Ala; Xaa at position 178 is Asp or Glu; Xaa at position 195 is Ala or Gly; Xaa at position 212 is Arg, Gly or Gln; Xaa at position 214 is Asn or Gln; Xaa at position 220 is Met or Leu; Xaa at position 228 is Met or Leu; Xaa at position 229 is Trp or Tyr; Xaa at position 235 is Val or Ile; Xaa at position 236 is Ala, Gly, Gln or Trp; Xaa at position 237 is Trp or Leu; Xaa at position 238 is Val, Gly or Pro; Xaa at position 239 is Lys, Ala, Asp, Glu, Gly or His; Xaa at position 240 is Leu, Ala, Asp, Glu, Gly or Val; Xaa at position 243 is Arg, Ala, Asp, Lys, Ser or Val; Xaa at position 245 is Pro or Ala; Xaa at position 248 is Arg or Lys; Xaa at position 249 is Arg or Pro; Xaa at position 251 is Met or Val; Xaa at position 255 is Asn, Ala, Leu, Met, Gln, Arg or Ser; Xaa at position 259 is His or Trp; Xaa at position 260 is Ile or Leu; Xaa at position 278 is Ile or Leu; Xaa at position 298 is Ser, Ala or Thr; Xaa at position 299 is Asp or Ala; Xaa at position 302 is Asn or Ala; Xaa at position 303 is Ala, Cys, Asp, Glu or Ser; Xaa at position 304 is Thr or Val; Xaa at position 312 is Val or Leu; Xaa at position 316 is Arg or Ser; Xaa at position 320 is Arg or Leu; Xaa at position 321 is Arg or Asn; Xaa at position 327 is Gly, Leu, Gln or Val; Xaa at position 328 is Ala, Cys, Asp, Arg, Ser, Thr or Val; wherein one or more amino acid(s) designated by Xaa in SEQ ID NO: 1041 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

In other embodiments, biologically active variants of a dicamba decarboxylase polypeptide (and the polynucleotide encoding the same) will have a percent identity across their full length of at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the polypeptide comprising:

```
(SEQ ID NO: 1042)
                                5 10
Met Ala Gln Gly Xaa Val Ala Leu Glu Glu His Phe
                15 20
Ala Ile Pro Xaa Thr Leu Xaa Asp Xaa Ala Xaa Phe
25 30 35
Val Pro Xaa Xaa Tyr Xaa Lys Glu Leu Gln His Arg
                        40 45
Leu Xaa Asp Xaa Gln Asp Xaa Arg Leu Xaa Xaa Met
        50 55 60
Asp Xaa His Xaa Ile Xaa Thr Met Xaa Leu Ser Leu
                                65 70
Xaa Ala Xaa Xaa Val Gln Xaa Ile Xaa Asp Arg Xaa
                75 80
Xaa Ala Ile Glu Xaa Ala Xaa Arg Ala Asn Asp Xaa
85 90 95
Leu Ala Glu Glu Xaa Ala Lys Arg Pro Xaa Arg Phe
                        100 105
Leu Ala Phe Ala Ala Leu Pro Xaa Gln Asp Xaa Xaa
        110 115 120
Ala Ala Xaa Xaa Glu Leu Gln Arg Xaa Val Xaa Xaa
                                125 130
Leu Gly Phe Val Gly Ala Xaa Val Asn Gly Phe Ser
                135 140
Xaa Glu Gly Asp Gly Gln Thr Pro Leu Tyr Tyr Asp
145 150 155
Leu Pro Gln Tyr Arg Pro Phe Trp Xaa Glu Val Glu
                        160 165
Lys Leu Asp Val Pro Phe Tyr Leu His Pro Arg Asn
        170 175 180
Pro Leu Pro Gln Asp Xaa Arg Ile Tyr Asp Gly His
                                185 190
Pro Trp Leu Leu Gly Pro Thr Trp Ala Phe Ala Gln
                195 200
Glu Thr Ala Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Met
        230 235 240
Xaa Arg Ile Asp His Arg Xaa Xaa Trp Val Xaa Xaa
                                245 250
Pro Pro Xaa Tyr Xaa Ala Lys Arg Arg Phe Met Asp
                255 260
Tyr Phe Xaa Glu Asn Phe Xaa Ile Thr Thr Ser Gly
265 270 275
Asn Phe Arg Thr Gln Thr Leu Ile Asp Ala Ile Leu
                        280 285
Glu Ile Gly Ala Asp Arg Ile Leu Phe Xaa Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Arg Thr Asn Ala Xaa Xaa Leu Phe Lys
325
Leu Asp Xaa Xaa
```

wherein
Xaa at position 5 is Lys or Leu; Xaa at position 16 is Glu or Ala; Xaa at position 19 is Gln or Asn; Xaa at position 21 is Ser or Ala; Xaa at position 23 is Gly or Asp; Xaa at position 27 is Gly or Ser; Xaa at position 28 is Asp, Cys or Glu; Xaa at position 30 is Trp or Leu; Xaa at position 38 is Leu or Met; Xaa at position 40 is Ile or Met; Xaa at position 43 is Thr, Glu or Gln; Xaa at position 46 is Lys, Asn or Arg; Xaa at position 47 is Leu or Glu; Xaa at position 50 is Ala, Lys or Arg; Xaa at position 52 is Gly, Glu or Gln; Xaa at position 54 is Glu or Gly; Xaa at position 57 is Ile or Val; Xaa at position 61 is Asn or Ala; Xaa at position 63 is Pro or Val; Xaa at position 64 is Ala or Gly; Xaa at position 67 is Ala, Gly or Ser; Xaa at position 69 is Pro, Gly or Val; Xaa at position 72 is Arg or Val; Xaa at position 73 is Lys, Glu or Gln; Xaa at position 77 is Ile or Leu; Xaa at position 79 is Arg or Lys; Xaa at position 84 is Val, Phe or Met; Xaa at position 89 is Cys or Val; Xaa at position 94 is Asp or Gly; Xaa at position 104 is Leu or Met; Xaa at position 107 is Pro or Val; Xaa at position 108 is Asp or Glu; Xaa at position 111 is Thr or Ser; Xaa at position 112 is Glu or Ser; Xaa at position 117 is Cys or Thr; Xaa at position 119 is Asn, Ala or Arg; Xaa at position 120 is Asp or Thr; Xaa at position 127 is Leu or Met; Xaa at position 133 is Gln or Val; Xaa at position 153 is Gly or Lys; Xaa at position 174 is Ser or Ala; Xaa at position 212 is Arg or Gly; Xaa at position 214 is Asn or Gln; Xaa at position 220 is Met or Leu; Xaa at position 229 is Trp or Tyr; Xaa at position 235 is Val or Ile; Xaa at position 236 is Ala or Gly; Xaa at position 239 is Lys, Glu or His; Xaa at position 240 is Leu, Ala or Glu; Xaa at position 243 is Arg or Asp; Xaa at position 245 is Pro or Ala; Xaa at position 255 is Asn or Leu; Xaa at position 259 is His or Trp; Xaa at position 286 is Ser or Ala; Xaa at position 298 is Ser, Ala or Thr; Xaa at position 299 is Asp or Ala; Xaa at position 302 is Asn or Ala; Xaa at position 303 is Ala or Glu; Xaa at position 304 is Thr or Ala; Xaa at position 312 is Val or Leu; Xaa at position 320 is Arg or Leu; Xaa at position 321 is Arg or Asn; Xaa at position 327 is Gly, Leu or Val; Xaa at position 328 is Ala, Asp, Arg, Ser or Thr; wherein one or more amino acid(s) designated by Xaa in SEQ ID NO: 1042 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

In other embodiments, biologically active variants of a dicamba decarboxylase polypeptide (and the polynucleotide encoding the same) will have at least a similarity score of or about 400, 420, 450, 480, 500, 520, 540, 548, 580, 590, 600, 620, 650, 675, 700, 710, 720, 721, 722, 723, 724, 725, 726, 728, 729, 730, 731, 732, 733, 734, 735, 736, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 828, 829, 830, 831, 832, 833, 834, 835, 836, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 900, 920, 940, 960, or greater to any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 or 129 as determined by sequence alignment programs and parameters described elsewhere herein.

The dicamba decarboxylase polypeptides and the active variants and fragments thereof may be altered in various ways including amino acid substitutions, deletions, truncations, and insertions and through rational design modeling as discussed elsewhere herein. Methods for such manipulations are generally known in the art. For example, amino acid sequence variants and fragments of the dicamba decarboxylase polypeptides can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found, Washington, D.C.), herein incorporated by reference in their entirety. Conservative substitutions, such as exchanging one amino acid with another having similar properties, may be optimal.

Obviously, the mutations that will be made in the DNA encoding the variant must not place the sequence out of reading frame and optimally will not create complementary regions that could produce secondary mRNA structure. See, EP Patent Application Publication No. 75,444.

Non-limiting examples of dicamba decarboxylases and active fragments and variants thereof are provided herein and can include dicamba decarboxylases comprising an active site having a catalytic residue geometry as set forth in Table 3 or having a substantially similar catalytic residue geometry and further comprises an amino acid sequence having at least 40%, 75% 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% percent identity to any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 or 129, wherein the polypeptide has dicamba decarboxylation activity.

Non-limiting examples of dicamba decarboxylases and active fragments and variants thereof are provided herein and can include dicamba decarboxylases comprising an active site having a catalytic residue geometry as set forth in Table 3 or having a substantially similar catalytic residue geometry and further comprises an amino acid sequence having at least 40%, 75% 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% percent identity to any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, and 1042, wherein the polypeptide has dicamba decarboxylation activity.

In other embodiments, the dicamba decarboxylases and active fragments and variants thereof are provided herein and can include a dicamba decarboxylase comprises an active site having a catalytic residue geometry as set forth in Table 3 or having a substantially similar catalytic residue geometry and further comprises an amino acid sequence having a similarity score of at least 400, 420, 450, 480, 500, 520, 540, 548, 580, 590, 600, 620, 650, 675, 700, 710, 720, 721, 722, 723, 724, 725, 726, 728, 729, 730, 731, 732, 733, 734, 735, 736, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 828, 829, 830, 831, 832, 833, 834, 835, 836, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 900, 920, 940, 960 or greater to any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 or 129, wherein the polypeptide has dicamba decarboxylation activity.

In other embodiments, the dicamba decarboxylases and active fragments and variants thereof are provided herein and can include a dicamba decarboxylase comprises an active site having a catalytic residue geometry as set forth in Table 3 or having a substantially similar catalytic residue geometry and further comprises an amino acid sequence having a similarity score of at least 400, 420, 450, 480, 500, 520, 540, 548, 580, 590, 600, 620, 650, 675, 700, 710, 720, 721, 722, 723, 724, 725, 726, 728, 729, 730, 731, 732, 733, 734, 735, 736, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 828, 829, 830, 831, 832, 833, 834, 835, 836, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 900, 920, 940, 960 or greater to any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, and 1042, wherein the polypeptide has dicamba decarboxylation activity.

In other embodiments, the dicamba decarboxylase comprises an active site having a catalytic residue geometry as set forth in Table 3or having a substantially similar catalytic residue geometry and further comprises (a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1; (b) an amino acid sequence having a similarity score of at least 400, 450, 480, 500, 520, 548, 580, 600, 620, 650, 670, 690, 710, 720, 730, 750, 780, 800, 820, 840, 860, 880, 900, 920, 940, 960, or higher for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1; (d) an amino acid sequence having at least 50%, 55%, 60%, 65%, 70%, 75%, 80%, 81%, 82%, 83%, 84%, 85%, 86%, 87%, 88%, 89%, 90%, 95%, 96%, 97%, 98%, 99%, or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; (e) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to any one of SEQ ID NOS: 46, 89, 19, 79, 81, 95, or 100; (f) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to any one of SEQ ID NOS: 117, 118, or 119; (g) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to any one of SEQ ID NOS: 120, 121, or 122; (h) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95% 96%, 97%, 98%, 99% or 100% sequence identity to any one of SEQ ID NOS:109, 110, 111, 112, 113, 114, 116 or 115; (i) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to SEQ ID NO: 116; (j) and/or an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 109, wherein (i) the amino acid residue in the encoded polypeptide that corresponds to amino acid position 27 of SEQ ID NO: 109 comprises alanine, serine, or threonine; (ii) the amino acid residue in the encoded polypeptide that corresponds to amino acid position 38 of SEQ ID NO: 109 comprises isoleucine; (iii) the amino acid residue in the encoded polypeptide that corresponds to amino acid position 42 of SEQ ID NO: 109 comprises alanine, methionine, or serine; (iv) the amino acid residue in the encoded polypeptide that corresponds to amino acid position 52 of SEQ ID NO: 109 comprises glutamic acid; (v) the amino acid residue in the encoded polypeptide that corresponds to amino acid position 61 of SEQ ID NO: 109 comprises alanine or serine; (vi) the amino acid residue in the encoded polypeptide that corresponds to amino acid position 64 of SEQ ID NO: 109 comprises glycine, or serine; (vii) the amino acid residue in the encoded polypeptide that corresponds to amino acid position 127 of SEQ ID NO: 109 comprises methionine; (iix) the amino acid residue in the encoded polypeptide that corresponds to amino acid position 238 of SEQ ID NO: 109 comprises glycine; (ix) the amino acid residue in the encoded polypeptide that corresponds to amino acid position 240 of SEQ ID NO: 109 comprises alanine, aspartic acid, or glutamic acid; (x) the amino acid residue in the encoded polypeptide that corresponds to amino acid position 298 of SEQ ID NO: 109 comprises alanine or threonine; (xi) the amino acid residue in the encoded polypeptide that corresponds to amino acid position 299 of SEQ ID NO: 109 comprises alanine; (xii) the amino acid residue in the encoded polypeptide that corresponds to amino acid position 303 of SEQ ID NO: 109 comprises cysteine, glutamic acid, or serine; (xiii) the amino acid residue in the encoded polypeptide that corresponds to amino acid position 327 of SEQ ID NO: 109 comprises leucine, glutamine, or valine; (ixv) the amino acid residue in the encoded polypeptide that corresponds to amino acid position 328 of SEQ ID NO: 109 comprises aspartic acid, arginine, or serine; and/or (xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

It is recognized that dicamba decarboxylases useful in the methods and compositions provided herein need not comprise catalytic residue geometry as set forth in Table 3, so long as the polypeptides retains dicamba decarboxylase activity. In such embodiments, the polypeptide having dicamba decarboxylase activity can comprise (a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1; (b) an amino acid sequence having a similarity score of at least 400, 450, 480, 500, 520, 548, 580, 600, 620, 650, 670, 690, 710, 720, 730, 750, 780, 800, 820, 840, 860, 880, 900, 920, 940, 960, or higher for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1; (d) an amino acid sequence having at least 50%, 55%, 60%, 65%, 70%, 75%, 80%, 81%, 82%, 83%, 84%, 85%, 86%, 87%, 88%, 89%, 90%, 95%, 96%, 97%, 98%, 99%, or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; (e) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to any one of SEQ ID NOS: 46, 89, 19, 79, 81, 95, or 100; (f) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to any one of SEQ ID NOS: 117, 118, or 119; (g) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to any one of SEQ ID NOS: 120, 121, or 122; (h) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95% 96%, 97%, 98%, 99% or 100% sequence identity to any one of SEQ ID NOS:109, 110, 111, 112, 113, 114, 116 or 115; (i) an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to SEQ ID NO: 116; (j) and/or an amino acid sequence having at least 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99% or 100% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129, wherein (i) the amino acid residue in the encoded polypeptide that corresponds to amino acid position 27 of SEQ ID NO: 109 comprises alanine, serine, or threonine; (ii) the amino acid residue in the encoded polypeptide that corresponds to amino acid position 38 of SEQ ID NO: 109 comprises isoleucine; (iii) the amino acid residue in the encoded polypeptide that corresponds to amino acid position 42 of SEQ ID NO: 109 comprises alanine, methionine, or serine; (iv) the amino acid residue in the encoded polypeptide that corresponds to amino acid position 52 of SEQ ID NO: 109 comprises glutamic acid; (v) the amino acid residue in the encoded polypeptide that corresponds to amino acid position 61 of SEQ ID NO: 109 comprises alanine or serine; (vi) the amino acid residue in the encoded polypeptide that corresponds to amino acid position 64 of SEQ ID NO: 109 comprises glycine, or serine; (vii) the amino acid residue in the encoded polypeptide that corresponds to amino acid position 127 of SEQ ID NO: 109 comprises methionine; (iix) the amino acid residue in the encoded polypeptide that corresponds to amino acid position 238 of SEQ ID NO: 109 comprises glycine; (ix) the amino acid residue in the encoded polypeptide that corresponds to amino acid position 240 of SEQ ID NO: 109 comprises alanine, aspartic acid, or glutamic acid; (x) the amino acid residue in the encoded polypeptide that corresponds to amino acid position 298 of SEQ ID NO: 109 comprises alanine or threonine; (xi) the amino acid residue in the encoded polypeptide that corresponds to amino acid position 299 of SEQ ID NO: 109 comprises alanine; (xii) the amino acid residue in the encoded polypeptide that corresponds to amino acid position 303 of SEQ ID NO: 109 comprises cysteine, glutamic acid, or serine; (xiii) the amino acid residue in the encoded polypeptide that corresponds to amino acid position 327 of SEQ ID NO: 109 comprises leucine, glutamine, or valine; (ixv) the amino acid residue in the encoded polypeptide that corresponds to amino acid position 328 of SEQ ID NO: 109 comprises aspartic acid, arginine, or serine; and/or (xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

As used herein, an “isolated” or “purified” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein.

As used herein, polynucleotide or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene is recombinant.

A “control” or “control plant” or “control plant cell” provides a reference point for measuring changes in phenotype of the subject plant or plant cell, and may be any suitable plant or plant cell. A control plant or plant cell may comprise, for example: (a) a wild-type or native plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell which is genetically identical to the subject plant or plant cell but which is not exposed to the same treatment (e.g., herbicide treatment) as the subject plant or plant cell; or (e) the subject plant or plant cell itself, under conditions in which the gene of interest is not expressed.

iii. Dicamba Decarboxylase Activity

Various assays can be used to measure dicamba decarboxylase activity. In one method, dicamba decarboxylase activity can be assayed by measuring CO₂ generated from enzyme reactions. See Example 1 which outlines in detail such assays. In other methods, dicamba decarboxylase activity can be assayed by measuring CO₂ product indirectly using a coupled enzyme assay which is also described in detail in Example 1. The overall catalytic efficiency of the enzyme can be expressed as k_{cat}/K_{M}. Alternatively, dicamba decarboxylase activity can be monitored by measuring decarboxylation products other than CO₂ using product detection methods. Each of the decarboxylation products of dicamba that can be assayed, including 2,5-dichloro anisole (2,5-dichloro phenol (the decarboxylated and demethylated product of dicamba) and 4-chloro-3-methoxy phenol (the decarboxylated and chloro hydrolyzed product) using the various methods as set forth in Example 1. In specific embodiments, the dicamba decarboxylase activity is assayed by expressing the sequence in a plant cell and detecting an increase tolerance of the plant cell to dicamba.

Thus, the various assays described herein can be used to determine kinetic parameters (i.e., K_{M}, k_{cat}, k_{cat}/K_{M}) for the dicamba decarboxylases. In general, a dicamba decarboxylase with a higher k_{cat} or k_{cat}/K_{M} is a more efficient catalyst than another dicamba decarboxylase with lower k_{cat} or k_{cat}/K_{M}. A dicamba decarboxylase with a lower K_{M} is a more efficient catalyst than another dicamba decarboxylase with a higher K_{M}. Thus, to determine whether one dicamba decarboxylase is more effective than another, one can compare kinetic parameters for the two enzymes. The relative importance of k_{cat}, k_{cat}/K_{M} and K_{M} will vary depending upon the context in which the dicamba decarboxylase will be expected to function, e.g., the anticipated effective concentration of dicamba relative to K_{M} for dicamba. Dicamba decarboxylase activity can also be characterized in terms of any of a number of functional characteristics, e.g., stability, susceptibility to inhibition or activation by other molecules, etc. Some dicamba decarboxylase polypeptides for use in decarboxylating dicamba have a k_{cat} of at least 0.01 min⁻¹, at least 0.1 min⁻¹, 1 min⁻¹, 10 min⁻¹, 100 min⁻¹, 1,000 min⁻¹, or 10,000 min⁻¹. Other dicamba decarboxylase polypeptides for use in conferring dicamba tolerance have a K_{M} no greater than 0.001 mM, 0.01 mM, 0.1 mM, 1 mM, 10 mM or 100 mM. Still other dicamba decarboxylase polypeptides for use in conferring dicamba tolerance have a k_{cat}/K_{M} of at least 0.0001 mM⁻¹ min⁻¹ or more, at least 0.001 mM⁻¹ min⁻¹, 0.01 mM⁻¹ min⁻¹, 0.1 mM⁻¹ min⁻¹, 1.0 mM⁻¹ min⁻¹, 10 mM⁻¹ min⁻¹, 100 mM⁻¹ min⁻¹, 1,000 mM⁻¹ min⁻¹, or 10,000 mM⁻¹ min⁻¹.

In specific embodiments, the dicamba decarboxylase polypeptide or active variant or fragment thereof has an activity that is at least equivalent to a native dicamba decarboxylase polypeptide or has an activity that is increased when compared to a native dicamba decarboxylase polypeptide. An “equivalent” dicamba decarboxylase activity refers to an activity level that is not statistically significantly different from the control as determined through any enzymatic kinetic parameter, including for example, via K_{M}, k_{cat}, or k_{cat}/K_{M}. An increased dicamba decarboxylase activity comprises any statistically significant increase in dicamba decarboxylase activity as determined through any enzymatic kinetic parameter, such as, for example, K_{M}, k_{cat}, or k_{cat}/K_{M}. In specific embodiments, an increase in activity comprises at least a 1.1, 1.2, 1.3, 1.4, 1.5, 1.6, 1.7, 1.8, 2, 3, 4, 5, 6, 7, 8, 9, or 10 fold or greater improvement in a given kinetic parameter when compared to a native sequence as set forth in SEQ ID NO:1-108. Methods to determine such kinetic parameters are known.

### III. Host Cells, Plants and Plant Parts

Host cells, plants, plant cells, plant parts, seeds, and grain having a heterologous copy of the dicamba decarboxylase sequences disclosed herein are provided. It is expected that those of skill in the art are knowledgeable in the numerous systems available for the introduction of a polypeptide or a nucleotide sequence disclosed herein into a host cell. No attempt to describe in detail the various methods known for providing sequences in prokaryotes or eukaryotes will be made.

By “host cell” is meant a cell which comprises a heterologous dicamba decarboxylase sequence. Host cells may be prokaryotic cells, such as E. coli, or eukaryotic cells such as yeast cells. Suitable host cells include the prokaryotes and the lower eukaryotes, such as fungi. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae and Nitrobacteraceae. Among eukaryotes are fungi, such as Phycomycetes and Ascomycetes, which includes yeast, such as Pichia pastoris, Saccharomyces and Schizosaccharomyces; and Basidiomycetes yeast, such as Rhodotorula, Aureobasidium, Sporobolomyces, and the like. Host cells can also be monocotyledonous or dicotyledonous plant cells.

In specific embodiments, the host cells, plants and/or plant parts have stably incorporated at least one heterologous polynucleotide encoding a dicamba decarboxylase polypeptide or an active variant or fragment thereof. Thus, host cells, plants, plant cells, plant parts and seed are provided which comprise at least one heterologous polynucleotide encoding a dicamba decarboxylase polypeptide of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 or 129 or active variant or fragments thereof. In other embodiments, the host cells, plants, plant cells, plant parts and seed are provided which comprise at least one heterologous polynucleotide encoding a dicamba decarboxylase polypeptide which comprises a catalytic residue geometry as set forth in Table 3 or a substantially similar geometry. Such sequences are discussed elsewhere herein.

In specific embodiments, host cells, plants, plant cells, plant parts and seed are provided which comprise at least one heterologous polynucleotide encoding a dicamba decarboxylase polypeptide of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, and 1042 or active variant or fragments thereof. In other embodiments, the host cells, plants, plant cells, plant parts and seed are provided which comprise at least one heterologous polynucleotide encoding a dicamba decarboxylase polypeptide which comprises a catalytic residue geometry as set forth in Table 3 or a substantially similar geometry. Such sequences are discussed elsewhere herein.

In specific embodiments, host cells, plants, plant cells, plant parts and seed are provided which comprise at least one heterologous polynucleotide encoding a dicamba decarboxylase polypeptide comprising:

```
(SEQ ID NO: 1041)
                                5 10
Met Ala Xaa Gly Lys Val Xaa Leu Glu Glu His Xaa
                15 20
Ala Ile Xaa Xaa Thr Leu Xaa Xaa Xaa Ala Xaa Phe
25 30 35
Val Pro Xaa Xaa Tyr Xaa Lys Xaa Leu Xaa His Arg
                        40 45
Leu Xaa Asp Xaa Gln Xaa Xaa Arg Leu Xaa Xaa Met
        50 55 60
Asp Xaa His Xaa Ile Xaa Xaa Met Xaa Leu Ser Leu
                                65 70
Xaa Ala Xaa Xaa Xaa Gln Xaa Xaa Xaa Xaa Arg Xaa
                75 80
Xaa Ala Xaa Xaa Xaa Ala Xaa Arg Xaa Asn Asp Xaa
85 90 95
Xaa Ala Glu Xaa Xaa Ala Xaa Xaa Xaa Xaa Arg Phe
                        100 105
Xaa Ala Phe Xaa Xaa Xaa Pro Xaa Xaa Asp Xaa Xaa
        110 115 120
Xaa Ala Xaa Xaa Glu Leu Gln Arg Xaa Val Xaa Xaa
                                125 130
Leu Gly Xaa Val Gly Ala Xaa Val Asn Gly Phe Ser
                135 140
Xaa Glu Gly Asp Xaa Xaa Thr Pro Leu Tyr Tyr Asp
145 150 155
Leu Pro Xaa Tyr Arg Pro Phe Trp Xaa Glu Val Glu
                        160 165
Lys Leu Asp Val Pro Phe Tyr Leu His Pro Xaa Asn
        170 175 180
Pro Leu Pro Gln Asp Xaa Arg Ile Tyr Xaa Gly His
                                185 190
Pro Trp Leu Leu Gly Pro Thr Trp Ala Phe Ala Gln
                195 200
Glu Thr Xaa Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Xaa
        230 235 240
Xaa Arg Ile Asp His Arg Xaa Xaa Xaa Xaa Xaa Xaa
                                245 250
Pro Pro Xaa Tyr Xaa Ala Lys Xaa Xaa Phe Xaa Asp
                255 260
Tyr Phe Xaa Glu Asn Phe Xaa Xaa Thr Thr Ser Gly
265 270 275
Asn Phe Arg Thr Gln Thr Leu Ile Asp Ala Ile Leu
                        280 285
Glu Xaa Gly Ala Asp Arg Ile Leu Phe Ser Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Xaa Thr Asn Ala Xaa Xaa Leu Phe Lys
325
Leu Asp Xaa Xaa,
```

wherein

Xaa at position 3 is Gln, Gly, Met or Pro; Xaa at position 7 is Ala or Cys; Xaa at position 12 is Phe, Met, Val or Trp; Xaa at position 15 is Pro or Thr; Xaa at position 16 is Glu or Ala; Xaa at position 19 is Gln, Glu or Asn; Xaa at position 20 is Asp, Cys, Phe, Met or Trp; Xaa at position 21 is Ser, Ala, Gly or Val; Xaa at position 23 is Gly or Asp; Xaa at position 27 is Gly, Ala, Asp, Glu, Pro, Arg, Ser, Thr or Tyr; Xaa at position 28 is Asp, Cys, Glu, Phe or Gly; Xaa at position 30 is Trp, Leu or Val; Xaa at position 32 is Glu or Val; Xaa at position 34 is Gln, Ala or Trp; Xaa at position 38 is Leu, Ile, Met, Arg, Thr or Val; Xaa at position 40 is Ile, Met, Ser or Val; Xaa at position 42 is Asp, Ala, Gly, Lys, Met, Ser or Thr; Xaa at position 43 is Thr, Cys, Asp, Glu, Gly, Met, Gln, Arg or Tyr; Xaa at position 46 is Lys, Gly, Asn or Arg; Xaa at position 47 is Leu, Cys, Glu, Lys or Ser; Xaa at position 50 is Ala, Lys, Arg, Ser, Thr or Val; Xaa at position 52 is Gly, Glu, Leu, Asn or Gln; Xaa at position 54 is Glu or Gly; Xaa at position 55 is Thr or Leu; Xaa at position 57 is Ile, Ala or Val; Xaa at position 61 is Asn, Ala, Gly, Leu or Ser; Xaa at position 63 is Pro or Val; Xaa at position 64 is Ala, Gly, His or Ser; Xaa at position 65 is Val or Cys; Xaa at position 67 is Ala or Ser; Xaa at position 68 is Ile or Gln; Xaa at position 69 is Pro, Gly, Arg, Ser or Val; Xaa at position 70 is Asp or His; Xaa at position 72 is Arg, Lys or Val; Xaa at position 73 is Lys, Glu, Gln or Arg; Xaa at position 75 is Ile or Arg; Xaa at position 76 is Glu or Gly; Xaa at position 77 is Ile, Met, Arg, Ser or Val; Xaa at position 79 is Arg or Gln; Xaa at position 81 is Ala or Ser; Xaa at position 84 is Val, Cys, Phe or Met; Xaa at position 85 is Leu or Ala; Xaa at position 88 is Glu or Lys; Xaa at position 89 is Cys, Ile or Val; Xaa at position 91 is Lys or Arg; Xaa at position 92 is Arg or Lys; Xaa at position 93 is Pro, Ala or Arg; Xaa at position 94 is Asp, Cys, Gly, Gln or Ser; Xaa at position 97 is Leu, Lys or Arg; Xaa at position 100 is Ala, Gly or Ser; Xaa at position 101 is Ala or Gly; Xaa at position 102 is Leu or Val; Xaa at position 104 is Leu or Met; Xaa at position 105 is Gln or Gly; Xaa at position 107 is Pro or Val; Xaa at position 108 is Asp or Glu; Xaa at position 109 is Ala, Gly, Met or Val; Xaa at position 111 is Thr, Ala, Cys, Gly, Ser or Val; Xaa at position 112 is Glu, Gly, Arg or Ser; Xaa at position 117 is Cys, Ala or Thr; Xaa at position 119 is Asn, Ala, Cys, Arg or Ser; Xaa at position 120 is Asp or Thr; Xaa at position 123 is Phe or Leu; Xaa at position 127 is Leu or Met; Xaa at position 133 is Gln or Val; Xaa at position 137 is Gly, Ala or Glu; Xaa at position 138 is Gln or Gly; Xaa at position 147 is Gln or Ile; Xaa at position 153 is Gly or Lys; Xaa at position 167 is Arg or Glu; Xaa at position 174 is Ser or Ala; Xaa at position 178 is Asp or Glu; Xaa at position 195 is Ala or Gly; Xaa at position 212 is Arg, Gly or Gln; Xaa at position 214 is Asn or Gln; Xaa at position 220 is Met or Leu; Xaa at position 228 is Met or Leu; Xaa at position 229 is Trp or Tyr; Xaa at position 235 is Val or Ile; Xaa at position 236 is Ala, Gly, Gln or Trp; Xaa at position 237 is Trp or Leu; Xaa at position 238 is Val, Gly or Pro; Xaa at position 239 is Lys, Ala, Asp, Glu, Gly or His; Xaa at position 240 is Leu, Ala, Asp, Glu, Gly or Val; Xaa at position 243 is Arg, Ala, Asp, Lys, Ser or Val; Xaa at position 245 is Pro or Ala; Xaa at position 248 is Arg or Lys; Xaa at position 249 is Arg or Pro; Xaa at position 251 is Met or Val; Xaa at position 255 is Asn, Ala, Leu, Met, Gln, Arg or Ser; Xaa at position 259 is His or Trp; Xaa at position 260 is Ile or Leu; Xaa at position 278 is Ile or Leu; Xaa at position 298 is Ser, Ala or Thr; Xaa at position 299 is Asp or Ala; Xaa at position 302 is Asn or Ala; Xaa at position 303 is Ala, Cys, Asp, Glu or Ser; Xaa at position 304 is Thr or Val; Xaa at position 312 is Val or Leu; Xaa at position 316 is Arg or Ser; Xaa at position 320 is Arg or Leu; Xaa at position 321 is Arg or Asn; Xaa at position 327 is Gly, Leu, Gin or Val; Xaa at position 328 is Ala, Cys, Asp, Arg, Ser, Thr or Val; wherein one or more amino acid(s) designated by Xaa in SEQ ID NO: 1041 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

In specific embodiments, host cells, plants, plant cells, plant parts and seed are provided which comprise at least one heterologous polynucleotide encoding a dicamba decarboxylase polypeptide comprising:

```
(SEQ ID NO: 1042)
                                5 10
Met Ala Gln Gly Xaa Val Ala Leu Glu Glu His Phe
                15 20
Ala Ile Pro Xaa Thr Leu Xaa Asp Xaa Ala Xaa Phe
25 30 35
Val Pro Xaa Xaa Tyr Xaa Lys Glu Leu Gln His Arg
                        40 45
Leu Xaa Asp Xaa Gln Asp Xaa Arg Leu Xaa Xaa Met
        50 55 60
Asp Xaa His Xaa Ile Xaa Thr Met Xaa Leu Ser Leu
                                65 70
Xaa Ala Xaa Xaa Val Gln Xaa Ile Xaa Asp Arg Xaa
                75 80
Xaa Ala Ile Glu Xaa Ala Xaa Arg Ala Asn Asp Xaa
85 90 95
Leu Ala Glu Glu Xaa Ala Lys Arg Pro Xaa Arg Phe
                        100 105
Leu Ala Phe Ala Ala Leu Pro Xaa Gln Asp Xaa Xaa
        110 115 120
Ala Ala Xaa Xaa Glu Leu Gln Arg Xaa Val Xaa Xaa
                                125 130
Leu Gly Phe Val Gly Ala Xaa Val Asn Gly Phe Ser
                135 140
Xaa Glu Gly Asp Gly Gln Thr Pro Leu Tyr Tyr Asp
145 150 155
Leu Pro Gln Tyr Arg Pro Phe Trp Xaa Glu Val Glu
                        160 165
Lys Leu Asp Val Pro Phe Tyr Leu His Pro Arg Asn
        170 175 180
Pro Leu Pro Gln Asp Xaa Arg Ile Tyr Asp Gly His
                                185 190
Pro Trp Leu Leu Gly Pro Thr Trp Ala Phe Ala Gln
                195 200
Glu Thr Ala Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Met
        230 235 240
Xaa Arg Ile Asp His Arg Xaa Xaa Trp Val Xaa Xaa
                                245 250
Pro Pro Xaa Tyr Xaa Ala Lys Arg Arg Phe Met Asp
                255 260
Tyr Phe Xaa Glu Asn Phe Xaa Ile Thr Thr Ser Gly
265 270 275
Asn Phe Arg Thr Gln Thr Leu Ile Asp Ala Ile Leu
                        280 285
Glu Ile Gly Ala Asp Arg Ile Leu Phe Xaa Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Arg Thr Asn Ala Xaa Xaa Leu Phe Lys
325
Leu Asp Xaa Xaa
```

wherein
Xaa at position 5 is Lys or Leu; Xaa at position 16 is Glu or Ala; Xaa at position 19 is Gln or Asn; Xaa at position 21 is Ser or Ala; Xaa at position 23 is Gly or Asp; Xaa at position 27 is Gly or Ser; Xaa at position 28 is Asp, Cys or Glu; Xaa at position 30 is Trp or Leu; Xaa at position 38 is Leu or Met; Xaa at position 40 is Ile or Met; Xaa at position 43 is Thr, Glu or Gln; Xaa at position 46 is Lys, Asn or Arg; Xaa at position 47 is Leu or Glu; Xaa at position 50 is Ala, Lys or Arg; Xaa at position 52 is Gly, Glu or Gln; Xaa at position 54 is Glu or Gly; Xaa at position 57 is Ile or Val; Xaa at position 61 is Asn or Ala; Xaa at position 63 is Pro or Val; Xaa at position 64 is Ala or Gly; Xaa at position 67 is Ala, Gly or Ser; Xaa at position 69 is Pro, Gly or Val; Xaa at position 72 is Arg or Val; Xaa at position 73 is Lys, Glu or Gln; Xaa at position 77 is Ile or Leu; Xaa at position 79 is Arg or Lys; Xaa at position 84 is Val, Phe or Met; Xaa at position 89 is Cys or Val; Xaa at position 94 is Asp or Gly; Xaa at position 104 is Leu or Met; Xaa at position 107 is Pro or Val; Xaa at position 108 is Asp or Glu; Xaa at position 111 is Thr or Ser; Xaa at position 112 is Glu or Ser; Xaa at position 117 is Cys or Thr; Xaa at position 119 is Asn, Ala or Arg; Xaa at position 120 is Asp or Thr; Xaa at position 127 is Leu or Met; Xaa at position 133 is Gln or Val; Xaa at position 153 is Gly or Lys; Xaa at position 174 is Ser or Ala; Xaa at position 212 is Arg or Gly; Xaa at position 214 is Asn or Gln; Xaa at position 220 is Met or Leu; Xaa at position 229 is Trp or Tyr; Xaa at position 235 is Val or Ile; Xaa at position 236 is Ala or Gly; Xaa at position 239 is Lys, Glu or His; Xaa at position 240 is Leu, Ala or Glu; Xaa at position 243 is Arg or Asp; Xaa at position 245 is Pro or Ala; Xaa at position 255 is Asn or Leu; Xaa at position 259 is His or Trp; Xaa at position 286 is Ser or Ala; Xaa at position 298 is Ser, Ala or Thr; Xaa at position 299 is Asp or Ala; Xaa at position 302 is Asn or Ala; Xaa at position 303 is Ala or Glu; Xaa at position 304 is Thr or Ala; Xaa at position 312 is Val or Leu; Xaa at position 320 is Arg or Leu; Xaa at position 321 is Arg or Asn; Xaa at position 327 is Gly, Leu or Val; Xaa at position 328 is Ala, Asp, Arg, Ser or Thr; wherein one or more amino acid(s) designated by Xaa in SEQ ID NO: 1042 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

The host cell, plants, plant cells and seed which express the heterologous polynucleotide encoding the dicamba decarboxylase polypeptide can display an increased tolerance to an auxin-analog herbicide. “Increased tolerance” to an auxin-analog herbicide, such as dicamba, is demonstrated when plants which display the increased tolerance to the auxin-analog herbicide are subjected to the auxin-analog herbicide and a dose/response curve is shifted to the right when compared with that provided by an appropriate control plant. Such dose/response curves have “dose” plotted on the x-axis and “percentage injury”, “herbicidal effect” etc. plotted on the y-axis. Plants which are substantially “resistant” or “tolerant” to the auxin-analog herbicide exhibit few, if any, significant negative agronomic effects when subjected to the auxin-analog herbicide at concentrations and rates which are typically employed by the agricultural community to kill weeds in the field.

In specific embodiments, the heterologous polynucleotide encoding the dicamba decarboxylase polypeptide or active variant or fragment thereof in the host cell, plant or plant part is operably linked to a constitutive, tissue-preferred, or other promoter for expression in the host cell or the plant of interest.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The polynucleotide encoding the dicamba decarboxylase polypeptide and active variants and fragments thereof may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis), and Poplar and Eucalyptus. In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are of interest.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

A “subject plant or plant cell” is one in which genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same germplasm, variety or line as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

### IV. Polynucleotide Constructs

The use of the term “polynucleotide” is not intended to limit the methods and compositions to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides employed herein also encompass all forms of sequences including, but not limited to, single-stranded forms, double-stranded forms, hairpins, stem-and-loop structures, and the like.

The polynucleotides encoding a dicamba decarboxylase polypeptide or active variant or fragment thereof can be provided in expression cassettes for expression in the plant of interest. The cassette can include 5′ and 3′ regulatory sequences operably linked to a polynucleotide encoding a dicamba decarboxylase polypeptide or an active variant or fragment thereof. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. Additional gene(s) can be provided on multiple expression cassettes. Such an expression cassette is provided with a plurality of restriction sites and/or recombination sites for insertion of the polynucleotide encoding a dicamba decarboxylase polypeptide or an active variant or fragment thereof to be under the transcriptional regulation of the regulatory regions.

The expression cassette can include in the 5′-3′ direction of transcription, a transcriptional and translational initiation region (i.e., a promoter), a polynucleotide encoding a dicamba decarboxylase polypeptide or an active variant or fragment thereof, and a transcriptional and translational termination region (i.e., termination region) functional in plants. The regulatory regions (i.e., promoters, transcriptional regulatory regions, and translational termination regions) and/or the polynucleotide encoding a dicamba decarboxylase polypeptide or an active variant or fragment thereof may be native/analogous to the host cell or to each other. Alternatively, the regulatory regions and/or the polynucleotide encoding the dicamba decarboxylase polypeptide of or an active variant or fragment thereof may be heterologous to the host cell or to each other. Moreover, as discussed in further detail elsewhere herein, the polynucleotide encoding the dicamba decarboxylase polypeptide can further comprise a polynucleotide encoding a “targeting signal” that will direct the dicamba decarboxylase polypeptide to a desired sub-cellular location.

As used herein, “heterologous” in reference to a sequence is a sequence that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are modified from their original form and/or genomic locus, or the promoter is not the native promoter for the operably linked polynucleotide.

While it may be optimal to express the sequences using heterologous promoters, the native promoter sequences may be used. Such constructs can change expression levels of the polynucleotide encoding a dicamba decarboxylase polypeptide in the host cell, plant or plant cell. Thus, the phenotype of the host cell, plant or plant cell can be altered.

The termination region may be native with the transcriptional initiation region, may be native with the operably linked polynucleotide encoding a dicamba decarboxylase polypeptide or active variant or fragment thereof, may be native with the host cell (i.e., plant cell), or may be derived from another source (i.e., foreign or heterologous) to the promoter, the polynucleotide encoding a dicamba decarboxylase polypeptide or active fragment or variant thereof, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed host cell (i.e., a microbial cell or a plant cell). In specific embodiments, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference in their entirety.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385. See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used to express the various dicamba decarboxylase sequences disclosed herein, including the native promoter of the polynucleotide sequence of interest. The promoters can be selected based on the desired outcome. Such promoters include, for example, constitutive, tissue-preferred, or other promoters for expression in plants.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026); and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the polynucleotide encoding the dicamba decarboxylase polypeptide within a particular plant tissue. Tissue-preferred promoters include those described in Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Meristem-preferred promoters can also be employed. Such promoter can drive expression in meristematic tissue, including, for example, the apical meristem, axillary buds, root meristems, cotyledon meristem and/or hypocotyl meristem. Non-limiting examples of meristem-preferred promoters include the shoot meristem specific promoter such as the Arabidopsis UFO gene promoter (Unusual Floral Organ) (USA6239329), the meristem-specific promoters of FTM1, 2, 3 and SVP1, 2, 3 genes as discussed in US Patent App. 20120255064, and the shoot meristem-specific promoter disclosed in U.S. Pat. No. 5,880,330. Each of these references is herein incorporated by reference in their entirety.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, sulfonylureas. Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et at (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference in their entirety. The above list of selectable marker genes is not meant to be limiting.

### V. Stacking Other Traits of Interest

In some embodiments, the polynucleotide encoding the dicamba decarboxylase polypeptide or an active variant or fragment thereof are engineered into a molecular stack. Thus, the various host cells, plants, plant cells and seeds disclosed herein can further comprise one or more traits of interest, and in more specific embodiments, the host cell, plant, plant part or plant cell is stacked with any combination of polynucleotide sequences of interest in order to create plants with a desired combination of traits. As used herein, the term “stacked” includes having the multiple traits present in the same plant (i.e., both traits are incorporated into the nuclear genome, one trait is incorporated into the nuclear genome and one trait is incorporated into the genome of a plastid, or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. In one embodiment, the molecular stack comprises at least one additional polynucleotide that confers tolerance to at least one additional auxin-analog herbicide and/or at least one additional polynucleotide that confers tolerance to a second herbicide.

Thus, in one embodiment, the host cell, plants, plant cells or plant part having the polynucleotide encoding the dicamba decarboxylase polypeptide or an active variant or fragment thereof is stacked with at least one other dicamba decarboxylase sequence. Alternatively, the host cell, plant, plant cells or seed having the heterologous polynucleotide encoding the dicamba decarboxylase polypeptide can have the dicamba decarboxylase sequence stacked with an additional sequence that confers tolerance to an auxin-analog herbicide via a different mode of action than that of the dicamba decarboxylase sequence. Such sequences include, but are not limited to, the aryloxyalkanoate dioxygenase polynucleotides which confer tolerance to 2,4-D and other phenoxy auxin herbicides, as well as, to aryloxyphenoxypropionate herbicides as described, for example, in WO2005/107437 and WO2007/053482. Additional sequence can further include dicamba-tolerance polynucleotides as described, for example, in Herman et al. (2005) J. Biol. Chem. 280: 24759-24767, U.S. Pat. Nos. 7,820,883; 8,088,979; 8,071,874; 8,119,380; 7,105,724; 7,855,3326; 8,084,666; 7,838,729; 5,670,454; US Application Publications 2012/0064539, 2012/0064540, 2011/0016591, 2007/0220629, 2001/0016890, 2003/0115626, WO2012/094555, WO2007/46706, WO2012024853, EP0716808, and EP1379539, and an acetyl coenzyme A carboxylase (ACCase) polypeptides, each of which is herein incorporated by reference in their entirety. Other sequences that confer tolerance auxin, such as methyltransferases, are set forth in US 2010/0205696 and WO 2010/091353, both of which are herein incorporated by reference in their entirety. Other auxin tolerance proteins are known and could be employed.

In another embodiment, the host cell, plant, plant cell or plant part having the polynucleotide encoding the dicamba decarboxylase polypeptide or an active variant or fragment thereof is stacked with at least one polynucleotide encoding a dicamba monooxygenase (DOM). See, for example, U.S. Pat. No. 8,207,092, which is herein incorporated by reference in its entirety.

In still other embodiments, host cells, plants, plant cells, explants and expression cassettes comprising the polynucleotide encoding the dicamba decarboxylase polypeptide or active variant or fragment thereof are stacked with a sequence that confers tolerance to HPPD inhibitors or an HPPD detoxification enzyme. For example, a P450 sequence could be employed which provides tolerance to HPPD-inhibitors by metabolism of the herbicide. Such sequences include, but are not limited to, the NSF1 gene. See, US 2007/0214515 and US 2008/0052797, both of which are herein incorporated by reference in their entirety. Additional HPPD target site genes that confer herbicide tolerance to plants include those set forth in U.S. Pat. No. 6,245,968 B1; 6,268,549; and 6,069,115; international publication WO 99/23886, US App Pub. 2012-0042413 and US App Pub 2012-0042414, each of which is herein incorporated by reference in their entirety.

In some embodiments, the host cell, plant or plant cell having the heterologous polynucleotide encoding a dicamba decarboxylase polypeptide or active variant or fragment thereof may be stacked with sequences that confer tolerance to glyphosate such as, for example, glyphosate N-acetyltransferase. See, for example, WO02/36782, US Publication 2004/0082770 and WO 2005/012515, U.S. Pat. No. 7,462,481, U.S. Pat. No. 7,405,074, each of which is herein incorporated by reference in their entirety. Additional glyphosate-tolerance traits include a sequence that encodes a glyphosate oxido-reductase enzyme as described more fully in U.S. Pat. Nos. 5,776,760 and 5,463,175. Other traits that could be combined with the polynucleotide encoding the dicamba decarboxylase polypeptide or active variant or fragment thereof include those derived from polynucleotides that confer on the plant the capacity to produce a higher level or glyphosate insensitive 5-enolpyruvylshikimate-3-phosphate synthase (EPSPS), for example, as more fully described in U.S. Pat. No. 6,248,876 B1; 5,627,061; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; RE 36,449; RE 37,287 E; and 5,491,288; and international publications WO 97/04103; WO 00/66746; WO 01/66704; and WO 00/66747, 6,040,497; 5,094,945; 5,554,798; 6,040,497; Zhou et al. (1995) Plant Cell Rep.: 159-163; WO 0234946; WO 9204449; 6,225,112; 4,535,060, and 6,040,497, which are incorporated herein by reference in their entireties for all purposes. Additional EPSP synthase sequences include, gdc-1 (U.S. App. Publication 20040205847); EPSP synthases with class III domains (U.S. App. Publication 20060253921); gdc-1 (U.S. App. Publication 20060021093); gdc-2 (U.S. App. Publication 20060021094); gro-1 (U.S. App. Publication 20060150269); grg23 or grg 51 (U.S. App. Publication 20070136840); GRG32 (U.S. App. Publication 20070300325); GRG33, GRG35, GRG36, GRG37, GRG38, GRG39 and GRG50 (U.S. App. Publication 20070300326); or EPSP synthase sequences disclosed in, U.S. App. Publication 20040177399; 20050204436; 20060150270; 20070004907; 20070044175; 2007010707; 20070169218; 20070289035; and, 20070295251; each of which is herein incorporated by reference in their entirety.

In other embodiments, the host cell, plant or plant cell or plant part having the heterologous polynucleotide encoding the dicamba decarboxylase polypeptide or an active variant or fragment thereof is stacked with, for example, a sequence which confers tolerance to an ALS inhibitor. As used herein, an “ALS inhibitor-tolerant polypeptide” comprises any polypeptide which when expressed in a plant confers tolerance to at least one ALS inhibitor. Varieties of ALS inhibitors are known and include, for example, sulfonylurea, imidazolinone, triazolopyrimidines, pryimidinyoxy(thio)benzoates, and/or sulfonylaminocarbonyltriazolinone herbicides. Additional ALS inhibitors are known and are disclosed elsewhere herein. It is known in the art that ALS mutations fall into different classes with regard to tolerance to sulfonylureas, imidazolinones, triazolopyrimidines, and pyrimidinyl(thio)benzoates, including mutations having the following characteristics: (1) broad tolerance to all four of these groups; (2) tolerance to imidazolinones and pyrimidinyl(thio)benzoates; (3) tolerance to sulfonylureas and triazolopyrimidines; and (4) tolerance to sulfonylureas and imidazolinones.

Various ALS inhibitor-tolerant polypeptides can be employed. In some embodiments, the ALS inhibitor-tolerant polynucleotides contain at least one nucleotide mutation resulting in one amino acid change in the ALS polypeptide. In specific embodiments, the change occurs in one of seven substantially conserved regions of acetolactate synthase. See, for example, Hattori et al. (1995) Molecular Genetics and Genomes 246:419-425; Lee et al. (1998) EMBO Journal 7:1241-1248; Mazur et al. (1989) Ann. Rev. Plant Phys. 40:441-470; and U.S. Pat. No. 5,605,011, each of which is incorporated by reference in their entirety. The ALS inhibitor-tolerant polypeptide can be encoded by, for example, the SuRA or SuRB locus of ALS. In specific embodiments, the ALS inhibitor-tolerant polypeptide comprises the C3 ALS mutant, the HRA ALS mutant, the S4 mutant or the S4/HRA mutant or any combination thereof. Different mutations in ALS are known to confer tolerance to different herbicides and groups (and/or subgroups) of herbicides; see, e.g., Tranel and Wright (2002) Weed Science 50:700-712. See also, U.S. Pat. Nos. 5,605,011, 5,378,824, 5,141,870, and 5,013,659, each of which is herein incorporated by reference in their entirety. The soybean, maize, and Arabidopsis HRA sequences are disclosed, for example, in WO2007/024782, herein incorporated by reference in their entirety.

In some embodiments, the ALS inhibitor-tolerant polypeptide confers tolerance to sulfonylurea and imidazolinone herbicides. The production of sulfonylurea-tolerant plants and imidazolinone-tolerant plants is described more fully in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, which are incorporated herein by reference in their entireties for all purposes. In specific embodiments, the ALS inhibitor-tolerant polypeptide comprises a sulfonamide-tolerant acetolactate synthase (otherwise known as a sulfonamide-tolerant acetohydroxy acid synthase) or an imidazolinone-tolerant acetolactate synthase (otherwise known as an imidazolinone-tolerant acetohydroxy acid synthase).

In further embodiments, the host cell, plants or plant cell or plant part having the heterologous polynucleotide encoding the dicamba decarboxylase polypeptide or an active variant or fragment thereof is stacked with, for example, a sequence which confers tolerance to an ALS inhibitor and glyphosate tolerance. In one embodiment, the polynucleotide encoding the dicamba decarboxylase polypeptide or active variant or fragment thereof is stacked with HRA and a glyphosate N-acetyltransferase. See, WO2007/024782, 2008/0051288 and WO 2008/112019, each of which is herein incorporated by reference in their entirety.

Other examples of herbicide-tolerance traits that could be combined with the host cell, plant or plant cell or plant part having the heterologous polynucleotide encoding the dicamba decarboxylase polypeptide or an active variant or fragment thereof include those conferred by polynucleotides encoding an exogenous phosphinothricin acetyltransferase, as described in U.S. Pat. Nos. 5,969,213; 5,489,520; 5,550,318; 5,874,265; 5,919,675; 5,561,236; 5,648,477; 5,646,024; 6,177,616; and 5,879,903. Plants containing an exogenous phosphinothricin acetyltransferase can exhibit improved tolerance to glufosinate herbicides, which inhibit the enzyme glutamine synthase. Other examples of herbicide-tolerance traits that could be combined with the plants or plant cell or plant part having the heterologous polynucleotide encoding the dicamba decarboxylase polypeptide or an active variant or fragment thereof include those conferred by polynucleotides conferring altered protoporphyrinogen oxidase (protox) activity, as described in U.S. Pat. Nos. 6,288,306 B1; 6,282,837 B1; and 5,767,373; and international publication WO 01/12825 or those that are protoporphorinogen detoxification enzyme. Plants containing such polynucleotides can exhibit improved tolerance to any of a variety of herbicides which target the protox enzyme (also referred to as “protox inhibitors”).

Other examples of herbicide-tolerance traits that could be combined with the host cell, plant or plant cell or plant part having the heterologous polynucleotide encoding the dicamba decarboxylase polypeptide or an active variant or fragment thereof include those conferring tolerance to at least one herbicide in a plant such as, for example, a maize plant or horseweed. Herbicide-tolerant weeds are known in the art, as are plants that vary in their tolerance to particular herbicides. See, e.g., Green and Williams (2004) “Correlation of Corn (Zea mays) Inbred Response to Nicosulfuron and Mesotrione,” poster presented at the WSSA Annual Meeting in Kansas City, Mo., Feb. 9-12, 2004; Green (1998) Weed Technology 12: 474-477; Green and Ulrich (1993) Weed Science 41: 508-516. The trait(s) responsible for these tolerances can be combined by breeding or via other methods with the plants or plant cell or plant part having the heterologous polynucleotide encoding the dicamba decarboxylase or an active variant or fragment thereof to provide a plant of the invention, as well as, methods of use thereof.

In still further embodiments, the polynucleotide encoding the dicamba decarboxylase polypeptide can be stacked with at least one polynucleotide encoding a homogentisate solanesyltransferase (HST). See, for example, WO2010023911 herein incorporated by reference in its entirety. In such embodiments, classes of herbicidal compounds—which act wholly or in part by inhibiting HST can be applied over the plants having the HTS polypeptide.

The host cell, plant or plant cell or plant part having the polynucleotide encoding the dicamba decarboxylase polypeptide or an active variant or fragment thereof can also be combined with at least one other trait to produce plants that further comprise a variety of desired trait combinations including, but not limited to, traits desirable for animal feed such as high oil content (e.g., U.S. Pat. No. 6,232,529); balanced amino acid content (e.g., hordothionins (U.S. Pat. Nos. 5,990,389; 5,885,801; 5,885,802; and 5,703,409; U.S. Pat. No. 5,850,016); barley high lysine (Williamson et al. (1987) Eur. J. Biochem. 165: 99-106; and WO 98/20122) and high methionine proteins (Pedersen et al. (1986) J. Biol. Chem. 261: 6279; Kirihara et al. (1988) Gene 71: 359; and Musumura et al. (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)); the disclosures of which are herein incorporated by reference in their entirety. Desired trait combinations also include LLNC (low linolenic acid content; see, e.g., Dyer et al. (2002) Appl. Microbiol. Biotechnol. 59: 224-230) and OLCH (high oleic acid content; see, e.g., Fernandez-Moya et al. (2005) J. Agric. Food Chem. 53: 5326-5330).

The host cell, plant or plant cell or plant part having the polynucleotide encoding the dicamba decarboxylase polypeptide or an active variant or fragment thereof can also be combined with other desirable traits such as, for example, fumonisim detoxification genes (U.S. Pat. No. 5,792,931), avirulence and disease resistance genes (Jones et al. (1994) Science 266: 789; Martin et al. (1993) Science 262: 1432; Mindrinos et al. (1994) Cell 78: 1089), and traits desirable for processing or process products such as modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference in their entirety. One could also combine herbicide-tolerant polynucleotides with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821); the disclosures of which are herein incorporated by reference in their entirety.

In other embodiments, the host cell, plant or plant cell or plant part having the polynucleotide encoding the dicamba decarboxylase polypeptide or an active variant or fragment thereof may be stacked with any other polynucleotides encoding polypeptides having pesticidal and/or insecticidal activity, such as Bacillus thuringiensis toxic proteins (described in U.S. Pat. Nos. 5,366,892; 5,747,450; 5,737,514; 5,723,756; 5,593,881; Geiser et al. (1986) Gene 48: 109; Lee et al. (2003) Appl. Environ. Microbiol. 69: 4648-4657 (Vip3A); Galitzky et al. (2001) Acta Crystallogr. D. Biol. Crystallogr. 57: 1101-1109 (Cry3Bb1); and Herman et al. (2004) J. Agric. Food Chem. 52: 2726-2734 (Cry1F)); lectins (Van Damme et al. (1994) Plant Mol. Biol. 24: 825, pentin (described in U.S. Pat. No. 5,981,722), and the like. The combinations generated can also include multiple copies of any one of the polynucleotides of interest.

In another embodiment, the host cell, plant or plant cell or plant part having the polynucleotide encoding the dicamba decarboxylase polypeptide or an active variant or fragment thereof can also be combined with the Rcgl sequence or biologically active variant or fragment thereof. The Rcgl sequence is an anthracnose stalk rot resistance gene in corn. See, for example, U.S. patent application Ser. Nos. 11/397,153, 11/397,275, and 11/397,247, each of which is herein incorporated by reference in their entirety.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference in their entirety. Additional systems can be used for site specific integration including, for example, various meganucleases systems as set forth in WO 2009/114321 (herein incorporated by reference in its entirety), which describes “custom” meganucleases. See, also, Gao et al. (2010) Plant Journal 1:176-187. Additional site specific integration systems include, but are not limited, to Zn Fingers, meganucleases, and TAL nucleases. See, for example, WO2010079430, WO2011072246, and US20110201118, each of which is herein incorporated by reference in their entirety.

### VI. Method of Introducing

Various methods can be used to introduce a sequence of interest into a host cell, plant or plant part. “Introducing” is intended to mean presenting to the host cell, plant, plant cell or plant part the polynucleotide or polypeptide in such a manner that the sequence gains access to the interior of a cell. The methods disclosed herein do not depend on a particular method for introducing a sequence into a host cell, plant or plant part, only that the polynucleotide or polypeptides gains access to the interior of at least one cell. Methods for introducing polynucleotides or polypeptides into plants are known in the art including, but not limited to, stable transformation methods, transient transformation methods, and virus-mediated methods.

“Stable transformation” is intended to mean that the nucleotide construct introduced into a host cell or plant integrates into the genome of the host cell or plant and is capable of being inherited by the progeny thereof. “Transient transformation” is intended to mean that a polynucleotide is introduced into the host cell or plant and does not integrate into the genome of the host cell or plant or a polypeptide is introduced into a host cell or plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference in their entirety.

In specific embodiments, the dicamba decarboxylase sequences or active variant or fragments thereof can be provided to a plant using a variety of transient transformation methods. Such transient transformation methods include, but are not limited to, the introduction of the dicamba decarboxylase protein or active variants and fragments thereof directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference in their entirety.

In other embodiments, the polynucleotide encoding the dicamba decarboxylase polypeptide or active variants or fragments thereof may be introduced into plants by contacting plants with a virus or viral nucleic acids. Generally, such methods involve incorporating a nucleotide construct of the invention within a DNA or RNA molecule. It is recognized that the an dicamba decarboxylase sequence may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference in their entirety.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference in their entirety. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome. Other methods to target polynucleotides are set forth in WO 2009/114321 (herein incorporated by reference in its entirety), which describes “custom” meganucleases produced to modify plant genomes, in particular the genome of maize. See, also, Gao et al. (2010) Plant Journal 1:176-187.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

Additional host cells of interest include, for example, prokaryotes including various strains of E. coli and other microbial strains. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived P L promoter and N-gene ribosome binding site (Shimatake et al. (1981) Nature 292:128). The inclusion of selection markers in DNA vectors transfected in E coli. is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva et al. (1983) Gene 22:229-235); Mosbach et al. (1983) Nature 302:543-545).

A variety of expression systems for yeast are known to those of skill in the art. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers. See, for Example, Sherman et al. (1982) Methods in Yeast Genetics, Cold Spring Harbor Laboratory.

### VII. Methods of Use

A. Methods for Increasing Expression and/or Concentration of at Least One Dicamba Decarboxylase Sequence or an Active Variant or Fragment Therefore in Host Cells

A method for increasing the activity and/or concentration of a dicamba decarboxylase polypeptide disclosed herein or an active variant or fragment thereof in a host cell, plant, plant cell, plant part, explant, or seed is provided. Methods for assaying for an increase in dicamba decarboxylase activity are discussed in detail elsewhere herein.

In further embodiments, the concentration/level of the dicamba decarboxylase polypeptide is increased in a host cell, a plant or plant part by at least 10%, 20%, 30%, 40%, 50%, 60%, 70%, 80%, 90%, 100%, 200%, 500%, 1000%, 5000%, or 10,000% relative to an appropriate control host cell, plant, plant part, or cell which did not have the dicamba decarboxylase sequence. In still other embodiments, the level of the dicamba decarboxylase polypeptide in the host cell, plant or plant part is increased by 10, 20, 30, 40, 50, 60, 70, 80, 90, 100 fold or more compared to the level of the native dicamba decarboxylase sequence. Such an increase in the level of the dicamba decarboxylase polypeptide can be achieved in a variety of ways including, for example, by the expression of multiple copies of one or more dicamba decarboxylase polypeptide and/or by employing a promoter to drive higher levels of expression of the sequence.

In specific embodiments, the polypeptide or the dicamba decarboxylase polynucleotide or active variant or fragment thereof is introduced into the host cell, plant, plant cell, explant or plant part. Subsequently, a host cell or plant cell having the introduced sequence of the invention is selected using methods known to those of skill in the art such as, but not limited to, Southern blot analysis, DNA sequencing, PCR analysis, or phenotypic analysis. When a plant or plant part is employed in the foregoing embodiments, the plant or plant cell is grown under plant forming conditions for a time sufficient to modulate the concentration and/or activity of the dicamba decarboxylase polypeptide in the plant. Plant forming conditions are well known in the art and discussed briefly elsewhere herein.

In one embodiment, a method of producing a dicamba tolerant host cell or plant cell is provided and comprises transforming a host cell or plant cell with the polynucleotide encoding a dicamba decarboxylase polypeptide or active variant or fragment thereof. In specific embodiments, the method further comprises selecting a host cell or plant cell which is resistant or tolerant to the dicamba.

B. Methods to Decarboxylate Auxin-Analogs

Methods and compositions are provided to decarboxylate auxin-analogs using a dicamba decarboxylase or an active variant or fragment thereof. In specific embodiments, an auxin-analog herbicide is used, and the decarboxylation of the auxin-analog herbicide detoxifies the auxin-analog herbicide.

As used herein, an “auxin-analog herbicide” or “synthetic auxin herbicide” are used interchangeably and comprises any auxinic or growth regulator herbicides, otherwise known as Group 4 herbicides (based on their mode of action), including the acids themselves or their agricultural esters and salts. These types of herbicides mimic or act like the natural plant growth regulators called auxins. The action of auxin-analog herbicide appears to affect cell wall plasticity and nucleic acid metabolism, which can lead to uncontrolled cell division and growth. See, for example, Cox et al. (1994) Journal of Pesticide Reform 14:30-35; Dayan et al. (2010) Weed Science 58:340-350; Davidonis et al. (1982) Plant Physiol 70:357-360; Mithila et al. (2011) Weed Science 59:445-457; Grossmann (2007) Plant Signalling and Behavior 2:421-423, U.S. Pat. No. 7,855,326; US App. Pub. 2012/0178627; US App. Pub. 2011/0124503; and U.S. Pat. No. 7,838,733, each of which is herein incorporated by reference in their entirety. An auxin-analog herbicide derivative includes any metabolic product of the auxin-analog herbicide. Such a metabolic product may or may not retain herbicidal activity.

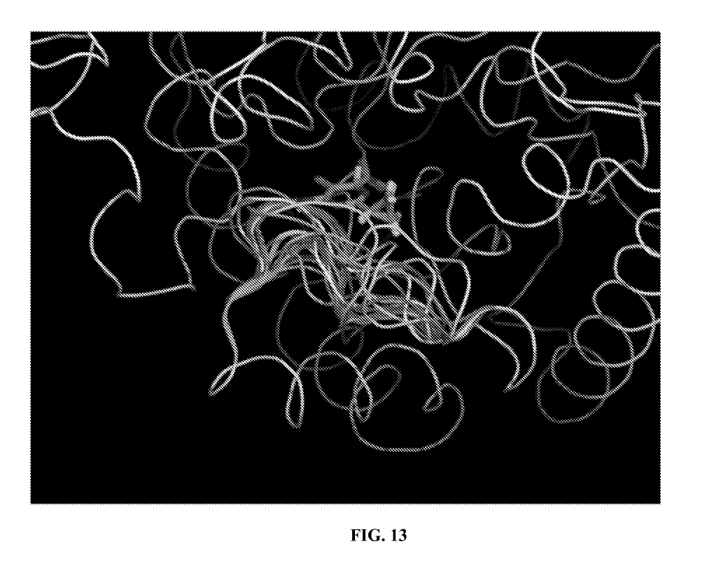

Auxin-analog herbicides include the chemical families: phenoxy-carboxylic-acid, pyridine carboxylic acid, benzoic acid, quinoline carboxylic acid, aminocyclopyrachlor (MAT28) and benazolin-ethyl and any of their acids or salts. The structures of various auxin-analog herbicides are set forth in FIG. 13. Phenoxy-carboxylic acid herbicides include (2,4-dichlorophenoxy)acetic acid (otherwise known as 2,4-D); 4-(2,4-dichlorophenoxy)butyric acid (2,4-DB); 2-(2,4-dichlorophenoxy)propanoic acid (2,4-DP), (2,4,5-trichlorophenoxy)acetic acid (2,4,5-T); 2-(2,4,5-Trichlorophenoxy)Propionic Acid (2,4,5-TP); 2-(2,4-dichloro-3-methylphenoxy)-N-phenylpropanamide (clomeprop); (4-chloro-2-methylphenoxy)acetic acid (MCPA); 4-(4-chloro-o-tolyloxy)butyric acid (MCPB); and 2-(4-chloro-2-methylphenoxy)propanoic acid (MCPP).

Other forms of auxin-analog herbicides include the pyridine carboxylic acid herbicides. Examples include 3,6-dichloro-2-pyridinecarboxylic acid (Clopyralid), 4-amino-3,5,6-trichloro-2-pyridinecarboxylic acid (picloram), (2,4,5-trichlorophenoxy) acetic acid (triclopyr), and 4-amino-3,5-dichloro-6-fluoro-2-pyridyloxyacetic acid (fluoroxypyr).

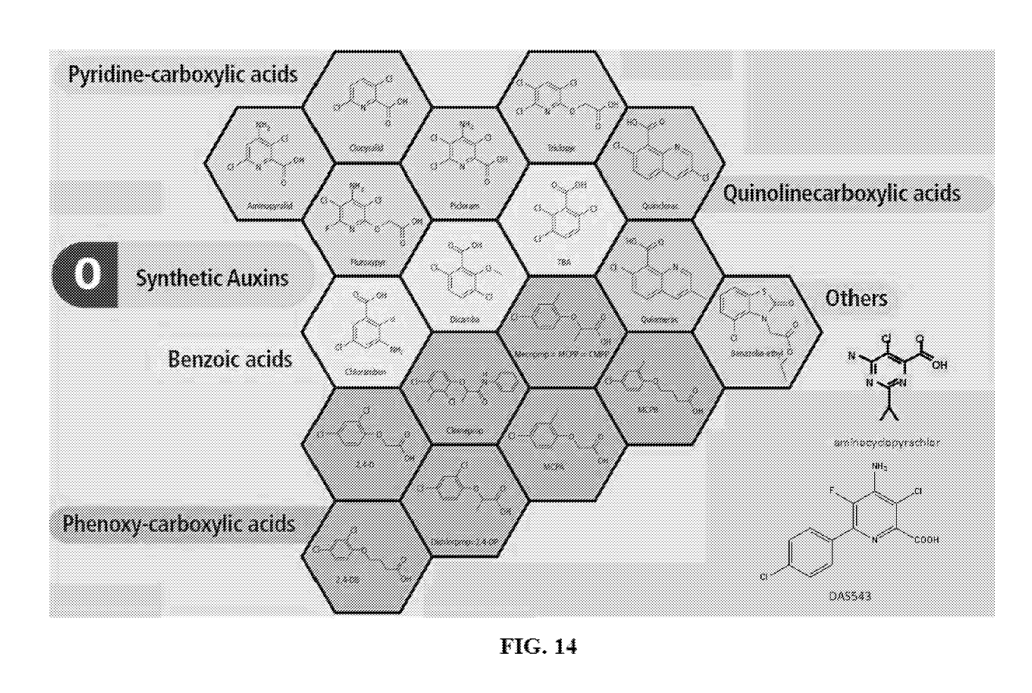

Examples of benzoic acids family of auxin-analog herbicides include 3,6-dichloro-o-anisic acid (dicamba) and 3-amino-2,5-dichlorobenzoic acid (choramben), and TBD, as shown in FIG. 14. Dicamba or active derivative thereof is a particularly useful herbicide for use in the methods and compositions disclosed herein.

The quinoline carboxylic acid family of auxin-analog herbicides includes 3,7-dichloro-8-quinolinecarboxylic acid (quinclorac). This herbicide is unique in that it also will control some grass weeds, unlike the other auxin-analog herbicide which essentially control only broadleaf or dicotyledonous plants. The other herbicide in this category is 7-chloro-3-methyl-8-quinolinecarboxylic acid (quinmerac). In other embodiments, the auxin-analog herbicide comprises aminocyclopyrachlor, aminopyralid benazolin-ethyl, chloramben, clomeprop, clopyralid, dicamba, 2,4-D, 2,4-DB, dichlorprop, fluroxypyr, mecoprop, MCPA, MCPB, 2,3,6-TBA, picloram, triclopyr, quinclorac, or quinmerac. See, for example, WO2010/046422, WO2011/161131, WO2012/033548, and US Application Publications 20110287935, 20100069248, and 20100048399, each of which is herein incorporated by reference in their entirety. Additional auxin-analog herbecides include those set forth in Heap et al. (2013) The International Survey of Herbecide Resistant Weeds. Online. Internet. at www.weedscience.com., the contents of which are herein incorporated by reference.

While any auxin-analog herbicide can be employed in the methods and compositions disclosed herein, in one embodiment, the auxin-analog herbicide comprises a member of the benzoic acid family of auxin-analog herbicides, a derivative of a benzoic acid auxin-analog herbicide, or a metabolic product of such a compound. Examples of benzoic acids family of the auxin-analog herbicides include 3,6-dichloro-o-anisic acid (dicamba) and 3-amino-2,5-dichlorobenzoic acid (chloramben), and 2, 3, 6-trichlorobenzoic acid (TBD or TCBA), as shown in FIG. 14. The terms “dicamba”, “choramben” and “TBD” include the acids themselves, or their agriculturally acceptable esters and salts.

As used herein, “dicamba” refers to 3,6-dichloro-o-anisic acid or 3,6-dichloro-2-methoxy benzoic acid (FIG. 14) and its acids and salts. Dicamba salts include, for example, isopropylamine, diglycoamine, dimethylamine, potassium and sodium. Examples of commercial formulations of dicamba include, without limitation, Banvel™ (as DMA salt), Clarity® (as DGA salt, BASF), VEL-58-CS-11™ and Vanquish™ (as DGA salt, BASF).

A derivative of dicamba is defined as a substituted benzoic acid, and biologically acceptable salts thereof. In specific embodiments, the dicamba derivative has herbicidal activity.

Derivatives of dicamba further include metabolic products of the herbicide. In specific embodiments, decarboxylation of the dicamba metabolite can further reduce the herbicidal activity of the dicamba metabolite. In other embodiments, the dicamba metabolite does not have herbicidal activity, and the dicamba decarboxylase or active variant or fragment thereof is employed to modify the dicamba by-product, which in some instances finds use in bioremediation as disclosed elsewhere herein.

Non-limiting examples of dicamba metabolic products include any metabolic product produced when employing a dicamba monooxygenase. Dicamba monooxygenases (DMOs) and the various DMO-mediated dicamba metabolic products are described, for example in, U.S. Pat. No. 8,207,092, which is herein incorporated by reference in its entirety. Such, dicamba metabolic products include 3,6-DCSA, or DCGA (5-0H DCSA, or DC-gentisic acid. In one non-limiting embodiment, the dicamba decarboxylase is employed to decarboxylate 3,6-DCSA.

Methods and compositions are provided to detoxify an auxin-analog herbicide or derivative or metabolic product thereof. As used herein, “detoxify” or “detoxifying” an auxin-analog herbicide comprises any modification to the auxin-analog herbicide, derivative or metabolic product thereof, which reduces the herbicidal effect of the compound. A “reduced” herbicidal effect comprises any statistically significant decrease in the sensitivity of the plant or plant cell to the modified auxin-analog. The reduced herbicidal activity of a modified auxin-analog herbicide can be assayed in a variety of ways including, for example, assaying for the decreased sensitivity of a plant, a plant cell, or plant explant to the presence of the modified auxin-analog. See, for example, Example 2 provided herein. In such instances, the plant, plant cell, or plant explant will display a decreased sensitivity to the modified auxin-analog when compared to a control plant, plant cell, or plant explant which was contacted with the non-modified auxin-analog herbicide. Thus, in one example, a “reduced herbicidal effect” is demonstrated when plants display the increased tolerance to a modified auxin-analog and a dose/response curve is shifted to the right when compared to when the non-modified auxin-analog herbicide is applied. Such dose/response curves have “dose” plotted on the x-axis and “percentage injury”, “herbicidal effect” etc. plotted on the y-axis.

In one embodiment, methods and compositions are provided to detoxify dicamba via decarboxylation. The various bi-products of such an enzymatic reaction are set forth in FIG. 1 and discussed in detail elsewhere herein. As shown in Example 4, while the reaction mechanism may not be the same for all dicamba decarboxylases, all dicamba decarboxylases will release a CO2 from the dicamba molecule.

Thus, in one embodiment, a method for detoxifying an auxin-analog herbicide, derivative or metabolic product thereof is provided. Such methods employ increasing the level of a dicamba decarboxylase polypeptide or an active variant or fragment thereof in a plant, plant cell, plant part, explant, seed and applying to the plant, plant cell or plant part at least one auxin-analog herbicide. In specific embodiments, the auxin-analog herbicide comprises dicamba, derivative or metabolic product thereof.

In another embodiment, a method of producing an auxin-analog herbicide tolerant host cell (ie., a microbial cell such as E. coli) is provided and comprises introducing into the host cell (ie., the microbial cell, such as E. coli) a polynucleotide encoding a dicamba decarboxylase polypeptide or an active variant or fragment thereof. Microbial host cells expressing such dicamba decarboxylase sequences find use in bioremediation.

As used herein, “bioremediation” is the use of micro-organism metabolism to remove a contaminating material. In such embodiments, an effective amount of the microbial host expressing the dicamba decarboxylase polypeptide is contacted with a contaminated material (ie., soil) having an auxin-analog herbicide (such as, for example, dicamba). The microbial host detoxifies the auxin-analog herbicide and thereby reduces the level of the contaminant in the material (ie., soil). Such methods can occur either in situ or ex situ. In situ bioremediation involves treating the contaminated material at the site, while ex situ involves the removal of the contaminated material to be treated elsewhere.

In still further embodiments, the dicamba decarboxylase is employed to decarboxylate any auxin-analog, derivative or metabolic product thereof. In such methods, the dicamba decarboxylate can be found within a host cell or plant cell or alternatively, an effective amount of the dicamba decarboxylase can be applied to a sample containing the auxin-analog substrate. By “contacting” is intended any method whereby an effective amount of the auxin-analog substrate is exposed to the dicamba decarboxylase. By “effective amount” of the dicamba decarboxylase is intended an amount of chemical ligand that is sufficient to allow for the desirable level of decarboxylation of the substrate (i.e., auxin-analog or dicamba or derivative or metabolic product thereof).

C. Method of Producing Crops and Controlling Weeds

Methods for controlling weeds in an area of cultivation, preventing the development or the appearance of herbicide resistant weeds in an area of cultivation, producing a crop, and increasing crop safety are provided. The term “controlling,” and derivations thereof, for example, as in “controlling weeds” refers to one or more of inhibiting the growth, germination, reproduction, and/or proliferation of; and/or killing, removing, destroying, or otherwise diminishing the occurrence and/or activity of a weed.

As used herein, an “area of cultivation” comprises any region in which one desires to grow a plant. Such areas of cultivations include, but are not limited to, a field in which a plant is cultivated (such as a crop field, a sod field, a tree field, a managed forest, a field for culturing fruits and vegetables, etc), a greenhouse, a growth chamber, etc.

As used herein, by “selectively controlled” it is intended that the majority of weeds in an area of cultivation are significantly damaged or killed, while if crop plants are also present in the field, the majority of the crop plants are not significantly damaged. Thus, a method is considered to selectively control weeds when at least 55%, 60%, 65%, 70%, 75%, 80%, 85%, 90%, 95%, or more of the weeds are significantly damaged or killed, while if crop plants are also present in the field, less than 45%, 40%, 35%, 30%, 25%, 20%, 15%, 10%, 5%, or 1% of the crop plants are significantly damaged or killed.

Methods provided comprise planting the area of cultivation with a plant or a seed having a heterologous polynucleotide encoding a dicamba decarboxylase polypeptide or an active variant or fragment thereof, and in specific embodiments, applying to the crop, seed, weed and/or area of cultivation thereof an effective amount of a herbicide of interest. It is recognized that the herbicide can be applied before or after the crop is planted in the area of cultivation. Such herbicide applications can include an application of an auxin-analog herbicide including, but not limited to, the various an auxin-analog herbicides discussed elsewhere herein, non-limiting examples appearing in FIG. 14. In specific embodiments, the auxin-analog herbicide comprises dicamba. Generally, the effective amount of herbicide applied to the field is sufficient to selectively control the weeds without significantly affecting the crop.

“Weed” as used herein refers to a plant which is not desirable in a particular area. Conversely, a “crop plant” as used herein refers to a plant which is desired in a particular area, such as, for example, a maize or soybean plant. Thus, in some embodiments, a weed is a non-crop plant or a non-crop species, while in some embodiments, a weed is a crop species which is sought to be eliminated from a particular area, such as, for example, an inferior and/or non-transgenic soybean plant in a field planted with a plant having the heterologous nucleotide sequence encoding the dicamba decarboxylase polypeptide or an active variant or fragment thereof.

Further provided is a method for producing a crop by growing a crop plant that is tolerant to an auxin-analog herbicide or derivative thereof (i.e., dicamba or derivative thereof) as a result of being transformed with a heterologous polynucleotide encoding a dicamba decarboxylase polypeptide or an active variant or fragment thereof, under conditions such that the crop plant produces a crop, and harvesting the crop. Preferably, an auxin-analog herbicide or derivative thereof (i.e., dicamba or derivative thereof) is applied to the plant, or in the vicinity of the plant, or in the area of cultivation at a concentration effective to control weeds without preventing the transgenic crop plant from growing and producing the crop. The application of the auxin-analog herbicide can be before planting, or at any time after planting up to and including the time of harvest. The auxin-analog herbicide or derivative thereof can be applied once or multiple times. The timing of the auxin-analog herbicide application, amount applied, mode of application, and other parameters will vary based upon the specific nature of the crop plant and the growing environment. The invention further provides the crop produced by this method.

Further provided are methods for the propagation of a plant containing a heterologous polynucleotide encoding a dicamba decarboxylase polypeptide or active variant or fragment thereof. The plant can be, for example, a monocot or a dicot. In one aspect, propagation entails crossing a plant containing the heterologous polynucleotide encoding a dicamba decarboxylase polypeptide transgene with a second plant, such that at least some progeny of the cross display auxin-analog herbicide (i.e. dicamba) tolerance.

The methods of the invention further allow for the development of herbicide applications to be used with the plants having the heterologous polynucleotides encoding the dicamba decarboxylase polypeptides or active variants or fragments thereof. In such methods, the environmental conditions in an area of cultivation are evaluated. Environmental conditions that can be evaluated include, but are not limited to, ground and surface water pollution concerns, intended use of the crop, crop tolerance, soil residuals, weeds present in area of cultivation, soil texture, pH of soil, amount of organic matter in soil, application equipment, and tillage practices. Upon the evaluation of the environmental conditions, an effective amount of a combination of herbicides can be applied to the crop, crop part, seed of the crop or area of cultivation.

Any herbicide or combination of herbicides can be applied to the plant having the heterologous polynucleotide encoding the dicamba decarboxylase polypeptide or active variant or fragment thereof disclosed herein or transgenic seed derived there from, crop part, or the area of cultivation containing the crop plant. As mentioned elsewhere herein, such plants may further contain a polynucleotide encoding a polypeptide that confers tolerance to dicamba or a derivative thereof via a different mechanism than the dicamba decarboxylase, or the plant may further contain a polynucleotide encoding a polypeptide that confers tolerance to a herbicide other than dicamba.

By “treated with a combination of” or “applying a combination of” herbicides to a crop, area of cultivation or field it is intended that a particular field, crop or weed is treated with each of the herbicides and/or chemicals indicated to be part of the combination so that a desired effect is achieved, i.e., so that weeds are selectively controlled while the crop is not significantly damaged. The application of each herbicide and/or chemical may be simultaneous or the applications may be at different times (sequential), so long as the desired effect is achieved. Furthermore, the application can occur prior to the planting of the crop.

Classifications of herbicides (i.e., the grouping of herbicides into classes and subclasses) are well-known in the art and include classifications by HRAC (Herbicide Resistance Action Committee) and WSSA (the Weed Science Society of America) (see also, Retzinger and Mallory-Smith (1997) Weed Technology 11: 384-393). An abbreviated version of the HRAC classification (with notes regarding the corresponding WSSA group) is set forth below in Table 1.

Herbicides can be classified by their mode of action and/or site of action and can also be classified by the time at which they are applied (e.g., preemergent or postemergent), by the method of application (e.g., foliar application or soil application), or by how they are taken up by or affect the plant or by their structure. “Mode of action” generally refers to the metabolic or physiological process within the plant that the herbicide inhibits or otherwise impairs, whereas “site of action” generally refers to the physical location or biochemical site within the plant where the herbicide acts or directly interacts. Herbicides can be classified in various ways, including by mode of action and/or site of action (see, e.g., Table 1).

In specific embodiments, the plants of the present invention can tolerate treatment with different types of herbicides (i.e., herbicides having different modes of action and/or different sites of action) thereby permitting improved weed management strategies that are recommended in order to reduce the incidence and prevalence of herbicide-tolerant weeds.

In still further methods, an auxin-analog herbicide can be applied alone or in combination with another herbicide of interest and can be applied to the plants having the heterologous polynucleotide encoding the dicamba decarboxylase polypeptide or active variant or fragment thereof or their area of cultivation.

Additional herbicide treatment that can be applied over the plants or seeds having the heterologous polynucleotide encoding the dicamba decarboxylate polypeptide or an active variant or fragment thereof include, but are not limited to: acetochlor, acifluorfen and its sodium salt, aclonifen, acrolein (2-propenal), alachlor, alloxydim, ametryn, amicarbazone, amidosulfuron, aminopyralid, aminocyclopyrachlor, amitrole, ammonium sulfamate, anilofos, asulam, atrazine, azimsulfuron, beflubutamid, benazolin, benazolin-ethyl, bencarbazone, benfluralin, benfuresate, bensulfuron-methyl, bensulide, bentazone, benzobicyclon, benzofenap, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clethodim, clodinafop-propargyl, clomazone, clomeprop, clopyralid, clopyralid-olamine, cloransulam-methyl, CUH-35 (2-methoxyethyl 2-[[[4-chloro-2-fluoro-5-[(1-methyl-2-propynyl)oxy]phenyl](3-fluorobenzoyl)amino]carbonyl]-1-cyclohexene-1-carboxylate), cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethofumesate, ethoxyfen, ethoxysulfuron, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupyrsulfuron-methyl and its sodium salt, flurenol, flurenol-butyl, fluridone, flurochloridone, fluroxypyr, flurtamone, fluthiacet-methyl, fomesafen, foramsulfuron, fosamine-ammonium, glufosinate, glufosinate-ammonium, glyphosate and its salts such as ammonium, isopropylammonium, potassium, sodium (including sesquisodium) and trimesium (alternatively named sulfosate) (See, WO2007/024782, herein incorporated by reference in its entirety), halosulfuron-methyl, haloxyfop-etotyl, haloxyfop-methyl, hexazinone, HOK-201 (N-(2,4-difluorophenyl)-1,5-dihydro-N-(1-methylethyl)-5-oxo-1-[(tetrahydro-2H-pyran-2-yOmethyl]-4H-1,2,4-triazole-4-carboxamide), imazamethabenz-methyl, imazamox, imazapic, imazapyr, imazaquin, imazaquin-ammonium, imazethapyr, imazethapyr-ammonium, imazosulfuron, indanofan, iodosulfuron-methyl, ioxynil, ioxynil octanoate, ioxynil-sodium, isoproturon, isouron, isoxaben, isoxaflutole, pyrasulfotole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, methabenzthiazuron, methylarsonic acid and its calcium, monoammonium, monosodium and disodium salts, methyldymron, metobenzuron, metobromuron, metolachlor, S-metholachlor, metosulam, metoxuron, metribuzin, metsulfuron-methyl, molinate, monolinuron, naproanilide, napropamide, naptalam, neburon, nicosulfuron, norflurazon, orbencarb, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxyamid, phenmedipham, picloram, picloram-potassium, picolinafen, pinoxaden, piperofos, pretilachlor, primisulfuron-methyl, prodiamine, profoxydim, prometon, prometryn, propachlor, propanil, propaquizafop, propazine, propham, propisochlor, propoxycarbazone, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxsulam, quinclorac, quinmerac, quinoclamine, quizalofop-ethyl, quizalofop-P-ethyl, quizalofop-P-tefuryl, rimsulfuron, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, thenylchlor, thiazopyr, thiencarbazone, thifensulfuron-methyl, thiobencarb, tiocarbazil, topramezone, tralkoxydim, tri-allate, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate.

Additional herbicides include those that are applied over plants having homogentisate solanesyltransferase (HST) polypeptide such as those described in WO2010029311(A2), herein incorporate by reference it its entirety.

Other suitable herbicides and agricultural chemicals are known in the art, such as, for example, those described in WO 2005/041654. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub. Combinations of various herbicides can result in a greater-than-additive (i.e., synergistic) effect on weeds and/or a less-than-additive effect (i.e. safening) on crops or other desirable plants. In certain instances, combinations of auxin-analog herbicides with other herbicides having a similar spectrum of control but a different mode of action will be particularly advantageous for preventing the development of resistant weeds.

The time at which a herbicide is applied to an area of interest (and any plants therein) may be important in optimizing weed control. The time at which a herbicide is applied may be determined with reference to the size of plants and/or the stage of growth and/or development of plants in the area of interest, e.g., crop plants or weeds growing in the area.

Ranges of the effective amounts of herbicides can be found, for example, in various publications from University Extension services. See, for example, Bernards et al. (2006) Guide for Weed Management in Nebraska (www.ianrpubs.url.edu/sendlt/ec130); Regher et al. (2005) Chemical Weed Control for Fields Crops, Pastures, Rangeland, and Noncropland, Kansas State University Agricultural Extension Station and Corporate Extension Service; Zollinger et al. (2006) North Dakota Weed Control Guide, North Dakota Extension Service, and the Iowa State University Extension at www.weeds.iastate.edu, each of which is herein incorporated by reference in its entirety.

Many plant species can be controlled (i.e., killed or damaged) by the herbicides described herein. Accordingly, the methods of the invention are useful in controlling these plant species where they are undesirable (i.e., where they are weeds). These plant species include crop plants as well as species commonly considered weeds, including but not limited to species such as: blackgrass (Alopecurus myosuroides), giant foxtail (Setaria faberi), large crabgrass (Digitaria sanguinalis), Surinam grass (Brachiaria decumbens), wild oat (Avena fatua), common cocklebur (Xanthium pensylvanicum), common lambsquarters (Chenopodium album), morning glory (Ipomoea coccinea), pigweed (Amaranthus spp.), common waterhemp (Amaranthus tuberculatus), velvetleaf (Abutilion theophrasti), common barnyardgrass (Echinochloa crus-galli), bermudagrass (Cynodon dactylon), downy brome (Bromus tectorum), goosegrass (Eleucine indica), green foxtail (Setaria viridis), Italian ryegrass (Lolium multflorum), Johnsongrass (Sorghum halepense), lesser canarygrass (Phalaris minor), windgrass (Apera spica-venti), wooly cupgrass (Erichloa villosa), yellow nutsedge (Cyperus esculentus), common chickweed (Stellaria media), common ragweed (Ambrosia artemisiifolia), Kochia scoparia, horseweed (Conyza canadensis), rigid ryegrass (Lolium rigidum), goosegrass (Eleucine indica), hairy fleabane (Conyza bonariensis), buckhorn plantain (Plantago lanceolata), tropical spiderwort (Commelina benghalensis), field bindweed (Convolvulus arvensis), purple nutsedge (Cyperus rotundus), redvine (Brunnichia ovata), hemp sesbania (Sesbania exaltata), sicklepod (Senna obtusifolia), Texas blueweed (Helianthus ciliaris), and Devil's claws (Proboscidea louisianica). In other embodiments, the weed comprises a herbicide-resistant ryegrass, for example, a glyphosate resistant ryegrass, a paraquat resistant ryegrass, a ACCase-inhibitor resistant ryegrass, and a non-selective herbicide resistant ryegrass.

In some embodiments, a plant having the heterologous polynucleotide encoding the dicamba decarboxylase polypeptide or an active variant or fragment thereof is not significantly damaged by treatment with an auxin-analog herbicide (i.e., dicamba) applied to that plant, whereas an appropriate control plant is significantly damaged by the same treatment.

Generally, an auxin-analog herbicide (i.e., dicamba) is applied to a particular field (and any plants growing in it) no more than 1, 2, 3, 4, 5, 6, 7, or 8 times a year, or no more than 1, 2, 3, 4, or 5 times per growing season. Thus, methods of the invention encompass applications of herbicide which are “preemergent,” “postemergent,” “preplant incorporation” and/or which involve seed treatment prior to planting.

In one embodiment, methods are provided for coating seeds. The methods comprise coating a seed with an effective amount of a herbicide or a combination of herbicides (as disclosed elsewhere herein). The seeds can then be planted in an area of cultivation. Further provided are seeds having a coating comprising an effective amount of a herbicide or a combination of herbicides (as disclosed elsewhere herein). In other embodiments, the seeds can be coated with at least one fungicide and/or at least one insecticide and/or at least one herbicide or any combination thereof.

“Preemergent” refers to a herbicide which is applied to an area of interest (e.g., a field or area of cultivation) before a plant emerges visibly from the soil. “Postemergent” refers to a herbicide which is applied to an area after a plant emerges visibly from the soil. In some instances, the terms “preemergent” and “postemergent” are used with reference to a weed in an area of interest, and in some instances these terms are used with reference to a crop plant in an area of interest. When used with reference to a weed, these terms may apply to only a particular type of weed or species of weed that is present or believed to be present in the area of interest. While any herbicide may be applied in a preemergent and/or postemergent treatment, some herbicides are known to be more effective in controlling a weed or weeds when applied either preemergence or postemergence. For example, rimsulfuron has both preemergence and postemergence activity, while other herbicides have predominately preemergence (metolachlor) or postemergence (glyphosate) activity. These properties of particular herbicides are known in the art and are readily determined by one of skill in the art. Further, one of skill in the art would readily be able to select appropriate herbicides and application times for use with the transgenic plants of the invention and/or on areas in which transgenic plants of the invention are to be planted. “Preplant incorporation” involves the incorporation of compounds into the soil prior to planting.

Thus, improved methods of growing a crop and/or controlling weeds such as, for example, “pre-planting burn down,” are provided wherein an area is treated with herbicides prior to planting the crop of interest in order to better control weeds. The invention also provides methods of growing a crop and/or controlling weeds which are “no-till” or “low-till” (also referred to as “reduced tillage”). In such methods, the soil is not cultivated or is cultivated less frequently during the growing cycle in comparison to traditional methods; these methods can save costs that would otherwise be incurred due to additional cultivation, including labor and fuel costs.

The term “safener” refers to a substance that when added to a herbicide formulation eliminates or reduces the phytotoxic effects of the herbicide to certain crops. One of ordinary skill in the art would appreciate that the choice of safener depends, in part, on the crop plant of interest and the particular herbicide or combination of herbicides. Exemplary safeners suitable for use with the presently disclosed herbicide compositions include, but are not limited to, those disclosed in U.S. Pat. Nos. 4,808,208; 5,502,025; 6,124,240 and U.S. Patent Application Publication Nos. 2006/0148647; 2006/0030485; 2005/0233904; 2005/0049145; 2004/0224849; 2004/0224848; 2004/0224844; 2004/0157737; 2004/0018940; 2003/0171220; 2003/0130120; 2003/0078167, the disclosures of which are incorporated herein by reference in their entirety. The methods of the invention can involve the use of herbicides in combination with herbicide safeners such as benoxacor, BCS (1-bromo-4-[(chloromethyl)sulfonyl]benzene), cloquintocet-mexyl, cyometrinil, dichlormid, 2-(dichloromethyl)-2-methyl-1,3-dioxolane (MG 191), fenchlorazole-ethyl, fenclorim, flurazole, fluxofenim, furilazole, isoxadifen-ethyl, mefenpyr-diethyl, methoxyphenone ((4-methoxy-3-methylphenyl)(3-methylphenyl)-methanone), naphthalic anhydride (1,8-naphthalic anhydride) and oxabetrinil to increase crop safety. Antidotally effective amounts of the herbicide safeners can be applied at the same time as the compounds of this invention, or applied as seed treatments. Therefore an aspect of methods disclosed herein relates to the use of a mixture comprising an auxin-analog herbicide, at least one other herbicide, and an antidotally effective amount of a herbicide safener.

Seed treatment is useful for selective weed control, because it physically restricts antidoting to the crop plants. Therefore in one embodiment, a method for selectively controlling the growth of weeds in a field comprising treating the seed from which the crop is grown with an antidotally effective amount of safener and treating the field with an effective amount of herbicide to control weeds.

An antidotally effective amount of a safener is present where a desired plant is treated with the safener so that the effect of a herbicide on the plant is decreased in comparison to the effect of the herbicide on a plant that was not treated with the safener; generally, an antidotally effective amount of safener prevents damage or severe damage to the plant treated with the safener. One of skill in the art is capable of determining whether the use of a safener is appropriate and determining the dose at which a safener should be administered to a crop.

As used herein, an “adjuvant” is any material added to a spray solution or formulation to modify the action of an agricultural chemical or the physical properties of the spray solution. See, for example, Green and Foy (2003) “Adjuvants: Tools for Enhancing Herbicide Performance,” in Weed Biology and Management, ed. Inderjit (Kluwer Academic Publishers, The Netherlands). Adjuvants can be categorized or subclassified as activators, acidifiers, buffers, additives, adherents, antiflocculants, antifoamers, defoamers, antifreezes, attractants, basic blends, chelating agents, cleaners, colorants or dyes, compatibility agents, cosolvents, couplers, crop oil concentrates, deposition agents, detergents, dispersants, drift control agents, emulsifiers, evaporation reducers, extenders, fertilizers, foam markers, formulants, inerts, humectants, methylated seed oils, high load COCs, polymers, modified vegetable oils, penetrators, repellants, petroleum oil concentrates, preservatives, rainfast agents, retention aids, solubilizers, surfactants, spreaders, stickers, spreader stickers, synergists, thickeners, translocation aids, uv protectants, vegetable oils, water conditioners, and wetting agents.

In addition, methods of the invention can comprise the use of a herbicide or a mixture of herbicides, as well as, one or more other insecticides, fungicides, nematocides, bactericides, acaricides, growth regulators, chemosterilants, semiochemicals, repellents, attractants, pheromones, feeding stimulants or other biologically active compounds or entomopathogenic bacteria, virus, or fungi to form a multi-component mixture giving an even broader spectrum of agricultural protection. Examples of such agricultural protectants which can be used in methods of the invention include: insecticides such as abamectin, acephate, acetamiprid, amidoflumet (S-1955), avermectin, azadirachtin, azinphos-methyl, bifenthrin, bifenazate, buprofezin, carbofuran, cartap, chlorfenapyr, chlorfluazuron, chlorpyrifos, chlorpyrifos-methyl, chromafenozide, clothianidin, cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyriprole, pyriproxyfen, rotenone, ryanodine, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tralomethrin, triazamate, trichlorfon and triflumuron; fungicides such as acibenzolar, aldimorph, amisulbrom, azaconazole, azoxystrobin, benalaxyl, benomyl, benthiavalicarb, benthiavalicarb-isopropyl, binomial, biphenyl, bitertanol, blasticidin-S, Bordeaux mixture (Tribasic copper sulfate), boscalid/nicobifen, bromuconazole, bupirimate, buthiobate, carboxin, carpropamid, captafol, captan, carbendazim, chloroneb, chlorothalonil, chlozolinate, clotrimazole, copper oxychloride, copper salts such as copper sulfate and copper hydroxide, cyazofamid, cyflunamid, cymoxanil, cyproconazole, cyprodinil, dichlofluanid, diclocymet, diclomezine, dicloran, diethofencarb, difenoconazole, dimethomorph, dimoxystrobin, diniconazole, diniconazole-M, dinocap, discostrobin, dithianon, dodemorph, dodine, econazole, etaconazole, edifenphos, epoxiconazole, ethaboxam, ethirimol, ethridiazole, famoxadone, fenamidone, fenarimol, fenbuconazole, fencaramid, fenfuram, fenhexamide, fenoxanil, fenpiclonil, fenpropidin, fenpropimorph, fentin acetate, fentin hydroxide, ferbam, ferfurazoate, ferimzone, fluazinam, fludioxonil, flumetover, fluopicolide, fluoxastrobin, fluquinconazole, fluquinconazole, flusilazole, flusulfamide, flutolanil, flutriafol, folpet, fosetyl-aluminum, fuberidazole, furalaxyl, furametapyr, hexaconazole, hymexazole, guazatine, imazalil, imibenconazole, iminoctadine, iodicarb, ipconazole, iprobenfos, iprodione, iprovalicarb, isoconazole, isoprothiolane, kasugamycin, kresoxim-methyl, mancozeb, mandipropamid, maneb, mapanipyrin, mefenoxam, mepronil, metalaxyl, metconazole, methasulfocarb, metiram, metominostrobin/fenominostrobin, mepanipyrim, metrafenone, miconazole, myclobutanil, neo-asozin (ferric methanearsonate), nuarimol, octhilinone, ofurace, orysastrobin, oxadixyl, oxolinic acid, oxpoconazole, oxycarboxin, paclobutrazol, penconazole, pencycuron, penthiopyrad, perfurazoate, phosphonic acid, phthalide, picobenzamid, picoxystrobin, polyoxin, probenazole, prochloraz, procymidone, propamocarb, propamocarb-hydrochloride, propiconazole, propineb, proquinazid, prothioconazole, pyraclostrobin, pryazophos, pyrifenox, pyrimethanil, pyrifenox, pyrolnitrine, pyroquilon, quinconazole, quinoxyfen, quintozene, silthiofam, simeconazole, spiroxamine, streptomycin, sulfur, tebuconazole, techrazene, tecloftalam, tecnazene, tetraconazole, thiabendazole, thifluzamide, thiophanate, thiophanate-methyl, thiram, tiadinil, tolclofos-methyl, tolyfluanid, triadimefon, triadimenol, triarimol, triazoxide, tridemorph, trimoprhamide tricyclazole, trifloxystrobin, triforine, triticonazole, uniconazole, validamycin, vinclozolin, zineb, ziram, and zoxamide; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including entomopathogenic bacteria, such as Bacillus thuringiensis subsp. Aizawai, Bacillus thuringiensis subsp. Kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

The methods of controlling weeds can further include the application of a biologically effective amount of a herbicide of interest or a mixture of herbicides, and an effective amount of at least one additional biologically active compound or agent and can further comprise at least one of a surfactant, a solid diluent or a liquid diluent. Examples of such biologically active compounds or agents are: insecticides such as abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenyl-amino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metomino-strobin/fenominostrobin (SSF-126), metrafenone (AC375839), myclobutanil, neo-asozin (ferric methane-arsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including entomopathogenic bacteria, such as Bacillus thuringiensis subsp. Aizawai, Bacillus thuringiensis subsp. Kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV. Methods of the invention may also comprise the use of plants genetically transformed to express proteins (such as Bacillus thuringiensis delta-endotoxins) toxic to invertebrate pests. In such embodiments, the effect of exogenously applied invertebrate pest control compounds may be synergistic with the expressed toxin proteins. General references for these agricultural protectants include The Pesticide Manual, 13th Edition, C. D. S. Tomlin, Ed., British Crop Protection Council, Farnham, Surrey, U. K., 2003 and The BioPesticide Manual, 2^{nd} Edition, L. G. Copping, Ed., British Crop Protection Council, Farnham, Surrey, U. K., 2001. In certain instances, combinations with other invertebrate pest control compounds or agents having a similar spectrum of control but a different mode of action will be particularly advantageous for resistance management. Thus, compositions of the present invention can further comprise a biologically effective amount of at least one additional invertebrate pest control compound or agent having a similar spectrum of control but a different mode of action. Contacting a plant genetically modified to express a plant protection compound (e.g., protein) or the locus of the plant with a biologically effective amount of a compound of this invention can also provide a broader spectrum of plant protection and be advantageous for resistance management.

Thus, methods of controlling weeds can employ a herbicide or herbicide combination and may further comprise the use of insecticides and/or fungicides, and/or other agricultural chemicals such as fertilizers. The use of such combined treatments of the invention can broaden the spectrum of activity against additional weed species and suppress the proliferation of any resistant biotypes.

Methods can further comprise the use of plant growth regulators such as aviglycine, N-(phenylmethyl)-1H-purin-6-amine, ethephon, epocholeone, gibberellic acid, gibberellin A₄ and A₇, hatpin protein, mepiquat chloride, prohexadione calcium, prohydrojasmon, sodium nitrophenolate and trinexapac-methyl, and plant growth modifying organisms such as Bacillus cereus strain BP01.

### IIX. Method of Detection

Methods for detecting a dicamba decarboxylase polypeptide or an active variant or fragment thereof are provided. Such methods comprise analyzing samples, including environmental samples or plant tissues to detect such polypeptides or the polynucleotides encoding the same. The detection methods can directly assay for the presence of the dicamba decarboxylase polypeptide or polynucleotide or the detection methods can indirectly assay for the sequences by assaying the phenotype of the host cell, plant, plant cell or plant explant expressing the sequence.

In one embodiment, the dicamba decarboxylase polypeptide is detected in the sample or the plant tissue using an immunoassay comprising an antibody or antibodies that specifically recognizes a dicamba decarboxylase polypeptide or active variant or fragment thereof. In specific embodiments, the antibody or antibodies which are used are raised to a dicamba decarboxylase polypeptide or active variant or fragment thereof as disclosed herein.

By “specifically or selectively binds” is intended that the binding agent has a binding affinity for a given dicamba decarboxylase polypeptide or fragment or variant disclosed herein, which is greater than 10%, 9%, 8%, 7%, 6%, 5%, 4%, 3%, 2% or 1% of the binding affinity for a known dicamba decarboxylase sequence. One of skill will be aware of the proper controls that are needed to carry out such a determination.

By “antibodies that specifically bind” is intended that the antibodies will not substantially cross react with another polypeptide. By “not substantially cross react” is intended that the antibody or fragment thereof has a binding affinity for the other polypeptide which is less than 10%, less than 5%, or less than 1%, of the binding affinity for the dicamba decarboxylase polypeptide or active fragment or variant thereof.

In still other embodiments, the dicamba decarboxylase polypeptide or active variant or fragment thereof can be detected in a sample or a plant tissue by detecting the presence of a polynucleotide encoding any of the various dicamba decarboxylase polypeptides or active variants and fragments thereof. In one embodiment, the detection method comprises assaying the sample or the plant tissue using PCR amplification.

As used herein, “primers” are isolated polynucleotides that are annealed to a complementary target DNA strand by nucleic acid hybridization to form a hybrid between the primer and the target DNA strand, then extended along the target DNA strand by a polymerase, e.g., a DNA polymerase. Primer pairs of the invention refer to their use for amplification of a target polynucleotide, e.g., by the polymerase chain reaction (PCR) or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference in their entirety).

Probes and primers are of sufficient nucleotide length to bind to the target DNA sequence and specifically detect and/or identify a polynucleotide encoding a dicamba decarboxylase polypeptide or active variant or fragment thereof as described elsewhere herein. It is recognized that the hybridization conditions or reaction conditions can be determined by the operator to achieve this result. This length may be of any length that is of sufficient length to be useful in a detection method of choice. Such probes and primers can hybridize specifically to a target sequence under high stringency hybridization conditions. Probes and primers according to embodiments of the present invention may have complete DNA sequence identity of contiguous nucleotides with the target sequence, although probes differing from the target DNA sequence and that retain the ability to specifically detect and/or identify a target DNA sequence may be designed by conventional methods. Accordingly, probes and primers can share about 80%, 85%, 90%, 91%, 92%, 93%, 94%, 95%, 96%, 97%, 98%, 99% or greater sequence identity or complementarity to the target polynucleotide.

Methods for preparing and using probes and primers are described, for example, in Molecular Cloning: A Laboratory Manual, 2.sup.nd ed, vol. 1-3, ed. Sambrook et al., Cold Spring Harbor Laboratory Press, Cold Spring Harbor, N. Y. 1989 (hereinafter, “Sambrook et al., 1989”); Current Protocols in Molecular Biology, ed. Ausubel et al., Greene Publishing and Wiley-Interscience, New York, 1992 (with periodic updates) (hereinafter, “Ausubel et al., 1992”); and Innis et al., PCR Protocols: A Guide to Methods and Applications, Academic Press: San Diego, 1990. PCR primer pairs can be derived from a known sequence, for example, by using computer programs intended for that purpose such as the PCR primer analysis tool in Vector NTI version 10 (Invitrogen); PrimerSelect (DNASTAR Inc., Madison, Wis.); and Primer (Version 0.5.COPYRGT., 1991, Whitehead Institute for Biomedical Research, Cambridge, Mass.). Additionally, the sequence can be visually scanned and primers manually identified using guidelines known to one of skill in the art.

### IX. Method of Identifying Dicamba Decarboxylase Variants

Various methods can be employed to identify further dicamba decarboxylase variants. The polynucleotides are optionally used as substrates for a variety of diversity generating procedures or for rational enzyme design.

i. Methods of Generating Diversity in Dicamba Decarboxylases

A variety of diversity generating procedures, e.g., mutation, recombination and recursive recombination reactions can be employed, in addition to their use in standard cloning methods as set forth in, e.g., Ausubel, Berger and Sambrook, i.e., to produce additional dicamba decarboxylase polynucleotides and polypeptides with desired properties. A variety of diversity generating protocols can be used. The procedures can be used separately, and/or in combination to produce one or more variants of a polynucleotide or set of polynucleotides, as well variants of encoded proteins. Individually and collectively, these procedures provide robust, widely applicable ways of generating diversified polynucleotides and sets of polynucleotides (including, e.g., polynucleotide libraries) useful, e.g., for the engineering or rapid evolution of polynucleotides, proteins, pathways, cells and/or organisms with new and/or improved characteristics. The process of altering the sequence can result in, for example, single nucleotide substitutions, multiple nucleotide substitutions, and insertion or deletion of regions of the nucleic acid sequence.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more polynucleotides, which can be selected or screened for polynucleotides that encode proteins with or which confer desirable properties. Following diversification by one or more of the methods herein, or otherwise available to one of skill, any polynucleotides that are produced can be selected for a desired activity or property, e.g. altered K_{M}, use of alternative cofactors, increased k_{cat}, etc. This can include identifying any activity that can be detected, for example, in an automated or automatable format, by any of the assays in the art. For example, modified dicamba decarboxylase polypeptides can be detected by assaying for dicamba decarboxylation activity. Assays to measure such activity are described elsewhere herein. A variety of related (or even unrelated) properties can be evaluated, in serial or in parallel, at the discretion of the practitioner.

Descriptions of a variety of diversity generating procedures, including family shuffling and methods for generating modified nucleic acid sequences encoding multiple enzymatic domains, are found in the following publications and the references cited therein: Soong N. et al. (2000) Nat Genet 25(4):436-39; Stemmer et al. (1999) Tumor Targeting 4:1-4; Ness et al. (1999) Nature Biotechnology 17:893-896; Chang et al. (1999) Nature Biotechnology 17:793-797; Minshull and Stemmer (1999) Current Opinion in Chemical Biology 3:284-290; Christians et al. (1999) Nature Biotechnology 17:259-264; Crameri et al. (1998) Nature 391:288-291; Crameri et al. (1997) Nature Biotechnology 15:436-438; Zhang et al. (1997) Proc. Natl. Acad. Sci. USA 94:4504-4509; Patten et al. (1997) Current Opinion in Biotechnology 8:724-733; Crameri et al. (1996) Nature Medicine 2:100-103; Crameri et al. (1996) Nature Biotechnology 14:315-319; Gates et al. (1996) Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) BioTechniques 18:194-195; Stemmer et al. (1995) Gene: 164:49-53; Stemmer (1995) Science 270: 1510; Stemmer (1995) Bio/Technology 13:549-553; Stemmer (1994) Nature 370:389-391; and Stemmer (1994) Proc. Natl. Acad. Sci. USA 91:10747-10751. See also WO2008/073877 and US 20070204369, both of which are herein incorporated by reference in their entirety.

Mutational methods of generating diversity include, for example, site-directed mutagenesis (Ling et al. (1997) Anal Biochem. 254(2): 157-178; Dale et al. (1996) Methods Mol. Biol. 57:369-374; Smith (1985) Ann. Rev. Genet. 19:423-462; Botstein & Shortle (1985) Science 229:1193-1201; Carter (1986) Biochem. J. 237:1-7; and Kunkel (1987) Nucleic Acids & Molecular Biology (Eckstein, F. and Lilley, D. M. J. eds., Springer Verlag, Berlin)); mutagenesis using uracil containing templates (Kunkel (1985) Proc. Natl. Acad. Sci. USA 82:488-492; Kunkel et al. (1987) Methods in Enzymol. 154, 367-382; and Bass et al. (1988) Science 242:240-245); oligonucleotide-directed mutagenesis (Methods in Enzymol. 100: 468-500 (1983); Methods in Enzymol. 154: 329-350 (1987); Zoller & Smith (1982) Nucleic Acids Res. 10:6487-6500; Zoller & Smith (1983) Methods in Enzymol. 100:468-500; and Zoller & Smith (1987) Methods in Enzymol. 154:329-350); phosphorothioate-modified DNA mutagenesis (Taylor et al. (1985) Nucl. Acids Res. 13: 8749-8764; Taylor et al. (1985) Nuc. Acids Res. 13: 8765-8787 (1985); Nakamaye & Eckstein (1986) Nucl. Acids Res. 14: 9679-9698; Sayers et al. (1988) Nucl. Acids Res. 16:791-802; and Sayers et al. (1988) Nucl. Acids Res. 16: 803-814); mutagenesis using gapped duplex DNA (Kramer et al. (1984) Nucl. Acids Res. 12: 9441-9456; Kramer & Fritz (1987) Methods in Enzymol. 154:350-367; Kramer et al. (1988) Nucl. Acids Res. 16: 7207; and Fritz et al. (1988) Nucl. Acids Res. 16: 6987-6999).

Additional suitable methods include, but are not limited to, point mismatch repair (Kramer et al. (1984) Cell 38:879-887), mutagenesis using repair-deficient host strains (Carter et al. (1985) Nucl. Acids Res. 13: 4431-4443; and Carter (1987) Methods in Enzymol. 154: 382-403), deletion mutagenesis (Eghtedarzadeh & Henikoff (1986) Nucl. Acids Res. 14: 5115), restriction-selection and restriction-purification (Wells et al. (1986) Phil. Trans. R. Soc. Lond. A 317: 415-423), mutagenesis by total gene synthesis (Nambiar et al. (1984) Science 223: 1299-1301; Sakamar and Khorana (1988) Nucl. Acids Res. 14: 6361-6372; Wells et al. (1985) Gene 34:315-323; and Grundström et al. (1985) Nucl. Acids Res. 13: 3305-3316), and double-strand break repair (Mandecki (1986); Arnold (1993) Current Opinion in Biotechnology 4:450-455 and Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793, U.S. Pat. No. 5,811,238, U.S. Pat. No. 5,830,721, U.S. Pat. No. 5,834,252, U.S. Pat. No. 5,837,458, WO 95/22625, WO 96/33207, WO 97/20078, WO 97/35966, WO 99/41402, WO 99/41383, WO 99/41369, WO 99/41368, EP 752008, EP 0932670, WO 99/23107, WO 99/21979, WO 98/31837, WO 98/27230, WO 98/13487, WO 00/00632, WO 00/09679, WO 98/42832, WO 99/29902, WO 98/41653, WO 98/41622, WO 98/42727, WO 00/18906, WO 00/04190, WO 00/42561, WO 00/42559, WO 00/42560, WO 01/23401, and, PCT/US01/06775. See, also WO20074303, herein incorporated by reference in their entirety.

In brief, several different general classes of sequence modification methods, such as mutation, recombination, etc. are applicable to the present invention and set forth, e.g., in the references above. That is, alterations to the component nucleic acid sequences to produced modified gene fusion constructs can be performed by any number of the protocols described, either before cojoining of the sequences, or after the cojoining step. The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants are described in several of the references above, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 and in PCT/US99/15972. Thus, any of these processes and techniques for recombination, recursive recombination, and whole genome recombination, alone or in combination, can be used to generate the modified nucleic acid sequences and/or modified gene fusion constructs of the present invention.

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561, WO 01/23401, WO 00/42560, and, WO 00/42559.

In silico methods of recombination can be affected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 and WO 00/42559.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408 and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) Nature Biotech 17:1205. This approach can be used to generate an initial a library of variants which can optionally serve as a substrate for one or more in vitro or in vivo recombination methods. See, also, Ostermeier et al. (1999) Proc. Natl. Acad. Sci. USA, 96: 3562-67; Ostermeier et al. (1999), Biological and Medicinal Chemistry 7: 2139-44.

Mutational methods which result in the alteration of individual nucleotides or groups of contiguous or non-contiguous nucleotides can be favorably employed to introduce nucleotide diversity into the nucleic acid sequences and/or gene fusion constructs of the present invention. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., U.S. Pat. No. 5,783,431 and U.S. Pat. No. 5,824,485) and their use to identify protein activities of interest has been proposed (In addition to the references noted above, see, U.S. Pat. No. 5,958,672. Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) Gene 215: 471) prior to diversifying according to any of the methods described herein.

Libraries can be biased towards nucleic acids which encode proteins with desirable enzyme activities. For example, after identifying a variant from a library which exhibits a specified activity, the variant can be mutagenized using any known method for introducing DNA alterations. A library comprising the mutagenized homologues is then screened for a desired activity, which can be the same as or different from the initially specified activity. An example of such a procedure is proposed in U.S. Pat. No. 5,939,250. Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived there from. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are found in WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) Biotechnology 10:297-300; Reidhaar-Olson et al. (1991) Methods Enzymol. 208:564-86; Lim and Sauer (1991) J. Mol. Biol. 219:359-76; Breyer and Sauer (1989) J. Biol. Chem. 264:13355-60); and U.S. Pat. Nos. 5,830,650 and 5,798,208, and EP Patent 0527809 B1.

It will readily be appreciated that any of the above described techniques suitable for enriching a library prior to diversification can also be used to screen the products, or libraries of products, produced by the diversity generating methods. Any of the above described methods can be practiced recursively or in combination to alter nucleic acids, e.g., dicamba decarboxylase encoding polynucleotides.

The above references provide many mutational formats, including recombination, recursive recombination, recursive mutation and combinations or recombination with other forms of mutagenesis, as well as many modifications of these formats. Regardless of the diversity generation format that is used, the nucleic acids of the present invention can be recombined (with each other, or with related (or even unrelated) sequences) to produce a diverse set of recombinant nucleic acids for use in the gene fusion constructs and modified gene fusion constructs of the present invention, including, e.g., sets of homologous nucleic acids, as well as corresponding polypeptides.

Many of the above-described methodologies for generating modified polynucleotides generate a large number of diverse variants of a parental sequence or sequences. In some embodiments, the modification technique (e.g., some form of shuffling) is used to generate a library of variants that is then screened for a modified polynucleotide or pool of modified polynucleotides encoding some desired functional attribute, e.g., maintained or improved dicamba decarboxylase activity.

One example of selection for a desired enzymatic activity entails growing host cells under conditions that inhibit the growth and/or survival of cells that do not sufficiently express an enzymatic activity of interest, e.g. the dicamba decarboxylase activity. Using such a selection process can eliminate from consideration all modified polynucleotides except those encoding a desired enzymatic activity. For example, in some embodiments of the invention host cells are maintained under conditions that inhibit cell growth or survival in the presence of sufficient levels of dicamba. Under these conditions, only a host cell harboring a dicamba decarboxylase enzymatic activity or activities that is able to decarboxylase the dicamba will survive and grow. Some embodiments of the invention employ multiples rounds of screening at increasing concentrations of dicamba.

For convenience and high throughput it will often be desirable to screen/select for desired modified nucleic acids in a microorganism, e.g., a bacteria such as E. coli. On the other hand, screening in plant cells or plants can in some cases be preferable where the ultimate aim is to generate a modified nucleic acid for expression in a plant system.

In some preferred embodiments of the invention throughput is increased by screening pools of host cells expressing different modified nucleic acids, either alone or as part of a gene fusion construct. Any pools showing significant activity can be deconvoluted to identify single variants expressing the desirable activity.

In high throughput assays, it is possible to screen up to several thousand different variants in a single day. For example, each well of a microtiter plate can be used to run a separate assay, or, if concentration or incubation time effects are to be observed, every 5-10 wells can test a single variant.

In addition to fluidic approaches, it is possible, as mentioned above, simply to grow cells on media plates that select for the desired enzymatic or metabolic function. This approach offers a simple and high-throughput screening method.

A number of well known robotic systems have also been developed for solution phase chemistries useful in assay systems. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, CA) which mimic the manual synthetic operations performed by a scientist. Any of the above devices are suitable for application to the present invention. The nature and implementation of modifications to these devices (if any) so that they can operate as discussed herein with reference to the integrated system will be apparent to persons skilled in the relevant art.

High throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization.

The manufacturers of such systems provide detailed protocols for the various high throughput devices. Thus, for example, Zymark Corp. provides technical bulletins describing screening systems for detecting the modulation of gene transcription, ligand binding, and the like. Microfluidic approaches to reagent manipulation have also been developed, e.g., by Caliper Technologies (Mountain View, Calif.).

### X. Sequence Comparisons

The following terms are used to describe the sequence relationships between two or more polynucleotides or polypeptides: (a) “reference sequence”, (b) “comparison window”, (c) “sequence identity”, and, (d) “percent sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence or protein sequence.

(b) As used herein, “comparison window” makes reference to a contiguous and specified segment of a polypeptide sequence, wherein the polypeptide sequence in the comparison window may comprise additions or deletions (i.e., gaps) compared to the reference sequence (which does not comprise additions or deletions) for optimal alignment of the two polypeptides. Generally, the comparison window is at least 5, 10, 15, or 20 contiguous amino acid in length, or it can be 30, 40, 50, 100, or longer. Those of skill in the art understand that to avoid a high similarity to a reference sequence due to inclusion of gaps in the polypeptide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. BLASTP protein searches can be performed using default parameters. See, blast.ncbi.nlm.nih.gov/Blast.cgi.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTP for proteins) can be used. See www.ncbi.nlm.nih.gov. Alignment may also be performed manually by inspection.

In one embodiment, sequence identity/similarity values provided herein refer to the value obtained using GAP Version 10 using the following parameters: % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity). When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percent sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percent sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

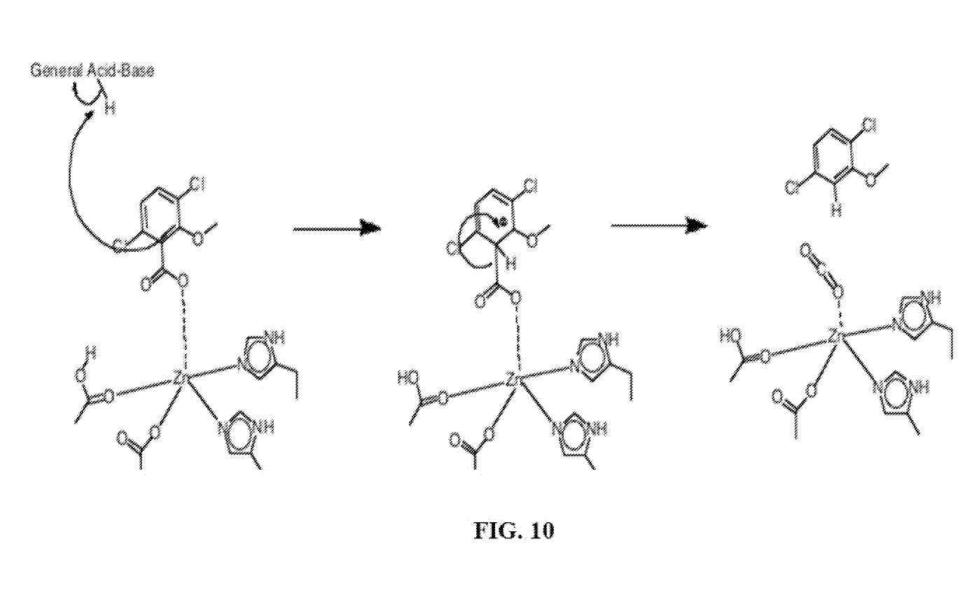

(e) Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acids substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found, Washington, D.C. and Henikoff et al. (1992) Proc. Natl. Acad. Sci. USA 89:10915-10919. The BLOSUM62 matrix (FIG. 10) is often used as a default scoring substitution matrix in sequence alignment protocols such as Gapped BLAST 2.0. The gap existence penalty is imposed for the introduction of a single amino acid gap in one of the aligned sequences, and the gap extension penalty is imposed for each additional empty amino acid position inserted into an already opened gap. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al, (1997) Nucleic Acids Res. 25:3389-3402, and made available to the public at the National Center for Biotechnology Information Website (http://www.ncbi.nlm.nih.gov). Optimal alignments, including multiple alignments, can be prepared using, e.g., PSI-BLAST, available through http://www.ncbi.nlm.nih.gov and described by Altschul et al, (1997) Nucleic Acids Res. 25:3389-3402.

As used herein, similarity score and bit score is determined employing the BLAST alignment used the BLOSUM62 substitution matrix, a gap existence penalty of 11, and a gap extension penalty of 1. For the same pair of sequences, if there is a numerical difference between the scores obtained when using one or the other sequence as query sequences, a greater value of similarity score is selected.

Non-limiting embodiments include:

1. A plant cell having stably incorporated into its genome a heterologous polynucleotide encoding a polypeptide having dicamba decarboxylase activity.

2. The plant cell of embodiment 1, wherein said polypeptide having dicamba decarboxylase activity comprises an active site having a catalytic residue geometry as set forth in Table 3 or having a substantially similar catalytic residue geometry.

3. The plant cell of embodiment 2, wherein said polypeptide having dicamba decarboxylase activity further comprises:

(a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1;

(b) an amino acid sequence having at least 60%, 70%, 75%, 80% 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129;

(c) an amino acid sequence having at least 60% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129, wherein
(i) the amino acid residue in the encoded polypeptide that corresponds to amino acid position 27 of SEQ ID NO: 109 comprises alanine, serine, or threonine;
(ii) the amino acid residue in the encoded polypeptide that corresponds to amino acid position 38 of SEQ ID NO: 109 comprises isoleucine;
(iii) the amino acid residue in the encoded polypeptide that corresponds to amino acid position 42 of SEQ ID NO: 109 comprises alanine, methionine, or serine;
(iv) the amino acid residue in the encoded polypeptide that corresponds to amino acid position 52 of SEQ ID NO: 109 comprises glutamic acid;
(v) the amino acid residue in the encoded polypeptide that corresponds to amino acid position 61 of SEQ ID NO: 109 comprises alanine or serine;
(vi) the amino acid residue in the encoded polypeptide that corresponds to amino acid position 64 of SEQ ID NO: 109 comprises glycine, or serine;
(vii) the amino acid residue in the encoded polypeptide that corresponds to amino acid position 127 of SEQ ID NO: 109 comprises methionine;
(iix) the amino acid residue in the encoded polypeptide that corresponds to amino acid position 238 of SEQ ID NO: 109 comprises glycine;
(ix) the amino acid residue in the encoded polypeptide that corresponds to amino acid position 240 of SEQ ID NO: 109 comprises alanine, aspartic acid, or glutamic acid;
(x) the amino acid residue in the encoded polypeptide that corresponds to amino acid position 298 of SEQ ID NO: 109 comprises alanine or threonine,
(xi) the amino acid residue in the encoded polypeptide that corresponds to amino acid position 299 of SEQ ID NO: 109 comprises alanine;
(xii) the amino acid residue in the encoded polypeptide that corresponds to amino acid position 303 of SEQ ID NO: 109 comprises cysteine, glutamic acid, or serine;
(xiii) the amino acid residue in the encoded polypeptide that corresponds to amino acid position 327 of SEQ ID NO: 109 comprises leucine, glutamine, or valine; or,
(ixv) the amino acid residue in the encoded polypeptide that corresponds to amino acid position 328 of SEQ ID NO: 109 comprises aspartic acid, arginine, or serine;
(xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

4. The plant cell of embodiment 1, wherein said polypeptide comprises:
(a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1;
(b) an amino acid sequence having at least 85%, 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; or,
(c) an amino acid sequence having at least 60% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129, and wherein
   (i) the amino acid residue in the encoded polypeptide that corresponds to amino acid position 27 of SEQ ID NO: 109 comprises alanine, serine, or threonine;
   (ii) the amino acid residue in the encoded polypeptide that corresponds to amino acid position 38 of SEQ ID NO: 109 comprises isoleucine;
   (iii) the amino acid residue in the encoded polypeptide that corresponds to amino acid position 42 of SEQ ID NO: 109 comprises alanine, methionine, or serine;
   (iv) the amino acid residue in the encoded polypeptide that corresponds to amino acid position 52 of SEQ ID NO: 109 comprises glutamic acid;
   (v) the amino acid residue in the encoded polypeptide that corresponds to amino acid position 61 of SEQ ID NO: 109 comprises alanine or serine;
   (vi) the amino acid residue in the encoded polypeptide that corresponds to amino acid position 64 of SEQ ID NO: 109 comprises glycine, or serine;
   (vii) the amino acid residue in the encoded polypeptide that corresponds to amino acid position 127 of SEQ ID NO: 109 comprises methionine;
   (iix) the amino acid residue in the encoded polypeptide that corresponds to amino acid position 238 of SEQ ID NO: 109 comprises glycine;
   (ix) the amino acid residue in the encoded polypeptide that corresponds to amino acid position 240 of SEQ ID NO: 109 comprises alanine, aspartic acid, or glutamic acid;
   (x) the amino acid residue in the encoded polypeptide that corresponds to amino acid position 298 of SEQ ID NO: 109 comprises alanine or threonine,
   (xi) the amino acid residue in the encoded polypeptide that corresponds to amino acid position 299 of SEQ ID NO: 109 comprises alanine;
   (xii) the amino acid residue in the encoded polypeptide that corresponds to amino acid position 303 of SEQ ID NO: 109 comprises cysteine, glutamic acid, or serine;
   (xiii) the amino acid residue in the encoded polypeptide that corresponds to amino acid position 327 of SEQ ID NO: 109 comprises leucine, glutamine, or valine;
   (ixv) the amino acid residue in the encoded polypeptide that corresponds to amino acid position 328 of SEQ ID NO: 109 comprises aspartic acid, arginine, or serine; and/or,
   (xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

5. The plant cell of any one of embodiments 1-4, wherein said polypeptide having dicamba decarboxylase activity has a k_{cat}/Kₘ of at least 0.0001 mM⁻¹ min⁻¹ for dicamba.

6. The plant cell of any one of embodiments 1-5, wherein the plant cell exhibits enhanced resistance to dicamba as compared to a wild type plant cell of the same species, strain or cultivar.

7. The plant cell of any one of embodiments 1-6, wherein said plant cell is from a monocot.

8. The plant cell of embodiment 7, wherein said monocot is maize, wheat, rice, barley, sugarcane, sorghum, or rye.

9. The plant cell of any one of embodiments 1-6, wherein said plant cell is from a dicot.

10. The plant cell of embodiment 9, wherein the dicot is soybean, Brassica, sunflower, cotton, or alfalfa.

11. A plant comprising a plant cell of any one of embodiments 1-10.

12. The plant of embodiment 11, wherein the plant exhibits tolerance to dicamba applied at a level effective to inhibit the growth of the same plant lacking the polypeptide having dicamba decarboxylase activity, without significant yield reduction due to herbicide application.

13. A plant explant comprising a plant cell of any one of embodiments 1-10.

14. The plant, the explant, or the plant cell of any one of embodiments 1-13, wherein the plant, the explant or the plant cell further comprises at least one polypeptide imparting tolerance to an additional herbicide.

15. The plant, the explant, or the plant cell of embodiment 14, wherein said at least one polypeptide imparting tolerance to an additional herbicide comprises:
(a) a sulfonylurea-tolerant acetolactate synthase;
(b) an imidazolinone-tolerant acetolactate synthase;
(c) a glyphosate-tolerant 5-enolpyruvylshikimate-3-phosphate synthase;
(d) a glyphosate-tolerant glyphosate oxido-reductase;
(e) a glyphosate-N-acetyltransferase;
(f) a phosphinothricin acetyl transferase;
(g) a protoporphyrinogen oxidase or a protoporphorinogen detoxification enzyme;
(h) an auxin enzyme or auxin tolerance protein;
(i) a P450 polypeptide;
(j) an acetyl coenzyme A carboxylase (ACCase);
(k) a high resistance allele of acetolactate synthase (HRA);
(l) a hydroxyphenylpyruvate dioxygenase (HPPD) or an HPPD detoxification enzyme; and/or,
(j) a dicamba monooxygenase.

16. The plant, the explant, or the plant cell of embodiment 14, wherein said at least one polypeptide imparting tolerance to an additional herbicide confers tolerance to 2,4 D or comprise an aryloxyalkanoate di-oxygenase.

17. The plant, the explant, or the plant cell of any one of embodiments 1-16, wherein the plant, the explant or the plant cell further comprises at least one additional polypeptide imparting tolerance to dicamba.

18. A transgenic seed produced by the plant of any one of embodiments 12 or 14-17.

19. A method of producing a plant cell having a heterologous polynucleotide encoding a polypeptide having dicamba decarboxylase activity comprising transforming said plant cell with a heterologous polynucleotide encoding a polypeptide having dicamba decarboxylase activity.

20. The method of embodiment 19, wherein said polypeptide having dicamba decarboxylase activity comprises an active site having a catalytic residue geometry as set forth in Table 3 or having a substantially similar catalytic residue geometry.

21. The method of embodiment 20, wherein said polypeptide having dicamba decarboxylase activity comprises

(a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1;

(b) an amino acid sequence having at least 60%, 70%, 75%, 80% 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; or,

(c) an amino acid sequence having at least 60% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129 and wherein
(i) the amino acid residue in the encoded polypeptide that corresponds to amino acid position 27 of SEQ ID NO: 109 comprises alanine, serine, or threonine;
(ii) the amino acid residue in the encoded polypeptide that corresponds to amino acid position 38 of SEQ ID NO: 109 comprises isoleucine;
(iii) the amino acid residue in the encoded polypeptide that corresponds to amino acid position 42 of SEQ ID NO: 109 comprises alanine, methionine, or serine;
(iv) the amino acid residue in the encoded polypeptide that corresponds to amino acid position 52 of SEQ ID NO: 109 comprises glutamic acid;
(v) the amino acid residue in the encoded polypeptide that corresponds to amino acid position 61 of SEQ ID NO: 109 comprises alanine or serine;
(vi) the amino acid residue in the encoded polypeptide that corresponds to amino acid position 64 of SEQ ID NO: 109 comprises glycine, or serine;
(vii) the amino acid residue in the encoded polypeptide that corresponds to amino acid position 127 of SEQ ID NO: 109 comprises methionine;
(iix) the amino acid residue in the encoded polypeptide that corresponds to amino acid position 238 of SEQ ID NO: 109 comprises glycine;
(ix) the amino acid residue in the encoded polypeptide that corresponds to amino acid position 240 of SEQ ID NO: 109 comprises alanine, aspartic acid, or glutamic acid;
(x) the amino acid residue in the encoded polypeptide that corresponds to amino acid position 298 of SEQ ID NO: 109 comprises alanine or threonine,
(xi) the amino acid residue in the encoded polypeptide that corresponds to amino acid position 299 of SEQ ID NO: 109 comprises alanine;
(xii) the amino acid residue in the encoded polypeptide that corresponds to amino acid position 303 of SEQ ID NO: 109 comprises cysteine, glutamic acid, or serine;
(xiii) the amino acid residue in the encoded polypeptide that corresponds to amino acid position 327 of SEQ ID NO: 109 comprises leucine, glutamine, or valine;
(ixv) the amino acid residue in the encoded polypeptide that corresponds to amino acid position 328 of SEQ ID NO: 109 comprises aspartic acid, arginine, or serine; and/or
(xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

22. The method of embodiment 19, wherein said polypeptide having dicamba decarboxylase activity comprises:
(a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1;
(b) an amino acid sequence having at least 85%, 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129,
(c) an amino acid sequence having at least 60% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129 and wherein
   (i) the amino acid residue in the encoded polypeptide that corresponds to amino acid position 27 of SEQ ID NO: 109 comprises alanine, serine, or threonine;
   (ii) the amino acid residue in the encoded polypeptide that corresponds to amino acid position 38 of SEQ ID NO: 109 comprises isoleucine;
   (iii) the amino acid residue in the encoded polypeptide that corresponds to amino acid position 42 of SEQ ID NO: 109 comprises alanine, methionine, or serine;
   (iv) the amino acid residue in the encoded polypeptide that corresponds to amino acid position 52 of SEQ ID NO: 109 comprises glutamic acid;
   (v) the amino acid residue in the encoded polypeptide that corresponds to amino acid position 61 of SEQ ID NO: 109 comprises alanine or serine;
   (vi) the amino acid residue in the encoded polypeptide that corresponds to amino acid position 64 of SEQ ID NO: 109 comprises glycine, or serine;
   (vii) the amino acid residue in the encoded polypeptide that corresponds to amino acid position 127 of SEQ ID NO: 109 comprises methionine;
   (iix) the amino acid residue in the encoded polypeptide that corresponds to amino acid position 238 of SEQ ID NO: 109 comprises glycine;
   (ix) the amino acid residue in the encoded polypeptide that corresponds to amino acid position 240 of SEQ ID NO: 109 comprises alanine, aspartic acid, or glutamic acid;
   (x) the amino acid residue in the encoded polypeptide that corresponds to amino acid position 298 of SEQ ID NO: 109 comprises alanine or threonine,
   (xi) the amino acid residue in the encoded polypeptide that corresponds to amino acid position 299 of SEQ ID NO: 109 comprises alanine;
   (xii) the amino acid residue in the encoded polypeptide that corresponds to amino acid position 303 of SEQ ID NO: 109 comprises cysteine, glutamic acid, or serine;
   (xiii) the amino acid residue in the encoded polypeptide that corresponds to amino acid position 327 of SEQ ID NO: 109 comprises leucine, glutamine, or valine;
   (ixv) the amino acid residue in the encoded polypeptide that corresponds to amino acid position 328 of SEQ ID NO: 109 comprises aspartic acid, arginine, or serine; and/or
   (xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

23. The method of any one of embodiments 19-22, wherein said polypeptide having dicamba decarboxylase activity has a k_{cat}/Kₘ of at least 0.001 mM⁻¹ min⁻¹ for dicamba.

24. The method of embodiments 19-23, further comprising selecting a plant cell which is resistant to dicamba by growing the transgenic plant or plant cell in the presence of a concentration of dicamba under conditions where the dicamba decarboxylase is expressed at an effective level, whereby the transgenic plant or plant cell grows at a rate that is discernibly greater than the plant or plant cell would grow if it did not contain the nucleic acid construct.

25. The method of embodiment 19-24, wherein said method further comprises regenerating a transgenic plant from said plant cell.

26. A method to decarboxylate dicamba, a derivative of dicamba or a metabolite of dicamba comprising applying to a plant, an explant, a plant cell or a seed as set forth in any one of embodiments 1-19 dicamba or an active derivative thereof, and wherein expression of the dicamba decarboxylase decarboxylates the dicamba, the active derivative thereof or the dicamba metabolite.

27. The method of embodiment 26, wherein expression of the dicamba decarboxylase reduces the herbicidal activity of said dicamba, said dicamba derivative or said dicamba metabolite.

28. A method for controlling weeds in a field containing a crop comprising:
(a) applying to an area of cultivation, a crop or a weed in an area of cultivation a sufficient amount of dicamba or an active derivative thereof to control weeds without significantly affecting the crop; and,
(b) planting the field with the transgenic seeds of embodiment 18 or the plant of any one of embodiments 12 or 14-17.

29. The method of embodiment 26, 27 or 28, wherein said dicamba is applied to the area of cultivation or to said plant.

30. The method of embodiment 28, wherein step (a) occurs before or simultaneously with or after step (b).

31. The method of embodiment 28, 29 or 30, further comprising applying to the crop and weeds in the field a sufficient amount of at least one additional herbicide comprising glyphosate, bialaphos, phosphinothricin, sulfosate, glufosinate, an HPPD inhibitor, an ALS inhibitor, a second auxin analog, or a protox inhibitor.

32. A method for detecting a dicamba decarboxylase polypeptide comprising analyzing plant tissues using an immunoassay comprising an antibody or antibodies that specifically recognizes a polypeptide having dicamba decarboxylase activity, wherein said antibody or antibodies are raised to a polypeptide or a fragment of a polypeptide having dicamba decarboxylase activity.

33. A method for detecting the presence of a polynucleotide encoding a polypeptide having dicamba decarboxylase activity comprising assaying plant tissue using PCR amplification and detecting said polynucleotide encoding a polypeptide having dicamba decarboxylase activity.

34. The method of embodiment 32 or 33, wherein said polypeptide having dicamba decarboxylase activity comprises an active site having a catalytic residue geometry as set forth in Table 3 or having a substantially similar catalytic residue geometry.

35. The method of embodiment 34, wherein said polypeptide having dicamba decarboxylase activity comprises:

(a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1;

(b) an amino acid sequence having at least 60%, 70%, 75%, 80% 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; or

(c) an amino acid sequence having at least 60% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129 and wherein
(i) the amino acid residue in the encoded polypeptide that corresponds to amino acid position 27 of SEQ ID NO: 109 comprises alanine, serine, or threonine;
(ii) the amino acid residue in the encoded polypeptide that corresponds to amino acid position 38 of SEQ ID NO: 109 comprises isoleucine;
(iii) the amino acid residue in the encoded polypeptide that corresponds to amino acid position 42 of SEQ ID NO: 109 comprises alanine, methionine, or serine;
(iv) the amino acid residue in the encoded polypeptide that corresponds to amino acid position 52 of SEQ ID NO: 109 comprises glutamic acid;
(v) the amino acid residue in the encoded polypeptide that corresponds to amino acid position 61 of SEQ ID NO: 109 comprises alanine or senile;
(vi) the amino acid residue in the encoded polypeptide that corresponds to amino acid position 64 of SEQ ID NO: 109 comprises glycine, or serine;
(vii) the amino acid residue in the encoded polypeptide that corresponds to amino acid position 127 of SEQ ID NO: 109 comprises methionine;
(iix) the amino acid residue in the encoded polypeptide that corresponds to amino acid position 238 of SEQ ID NO: 109 comprises glycine;
(ix) the amino acid residue in the encoded polypeptide that corresponds to amino acid position 240 of SEQ ID NO: 109 comprises alanine, aspartic acid, or glutamic acid;
(x) the amino acid residue in the encoded polypeptide that corresponds to amino acid position 298 of SEQ ID NO: 109 comprises alanine or threonine,
(xi) the amino acid residue in the encoded polypeptide that corresponds to amino acid position 299 of SEQ ID NO: 109 comprises alanine;
(xii) the amino acid residue in the encoded polypeptide that corresponds to amino acid position 303 of SEQ ID NO: 109 comprises cysteine, glutamic acid, or serine;
(xiii) the amino acid residue in the encoded polypeptide that corresponds to amino acid position 327 of SEQ ID NO: 109 comprises leucine, glutamine, or valine;
(ixv) the amino acid residue in the encoded polypeptide that corresponds to amino acid position 328 of SEQ ID NO: 109 comprises aspartic acid, arginine, or serine; and/or,
(xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

36. The method of embodiment 32 or 33, wherein said polypeptide having dicamba decarboxylase activity comprises:
(a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1;
(b) an amino acid sequence having at least 85%, 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; or,
(c) an amino acid sequence having at least 60% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129, wherein
   (i) the amino acid residue in the encoded polypeptide that corresponds to amino acid position 27 of SEQ ID NO: 109 comprises alanine, serine, or threonine;
   (ii) the amino acid residue in the encoded polypeptide that corresponds to amino acid position 38 of SEQ ID NO: 109 comprises isoleucine;
   (iii) the amino acid residue in the encoded polypeptide that corresponds to amino acid position 42 of SEQ ID NO: 109 comprises alanine, methionine, or serine;
   (iv) the amino acid residue in the encoded polypeptide that corresponds to amino acid position 52 of SEQ ID NO: 109 comprises glutamic acid;
   (v) the amino acid residue in the encoded polypeptide that corresponds to amino acid position 61 of SEQ ID NO: 109 comprises alanine or serine;
   (vi) the amino acid residue in the encoded polypeptide that corresponds to amino acid position 64 of SEQ ID NO: 109 comprises glycine, or serine;
   (vii) the amino acid residue in the encoded polypeptide that corresponds to amino acid position 127 of SEQ ID NO: 109 comprises methionine;
   (iix) the amino acid residue in the encoded polypeptide that corresponds to amino acid position 238 of SEQ ID NO: 109 comprises glycine;
   (ix) the amino acid residue in the encoded polypeptide that corresponds to amino acid position 240 of SEQ ID NO: 109 comprises alanine, aspartic acid, or glutamic acid;
   (x) the amino acid residue in the encoded polypeptide that corresponds to amino acid position 298 of SEQ ID NO: 109 comprises alanine or threonine,
   (xi) the amino acid residue in the encoded polypeptide that corresponds to amino acid position 299 of SEQ ID NO: 109 comprises alanine;
   (xii) the amino acid residue in the encoded polypeptide that corresponds to amino acid position 303 of SEQ ID NO: 109 comprises cysteine, glutamic acid, or serine;
   (xiii) the amino acid residue in the encoded polypeptide that corresponds to amino acid position 327 of SEQ ID NO: 109 comprises leucine, glutamine, or valine;
   (ixv) the amino acid residue in the encoded polypeptide that corresponds to amino acid position 328 of SEQ ID NO: 109 comprises aspartic acid, arginine, or serine; and/or,
   (xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

37. The method of embodiment 36, wherein said polypeptide having dicamba decarboxylase activity comprises an active site having a catalytic residue geometry as set forth in Table 3 or having a substantially similar catalytic residue geometry.

38. The method of embodiment 37, wherein said polypeptide having dicamba decarboxylase activity comprises:

(a) an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1; or,

(b) an amino acid sequence having at least 60%, 70%, 75%, 80% 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; or,

(c) an amino acid sequence having at least 60% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129, wherein
(i) the amino acid residue in the encoded polypeptide that corresponds to amino acid position 27 of SEQ ID NO: 109 comprises alanine, serine, or threonine;
(ii) the amino acid residue in the encoded polypeptide that corresponds to amino acid position 38 of SEQ ID NO: 109 comprises isoleucine;
(iii) the amino acid residue in the encoded polypeptide that corresponds to amino acid position 42 of SEQ ID NO: 109 comprises alanine, methionine, or serine;
(iv) the amino acid residue in the encoded polypeptide that corresponds to amino acid position 52 of SEQ ID NO: 109 comprises glutamic acid;
(v) the amino acid residue in the encoded polypeptide that corresponds to amino acid position 61 of SEQ ID NO: 109 comprises alanine or serine;
(vi) the amino acid residue in the encoded polypeptide that corresponds to amino acid position 64 of SEQ ID NO: 109 comprises glycine, or serine;
(vii) the amino acid residue in the encoded polypeptide that corresponds to amino acid position 127 of SEQ ID NO: 109 comprises methionine;
(iix) the amino acid residue in the encoded polypeptide that corresponds to amino acid position 238 of SEQ ID NO: 109 comprises glycine;
(ix) the amino acid residue in the encoded polypeptide that corresponds to amino acid position 240 of SEQ ID NO: 109 comprises alanine, aspartic acid, or glutamic acid;
(x) the amino acid residue in the encoded polypeptide that corresponds to amino acid position 298 of SEQ ID NO: 109 comprises alanine or threonine,
(xi) the amino acid residue in the encoded polypeptide that corresponds to amino acid position 299 of SEQ ID NO: 109 comprises alanine;
(xii) the amino acid residue in the encoded polypeptide that corresponds to amino acid position 303 of SEQ ID NO: 109 comprises cysteine, glutamic acid, or serine;
(xiii) the amino acid residue in the encoded polypeptide that corresponds to amino acid position 327 of SEQ ID NO: 109 comprises leucine, glutamine, or valine;
(ixv) the amino acid residue in the encoded polypeptide that corresponds to amino acid position 328 of SEQ ID NO: 109 comprises aspartic acid, arginine, or serine; and/or,
(xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

Additional non-limiting embodiments include:

1. An isolated or recombinant polypeptide having dicamba decarboxylase activity comprising:

(a) a polypeptide having a catalytic residue geometry as set forth in Table 3 or having a substantially similar catalytic residue geometry and further comprising an amino acid sequence having a similarity score of at least 548 for any one of SEQ ID NO: 51, 89, 79, 81, 95, or 100, wherein said similarity score is generated using the BLAST alignment program, with the BLOSUM62 substitution matrix, a gap existence penalty of 11, and a gap extension penalty of 1;

(b) a polypeptide having a catalytic residue geometry as set forth in Table 3 or having a substantially similar catalytic residue geometry and further comprising an amino acid sequence having at least 60%, 70%, 75%, 80% 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; or,

(c) a polypeptide having a catalytic residue geometry as set forth in Table 3 or having a substantially similar catalytic residue geometry and further comprising an amino acid sequence having at least 60% 70%, 75%, 80% 90%, or 95% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129, wherein
(i) the amino acid residue in the encoded polypeptide that corresponds to amino acid position 27 of SEQ ID NO: 109 comprises alanine, serine, or threonine;
(ii) the amino acid residue in the encoded polypeptide that corresponds to amino acid position 38 of SEQ ID NO: 109 comprises isoleucine;
(iii) the amino acid residue in the encoded polypeptide that corresponds to amino acid position 42 of SEQ ID NO: 109 comprises alanine, methionine, or serine;
(iv) the amino acid residue in the encoded polypeptide that corresponds to amino acid position 52 of SEQ ID NO: 109 comprises glutamic acid;
(v) the amino acid residue in the encoded polypeptide that corresponds to amino acid position 61 of SEQ ID NO: 109 comprises alanine or serine;
(vi) the amino acid residue in the encoded polypeptide that corresponds to amino acid position 64 of SEQ ID NO: 109 comprises glycine, or serine;
(vii) the amino acid residue in the encoded polypeptide that corresponds to amino acid position 127 of SEQ ID NO: 109 comprises methionine;
(iix) the amino acid residue in the encoded polypeptide that corresponds to amino acid position 238 of SEQ ID NO: 109 comprises glycine;
(ix) the amino acid residue in the encoded polypeptide that corresponds to amino acid position 240 of SEQ ID NO: 109 comprises alanine, aspartic acid, or glutamic acid;
(x) the amino acid residue in the encoded polypeptide that corresponds to amino acid position 298 of SEQ ID NO: 109 comprises alanine or threonine,
(xi) the amino acid residue in the encoded polypeptide that corresponds to amino acid position 299 of SEQ ID NO: 109 comprises alanine;
(xii) the amino acid residue in the encoded polypeptide that corresponds to amino acid position 303 of SEQ ID NO: 109 comprises cysteine, glutamic acid, or serine;
(xiii) the amino acid residue in the encoded polypeptide that corresponds to amino acid position 327 of SEQ ID NO: 109 comprises leucine, glutamine, or valine;
(ixv) the amino acid residue in the encoded polypeptide that corresponds to amino acid position 328 of SEQ ID NO: 109 comprises aspartic acid, arginine, or serine; and/or,
(xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

2. The isolated polypeptide of embodiment 1, wherein said polypeptide having dicamba decarboxylase activity has a k_{cat}/Kₘ of at least 0.0001 mM⁻¹ min⁻¹ for dicamba.

3. An isolated or recombinant polynucleotide comprising a nucleotide sequence encoding a polypeptide as set forth in embodiment 1 or 2.

4. A nucleic acid construct comprising the isolated or recombinant polynucleotide of embodiment 3.

5. The nucleic acid construct of embodiment 4, further comprising a promoter operably linked to said polynucleotide.

6. A cell comprising at least one polynucleotide of embodiment 3 or the nucleic acid construct of any one of embodiments 4-5, wherein said polynucleotide is heterologous to the cell.

7. The cell of embodiment 6, wherein said cell comprises a microbial cell.

8. A method of producing a host cell having a heterologous polynucleotide encoding a polypeptide having dicamba decarboxylase activity comprising transforming a host cell with a heterologous polynucleotide as set forth in embodiment 3 or a heterologous nucleic acid construct as set forth in embodiments 4 or 5.

9. The method of embodiment 8, wherein said cell comprises a microbial cell.

10. A method to decarboxylate dicamba, a dicamba derivative or a dicamba metabolite comprising contacting said dicamba, dicamba derivative or dicamba metabolite with a composition comprising an effective amount of the polypeptide of any one of embodiments 1 or 2 or an effective amount of the host cell of embodiment 6 or 7, wherein said effective amount is sufficient to decarboxylate said dicamba, said dicamba derivative or said dicamba metabolite.

11. The method of embodiment 10, wherein said composition is contacted with dicamba.

12. A method for detecting a polypeptide comprising using an immunoassay comprising an antibody or antibodies that specifically recognizes a polypeptide having dicamba decarboxylase activity, wherein said antibody or antibodies are raised to a polypeptide having dicamba decarboxylase activity or a fragment of said polypeptide and said polypeptide having dicamba decarboxylase activity comprises a polypeptide of embodiment 1.

13. A method for detecting the presence of a polynucleotide encoding a polypeptide having dicamba decarboxylase activity comprising using PCR amplification and detecting said polynucleotide encoding a polypeptide of embodiment 1.

### EXPERIMENTAL

### Example 1

### Methods for Measuring Dicamba Decarboxylase Activities

Decarboxylation refers to the removal of the COOH (carboxyl group), releasing carbon dioxide (CO₂), and its replacement with a proton. Thus, the first method of choice to measure dicamba decarboxylase activity is to measure CO₂ generated from enzyme reactions. Two methods of measuring CO₂ product were adapted from the literature. The first is a direct measurement of ¹⁴CO₂ formed from [¹⁴C]-carboxyl-labeled dicamba through CO₂ capture. Methods describing such measurement can be found in the literature (Oldham, 1992, in Enzyme Assays: A Practical Approach (Elsenthal, R., and Danson, M. J., Eds.), pp. 93-122, IRL Press, New York). The assay procedure called ¹⁴C assay was adapted and modified from Zhang et al. (Analytical Biochemistry 271, 137-142, 1999). Briefly, [¹⁴C]-carboxyl-labeled dicamba (custom synthesized from PerkinElmer) is used as the substrate and the product, ¹⁴CO₂, is trapped at the top of the microtiter plate by a filter paper impregnated with calcium hydroxide (Ca(OH)₂), a CO₂-absorbing agent. A typical reaction is composed of 2 mM [¹⁴C]-carboxyl-labeled dicamba, 100 mM phosphate buffer (pH 7.0), 50 mM KCl, 100 uM ZnCl₂, and appropriate amount of purified protein. Buffer components and purified protein are premixed and dispensed into wells in a 96-well or 384-well raised-rim, V-bottomed polypropylene microtiter plate. The radioactive substrate is then added to initiate the reaction. The assay plate is promptly covered by a filter paper pre-soaked in 20 mM Ca(OH)₂ solution. A sheet of adhesive tape (Qiagen catalog #1018104), slightly larger than the filter paper, is placed on top to seal the filter paper onto the plate. With a plate sealer, the filter paper is pressed against the reaction plate to prevent the escape of CO₂. One piece of acrylic spacer and one piece of rubber sheet are added sequentially on top of the plate to complete the reaction assembly, which is then clamped using a book press. When the reaction is completed, the pressure from the book press is released and plate removed. The reaction assembly is dissembled and filter paper cut and removed with a standard razor blade. The CO₂-capturing filter paper is then wrapped with Saran Wrap plastic membrane and exposed to a phosphoimage cassette overnight. The phosphoimage cassette is scanned using a Typhoon Trio+Variable Mode Imager (GE Healthcare—Life Sciences). Image analysis is performed with Image Quant TL image analysis software (GE Healthcare—Life Sciences).

The second method measuring CO₂ product is an indirect measurement using a coupled enzyme assay. When CO₂ is produced in the reaction buffer, it exits in chemical equilibrium producing carbonic acid which in turn rapidly dissociates to form hydrogen ions and bicarbonate by simple proton dissociation/association. Using Infinity™ Carbon Dioxide Liquid Stable Reagent 2×125 mL (Thermo Scientific catalog number TR28321), the amount of CO₂ product is monitored spectrophotometrically at 375 nm by coupling the production of bicarbonate to oxidation of NADH through phosphoenolpyruvate carboxylase (PEPC) and malate dehydrogenase (MDH) provided in the reagent kit. PEPC utilizes CO₂-generated bicarbonate in the sample to produce oxaloacetate and phosphate. MDH then catalyses the reduction of oxaloacetate to malate and the oxidation of NADH to NAD⁺. The resulting decrease in absorbance can be measured at 375 nm and is proportional to the amount of bicarbonate produced from CO₂ present in the sample. Prior to the assay, the pH of the reagent is adjusted to 7.0 using 1N HCL. 260 uL reagent (pH7.0) is added into a Greiner Bio-One flat bottom 96-well plate well containing 30 uL 10× concentrated dicamba stock solution for a final concentration of 0.5 mM to 20 mM. Then 10 uL (1-10 ug) enzyme is added to the mixture and mixed immediately for spectrum monitoring. The reaction plate is measured using a SpectraMax Plus 384 device (Molecular Devices) for changes in absorbance at 375 nm every 10 s for 30 minutes at room temperature. Measured absorbance is then converted to velocity by least squares fitting of each curve using the accompanying program SOFTmax PRO 5.4 with manual assessment/confirmation of the linear range. The velocity of a no-enzyme control is subtracted. An extinction coefficient of 6.22 mM⁻¹ cm⁻¹ for NADH is used to convert velocity values from milli-absorbance units/min to micromolar/min. Kinetic parameters are estimated by fitting initial velocity values to the Michaelis-Menten equation. The overall catalytic efficiency of an enzyme is expressed as k_{cat}/K_{M}.

Alternatively, dicamba decarboxylase activity can be monitored by measuring decarboxylation products other than CO₂ using product detection methods. The decarboxylation product of dicamba, 2,5-dichloro anisole or 2,5-DCA (FIG. 1C), is a volatile compound with a flash point of 21° C. To capture this volatile compound for detection, 140 ul of toluene solution is added on top of 1 ml reaction mixture to form a trapping layer in a 1.5 ml eppendorf tube. The reaction mixture contains 2 mM dicamba, 100 mM potassium phosphate (pH7.0), 50 mM KCl, 100 uM ZnCl₂, and appropriate amount of purified 100 ug protein. The reaction is kept still at room temperature overnight before being vortex mixed and centrifuged at 14,000 rpm for 15 minutes. The top toluene phase is carefully removed using a micropipette and transferred into a 12×32 mm polypropylene vial (Vial 11 mm) from MicroLiter Analytical Supplies, Inc. (catalog number 11-5300-100). The vial is sealed with Crimp seal (11 mm with FEP/Nat Rubber) from MicroLiter Analytical Supplies, Inc. (catalog number 11-0020A) using a E-Z Crimper™ 11 mm from Wheaton Inc. lul of the toluene mixture is taken from the sealed vials and injected in splitless mode into a GC/MS system for sample analysis (Agilent GC/MS system with a 6890A GC, a 5973N MSD and a CTC CombiPAL auto-sampler or with a 7890A GC, a 5975C MSD and an Agilent GC Sampler 80 auto-sampler). The GC parameters are: Agilent DB-5MS column (30 m length, 0.25 mm diameter, 0.25 um film) or equivalent; The GC inlet temperature, 250° C.; Carry gas, helium in constant flow mode (1.2 mL/min); The GC oven temperature program, initial temperature at 70° C. for 1 min, ramping to 200° C. at 15° C./min, and then ramping to 250° C. at 30° C./min. MS data acquisition is done in SIM (selected ion monitoring) mode, monitoring the positive ion at M/Z 176 for the molecular ion of 2,4-DCA. The solvent delay for MS acquisition is set at 4 min. Another method for detection of 2,5-DCA is a head-space GC/MS method. Briefly, reaction mixtures in 500 ul reaction volume are prepared in 1.5 ml 12×32 mm glass vials (Microliter Analytical Supplies, Cat#11-1200) for head space analysis. Glass vials are sealed with magnetic cap from MicroLiter Analytical Supplies, Inc. (catalog number 11-0030AT) using a E-Z Crimper™ 11 mm from Wheaton Industries Inc. The reaction is carried out at room temperature for various amount of time and stopped by heating at 95° C. for 5 min. The reaction vial is transferred to a agitator for incubation at 80° C. for 5 min at 500 rpm. With a syringe preheated at 80° C., 1000 uL of head space is injected with sample fill speed at 100 uL/sec. GC/MS parameters for headspace analysis are the same as for liquid sample analysis.

The decarboxylated and chloro hydrolyzed product, 4-chloro-3-methoxy phenol (FIG. 1D), is measured using a LC-MS/MS analytical procedure. Briefly, reaction mixtures containing various amounts of dicamba, 100 mM potassium phosphate (pH7.0), 50 mM KCl, 100 uM ZnCl₂, and appropriate amount of protein in 100 ul reaction volume were incubated at 30° C. for various times. 10 ul is removed from the reaction mixture and mixed with 90 ul pre-chilled methanol followed by centrifugation at 14,000 rpm for 15 min at 4° C. 10 ul of the supernatant is then transferred into 170 ul ddH2O to achieve 5% methanol solution for injection. 50 ul of the prepared sample is injected into a 4000 Q Trap LC-MS/MS system for sample analysis. LC-MS/MS parameters are: Mobile Phase A, 2 mM ammonium acetate in water; Mobile Phase B, 2 mM ammonium acetate in methanol; Column, Aquasil, 100×2.1 mm, 3 μm, C18 column; Flow Rate, 0.6 ml/min. The MS/MS fragment 157/142 which is common to 4-chloro-3-methoxy phenol, 2-chloro-5-methoxy phenol, and 3-chloro-5-methoxy phenol is monitored at a retention time of 2.88 min.

The decarboxylated and demethylated product of dicamba, 2,5-dichloro phenol or 2,5-DCP (FIG. 1E) is measured using a GC/MS analytical procedure with either liquid injection after liquid/liquid extraction using toluene as the extraction solvent or gas injection using head space method. The head space sample analysis is carried out on an Agilent GC/MS system with a 6890A GC, a 5973N MSD and a CTC CombiPAL auto-sampler or with a 7890A GC, a 5975C MSD and an Agilent GC Sampler 80 auto-sampler with Phenomenex ZB-MultiResidue-1 column (30 m length, 0.25 mm diameter, 0.25 um film) or equivalent. GC/MS parameters are: GC inlet temperature, 200° C.; Carry gas, helium in constant flow mode (1.2 mL/min); Oven temperature program, 70° C. for 1 min and then ramp to 275° C. at 40° C./min. Protein reactions are carried out in a 1.5 ml 12×32 mm glass vials for head space analysis as described previously. The reaction vial is transferred to a agitator for incubation at 90° C. for 4 min at 500 rpm. With a syringe preheated at 110° C., 1000 uL of head space is injected with sample fill speed at 100 uL/sec. A 2-mm diameter liner is used in sample inlet. The MS data acquisition is done in SIM (selected ion monitoring) mode. The positive ion at M/Z 162 for the molecular ion of 2,-5-DCP is monitored at retention time of 4.06 min. Solvent delay for MS acquisition is set at 3 min. GC/MS parameters for liquid sample analysis are the same as those for head space analysis, except that the volume of liquid injection is 1 uL.

Kinetic determination for dicamba decarboxylases can be achieved by measuring 2,5-DCP using the above GC/MS method. Briefly, a series of dicamba substrate ranging from 0 to 20 mM is used in 7.5 ml decarboxylation reaction mixture described previously. At time 0, 1.5 mL is removed and added to 150 uL 1N HCL. To the remaining 6 mL reaction, a suitable amount of protein is added to start the reaction. At different time points, 1.5 mL reaction is removed and added to 150 uL 1N HCL to stop the reaction. In total, 5 time point samples including time 0 are taken. To neutralize the pH back to 7.0, 150 ul 1N NaOH is added and mixed for 5 minutes. 0.5 mL each sample is transferred to a 1.5 ml 12×32 mm glass vials, sealed, and analyzed as described previously. A series of 2,5-DCP samples is included as standards to determine the molar amount of 2,5-DCP product in the reaction samples. Velocity is calculated by dividing product produced by the time the reaction proceeded. Kinetic parameters are estimated by fitting initial velocity values to the Michaelis-Menten equation.

### Example 2

### Phytotoxicity Evaluation of Decarboxylation Products of Dicamba

To evaluate whether dicamba decarboxylated product 2,5-DCA is herbicidal to plants, the compound was purchased from Acros Organics (USA, catalog number 264180250) and tested during soybean germination.

2,5-DCA was dissolved in ddH₂O to obtain a 10 mM stock solution, and filter sterilized. Soybean seeds of a Pioneer elite germplasm were sterilized with chlorine gas as following: a) two layers of seeds were placed in a 100×25 mm plastic Petri dish; b) in an exhaust fume hood, seeds were placed into a glass desiccator with a 250 mL beaker containing 100 mL bleach (5% NaOCl) and 3.5 mL 12N HCl was slowly added to the beaker; c) the lid was sealed closed on the desiccator and the seeds sterilized for at least 24 hr.

Sterilized soybean seeds were then imbibed in ddH₂O under sterile conditions at 25° C. for 24 hours before the germination test. For the germination test, 6-8 imbibed seeds were placed on a 100×25 mm deep Petri dish plate containing 50 ml germination media supplemented with or without modified auxin compounds. 1L seed germination media contains 3.21 g GAMBORG B-5 basal medium (PhytoTech), 20 g sucrose, 5 g tissue culture agar, and was pH adjusted to 5.7. Media was autoclaved at 121° C. for 25 min and cooled to 60° C. before the addition of auxin product compounds. Germination was carried out in a Percival growth chamber at 25° C. under 18 hr light and 6 hr dark cycle at 90 to 150 μE/m2/s for 16 days.

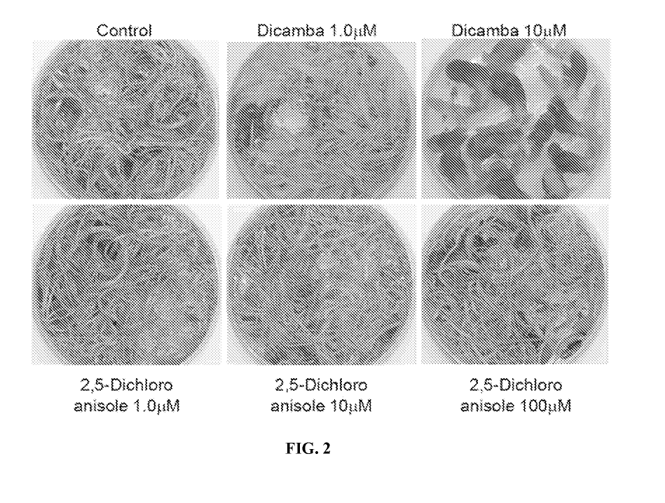

Soybean seeds germinated and grew very well in the media containing no supplemented auxin herbicides. After 16 days, both primary and secondary roots grew very well and elongated deep in the media (control in FIG. 2). In plates where 1 μM dicamba was added, seed germination was arrested as evident by bleaching of cotyledons and malformed and growth arrested roots. Emergence of true leaves and formation of secondary roots was not observed from these seeds. In plates where 10 μM dicamba was added, seed germination did not take place. Instead of root or leaf organ formation, seeds started to produce callus (FIG. 2). In comparison, in plates containing 1 μM or 10 μM of decarboxylated dicamba product 2,5-DCA, seed germination and growth were normal, similar to that of the control plates. Even at 100 μM, 2,5-DCA still did not have any obvious impact on soybean germination and growth (FIG. 2). The results indicate that the decarboxylated dicamba product is not phytotoxic to soybean and that decarboxylation of dicamba can be a mechanism for plants to detoxify dicamba herbicide.

Phytotoxicity of other major dicamba decarboxylaed products was evaluated using Arabidopsis root growth inhibition assay. 4-chlro-3-methoxy phenol was purchased from Biogene Organics, Inc. (catalog number U06-642-79). 2,5-dichloro phenol was purchased from Sigma-Aldrich (catalog number D70007). Briefly, seeds of Arabidopsis ecotype Columbia (Col-0) were surface sterilized with 70% (v/v) ethanol for 5 minutes and 10% (v/v) bleach for 15 minutes. After being washed three times with distilled water, the seeds were germinated on 1× Murashige and Skoog (MS) medium with a pH of 5.7, 3% (w/v) sucrose, and 0.8% (w/v) agar. After incubation for 3.5 days, the seedlings were transferred to 1× MS medium containing B5 vitamin, 3% (w/v) sucrose, 1.2% (w/v) agar, and filter sterilized compounds was added to the media at 60° C. The concentrations of compounds including dicamba were 0 μM, 1.0 μM, and 10 μM. The seedlings were placed vertically, and the temperature maintained at 23° C. to allow root growth along the surface of the agar, with a photoperiod of 16 h of light and 8 h of dark.

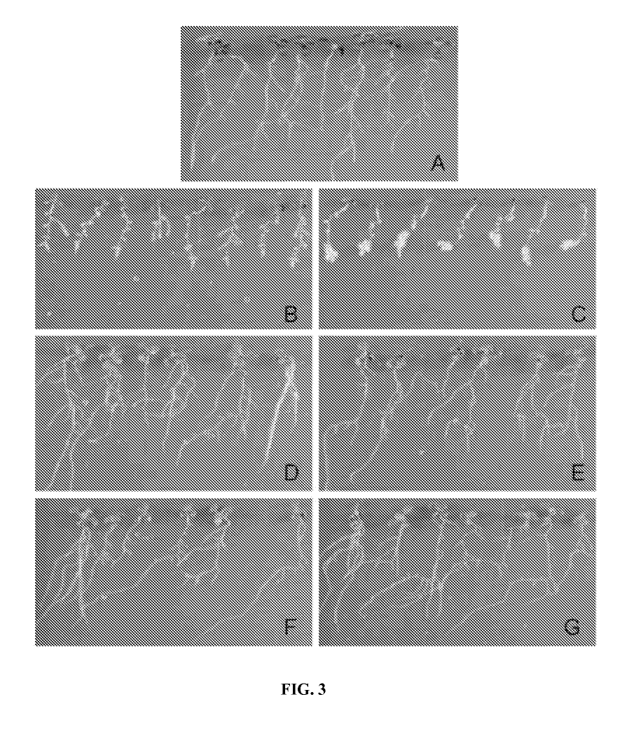

After 6 days on media, root growth was evaluated. In wild type Arabidopsis, root growth inhibition is expected from auxin herbicide treatment. As shown in FIGS. 3 (B and C), Arabidopsis root growth was greatly affected with dicamba treatment. At 1.0 uM, dicamba arrested the elongation of primary root and the formation of secondary roots. At 10 uM, the inhibitory effect of dicamba on root growth became more severe. Instead of formation of secondary root organ, callus was induced from the roots. Treatment with 4-chloro-3-methoxy phenol at 1.0 uM (FIG. 3D) and 10 uM (FIG. 3E) or 2,5-dichloro phenol at 1.0 uM (FIG. 3F) and 10 uM (FIG. 3G) did not have any effect on the growth of Arabidopsis roots when compared with the control in FIG. 3A.

### Example 3

### Activity and Phylogenetic Relationship of Dicamba Decarboxylase Candidate Proteins

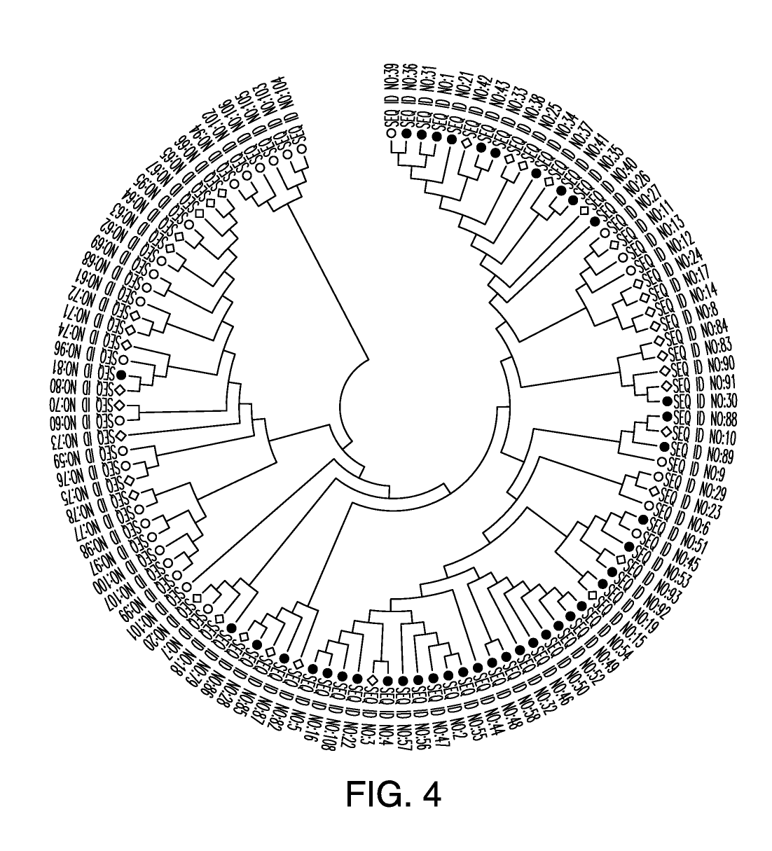

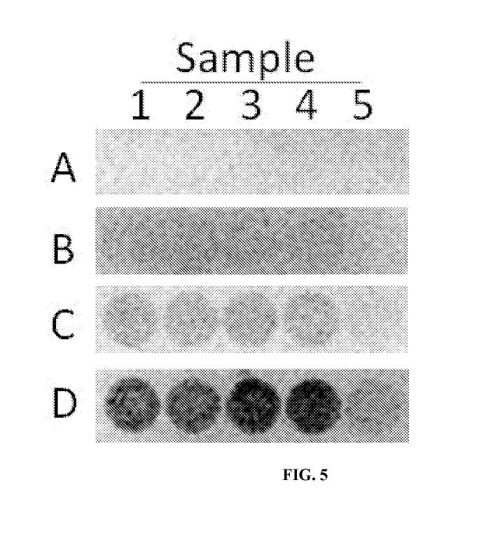

A total of 108 protein sequences, SEQ ID NO:1 to SEQ ID NO:108 (Table 2), were selected from GenBank analysis (NCBI, www.ncbi.nlm.nih.gov/). The phylogenetic relationship of these sequences was analyzed using CLUSTAL W followed by Neighbor-Joining method as shown in FIG. 4. Coding sequences were designed for expression in E. coli based on the protein sequences and synthesized. Synthesized coding sequences along with N-terminal His-tag coding sequences were cloned into a pET24a-based E. coli expression vector (Invitrogen). The E. coli expression vectors were transformed into BL21 Gold (DE3) (Stratagene) for protein expression. Recombinant E. coli strains were inoculated into 5 ml LB media supplemented with 40 mg/L kanamycin and cultured overnight at 37° C. 0.5 ml of overnight culture was inoculated into 50 mL LB medium plus 40 mg/L kanamycin and grown at 30° C. until OD₆₀₀ reached 0.6. The cultures were induced with 0.2 mM IPTG at 16° C., 230 rpm overnight. The cell cultures were used for dicamba decarboxylation assay directly measuring the formation of ¹⁴CO₂ from decarboxylation of [¹⁴C]-carboxyl-labeled dicamba. A typical cell assay composed of 45 ul induced recombinant cells and 5 ul 20 mM dicamba substrate (50:50 mixture (v:v) of [¹⁴C]-carboxyl-labeled dicamba and non-labeled cold dicamba). ¹⁴CO₂ was captured on Ca(OH)₂-soaked filter paper which was then exposed to a phosphoimage cassette as described in Example 1. The assay results are summarized in Table 2. In total, among the 108 sequences tested, 40 proteins (SEQ ID NO:1, 2, 4, 5, 16, 19, 21, 22, 26, 28, 30, 31, 32, 33, 34, 35, 36, 41, 43, 44, 46, 47, 48, 49, 50, 51, 52, 53, 54, 55, 56, 57, 58, 79, 81, 87, 88, 89, 92, 108) showed decarboxylation activity of dicamba. In FIG. 5 is shown results of a series of ¹⁴CO₂ accumulation over a time course from dicamba decarboxylation reactions using E. coli cells transformed with SEQ ID NO:1.

To obtain purified protein for activity assays, IPTG-induced cells were harvested by centrifugation at 7,000 rpm for 10 mins Cell pellet from 50 mL of cell culture was frozen and thawed twice and then lysed in 8004 lysis buffer consisting of 50 mM potassium phosphate buffer (pH7.0), 50 μM ZnSO₄, 5% EG, 50 mM KCl, 1 mM DTT, 0.2 mg/ml lysozyme, 1/200 protease inhibitor cocktail (EMD set3, EDTA free), and 1/2,000 endonuclease. Lysate was then centrifuged at 13,000 rpm for 45 min at 4° C. Supernatant was loaded onto 200 μL Ni-NTA columns pre-equilibrated with 10 mM His Buffer containing 25 mM potassium phosphate buffer pH7, 50 μM ZnSO₄, 5% EG, 200 mM KCl, and 10 mM histidine. The columns were let sit at 4° C. until the entire supernatant passed through. Each column was then washed with 200 ul 10 mM His Buffer twice and then 4 times with 800 ul loading buffer consisting of 25 mM potassium phosphate buffer pH7, 50 μM ZnSO₄, 5% EG, 200 mM KCl. Protein was eluted with 1504 of Elution Buffer consisting of 25 mM potassium phosphate buffer pH7, 50 μM ZnSO₄, 5% EG, 100 mM KCl, 100 mM histidine, 10% glycerol. The protein concentration was measured by Bradford assay. Purified protein was used for dicamba decarboxylase activity measurement as described in Example 1. Enzyme kinetic characterization of selected dicamba decarboxylases was determined through GC/MS measurement of 2,5-DCP or PEPC coupled assay as described in Example 1.

TABLE 2
Summary of dicamba decarboxylase activity for SEQ ID NO 1-108^{a}

| SEQ | GeneBank | | | Dicamba |
|---|---|---|---|---|
| ID | Accession | | | Decarboxylase |
| NO | Number | Gene Name | Organism | Activity^{b} |
| 1 | gi: 116667102 | 2,6-Dihydroxybenzoate | Rhizobium sp. | High |
| | | Decarboxylase | MTP-10005 | |
| 2 | gi\|333928717 | o-pyrocatechuate | Serratia sp. | High |
| | | decarboxylase | AS12 | |
| 3 | gi\|300769319 | possible o- | Lactobacillus | Low |
| | | pyrocatechuate | plantarum | |
| | | decarboxylase | subsp. | |
| | | | plantarum | |
| | | | ATCC 14917 | |
| 4 | gi\|331700448 | o-pyrocatechuate | Lactobacillus | High |
| | | decarboxylase | buchneri NRRL | |
| | | | B-30929 | |
| 5 | gi\|297589344 | possible o- | Staphylococcus | High |
| | | pyrocatechuate | aureus subsp. | |
| | | decarboxylase | aureus MN8 | |
| 6 | gi\|332297680 | o-pyrocatechuate | Treponema | No |
| | | decarboxylase | brennaborense | |
| | | | DSM 12168 | |
| 7 | gi\|307611400 | 5-carboxyvanillate | Legionella | No |
| | | decarboxylase | pneumophila | |
| | | | 130b | |
| 8 | gi\|322710070 | 2,3-dihydroxybenzoic | Metarhizium | Low |
| | | acid decarboxylase, | anisopliae | |
| | | putat | ARSEF 23 | |
| 9 | gi\|254450691 | 2,3-dihydroxybenzoic | Octadecabacter | No |
| | | acid decarboxylase | antarcticus 238 | |
| 10 | gi\|298291129 | o-pyrocatechuate | Starkeya novella | Low |
| | | decarboxylase | DSM 506 | |
| 11 | gi\|145237288 | 2,3-dihydroxybenzoic | Aspergillus | Low |
| | | acid decarboxylase | niger CBS | |
| | | | 513.88 | |
| 12 | gi\|339471266 | 2,3 dihydroxybenzoic | Zymoseptoria | No |
| | | acid decarboxylase-like | tritici IPO323 | |
| | | protein | | |
| 13 | gi\|322699386 | 2,3-dihydroxybenzoic | Metarhizium | No |
| | | acid decarboxylase | acridum CQMa | |
| | | dhbD | 102 | |
| 14 | gi\|212530386 | 2,3-dihydroxybenzoic | Talaromyces | Low |
| | | acid decarboxylase, | marneffei | |
| | | putative | ATCC 18224 | |
| 15 | gi\|322702683 | 2,3-dihydroxybenzoic | Metarhizium | Low |
| | | acid decarboxylase, | anisopliae | |
| | | putative | ARSEF 23 | |
| 16 | gi\|312437002 | possible o- | Staphylococcus | High |
| | | pyrocatechuate | aureus subsp. | |
| | | decarboxylase | aureus TCH60 | |
| 17 | gi\|145232495 | 2,3-dihydroxybenzoic | Aspergillus | Low |
| | | acid decarboxylase | niger CBS | |
| | | | 513.88 | |
| 18 | gi\|148360001 | 5-carboxyvanillate | Legionella | Low |
| | | decarboxylase | pneumophila str. | |
| | | | Corby | |
| 19 | gi\|212546025 | 2,3-dihydroxybenzoic | Talaromyces | High |
| | | acid decarboxylase, | marneffei | |
| | | putative | ATCC 18224 | |
| 20 | gi\|52842745 | 5-carboxyvanillate | Legionella | Low |
| | | decarboxylase | pneumophila | |
| | | | subsp. | |
| | | | pneumophila str. | |
| | | | Philadelphia 1 | |
| 21 | gi\|54290091 | reversible 2,6- | Agrobacterium | High |
| | | dihydroxybenzoic acid | tumefaciens | |
| | | decarboxylase | | |
| 22 | gi\|242372227 | possible o- | Staphylococcus | High |
| | | pyrocatechuate | epidermidis | |
| | | decarboxylase | M23864:W1 | |
| 23 | gi\|336041448 | putative 2,3- | Aplysina | Low |
| | | dihydroxybenzoic acid | aerophoba | |
| | | decarboxylase | bacterial | |
| | | | symbiont clone | |
| | | | AANRPS | |
| 24 | gi\|145254185 | 2,3-dihydroxybenzoic | Aspergillus | Low |
| | | acid decarboxylase | niger CBS | |
| | | | 513.88 | |
| 25 | gi\|326318924 | o-pyrocatechuate | Acidovorax | Low |
| | | decarboxylase | avenae subsp. | |
| | | | avenae ATCC | |
| | | | 19860 | |
| 26 | gi\|319795730 | o-pyrocatechuate | Variovorax | High |
| | | decarboxylase | paradoxus EPS | |
| 27 | gi\|169766084 | 2,3-dihydroxybenzoic | Aspergillus | No |
| | | acid decarboxylase | oryzae RIB40 | |
| 28 | gi\|19110430 | 5-carboxyvanillate | Sphingomonas | High |
| | | decarboxylase | paucimobilis | |
| 29 | gi\|254470775 | 2,3-dihydroxybenzoic | Pseudovibrio sp. | No |
| | | acid decarboxylase | JE062 | |
| 30 | gi\|336248046 | o-pyrocatechuate | Enterobacter | High |
| | | decarboxylase | aerogenes | |
| | | | KCTC 2190 | |
| 31 | gi\|325293881 | reversible 2,6- | Agrobacterium | High |
| | | dihydroxybenzoic acid | sp. H13-3 | |
| | | decarboxylase | | |
| 32 | gi\|307323742 | o-pyrocatechuate | Streptomyces | High |
| | | decarboxylase | violaceusniger | |
| | | | Tu 4113 | |
| 33 | gi\|116248886 | amidohydrolase | Rhizobium | High |
| | | | leguminosarum | |
| | | | bv. viciae 3841 | |
| 34 | gi\|339329031 | amidohydrolase | Cupriavidus | High |
| | | | necator N-1 | |
| 35 | gi\|323524953 | amidohydrolase | Burkholderia sp. | High |
| | | | CCGE1001 | |
| 36 | gi\|335034641 | hypothetical protein | Agrobacterium | High |
| | | AGRO_1970 | sp. ATCC | |
| | | | 31749 | |
| 37 | gi\|330820952 | amidohydrolase 2 | Burkholderia | Low |
| | | | gladioli BSR3 | |
| 38 | gi\|239819994 | amidohydrolase 2 | Variovorax | Low |
| | | | paradoxus S110 | |
| 39 | gi\|15889794 | conserved hypothetical | Agrobacterium | No |
| | | protein | fabrum str. C58 | |
| 40 | gi\|111018856 | hypothetical protein | Rhodococcus | Low |
| | | RHA1_ro01859 | jostii RHA1 | |
| 41 | gi\|91787937 | amidohydrolase 2 | Polaromonas sp. | High |
| | | | JS666 | |
| 42 | gi\|222080955 | metal dependent | Agrobacterium | Low |
| | | hydrolase | radiobacter K84 | |
| 43 | gi\|209546111 | amidohydrolase | Rhizobium | High |
| | | | leguminosarum | |
| | | | bv. trifolii | |
| | | | WSM2304 | |
| 44 | gi: 118462508 | amidohydrolase | Mycobacterium | High |
| | | | avium 104 | |
| 45 | gi: 126437094 | amidohydrolase 2 | Mycobacterium | No |
| | | | sp. JLS | |
| 46 | gi: 226364748 | decarboxylase | Rhodococcus | High |
| | | | opacus B4 | |
| 47 | gi: 270265324 | hypothetical protein | Serratia | High |
| | | SOD_m00560 | odorifera 4Rx13 | |
| 48 | gi: 300787436 | amidohydrolase | Amycolatopsis | High |
| | | | mediterranei | |
| | | | U32 | |
| 49 | gi: 302521182 | amidohydrolase 2 | Streptomyces | High |
| | | | sp. SPB78 | |
| 50 | gi: 302526758 | hypothetical protein | Streptomyces | High |
| | | SSMG_03140 | sp. AA4 | |
| 51 | gi: 315441546 | TIM-barrel fold metal- | Mycobacterium | High |
| | | dependent hydrolase | gilvum Spyr1 | |
| 52 | gi: 318057865 | putative decarboxylase | Streptomyces | High |
| | | | sp. SA3_actG | |
| 53 | gi: 322433076 | amidohydrolase | Granulicella | High |
| | | | tundricola | |
| | | | MP5ACTX9 | |
| 54 | gi: 333025132 | putative decarboxylase | Streptomyces | High |
| | | | sp. Tu6071 | |
| 55 | gi: 333928717 | o-pyrocatechuate | Serratia sp. | High |
| | | decarboxylase | AS12 | |
| 56 | gi: 336250281 | hypothetical protein | Enterobacter | High |
| | | EAE_19025 | aerogenes | |
| | | | KCTC 2190 | |
| 57 | gi: 340788176 | amidohydrolase | Collimonas | High |
| | | | fungivorans | |
| | | | Ter331 | |
| 58 | gi: 342859160 | amidohydrolase 2 | Mycobacterium | High |
| | | | colombiense | |
| | | | CECT 3035 | |
| 59 | gi: 163798099 | Aminocarboxymuconate- | alpha | No |
| | | semialdehyde | proteobacterium | |
| | | decarboxylase | BAL199 | |
| 60 | gi: 256396244 | amidohydrolase | Catenulispora | No |
| | | | acidiphila DSM | |
| | | | 44928 | |
| 61 | gi: 359423481 | putative 2-amino-3- | Gordonia | No |
| | | carboxymuconate-6- | amarae NBRC | |
| | | semialdehyde | 15530 | |
| | | decarboxylase | | |
| 62 | gi: 228914687 | 2-amino-3- | Bacillus | No |
| | | carboxymuconate-6- | thuringiensis | |
| | | semialdehyde | serovar | |
| | | decarboxylase | pulsiensis | |
| | | | BGSC 4CC1 | |
| 63 | gi: 238502329 | 2-amino-3- | Aspergillus | Low |
| | | carboxymuconate-6- | flavus | |
| | | semialdehyde | NRRL3357 | |
| | | decarboxylase, putative | | |
| 64 | gi: 293607565 | 2-amino-3- | Achromobacter | Low |
| | | carboxylmuconate-6- | piechaudii | |
| | | semialdehyde | ATCC 43553 | |
| | | decarboxylase | | |
| 65 | gi: 301770693 | PREDICTED: 2-amino- | Ailuropoda | Low |
| | | 3-carboxymuconate-6- | melanoleuca | |
| | | semialdehyde | | |
| | | decarboxylase-like | | |
| 66 | gi: 340375146 | PREDICTED: 2-amino- | Amphimedon | Low |
| | | 3-carboxymuconate-6- | queenslandica | |
| | | semialdehyde | | |
| | | decarboxylase-like | | |
| 67 | gi: 346471897 | hypothetical protein | Amblyomma | Low |
| | | | maculatum | |
| 68 | gi: 163759841 | Aminocarboxymuconate- | Hoeflea | No |
| | | semialdehyde | phototrophica | |
| | | decarboxylase | DFL-43 | |
| 69 | gi: 323358195 | metal-dependent | Microbacterium | No |
| | | hydrolase of the TIM- | testaceum | |
| | | barrel fold | StLB037 | |
| 70 | gi: 339289334 | amidohydrolase 2 | Alicyclobacillus | Low |
| | | | acidocaldarius | |
| | | | subsp. | |
| | | | acidocaldarius | |
| | | | Tc-4-1 | |
| 71 | gi: 254255373 | Aminocarboxymuconate- | Burkholderia | Low |
| | | semialdehyde | dolosa AUO158 | |
| | | decarboxylase | | |
| 72 | gi: 339321612 | unnamed protein | Cupriavidus | Low |
| | | product | necator N-1 | |
| 73 | gi: 269836141 | amidohydrolase 2 | Sphaerobacter | Low |
| | | | thermophilus | |
| | | | DSM 20745 | |
| 74 | gi: 337277884 | hypothetical protein | Ramlibacter | Low |
| | | Rta_02710 | tataouinensis | |
| | | | TTB310 | |
| 75 | gi: 299473403 | conserved unknown | Ectocarpus | Low |
| | | protein | siliculosus | |
| 76 | gi: 328542675 | 4-oxalomesaconate | Polymorphum | No |
| | | hydratase | gilvum SL003B- | |
| | | | 26A1 | |
| 77 | gi: 91780635 | hypothetical protein | Burkholderia | No |
| | | Bxe_C0594 | xenovorans | |
| | | | LB400 | |
| 78 | gi: 311692937 | amidohydrolase 2 | Marinobacter | Low |
| | | | adhaerens HP15 | |
| 79 | gi: 330938296 | hypothetical protein | Pyrenophora | High |
| | | PTT_18638 | teres f. teres 0-1 | |
| 80 | gi: 346327198 | uracil-5-carboxylate | Cordyceps | Low |
| | | decarboxylase | militaris CM01 | |
| 81 | gi: 346975906 | 2-amino-3- | Verticillium | High |
| | | carboxymuconate-6- | dahliae VdLs.17 | |
| | | semialdehyde | | |
| | | decarboxylase | | |
| 82 | gi: 86750218 | amidohydrolase 2 | Rhodopseudomonas | Low |
| | | | palustris | |
| | | | HaA2 | |
| 83 | gi: 353188507 | o-pyrocatechuate | Mycobacterium | Low |
| | | decarboxylase | rhodesiae JS60 | |
| 84 | gi: 359823113 | putative TIM-barrel | Mycobacterium | Low |
| | | fold metal-dependent | rhodesiae NBB3 | |
| | | hydrolase | | |
| 85 | gi: 84685620 | hypothetical protein | Maritimibacter | Low |
| | | 1099457000253_RB2654_06604 | alkaliphilus | |
| | | | HTCC2654 | |
| 86 | gi: 103485558 | amidohydrolase 2 | Sphingopyxis | Low |
| | | | alaskensis | |
| | | | RB2256 | |
| 87 | gi: 334140714 | amidohydrolase | Novosphingobium | High |
| | | | sp. PP1Y | |
| 88 | gi: 298291129 | o-pyrocatechuate | Starkeya novella | High |
| | | decarboxylase | DSM 506 | |
| 89 | gi: 300717179 | amidohydrolase | Erwinia | High |
| | | | billingiae Eb661 | |
| 90 | gi: 189199586 | amidohydrolase 2 | Pyrenophora | Low |
| | | | tritici-repentis | |
| | | | Pt-1C-BFP | |
| 91 | gi: 347828445 | hypothetical protein | Botryotinia | Low |
| | | | fuckeliana | |
| 92 | gi: 256423327 | amidohydrolase 2 | Chitinophaga | Yes |
| | | | pinensis DSM | |
| | | | 2588 | |
| 93 | gi: 312888301 | amidohydrolase 2 | Mucilaginibacter | Low |
| | | | paludis DSM | |
| | | | 18603 | |
| 94 | gi\|118476039 | phosphoribosylaminoimidazole | Bacillus | No |
| | | carboxylase | thuringiensis str. | |
| | | | A1 Hakam | |
| 95 | gi\|116667627 | Alpha-Amino-Beta- | Pseudomonas | No |
| | | Carboxymuconate- | fluorescens | |
| | | Epsilon-Semialdehyde- | | |
| | | Decarboxylase | | |
| 96 | gi\|67515537 | hypothetical protein | Aspergillus | No |
| | | AN0050.2 | nidulans FGSC | |
| | | | A4 | |
| 97 | gi\|347527637 | 4-oxalomesaconate | Sphingobium sp. | No |
| | | hydrat | SYK-6 | |
| 98 | gi\|21233454 | 4-oxalomesaconate | Xanthomonas | No |
| | | hydratase | campestris pv. | |
| | | | Campestris str. | |
| | | | ATCC 33913 | |
| 99 | gi\|83747590 | 4-oxalomesaconate | Ralstonia | No |
| | | hydratase | solanacearum | |
| | | | UW551 | |
| 100 | gi\|88799832 | 4-Oxalomesaconate | Reinekea | No |
| | | hydratase | blandensis | |
| | | | MED297 | |
| 101 | gi\|15605994 | phenylacrylic acid | Aquifex | No |
| | | decarboxylase | aeolicus VF5 | |
| 102 | gi\|254558099 | p-coumaric acid | Lactobacillus | No |
| | | decarboxylase | plantarum | |
| | | | JDM1 | |
| 103 | gi\|83285917 | adenosine deaminase | Plasmodium | No |
| | | | yoelii yoelii | |
| | | | 17XNL | |
| 104 | gi\|259090145 | Adenosine Deaminase | Plasmodial | No |
| | | | Vivax | |
| 105 | gi\|10957545 | hypothetical protein | Deinococcus | No |
| | | DR_C0006 | radiodurans R1 | |
| 106 | gi\|14590967 | hypothetical protein | Pyrococcus | No |
| | | PH1139 | horikoshii OT3 | |
| 107 | gi\|39937755 | 4-oxalomesaconate | Rhodopseudomonas | No |
| | | hydratase | palustris | |
| | | | CGA009 | |
| 108 | gi\|15925570 | hypothetical protein | Staphylococcus | High |
| | | SAV2580 | aureus subsp. | |
| | | | aureus Mu50 | |

^{a}Amino acid “Alanine” was added to all proteins at position 2 to facilitate cloning into the expression vector.
^{b}Dicamba decarboxylation activity description: High, dicamba decarboxylation activity was detected at relatively high level; No, dicamba decarboxylation activity was not detected; Low, dicamba decarboxylation activity was detected at a low level.

### Example 4

### Detection of Various Decarboxylated Products from Reactions with Selected Dicamba Decarboxylases

Enzymatic decarboxylation reactions, with the exception of orotidine decarboxylase, have not been studied or researched in detail. There is little information about their mechanism or enzymatic rates and no significant work done to improve their catalytic efficiency nor their substrate specificity. Decarboxylation reactions catalyze the release of CO₂ from their substrates which is quite remarkable given the energy requirements to break a carbon-carbon sigma bond, one of the strongest known in nature.

In examining the structure of dicamba, the carboxylate (—CO₂— or —CO₂H) is of utmost importance to its function. Enzymes were designed that would remove the carboxylate moiety efficiently rendering a significantly different product than dicamba (FIG. 1). Due to a variety of factors during the reaction including stereochemistry and location of general acids and bases as well as longevity of high energy intermediates, multiple products in addition to the simple decarboxylation are possible (FIG. 1). C is the simplest decarboxylation where the CO₂ is replaced by a proton, D is the product after decarboxylation and chlorohydrolase activity, and E is the product after decarboxylation and demethylase or methoxyhydrolase activity. The class of enzymes that was most similar to the desired dicamba decarboxylation was metal-catalyzed nonoxidative decarboxylases (Liu and Zhang, Biochemistry, 45:10407, 2006). This family of enzymes is relatively small but well conserved structurally and catalyzes the decarboxylation of aromatic acids or vinyl acids utilizing an enol stabilizing intermediate (that is not similarly possible to form with dicamba). While mechanisms have been hypothesized based upon the sequence similarity to deaminases (Crystal Structures of Nonoxidative Zinc-dependent 2,6-Dihydroxybenzoate (gamma-Resorcylate) Decarboxylase from Rhizobium sp. Strain MTP-10005″, Journal Biol. Chem. 281:34365-34373 (2006)) as well as from crystallized inhibitors, no work further elucidating the mechanism has been published.

Dicamba decarboxylases were expressed in E. coli cells and purified as His-tag proteins. Purified proteins were then incubated with dicamba substrate in the reaction buffer for product analysis as described in Example 1. For ¹⁴C assay, [¹⁴C]-carboxyl-labeled dicamba was used as substrate. Non-labeled dicamba was used for all other assays. Formation of four enzymatic reaction products (FIG. 1) was discovered using purified protein of SEQ ID NO:1. The first product is CO₂ which was detected in ¹⁴C assay using [¹⁴C]-carboxyl-labeled dicamba as substrate. The second is the predicted decarboxylated product, 2,5-DCA, which was detected using toluene capturing method followed by GC/MS analysis. The third is a decarboxylated and chlorohydrolyzed product, 4-chloro-3-methoxy phenol, which was detected using LC-MS/MS detection procedure. The fourth product is a decarboxylated and demethylated product, 2,5-DCP, which was detected by GC/MS analysis. Compared to the estimated amount of CO₂ formation (100%) in the reaction using ¹⁴C assay, the relative amount of 2,5-DCA, 4-chloro-3-methoxy phenol, and 2,5-DCP is approximately <1%, <10%, and >80%, respectively. Other dicamba decarboxylases with three major products (CO₂, 4-chloro-3-methoxy phenol, and 2,5-DCP) detected are SEQ ID NO:32, 41, 108, 109, 110, 111, 112, 113, 114, 115, and 116. These proteins were found to catalyze similar reactions of SEQ ID NO:1. The minor decarboxylation product 2,5-DCA was detected from reactions with protein SEQ ID NO:117, 118, 119, 120, 121, or 122, but other products were not detected from these protein reactions. Thus, the reaction mechanism may not be the same for all dicamba decarboxylases.

### Example 5

### Using Rational Design Approach to Obtain or Improve Enzyme Activity for Dicamba Decarboxylation

### A. Developing the Minimal Requirements and Constraints for Dicamba Decarboxylase Active Site and General Computational Design Methods.

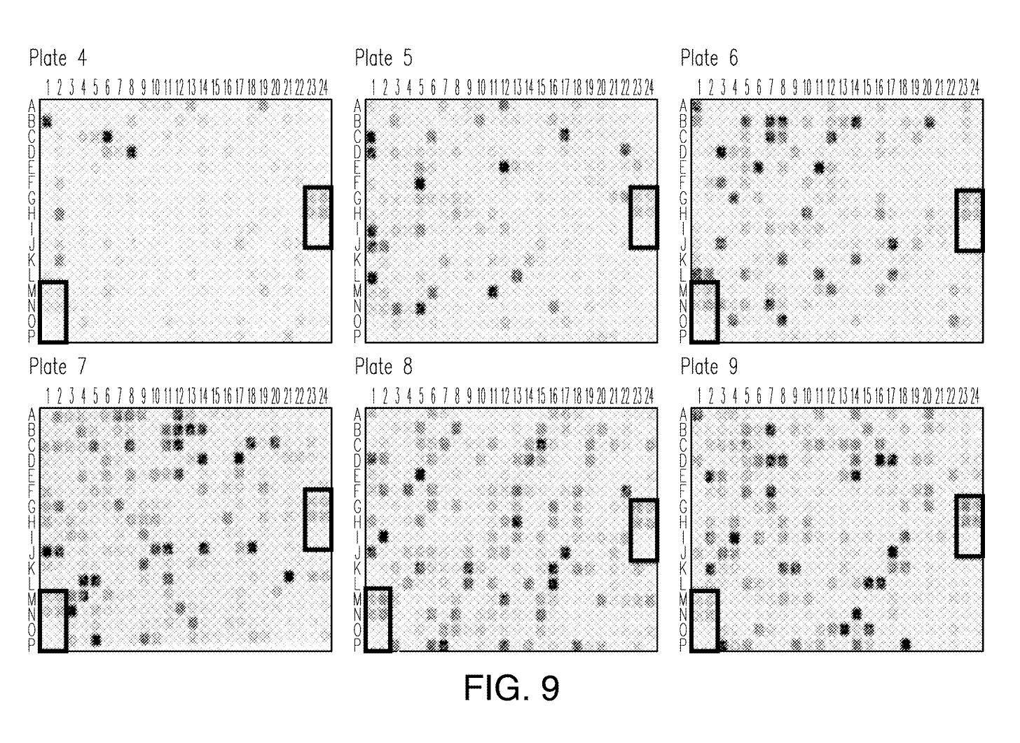

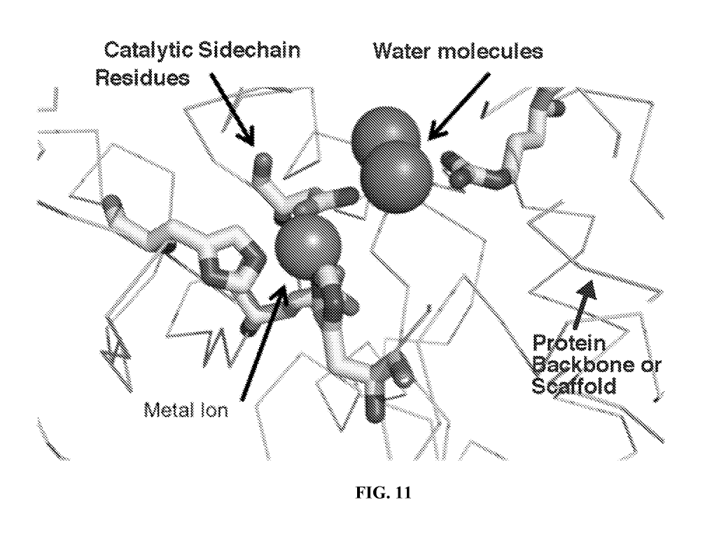

In order to achieve the best dicamba decarboxylase efficiency, computational methods were employed to design the active site to satisfy as many as possible the criteria of catalytic residues as well as substrate binding. Multiple approaches were utilized resulting in many active enzymes across multiple different protein backbones. All of the design calculations were begun utilizing an active site model as seen in FIGS. 9 and 11. This active site model is based on the natural class of transition metal-catalyzing nonoxidative decarboxylases and utilizes a zinc ion along with 4 coordinating side chains. The zinc ion can be replaced by cobalt, iron, nickel, or copper ions as the naturally occurring metal is not conclusively known for all of the enzymes (Huo, et al. Biochemistry. 2012 51:5811-21; Glueck, et al, Chem. Soc. Rev., 2010, 39, 313-328; Liu, et al, Biochemistry. 2006 45:10407-10411; Li, et al, Biochemistry 2006, 45:6628-6634).

Additionally, while FIG. 10 demonstrates two histidines and two aspartic or glutamic acid side chains, another possibility utilizing three histidines and one aspartate/glutamate was also tested. There are other sidechains in addition to histidine, asparate, and glutamate which can be used to chelate the metal including asparagine, glutamine, cysteine, cysteine and even tyrosine, threonine, and serine. Any combination of these could be used to chelate the metal and make the required catalytic geometry as seen in Table 3. The four side chain-chelated metal complex binds to the carboxylate of dicamba. This weakens the C—C bond enabling the addition of a proton. The proton is donated by the fifth catalytic residue which can be any hydrogen bond donating side-chain similar to the list above plus arginine and is often histidine. Stabilization by the other groups around the ring allows the C—C bond to break, fully releasing the CO₂ and regenerating the enzyme.

These combinations of histidines and acid were found initially in naturally existing enzyme scaffold proteins and correctly oriented to bind the necessary metal as the enzymes were designed within the naturally occurring decarboxylase family of proteins (Table 2). Substrate and product models were generated using state-of-art small-molecule building software packages such as, but not limited to, SPARTAN, Avogadro and Pymol, starting from equilibrium geometries for molecular parameters including, but not limited to, bond lengths, angles, dihedral angles and atom radii. The dicamba structure, the transition state geometry, and the orientation of the ligands relative to the metal and each other were further minimized using a molecular mechanics force-field such as MMFF94. Additionally, quantum mechanical calculations were performed to obtain the sensitivity of each degree of freedom within the transition state using quantum chemistry software packages such as SPARTAN or Gamess and exploring energies up to 5 kcal/mol higher than the global lowest transition state. This process explored the flexibility, or plasticity, of the transition state for the reaction during the subsequent design steps. The three-dimensional representation of one possible set of catalytic residues and the metal is shown in FIG. 11. The protein scaffold, or backbone, is shown in thin lines. The catalytic residues are shown in a thicker tube representation and the metal is shown as a sphere. There are two other spheres representing either water molecules or the position of the carboxylate oxygens from a dicamba molecule. The hydrogen bond donor depicted is arginine off to the right of the remainder of the active site.

B. Design of Related Sequences without Dicamba Decarboxylase Activity to Now Exhibit Enzymatic Activity.

In addition to improving already active enzymes, computational design was utilized to introduce activity not present in a wild-type scaffold (Table 4). No starting structure of SEQ ID NO:100 (from x-ray crystallography, NMR, etc.) exists, so it was necessary to build a starting model from the closest homolog with an available structure. Using state-of-the-art sequence search and analysis tools (including, but not limited to, heuristic methods, such as BLAST and its related variants and hidden Markov model methods, such as HMMER and its variants, a close homolog with a structure: SEQ ID NO:104 was identified. Using the sequence alignment of SEQ ID NO:100 to SEQ ID NO:104 given by the sequence search tool, initial threaded models were built, transferring the SEQ ID NO:100 sequence onto the SEQ ID NO:104 backbone, with insertions and deletions in the sequence alignment temporarily left un-modeled and instead representing those regions by backbone that were cut or left out of the model. The threaded models were built by iterating several times across (1) fixed backbone repacking+sidechain minimization followed by (2) tightly constrained minimization over the entire (cut) threaded model where constraints represented by, but not limited to, harmonic or similar types of potential functions, were applied between subsets of nearby heavy atoms. The best, or most successful, threaded models were selected by a feature cutoff (such as total energy) and manual inspection.

These threaded models were then taken as the starting point for full scale homology modeling, in which the cut regions from insertions/deletions were modeled, or built, using loop modeling techniques. ‘Loop’ here does not refer to coiled or non-structured protein secondary structure. ‘Loop’ refers to a stretch of protein backbone that must critically maintain appropriate geometic and chemical connection between two fixed stretches of backbone, one upstream, and one downstream in the linear sequence. It is important to note that SEQ ID NO:100 (and SEQ ID NO:104 and suspect that most of the sequences presented herein) is a dimer, so this full reconstruction was done as a dimer. To reduce computational costs, loops were only built on one monomer in the presence of the other monomer; this was valid in the case of SEQ ID NO:100 since the distance between the active sites and the dimer interface ensured that the loops did not interact between monomers, otherwise modeling the loops on both monomers simultaneously would likely have been a necessity. For SEQ ID NO:100, the primary loops to be modeled were the two loops at the active site. Loops were built using state-of-the-art loop modeling techniques including, but not limited to, algorithms inspired from the robotics field such as, analytical loop closure, as well as, fragment insertion based techniques. Models were built and subsequently clustered based on the loop positions, and best models were picked by feature cutoff including, but not limited to, total energy, energies of the loop, measures of reasonable loop geometry) and manual inspection. These models were used as starting structures for probing SEQ ID NO:100 further as well as for design.

For loop based designs, two approaches were used pursued; (1) the best full homology models were taken for substrate/transition state docking and fixed backbone design and (2) the substrate was docked into either the (cut) threaded model or a full homology model based on reaction specific constraints followed by building or rebuilding of loops of native and non-native lengths in combination with sequence design to accommodate and stabilize the docked substrate/transition state. Both of these approaches were followed by additional rounds of refinement through computational enzyme design. To narrow the search space for loops, initial scanning of loop lengths was performed using a lower resolution model and lower resolution scoring function—loops of different lengths were built and evaluated based on measures including, but not limited to, degree of successful closure and reasonable geometries of the loop. These lengths were then used as the lengths for approach (2). SEQ ID NO:95 had an existing crystal structure (PDB IDs:2hbv and 2hbx) but was not active for dicamba decarboxylation so its crystal structures were used used directly as the basis for the design of the active site.

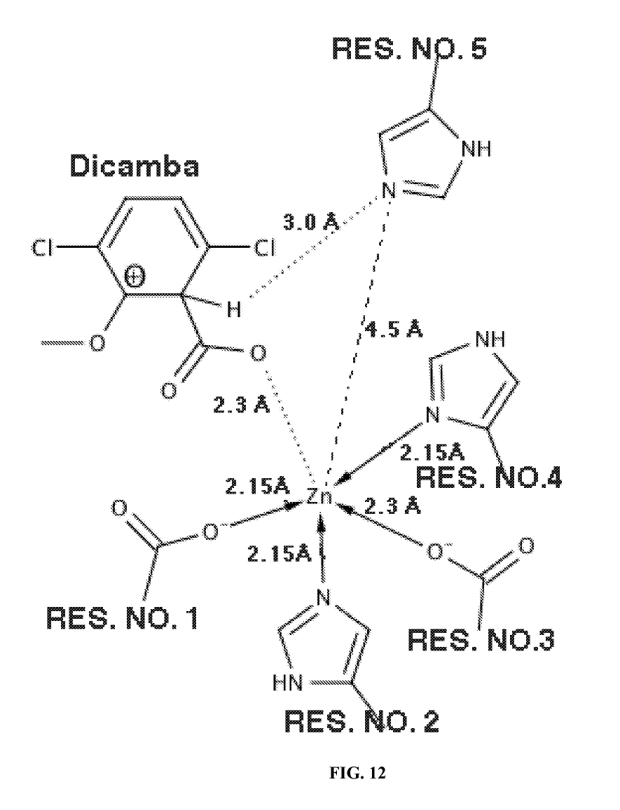

Sequence design steps, including computational enzyme design, proceeded in the following manner. The amino acid identities of the sidechains within and surrounding the active site (not included in the five catalytic residues) were optimized using a design algorithm utilizing a Monte Carlo optimization with a high resolution scoring function and employing a discrete rotamer representation of the sidechains using an extended version of the Dunbrack rotamer library similar to that used for 8,340,951 and US Application Publication No. US2009/0191607, both of which are herein incorporated by reference in their entirity. During this optimization, we impose different allowed behaviors on several subsets of residues: the subset of residues whose amino acid identities and sidechain conformations are allowed to vary are termed as “redesigned,” while a second subset of residues whose amino acid identities are kept fixed but whose sidechain conformations are allowed to vary are term as “repacked,” while those residues whose amino acid identity and sidechain conformations are maintained are termed “fixed.” We iterate between this discrete sequence optimization and a continuous optimization with a high resolution scoring function in which the dicamba rigid body degrees of freedom and the sidechain torsion angle degrees of freedom of the amino acids are allowed to vary simultaneously. In both discrete sequence optimization and the continuous optimization, we critically include in the high resolution scoring function a series of catalytic constraint functions utilizing the constraints observed in FIG. 12 and Table 3. We note here that the continuous optimization is essential to the subsequent assessment of the catalytic efficacy of the design.

To further optimize interactions (H-bonding or packing) that may still missing at the end of the normal design process, we generate additional design variants by introducing small perturbations to the dicamba degrees of freedom to explore slightly different rigid body orientations. Since these perturbations change the orientation of the dicamba to the catalytic sidechains, the conformations of the catalytic sidechains are re-optimized to ensure they are still within the defined geometric constraints. The remaining pocket is subsequently redesigned and refined as described above using the amino acid identities of the pre-perturbed design as the starting sequence. These perturbed and refined designs provide slight variations on the initial design which may have optimized properties. We iterate this process multiple times: small docking perturbations, pocket design and refinement in order to improve hydrogen bonding and packing interactions. Results of this approach include SEQ ID NOS: 117-122.

c. Design of Low Level Natural Enzymes with Dicamba Decarboxylase Activity to Higher Activity Levels.

For one set of the designed enzymes, simple computational design was done to improve the catalytic activity (for example SEQ ID NO: 109; Table 5). In this case, computational docking of the active site as shown in FIGS. 9 and 10 into SEQ ID NO: 1 is done while the identities of protein residues (excluding functional residues) are altered as to stabilize the resulting protein and/or provide additional favorable atomic contacts to the placed ligand and/or transition state or buttress the position of functional residues. This design methodology and technology are covered substantially in U.S. Pat. No. 8,340,951 and US Application Publication No. US2009/0191607, both of which are herein incorporated by reference.

At the end of the computational docking or computational docking and design steps, the structural protein models are ranked by score and/or structural features, and their amino acid sequences selected for further experimental characterization. This process resulted in sequences like SEQ ID NO:109 which were more active than their parent sequence. The dicamba molecule shows a change in orientation within the active site probably related to the improved activity. The designed mutation is asparagine 235 to valine (N235V). On the face of it, this mutation may not seem dramatic; however, using computational modeling and design it becomes clear that the shape of the pocket changes significantly and thus favors product formation for dicamba.

### D. Use of Computational Protein Backbone Structural Redesign in Order to Improve or Enable Enzymatic Activity.

In addition to homolog modeling and using computational design techniques to introduce dicamba decarboxylase activity where the parent enzyme scaffold did not have activity, we applied additional computational modeling and design methods including loop remodeling and redesign (restructuring loops to bind the substrate more tightly) and loop grafting (for example, up to 35 amino acids transferred) to introduce the necessary interactions for substrate recognition. In SEQ ID NO:1 we had the advantage of knowing more information: the crystal structure of the native protein, so no homology model needed to be built, and a more accurate picture of how the substrate/transition state fit into the active site. We identified (similar to SEQ ID NO:100), two (interacting) loops in the active site amenable to flexible backbone design. Here we took as the starting model the native SEQ ID NO:100 crystal structure (PDB ID:2gwg) with our transition state docked, and built (or rebuilt) those two loops with native and non-native lengths to accommodate and stabilize the docked substrate/transition state. Several of the possible loops sampled are shown in FIG. 13. This was followed by additional rounds of refinement using computational enzyme design resulting in, for example, SEQ ID NO: 110-115. Similarly as above, we used low resolution scanning of appropriate loop lengths to narrow the search space. For SEQ ID NO: 116 computational design modeled and designed a new 35 amino acid N-terminal loop based on SEQ ID NO:100 and were able to introduce improved dicamba decarboxylase activity into a parent enzyme (SEQ ID NO:41) possessing natural activity (Table 5). In total using computational design, we successfully introduced novel activity or improved the enzyme efficiency in five enzyme backbones introducing anywhere between 1 and 35 mutations to the parent sequence.

TABLE 4
Protein variants designed to introduce dicamba decarboxylation activity

| | | | Dicamba |
|---|---|---|---|
| SEQ ID | | | Decarboxylation |
| NO | Alias | Description | activity |
| 95 | DC.5.001 | Alpha-Amino-Beta- | No |
| | | Carboxymuconate-Epsilon- | |
| | | Semialdehyde-Decarboxylase | |
| 117 | DC.5.008 | Design variant of | Yes |
| | | SEQ ID NO: 95 | |
| 118 | DC.5.033 | Design variant of | Yes |
| | | SEQ ID NO: 95 | |
| 119 | DC.5.034 | Design variant of | Yes |
| | | SEQ ID NO: 95 | |
| 100 | DC.12.001 | 4-Oxalomesaconate | No |
| | | hydratase | |
| 120 | DC.12.002 | Design variant of | Yes |
| | | SEQ ID NO: 100 | |
| 121 | DC.12.014 | Design variant of | Yes |
| | | SEQ ID NO: 100 | |
| 122 | DC.12.103 | Design variant of | Yes |
| | | SEQ ID NO: 100 | |

TABLE 5
Designed protein variants with improved dicamba decarboxylase enzymatic
activity

| | | | | Percent Activity |
|---|---|---|---|---|
| | | | Dicamba | Improvement |
| SEQ ID | | | Decarboxylation | Over Parent |
| NO | Alias | Description | activity | (%) |
| 1 | DC.4.001 | 2,6-Dihydroxybenzoate Decarboxylase | Yes | 100 |
| 109 | DC.4.032 | Design variant of SEQ ID NO: 1 | Yes | 234 |
| 110 | DC.4.111 | Design variant of SEQ ID NO: 1 | Yes | 277 |
| 111 | DC.4.112 | Design variant of SEQ ID NO: 1 | Yes | 237 |
| 112 | DC.4.113 | Design variant of SEQ ID NO: 1 | Yes | 219 |
| 113 | DC.4.114 | Design variant of SEQ ID NO: 1 | Yes | 224 |
| 114 | DC.4.116 | Design variant of SEQ ID NO: 1 | Yes | 221 |
| 115 | DC.4.161 | Design variant of SEQ ID NO: 1 | Yes | 202 |
| 41 | DC.30.001 | amidohydrolase 2 | Yes | 100 |
| 116 | DC.30.007 | Design variant of SEQ ID NO: 41 | Yes | 220 |

Table 6 lists the important and conserved catalytic residues for activity within the sequences according to sequence alignment algorithms. Catalytic Residues #1-4 serve primarily to coordinate the metal within the active site. Most frequently they are histidine, aspartic acid, and glutamic acid. Catalytic Residue #5 serves as the proton donor which adds the proton to the aromatic ring displacing the carboxylate. These five catalytic residues are critical to the dicamba decarboxylase activity.

TABLE 6

| | | | | | | | | | | Cat. Residue #5 | |
|---|---|---|---|---|---|---|---|---|---|---|---|
| | Enzymatic | Cat. Residue #1 | | Cat. Residue #2 | | Cat. Residue #3 | | Cat. Residue #4 | | (Proton Donor) | |
| SEQ | Detection | | Residue | | Residue | | Residue | | Residue | | Residue |
| NO. | level | Identity | No. | Identity | No. | Identity | No. | Identity | No. | Identity | No. |
| 1 | High | GLU | 9 | HIS | 11 | HIS | 165 | ASP | 287 | HIS | 219 |
| 2 | High | GLU | 8 | HIS | 10 | HIS | 181 | ASP | 305 | HIS | 241 |
| 3 | Low | GLU | 8 | HIS | 10 | HIS | 171 | ASP | 296 | HIS | 233 |
| 4 | High | GLU | 8 | HIS | 10 | HIS | 173 | ASP | 298 | HIS | 235 |
| 5 | High | GLU | 17 | HIS | 19 | HIS | 181 | ASP | 304 | HIS | 242 |
| 6 | No | — | | — | | HIS | 95 | ASP | 216 | HIS | 155 |
| 7 | No | GLU | 7 | HIS | 9 | HIS | 181 | ASP | 302 | HIS | 233 |
| 8 | Low | GLU | 9 | ALA* | 11 | HIS | 170 | ASP | 298 | HIS | 225 |
| 9 | No | GLU | 9 | HIS | 11 | HIS | 161 | ASP | 280 | HIS | 214 |
| 10 | Low | GLU | 9 | HIS | 11 | HIS | 160 | ASP | 280 | HIS | 213 |
| 11 | Low | GLU | 9 | ALA | 11 | HIS | 168 | ASP | 294 | HIS | 223 |
| 12 | No | GLU | 9 | ALA | 11 | HIS | 168 | ASP | 292 | HIS | 223 |
| 13 | No | GLU | 9 | ALA | 11 | HIS | 166 | ASP | 290 | HIS | 221 |
| 14 | Low | GLU | 9 | ALA | 11 | HIS | 170 | ASP | 299 | HIS | 225 |
| 15 | Low | — | | — | | HIS | 79 | ASP | 204 | HIS | 140 |
| 16 | High | GLU | 15 | HIS | 17 | HIS | 181 | ASP | 305 | HIS | 242 |
| 17 | Low | GLU | 9 | ALA | 11 | HIS | 171 | ASP | 302 | HIS | 228 |
| 18 | Low | GLU | 7 | HIS | 9 | HIS | 181 | ASP | 303 | HIS | 233 |
| 19 | High | GLU | 9 | HIS | 11 | HIS | 151 | ASP | 276 | HIS | 213 |
| 20 | Low | GLU | 7 | HIS | 9 | HIS | 181 | ASP | 303 | HIS | 233 |
| 21 | High | GLU | 9 | HIS | 11 | HIS | 165 | ASP | 288 | HIS | 219 |
| 22 | High | GLU | 6 | HIS | 8 | HIS | 172 | ASP | 296 | HIS | 233 |
| 23 | Low | GLU | 60 | HIS | 62 | HIS | 207 | ASP | 334 | HIS | 268 |
| 24 | Low | GLU | 9 | ALA | 11 | HIS | 170 | ASP | 299 | HIS | 225 |
| 25 | Low | GLU | 9 | HIS | 11 | HIS | 165 | ASP | 288 | HIS | 219 |
| 26 | High | GLU | 15 | HIS | 17 | HIS | 171 | ASP | 292 | HIS | 225 |
| 27 | No | GLU | 9 | ALA | 11 | HIS | 168 | ASP | 294 | HIS | 223 |
| 28 | High | GLU | 8 | ALA | 10 | HIS | 174 | ASP | 297 | HIS | 227 |
| 29 | No | GLU | 45 | HIS | 47 | HIS | 196 | ASP | 323 | HIS | 257 |
| 30 | High | GLU | 9 | HIS | 11 | HIS | 170 | ASP | 295 | HIS | 225 |
| 31 | High | GLU | 9 | HIS | 11 | HIS | 165 | ASP | 288 | HIS | 219 |
| 32 | High | GLU | 8 | HIS | 10 | HIS | 169 | ASP | 295 | HIS | 230 |
| 33 | High | GLU | 9 | HIS | 11 | HIS | 165 | ASP | 288 | HIS | 219 |
| 34 | High | GLU | 9 | HIS | 11 | HIS | 165 | ASP | 288 | HIS | 219 |
| 35 | High | GLU | 12 | HIS | 14 | HIS | 168 | ASP | 291 | HIS | 222 |
| 36 | High | GLU | 9 | HIS | 11 | HIS | 165 | ASP | 288 | HIS | 219 |
| 37 | Low | GLU | 13 | HIS | 15 | HIS | 168 | ASP | 291 | HIS | 222 |
| 38 | Low | GLU | 9 | HIS | 11 | HIS | 165 | ASP | 288 | HIS | 219 |
| 39 | No | GLU | 9 | HIS | 11 | HIS | 165 | ASP | 288 | HIS | 219 |
| 40 | Low | GLU | 9 | HIS | 11 | HIS | 168 | ASP | 291 | HIS | 222 |
| 41 | High | GLU | 9 | HIS | 11 | HIS | 165 | ASP | 288 | HIS | 219 |
| 42 | Low | GLU | 9 | HIS | 11 | HIS | 165 | ASP | 288 | HIS | 219 |
| 43 | High | GLU | 9 | HIS | 11 | HIS | 165 | ASP | 288 | HIS | 219 |
| 44 | High | GLU | 8 | HIS | 10 | HIS | 166 | ASP | 292 | HIS | 227 |
| 45 | No | — | | — | | HIS | 80 | ASP | 204 | HIS | 140 |
| 46 | High | GLU | 8 | HIS | 10 | HIS | 169 | ASP | 294 | HIS | 229 |
| 47 | High | GLU | 8 | HIS | 10 | HIS | 181 | ASP | 306 | HIS | 241 |
| 48 | High | GLU | 10 | HIS | 12 | HIS | 167 | ASP | 290 | HIS | 227 |
| 49 | High | GLU | 8 | HIS | 10 | HIS | 169 | ASP | 295 | HIS | 230 |
| 50 | High | GLU | 8 | HIS | 10 | HIS | 168 | ASP | 294 | HIS | 229 |
| 51 | High | GLU | 8 | HIS | 10 | HIS | 159 | ASP | 283 | HIS | 219 |
| 52 | High | GLU | 8 | HIS | 10 | HIS | 169 | ASP | 295 | HIS | 230 |
| 53 | High | GLU | 8 | HIS | 10 | HIS | 159 | ASP | 283 | HIS | 219 |
| 54 | High | GLU | 8 | HIS | 10 | HIS | 169 | ASP | 295 | HIS | 230 |
| 55 | High | GLU | 8 | HIS | 10 | HIS | 181 | ASP | 306 | HIS | 241 |
| 56 | High | GLU | 8 | HIS | 10 | HIS | 181 | ASP | 306 | HIS | 241 |
| 57 | High | GLU | 8 | HIS | 10 | HIS | 182 | ASP | 307 | HIS | 242 |
| 58 | High | GLU | 8 | HIS | 10 | HIS | 155 | ASP | 280 | HIS | 215 |
| 59 | No | HIS | 9 | HIS | 11 | HIS | 174 | ASP | 296 | ASN | 234 |
| 60 | No | HIS | 33 | HIS | 35 | HIS | 188 | ASP | 302 | HIS | 239 |
| 61 | No | HIS | 27 | HIS | 29 | HIS | 194 | ASN | 317 | HIS | 249 |
| 62 | No | — | | — | | HIS | 136 | ASP | 51 | HIS | 186 |
| 63 | Low | — | | — | | HIS | 171 | ASP | 296 | HIS | 225 |
| 64 | Low | HIS | 9 | HIS | 11 | HIS | 177 | ASP | 294 | HIS | 228 |
| 65 | Low | HIS | 7 | HIS | 9 | HIS | 175 | ASP | 292 | HIS | 225 |
| 66 | Low | HIS | 10 | HIS | 12 | HIS | 178 | ASP | 295 | HIS | 228 |
| 67 | Low | HIS | 16 | HIS | 18 | HIS | 185 | ASP | 302 | HIS | 235 |
| 68 | No | HIS | 7 | HIS | 9 | HIS | 174 | ASP | 290 | HIS | 224 |
| 69 | No | HIS | 14 | HIS | 16 | HIS | 185 | ASP | 300 | HIS | 235 |
| 70 | Low | HIS | 12 | HIS | 14 | HIS | 179 | ASP | 294 | HIS | 228 |
| 71 | Low | — | | — | | HIS | 241 | ASP | 356 | HIS | 291 |
| 72 | Low | HIS | 53 | HIS | 55 | HIS | 219 | ASP | 334 | HIS | 269 |
| 73 | Low | HIS | 7 | HIS | 9 | HIS | 172 | ASP | 287 | HIS | 222 |
| 74 | Low | HIS | 8 | HIS | 10 | HIS | 172 | ASP | 290 | HIS | 224 |
| 75 | Low | TYR | 7 | HIS | 9 | HIS | 163 | ASP | 285 | HIS | 220 |
| 76 | No | PHE | 8 | HIS | 10 | HIS | 163 | ASP | 294 | HIS | 218 |
| 77 | No | HIS | 7 | HIS | 9 | HIS | 191 | ASN | 310 | HIS | 245 |
| 78 | Low | HIS | 7 | HIS | 9 | HIS | 195 | ASN | 313 | HIS | 249 |
| 79 | High | GLU | 15 | HIS | 17 | GLU | 160 | ASN | 285 | HIS | 219 |
| 80 | Low | HIS | 13 | HIS | 15 | HIS | 196 | ASP | 326 | HIS | 252 |
| 81 | High | HIS | 13 | HIS | 15 | HIS | 196 | ASP | 326 | HIS | 253 |
| 82 | Low | GLU | 12 | HIS | 14 | HIS | 158 | ASP | 281 | HIS | 217 |
| 83 | Low | GLU | 7 | HIS | 9 | HIS | 158 | ASP | 284 | HIS | 215 |
| 84 | Low | GLU | 8 | HIS | 10 | HIS | 159 | ASP | 285 | HIS | 216 |
| 85 | Low | GLU | 13 | GLY | 15 | HIS | 169 | ASP | 292 | HIS | 222 |
| 86 | Low | GLU | 27 | ALA | 29 | HIS | 198 | ASP | 321 | HIS | 251 |
| 87 | High | GLU | 25 | ALA | 27 | HIS | 194 | ASP | 320 | HIS | 247 |
| 88 | High | GLU | 8 | HIS | 10 | HIS | 160 | ASP | 281 | HIS | 213 |
| 89 | High | GLU | 49 | HIS | 51 | HIS | 202 | ASP | 322 | HIS | 255 |
| 90 | Low | GLU | 36 | HIS | 38 | HIS | 206 | ASP | 336 | HIS | 267 |
| 91 | Low | GLU | 55 | HIS | 57 | HIS | 227 | ASP | 359 | HIS | 281 |
| 92 | High | GLU | 8 | HIS | 10 | HIS | 162 | ASP | 290 | HIS | 224 |
| 93 | Low | GLU | 20 | HIS | 22 | HIS | 174 | ASP | 302 | HIS | 236 |
| 94 | No | — | | — | | VAL | 94 | ASP | 301 | LYS | 126 |
| 95 | No | HIS | 10 | HIS | 12 | HIS | 178 | ASP | 295 | HIS | 229 |
| 96 | No | HIS | 9 | HIS | 11 | HIS | 201 | ASP | 332 | HIS | 259 |
| 97 | No | HIS | 9 | HIS | 11 | HIS | 179 | GLU | 285 | HIS | 224 |
| 98 | No | HIS | 7 | HIS | 9 | HIS | 178 | GLU | 284 | HIS | 223 |
| 99 | No | HIS | 7 | HIS | 9 | HIS | 179 | GLU | 285 | HIS | 224 |
| 100 | No | HIS | 7 | HIS | 9 | HIS | 180 | GLU | 286 | HIS | 225 |
| 101 | No | — | | — | | VAL | 89 | VAL | 171 | GLU | 113 |
| 102 | No | — | | — | | HIS | 42 | ASP | 143 | HIS | 331 |
| 103 | No | — | | — | | HIS | 147 | ASP | 312 | HIS | 228 |
| 104 | No | — | | — | | HIS | 146 | ASP | 311 | HIS | 227 |
| 105 | No | HIS | 6 | HIS | 8 | HIS | 107 | ASP | 195 | TYR | 149 |
| 106 | No | TYR | 29 | SER | 31 | TYR | 251 | ASP | 417 | ALA | 332 |
| 107 | No | HIS | 7 | HIS | 9 | HIS | 179 | GLU | 285 | HIS | 224 |
| 108 | High | GLU | 7 | HIS | 9 | HIS | 171 | ASP | 295 | HIS | 232 |
| 109 | High | GLU | 9 | HIS | 11 | HIS | 165 | ASP | 287 | HIS | 219 |
| 110 | High | GLU | 9 | HIS | 11 | HIS | 165 | ASP | 287 | HIS | 219 |
| 111 | High | GLU | 9 | HIS | 11 | HIS | 165 | ASP | 287 | HIS | 219 |
| 112 | High | GLU | 9 | HIS | 11 | HIS | 165 | ASP | 287 | HIS | 219 |
| 113 | High | GLU | 9 | HIS | 11 | HIS | 165 | ASP | 287 | HIS | 219 |
| 114 | High | GLU | 9 | HIS | 11 | HIS | 165 | ASP | 287 | HIS | 219 |
| 115 | High | GLU | 9 | HIS | 11 | HIS | 163 | ASP | 285 | HIS | 217 |
| 116 | High | GLU | 9 | HIS | 11 | HIS | 165 | ASP | 287 | HIS | 219 |
| 117 | High | HIS | 7 | HIS | 9 | HIS | 178 | ASP | 294 | GLY*** | 229 |
| 118 | High | HIS | 7 | HIS | 9 | HIS | 178 | ASP | 294 | HIS | 229 |
| 119 | High | HIS | 7 | HIS | 9 | HIS | 178 | ASP | 294 | HIS | 229 |
| 120 | High | HIS | 7 | HIS | 9 | HIS | 180 | GLU | 286 | HIS | 225 |
| 121 | High | HIS | 7 | HIS | 9 | HIS | 180 | GLU | 286 | HIS | 225 |
| 122 | High | HIS | 7 | HIS | 9 | HIS | 180 | ASP | 286 | HIS | 225 |
| 123 | High | GLU | 9 | HIS | 11 | HIS | 165 | ASP | 287 | HIS | 219 |
| 124 | High | GLU | 9 | HIS | 11 | HIS | 165 | ASP | 287 | HIS | 219 |
| 125 | High | GLU | 9 | HIS | 11 | HIS | 165 | ASP | 287 | HIS | 219 |
| 126 | High | GLU | 9 | HIS | 11 | HIS | 165 | ASP | 287 | HIS | 219 |
| 127 | High | GLU | 9 | HIS | 11 | HIS | 165 | ASP | 287 | HIS | 219 |
| 128 | High | GLU | 9 | HIS | 11 | HIS | 165 | ASP | 287 | HIS | 219 |
| 129 | High | GLU | 9 | HIS | 11 | HIS | 165 | ASP | 287 | HIS | 219 |

Table 3 provides the distance constraints are the inter-atomic distances between the Nδ (ND) or Nε (NE) of histidine or the Oδ (OD) of aspartate or Oε (OE) of glutamate and the transition metal (often, Zn²⁺) in the active site. For Residue #5 which donates the proton to the aromatic ring during the decarboxylation step, the distance constraints are between the Nδ (ND) or Nε (NE) of histidine or the Oδ (OD) of aspartate or Oε (OE) of glutamate and the metal as well the distance to the water in the public crystal structures or the presumed dicamba carboxylate oxygen when the enzymes are binding and acting upon dicamba. The general case and natural diversity is shown first followed by examples of six structures in the Protein Data Bank that exhibit the needed dicamba decarboxylase catalytic geometry.

TABLE 3

| General | | | | | |
|---|---|---|---|---|---|
| Constraints for | | | | | |
| dicamba | | | | | |
| decarboxylases | RESIDUE #1 | RESIDUE #2 | RESIDUE #3 | RESIDUE #4 | RESIDUE #5 |
| | GLU | HIS | HIS | ASP | HIS |
| | HIS | ASP | ASP | GLU | ASP |
| | ASP | GLU | GLU | HIS | GLU |
| | TYR | | | | |
| Median | 2.15 | 2.15 | 2.30 | 2.15 | 4.5 |
| distance to | | | | | |
| metal atom | | | | | |
| (Angstroms) | | | | | |
| Observed | 2.00-3.10 | 2.00-3.20 | 2.00-2.50 | 2.00-3.50 | 3.3-4.9 |
| Values | | | | | |

TABLE 10
Geometries from a publicly available database (The RCSB Protein Data Bank):

| | | | RESI- | | RESI- | | RESI- | | RESI- | | RESI- | RESI- |
|---|---|---|---|---|---|---|---|---|---|---|---|---|
| | | | DUE #1 | | DUE #2 | | DUE #3 | | DUE #4 | | DUE #5 | DUE #5 |
| | | | Distance | | Distance | | Distance | | Distance | | Distance | Distance to |
| | | RESI- | to metal | RESI- | to metal | RESI- | to metal | RESI- | to metal | RESI- | to metal | 5^{th} |
| | | DUE #1 | atom | DUE #2 | atom | DUE #3 | atom | DUE #4 | atom | DUE #5 | atom | coordination |
| | | Amino | (Ang- | Amino | (Ang- | Amino | (Ang- | Amino | (Ang- | Amino | (Ang- | atom** |
| SEQ ID | PDB ID | Acid ID | stroms) | Acid ID | stroms) | Acid ID | stroms) | Acid ID | stroms) | Acid ID | stroms) | (Angstroms) |
| 1 | 2dvt | GLU 8 | 2.02 | HIS 10 | 2.18 | HIS 164 | 2.12 | ASP 287 | 2.33 | HIS 218 | 4.37 | 5.05 |
| 95 | 2hbv | HIS 9 | 2.11 | HIS 11 | 2.19 | HIS 177 | 2.16 | ASP 294 | 2.13 | HIS 228 | 3.26 | 2.52 |
| 130 | 3nur* | GLU 28 | 2.15 | HIS 30 | 2.34 | HIS 192 | 2.34 | ASP 316 | 2.13 | HIS 253 | 4.98 | 3.22 |
| 131 | 3ij6 | TYR 6 | 3.07 | HIS 10 | 3.16 | HIS 160 | 2.36 | ASP 262 | 2.10 | HIS 205 | 4.83 | 2.92 |
| 107 | 2gwg | HIS 6 | 2.23 | HIS 8 | 2.20 | HIS 178 | 2.45 | GLU 284 | 2.45 | HIS 223 | 4.87 | 2.88 |
| 132 | 2imr | HIS 97 | 2.13 | HIS 99 | 2.07 | HIS 238 | 2.08 | ASP 352 | 3.35 | HIS 301 | 4.40 | 3.02 |

*3nur has a Ca++ metal in the active site and is nearly identical to SEQ ID NOS: 5, 16, and 108
**Distance measured from the side-chain atom to the Oxygen atom from the water molecule filling the 5^{th} coordination position on the Zn-atom in the crystal structure

In FIG. 12, the constraints for the distances between the key atoms of each sidechain, metal, and dicamba transition state are shown. The angles and torsions are difficult to render within one flat figure, but can be easily viewed for each interaction in Table 3. The represented distances represent the ideal distance as calculated from existing enzyme structures in combination with quantum mechanical calculations. In addition to the ideal value, calculations are done to estimate how far from the ideal each geometric parameter/constraint is allowed to diverge. These tolerances are shown in Table 3. The angles and torsions are similarly allowed to deviate somewhat from their ideal geometries in order to account for small changes in protein structure. The x-ray crystal structure for SEQ ID NO: 1 agrees closely with these values. The other dicamba decarboxylases may have slightly different catalytic residue identities, but the geometry of the active sites are very tightly conserved for all of the active enzymes as seen from the residue information in Table 6 as well as the computationally designed decarboxylases SEQ ID NO: 109-122 which use this idealized geometry during the enzyme design process.

### Example 6

### Saturated Mutagenesis of Dicamba Decarboxylase SEQ ID NO: 109

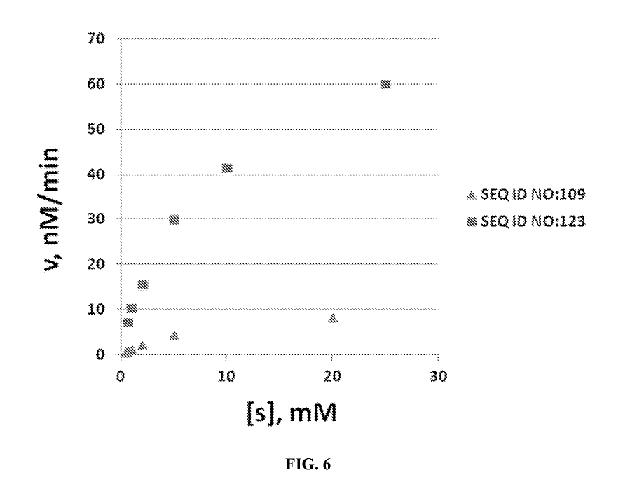

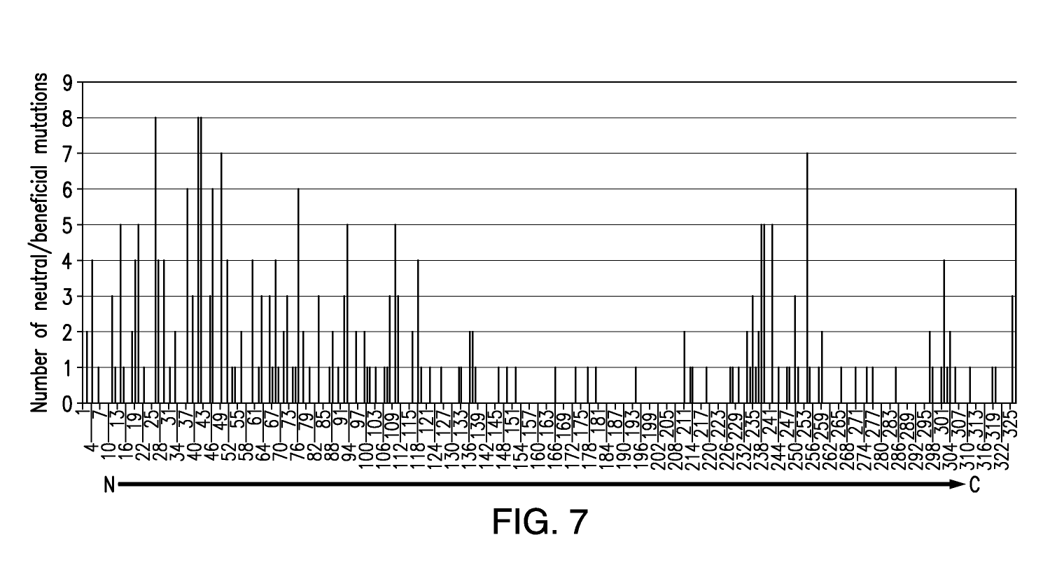

To discover amino acid positions on SEQ ID NO:109 where point mutations increase the activity of dicamba decarboxylation, saturation mutagenesis using NNK codons (N=A, T, G, or C; K=G or T) was performed along the entire length of the gene. NNK codons are used frequently for saturation mutagenesis to yield 32 possible codons to encode all 20 amino acids while minimizing the stop codons introduced. A total of 15,088 point mutants (46 randomly picked point mutants per amino acid position) were selected and the resulting protein variants were examined for their dicamba decarboxylation activity. Among the variants, 268 point mutations at 116 amino acid positions resulted in a 0.7- to 2.7-fold increase in dicamba decarboxylation activity (Table 7). 0.7-fold activity was used as the cut-off activity level because it represents one standard deviation below the average activity of SEQ ID NO:109. The top 30 point mutations from 14 amino acid positions resulted in more than 2.0-fold higher activity compared to SEQ ID NO:109. These 30 point mutations are: G27A, G27S, G27T, L38I, D42A, D42M, D42S, G52E, N61A, N61G, N61S, A64G, A64S, L127M, V238G, L240A, L240D, L240E, S298A, S298T, D299A, A303C, A303E, A303S, G327L, G327Q, G327V, A328D, A328R, and A328S. N61A was found to be 17-fold more active in k_{cat} while keeping K_{M} unchanged as compared with the template SEQ ID NO:109 (FIG. 6). The distribution of all 268 neutral/beneficial changes is shown in FIG. 7. Flexible positions and regions were discovered where multiple neutral or beneficial amino acid changes were found. For example, 8 neutral/beneficial amino acid changes were found at amino acid positions 27, 42, and 43 on SEQ ID NO:109. Positions in the N-terminal region are in general more amenable to amino acid changes. Other untested amino acid changes may also increase activity.

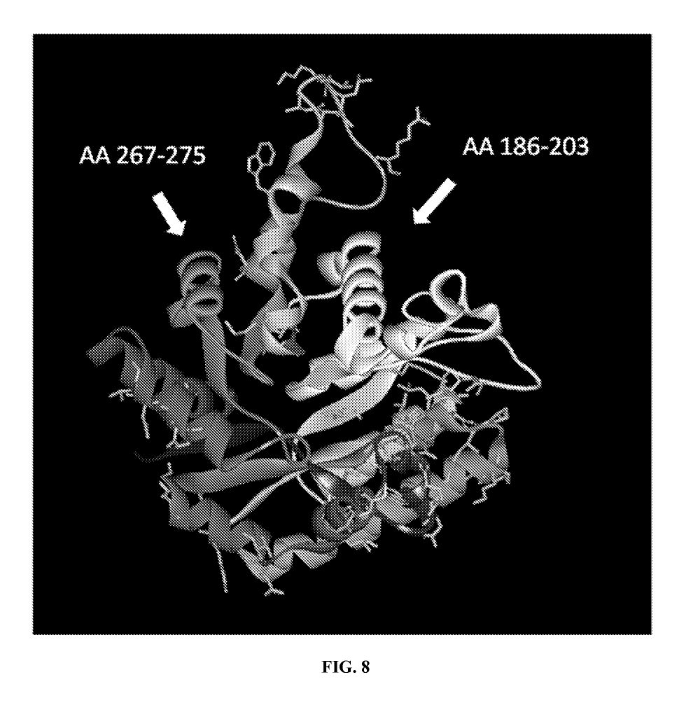

In some positions, only one point mutation was found to increase the protein activity (Table 7). For example, E16A, P63V, L104M, P107V, L127M, N214Q, V235I, D299A, N302A, and V312L each represent the only beneficial amino acid changes at their respective amino acid position. While these changes are beneficial for dicamba decarboxylation activity of greater than 1.8-fold as compared to the unchanged template SEQ ID NO:109, the other point mutations evaluated at these positions had a negative impact on the activity. The middle part of the protein is in general less amenable to amino acid changes as compared with the N-terminal end or the C-terminal end of the protein. For example, one region with a span of 72 AA positions in the middle part of the protein (position 139-210) did not tolerate much change as only 8 neutral/beneficial changes were found. Some regions in the protein, i.e. position 154-166 and 196-211 did not tolerate mutations as all variants showed much reduced activity. Region 267-275, a helix on the protein structure (FIG. 8) involved in the formation of the functional tetramer protein, theoretically would not tolerate much change. In fact, only one amino acid change in this region was found in I272V with 0.8-fold activity of the SEQ ID NO:109.

TABLE 7
Neutral or beneficial point mutations for SEQ ID NO: 109

| Amino | | Altered | | STDEV of | Variant |
|---|---|---|---|---|---|
| Acid | Amino Acid of | Amino | Average Activity (Fold | Average | Ranking by |
| Position | SEQ ID NO: 109 | Acid | of SEQ ID NO: 109) | Activity | Activity |
| 3 | Q | G | 1.2 | 0.2 | 181 |
| 3 | Q | M | 1.1 | 0.2 | 201 |
| 5 | K | E | 0.9 | 0.2 | 245 |
| 5 | K | I | 1.0 | 0.0 | 234 |
| 5 | K | L | 0.8 | 0.0 | 255 |
| 5 | K | W | 0.9 | 0.1 | 236 |
| 7 | A | C | 1.3 | 0.1 | 151 |
| 12 | F | M | 1.3 | 0.7 | 158 |
| 12 | F | V | 1.2 | 0.0 | 187 |
| 12 | F | W | 1.2 | 0.2 | 183 |
| 13 | A | C | 1.0 | 0.2 | 229 |
| 15 | P | A | 0.9 | 0.3 | 248 |
| 15 | P | D | 1.0 | 0.1 | 220 |
| 15 | P | E | 1.0 | 0.1 | 224 |
| 15 | P | Q | 1.0 | 0.1 | 232 |
| 15 | P | T | 1.1 | 0.2 | 212 |
| 16 | E | A | 1.8 | 0.5 | 49 |
| 19 | Q | E | 1.2 | 0.2 | 198 |
| 19 | Q | N | 1.6 | 0.6 | 78 |
| 20 | D | C | 1.8 | 0.0 | 48 |
| 20 | D | F | 1.9 | 0.2 | 32 |
| 20 | D | M | 1.6 | 0.5 | 96 |
| 20 | D | W | 1.5 | 0.1 | 129 |
| 21 | S | A | 1.6 | 1.0 | 99 |
| 21 | S | C | 1.0 | 0.6 | 227 |
| 21 | S | G | 1.2 | 0.7 | 182 |
| 21 | S | L | 1.0 | 0.2 | 221 |
| 21 | S | V | 1.2 | 0.6 | 196 |
| 23 | G | D | 1.5 | 0.2 | 118 |
| 27 | G | A | 2.0 | 0.5 | 25 |
| 27 | G | D | 1.7 | 0.4 | 50 |
| 27 | G | E | 1.5 | 0.2 | 106 |
| 27 | G | P | 1.6 | 0.1 | 95 |
| 27 | G | R | 1.6 | 0.4 | 90 |
| 27 | G | S | 2.2 | 0.2 | 19 |
| 27 | G | T | 2.0 | 0.3 | 26 |
| 27 | G | Y | 1.6 | 0.1 | 87 |
| 28 | D | C | 1.8 | 0.6 | 38 |
| 28 | D | E | 1.6 | 0.2 | 81 |
| 28 | D | F | 1.4 | 0.1 | 136 |
| 28 | D | G | 1.5 | 0.2 | 108 |
| 30 | W | L | 1.7 | 0.0 | 63 |
| 30 | W | Q | 1.0 | 0.1 | 225 |
| 30 | W | S | 0.7 | 0.1 | 261 |
| 30 | W | V | 1.7 | 0.2 | 56 |
| 32 | E | V | 1.1 | 0.2 | 202 |
| 34 | Q | A | 1.2 | 0.2 | 178 |
| 34 | Q | W | 1.5 | 0.4 | 105 |
| 38 | L | I | 2.0 | 0.0 | 30 |
| 38 | L | M | 1.7 | 0.3 | 64 |
| 38 | L | R | 1.7 | 0.3 | 61 |
| 38 | L | T | 1.9 | 0.3 | 36 |
| 38 | L | V | 1.6 | 0.1 | 100 |
| 40 | I | M | 1.4 | 0.2 | 149 |
| 40 | I | S | 1.5 | 0.1 | 121 |
| 40 | I | V | 1.3 | 0.1 | 169 |
| 42 | D | A | 2.0 | 0.5 | 23 |
| 42 | D | G | 1.5 | 0.2 | 123 |
| 42 | D | H | 0.9 | 0.0 | 237 |
| 42 | D | K | 1.6 | 0.1 | 73 |
| 42 | D | M | 2.4 | 0.4 | 10 |
| 42 | D | R | 1.0 | 0.3 | 219 |
| 42 | D | S | 2.0 | 0.5 | 29 |
| 42 | D | T | 1.8 | 0.0 | 45 |
| 43 | T | C | 1.7 | 0.3 | 58 |
| 43 | T | D | 1.6 | 0.0 | 98 |
| 43 | T | E | 1.3 | 0.0 | 157 |
| 43 | T | G | 1.3 | 0.3 | 164 |
| 43 | T | M | 1.3 | 0.1 | 163 |
| 43 | T | Q | 1.7 | 0.3 | 72 |
| 43 | T | R | 1.5 | 0.1 | 114 |
| 43 | T | Y | 1.2 | 0.2 | 192 |
| 46 | K | G | 1.2 | 0.1 | 174 |
| 46 | K | N | 1.4 | 0.1 | 145 |
| 46 | K | R | 1.7 | 0.5 | 52 |
| 47 | L | C | 1.1 | 0.2 | 208 |
| 47 | L | E | 1.3 | 0.2 | 172 |
| 47 | L | K | 1.1 | 0.1 | 218 |
| 47 | L | N | 0.9 | 0.2 | 246 |
| 47 | L | R | 0.8 | 0.1 | 259 |
| 47 | L | S | 1.2 | 0.0 | 189 |
| 50 | A | I | 0.9 | 0.0 | 240 |
| 50 | A | K | 1.9 | 0.0 | 35 |
| 50 | A | L | 1.0 | 0.0 | 223 |
| 50 | A | R | 1.4 | 0.2 | 134 |
| 50 | A | S | 1.4 | 0.1 | 131 |
| 50 | A | T | 1.4 | 0.1 | 132 |
| 50 | A | V | 1.3 | 0.2 | 152 |
| 52 | G | E | 3.1 | 1.2 | 1 |
| 52 | G | L | 1.7 | 0.7 | 65 |
| 52 | G | N | 1.6 | 0.3 | 83 |
| 52 | G | Q | 1.7 | 0.0 | 59 |
| 54 | E | G | 1.6 | 0.5 | 79 |
| 55 | T | L | 1.5 | 0.1 | 124 |
| 57 | I | A | 1.4 | 0.4 | 140 |
| 57 | I | V | 1.1 | 0.1 | 199 |
| 61 | N | A | 2.9 | 0.9 | 3 |
| 61 | N | G | 2.3 | 1.3 | 15 |
| 61 | N | L | 1.7 | 0.7 | 71 |
| 61 | N | S | 2.5 | 0.2 | 7 |
| 63 | P | V | 1.8 | 0.6 | 42 |
| 64 | A | G | 2.6 | 0.2 | 6 |
| 64 | A | H | 1.7 | NA | 67 |
| 64 | A | S | 2.1 | 0.4 | 20 |
| 67 | A | E | 0.9 | 0.0 | 239 |
| 67 | A | G | 0.8 | 0.0 | 257 |
| 67 | A | S | 1.7 | 0.1 | 54 |
| 68 | I | Q | 1.6 | 0.0 | 77 |
| 69 | P | G | 1.6 | 0.2 | 91 |
| 69 | P | R | 1.1 | 0.0 | 204 |
| 69 | P | S | 1.2 | 0.1 | 191 |
| 69 | P | V | 1.2 | 0.0 | 188 |
| 70 | D | H | 1.4 | 0.0 | 142 |
| 72 | R | K | 1.6 | 0.1 | 103 |
| 72 | R | V | 1.6 | 0.3 | 85 |
| 73 | K | E | 1.5 | 0.6 | 128 |
| 73 | K | Q | 1.8 | 0.6 | 39 |
| 73 | K | R | 1.4 | 0.1 | 133 |
| 75 | I | R | 1.6 | 0.0 | 101 |
| 76 | E | G | 1.3 | 0.3 | 156 |
| 77 | I | C | 1.0 | 0.4 | 233 |
| 77 | I | L | 0.9 | 0.1 | 249 |
| 77 | I | M | 1.3 | 0.1 | 171 |
| 77 | I | R | 1.4 | 0.4 | 146 |
| 77 | I | S | 1.5 | 0.5 | 113 |
| 77 | I | V | 1.2 | 0.2 | 194 |
| 79 | R | K | 0.7 | NA | 265 |
| 79 | R | Q | 1.2 | 0.0 | 177 |
| 81 | A | S | 1.4 | 0.0 | 135 |
| 84 | V | C | 1.2 | 0.2 | 175 |
| 84 | V | F | 1.6 | 0.1 | 89 |
| 84 | V | M | 1.6 | 0.0 | 74 |
| 88 | E | K | 1.3 | 0.2 | 170 |
| 89 | C | I | 1.5 | 0.2 | 126 |
| 89 | C | V | 1.5 | 0.1 | 116 |
| 91 | K | R | 1.2 | 0.0 | 184 |
| 93 | P | A | 1.1 | 0.2 | 203 |
| 93 | P | K | 0.7 | NA | 260 |
| 93 | P | R | 1.4 | 0.7 | 148 |
| 94 | D | C | 1.1 | 0.1 | 207 |
| 94 | D | G | 1.1 | 0.1 | 213 |
| 94 | D | N | 1.0 | 0.2 | 231 |
| 94 | D | Q | 1.2 | 0.0 | 197 |
| 94 | D | S | 1.2 | 0.0 | 185 |
| 97 | L | K | 1.2 | 0.1 | 186 |
| 97 | L | R | 1.3 | 0.1 | 153 |
| 100 | A | G | 1.3 | 0.0 | 154 |
| 100 | A | S | 1.5 | 0.0 | 127 |
| 101 | A | G | 1.6 | 0.0 | 75 |
| 102 | L | V | 1.4 | 0.2 | 143 |
| 104 | L | M | 1.9 | 0.9 | 31 |
| 107 | P | V | 1.8 | 0.5 | 47 |
| 108 | D | E | 1.7 | 0.1 | 60 |
| 109 | A | G | 1.3 | 0.2 | 155 |
| 109 | A | M | 1.5 | 0.3 | 104 |
| 109 | A | V | 1.5 | 0.1 | 125 |
| 111 | T | A | 1.4 | 0.6 | 147 |
| 111 | T | C | 1.6 | 0.6 | 88 |
| 111 | T | G | 1.5 | 0.4 | 120 |
| 111 | T | S | 1.7 | 0.4 | 55 |
| 111 | T | V | 1.5 | 0.5 | 112 |
| 112 | E | G | 1.4 | 0.6 | 138 |
| 112 | E | R | 1.5 | 0.6 | 110 |
| 112 | E | S | 1.5 | 0.3 | 115 |
| 117 | C | A | 1.7 | 0.7 | 51 |
| 117 | C | T | 1.8 | 1.0 | 43 |
| 119 | N | A | 1.4 | 0.3 | 139 |
| 119 | N | C | 1.3 | 0.5 | 167 |
| 119 | N | R | 1.5 | 0.5 | 111 |
| 119 | N | S | 1.3 | 0.5 | 168 |
| 120 | D | T | 1.7 | 0.8 | 66 |
| 123 | F | L | 1.3 | 0.3 | 160 |
| 127 | L | M | 2.4 | 1.0 | 8 |
| 133 | Q | V | 1.6 | 0.7 | 76 |
| 134 | E | G | 0.8 | NA | 258 |
| 137 | G | A | 1.2 | 0.4 | 173 |
| 137 | G | E | 1.2 | 0.3 | 180 |
| 138 | Q | G | 1.1 | NA | 200 |
| 138 | Q | L | 0.9 | NA | 243 |
| 139 | T | E | 0.7 | NA | 264 |
| 147 | Q | I | 1.1 | NA | 211 |
| 150 | P | G | 0.9 | NA | 238 |
| 153 | G | K | 1.6 | 0.4 | 93 |
| 167 | R | E | 1.6 | 0.3 | 92 |
| 174 | S | A | 1.2 | 0.1 | 179 |
| 178 | D | E | 1.2 | 0.2 | 193 |
| 181 | P | E | 0.9 | 0.0 | 242 |
| 195 | A | G | 1.2 | 0.2 | 176 |
| 212 | R | G | 1.6 | 0.1 | 97 |
| 212 | R | Q | 1.7 | 0.0 | 53 |
| 214 | N | Q | 1.8 | 0.1 | 41 |
| 215 | I | V | 0.8 | 0.0 | 252 |
| 220 | M | L | 1.7 | 0.1 | 69 |
| 228 | M | L | 1.4 | 0.1 | 141 |
| 229 | W | Y | 1.7 | 0.1 | 68 |
| 231 | I | M | 0.8 | 0.2 | 254 |
| 234 | R | H | 0.9 | 0.0 | 247 |
| 234 | R | K | 1.0 | 0.0 | 235 |
| 235 | V | I | 1.8 | 0.0 | 44 |
| 236 | A | G | 1.6 | 0.3 | 94 |
| 236 | A | Q | 1.2 | 0.2 | 190 |
| 236 | A | W | 1.4 | 0.1 | 137 |
| 237 | W | L | 1.1 | 0.3 | 209 |
| 238 | V | G | 2.0 | 0.2 | 27 |
| 238 | V | P | 1.3 | 0.1 | 166 |
| 239 | K | A | 1.7 | 0.1 | 62 |
| 239 | K | D | 1.3 | 0.0 | 162 |
| 239 | K | E | 1.5 | 0.1 | 107 |
| 239 | K | G | 1.6 | 0.1 | 80 |
| 239 | K | H | 1.8 | 0.1 | 46 |
| 240 | L | A | 2.3 | 0.5 | 12 |
| 240 | L | D | 2.2 | 0.2 | 18 |
| 240 | L | E | 2.1 | 0.1 | 22 |
| 240 | L | G | 1.5 | 0.0 | 122 |
| 240 | L | V | 1.6 | 0.1 | 86 |
| 243 | R | A | 1.8 | 0.4 | 37 |
| 243 | R | D | 1.6 | 0.1 | 102 |
| 243 | R | K | 1.5 | 0.0 | 119 |
| 243 | R | S | 1.4 | 0.0 | 144 |
| 243 | R | V | 1.4 | 0.0 | 130 |
| 245 | P | A | 1.5 | 0.1 | 109 |
| 248 | R | K | 1.1 | 0.1 | 205 |
| 249 | R | P | 1.1 | 0.0 | 206 |
| 251 | M | G | 0.9 | 0.1 | 251 |
| 251 | M | V | 1.3 | 0.1 | 150 |
| 252 | D | E | 1.0 | 0.1 | 230 |
| 255 | N | A | 1.3 | 0.4 | 159 |
| 255 | N | L | 1.6 | 0.4 | 82 |
| 255 | N | M | 1.2 | 0.1 | 195 |
| 255 | N | Q | 1.1 | 0.0 | 216 |
| 255 | N | R | 1.3 | 0.3 | 161 |
| 255 | N | S | 1.3 | 0.1 | 165 |
| 256 | E | A | 0.9 | 0.1 | 244 |
| 259 | H | W | 1.1 | 0.2 | 217 |
| 260 | I | L | 1.1 | 0.1 | 210 |
| 260 | I | V | 1.0 | 0.1 | 228 |
| 267 | R | C | 1.0 | 0.0 | 226 |
| 272 | I | V | 0.8 | 0.0 | 253 |
| 276 | L | G | 0.8 | 0.1 | 256 |
| 278 | I | L | 1.1 | 0.0 | 214 |
| 286 | S | A | 0.9 | 0.1 | 241 |
| 298 | S | A | 2.1 | 0.1 | 21 |
| 298 | S | T | 2.3 | 0.5 | 14 |
| 299 | D | A | 2.0 | 0.4 | 28 |
| 302 | N | A | 1.9 | 0.2 | 33 |
| 303 | A | C | 2.0 | 0.9 | 24 |
| 303 | A | D | 1.5 | 0.4 | 117 |
| 303 | A | E | 2.3 | 0.8 | 16 |
| 303 | A | S | 2.6 | 1.0 | 5 |
| 304 | T | A | 0.7 | NA | 262 |
| 305 | S | A | 1.0 | NA | 222 |
| 305 | S | G | 0.7 | NA | 263 |
| 307 | A | S | 0.9 | NA | 250 |
| 312 | V | L | 1.9 | 0.8 | 34 |
| 320 | R | L | 1.1 | 0.3 | 215 |
| 321 | R | N | 1.7 | 0.1 | 70 |
| 327 | G | L | 2.4 | 0.3 | 9 |
| 327 | G | Q | 2.8 | 0.2 | 4 |
| 327 | G | V | 2.4 | 0.1 | 11 |
| 328 | A | C | 1.7 | 1.0 | 57 |
| 328 | A | D | 2.3 | 0.4 | 13 |
| 328 | A | R | 3.0 | 2.2 | 2 |
| 328 | A | S | 2.2 | 0.9 | 17 |
| 328 | A | T | 1.6 | 1.2 | 84 |
| 328 | A | V | 1.8 | 0.5 | 40 |

### Example 7

### DNA Shuffling to Create Dicamba Decarboxylase Variants with Improved Enzymatic Activity

DNA shuffling is a way to rapidly propagate improved variants in a directed evolution experiment to harness the power of selection to evolve protein function. Through multiple cycles or rounds of DNA shuffling, a large number of beneficial sequence variations are recombined to create functionally improved shuffled variants. Each round of shuffling consists of a parent template and diversity selection, library construction, activity assay, and hit selection. Amino acid changes from the best hits from one round are selected for inclusion in the diversity for library construction in the next round. The initial set of sequences or substitutions on a backbone sequence for shuffling are obtained through several avenues including: 1) natural variation in homologs; 2) saturation mutagenesis; 3) random or site directed mutagenesis; 4) rational design through computational modeling based on structure models.

Using the pre-screened neutral/beneficial amino acid substitutions found from saturation mutagenesis, dicamba decarboxylase DNA shuffling was performed. Shuffled libraries were constructed using techniques including family shuffling, single-gene shuffling, back-crossing, semi-synthetic and synthetic shuffling (Zhang J-H et al. (1997) Proc Natl Acad Sci 94, 4504-4509; Crameri et al. (1998) Nature 391: 288-291; Ness et al. (2002) Nat Biotech 20:1251-1255). Genes coding for shuffled variants of dicamba decarboxylase were cloned into the expression vector specified in Example 2 and introduced into E. coli. The library was plated out on rich agar medium, then individual colonies were picked and grown in magic medium (Invitrogen) in 96-well format at 30° C. overnight. Variants from four 96-well plates were then combined into 384-well assay plates for ¹⁴CO₂ capturing assay as described in Example 1. Variants with higher dicamba decarboxylase activity produce more ¹⁴CO₂ leading to higher intensity spots after exposure, image scanning, and image analysis. Proteins from these cells were then purified for detailed analysis as described in Example 1. Characteristics of k_{cat} and K_{M} were determined as described previously in Example 1. The first round of DNA shuffling incorporated approximately 5 amino acid substitutions from the 30 selected amino acids listed in Table 8 into each progeny variant. Shuffled gene variant libraries were made based on SEQ ID NO:123. Many shuffled variants showed similar or higher dicamba decarboxylase activity compared to the SEQ ID NO:123 (FIG. 9). Shuffled variants with improvement in enzyme characteristics are included in Table 9. Three shuffled variants (SEQ ID NO:125; SEQ ID NO:126; and SEQ ID NO:128) showed greater than 2-fold improvement in k_{cat}/K_{M} as compared with the backbone from this round of shuffling (Table 9). Amino acid substitutions for each improved variant are also displayed in Table 9. Iterative rounds of shuffling continued with the diversity created by mutagenesis and selected by screening.

TABLE 8
30 amino acid changes selected for round one DNA shuffling

| | | | Average | | |
|---|---|---|---|---|---|
| | Amino | | Activity | | |
| Amino | Acid of | | (Fold of | STDEV of | Variant |
| Acid | SEQ ID | Designed | SEQ ID | Average | Ranking by |
| Position | NO: 109 | Alteration | NO: 109) | Activity | Activity |
| 20 | D | F | 1.9 | 0.2 | 32 |
| 27 | G | S | 2.2 | 0.2 | 19 |
| 30 | W | L | 1.7 | 0.0 | 63 |
| 38 | L | I | 2.0 | 0.0 | 30 |
| 42 | D | M | 2.4 | 0.4 | 10 |
| 43 | T | C | 1.7 | 0.3 | 58 |
| 50 | A | K | 1.9 | 0.0 | 35 |
| 52 | G | E | 3.1 | 1.2 | 1 |
| 61 | N | A | 2.9 | 0.9 | 3 |
| 61 | N | S | 2.5 | 0.2 | 7 |
| 64 | A | G | 2.6 | 0.2 | 6 |
| 67 | A | S | 1.7 | 0.1 | 54 |
| 68 | I | Q | 1.6 | 0.0 | 77 |
| 84 | V | F | 1.6 | 0.1 | 89 |
| 101 | A | G | 1.6 | 0.0 | 75 |
| 108 | D | E | 1.7 | 0.1 | 60 |
| 127 | L | M | 2.4 | 1.0 | 8 |
| 212 | R | Q | 1.7 | 0.0 | 53 |
| 214 | N | Q | 1.8 | 0.1 | 41 |
| 229 | W | Y | 1.7 | 0.1 | 68 |
| 235 | V | I | 1.8 | 0.0 | 44 |
| 238 | V | G | 2.0 | 0.2 | 27 |
| 239 | K | H | 1.8 | 0.1 | 46 |
| 240 | L | E | 2.1 | 0.1 | 22 |
| 243 | R | A | 1.8 | 0.4 | 37 |
| 298 | S | A | 2.1 | 0.1 | 21 |
| 302 | N | A | 1.9 | 0.2 | 33 |
| 303 | A | S | 2.6 | 1.0 | 5 |
| 321 | R | N | 1.7 | 0.1 | 70 |
| 327 | G | Q | 2.8 | 0.2 | 4 |
| 328 | A | D | 2.3 | 0.4 | 13 |

TABLE 9
Variants with enzyme kinetic characteristics improved from SEQ ID NO: 1.

| SEQ | | | | | | | | | | | | | |
|---|---|---|---|---|---|---|---|---|---|---|---|---|---|
| ID | Sequence | Amino acid position of SEQ ID NO: 1 | | | | | | | | | | | |
| NO | Description | 20 | 27 | 30 | 61 | 84 | 212 | 214 | 229 | 235 | 238 | 239 | 240 |
| 1 | 2,6- | D | G | W | N | V | R | N | W | N | V | K | L |
| | Dihydroxybenzoate | | | | | | | | | | | | |
| | Decarboxylase | | | | | | | | | | | | |
| 109 | Designed variant of | _{—} | _{—} | _{—} | _{—} | _{—} | _{—} | _{—} | _{—} | V | _{—} | _{—} | _{—} |
| | SEQ ID NO: 1 | | | | | | | | | | | | |
| 123 | N61A of SEQ ID | _{—} | _{—} | _{—} | A | _{—} | _{—} | _{—} | _{—} | V | _{—} | _{—} | _{—} |
| | NO: 109 | | | | | | | | | | | | |
| 124 | Shuffled variant of | _{—} | _{—} | _{—} | A | F | Q | _{—} | _{—} | _{—} | G | _{—} | _{—} |
| | SEQ ID NO: 123 | | | | | | | | | | | | |
| 125 | Shuffled variant of | _{—} | _{—} | _{—} | A | F | _{—} | Q | Y | I | _{—} | H | E |
| | SEQ ID NO: 123 | | | | | | | | | | | | |
| 126 | Shuffled variant of | _{—} | _{—} | _{—} | A | F | _{—} | _{—} | Y | I | _{—} | _{—} | _{—} |
| | SEQ ID NO: 123 | | | | | | | | | | | | |
| 127 | Shuffled variant of | _{—} | S | _{—} | A | _{—} | _{—} | _{—} | _{—} | _{—} | _{—} | _{—} | _{—} |
| | SEQ ID NO: 123 | | | | | | | | | | | | |
| 128 | Shuffled variant of | F | _{—} | _{—} | A | _{—} | _{—} | Q | Y | I | _{—} | _{—} | P |
| | SEQ ID NO: 123 | | | | | | | | | | | | |
| 129 | Shuffled variant of | _{—} | _{—} | L | A | _{—} | _{—} | _{—} | Y | I | _{—} | _{—} | E |
| | SEQ ID NO: 123 | | | | | | | | | | | | |

| SEQ | | | | | | Kinetic characteristics | | |
|---|---|---|---|---|---|---|---|---|
| ID | Amino acid position of SEQ ID NO: 1 | | | | | K_{M} | kcat | kcat/K_{M} |
| NO | 243 | 298 | 302 | 303 | 328 | (mM) | (min⁻¹) | (min⁻¹mM⁻¹) |
| 1 | R | S | N | A | A | 15.000 | 0.020 | 0.001 |
| 109 | _{—} | _{—} | _{—} | _{—} | _{—} | 4.660 | 0.032 | 0.007 |
| 123 | _{—} | _{—} | _{—} | _{—} | _{—} | 4.860 | 0.560 | 0.115 |
| 124 | A | _{—} | _{—} | _{—} | D | 1.990 | 0.190 | 0.096 |
| 125 | _{—} | _{—} | _{—} | _{—} | _{—} | 6.650 | 1.640 | 0.247 |
| 126 | _{—} | A | _{—} | _{—} | _{—} | 8.080 | 2.380 | 0.295 |
| 127 | _{—} | _{—} | _{—} | _{—} | D | 6.740 | 0.920 | 0.136 |
| 128 | _{—} | _{—} | A | S | _{—} | 2.790 | 0.660 | 0.238 |
| 129 | _{—} | A | _{—} | _{—} | _{—} | 15.910 | 3.040 | 0.191 |

### Example 9

### Use of ProSAR-Driven DNA Shuffling to Create Dicamba Decarboxylase Variants with Improved Enzymatic Activity

The contributions of individual amino acid substitutions toward the activity of dicamba decarboxylastion depend on the backbone sequence. Through the process of DNA shuffling, the backbone is changed each round. For positions that are strong determinants of a particular property, substitutions in those positions may have an effect in multiple sequence contexts. For positions that are weak determinants, however, the expected effect of substitution may change from one protein sequence context to the next. The statistical learning tool ProSAR (Protein Sequence Activity Relationship) developed by Fox R et al (2003, Protein Engineering 16, 589-597) was chosen to facilitate the design of shuffling libraries. The creation of ProSAR models that can be used to infer the contributions of mutational effects on protein function provides the basis for ProSAR-driven DNA shuffling. In principal, this iterative process of DNA shuffling is done by statistical analysis through linear regression on training sets derived from one or more combinatorial libraries per round. At the end of each round, the best variant is selected to serve as the backbone for the next round. Amino acid substitutions are selected as variation for the next round based on the prediction of ProSAR analysis on the current backbone protein sequence. Within a given training set consisting of one or more combinatorial libraries, statistical learning is achieved by formulating an equation that correlates mutations with protein function in the following manner: y=c₁ₐx₁ₐ+c_{1b}x_{1b}+c₂ₐx₂ₐ+c_{2b}x_{2b}+ . . . +cⱼₐxⱼₐ+c_{jb}x_{jb} . . . where y is the predicted function (activity) of the protein sequence, cⱼₐ is the regression coefficient corresponding to the mutational effect of having residue choice a present at variable position j, and xⱼₐ is a variable indicating the presence (xⱼₐ=1) or absence (xⱼₐ=0) of residue a at position j (Fox et al., 2007. Nature Biotechnology 25(3): 338-344). In general, it is assumed that the mutational effects are mostly additive and that only linear terms corresponding to each mutation's independent effect on function appear in equation. When needed, nonlinear terms can be added to capture putatively important interactions between mutations.

### Example 10

### Transformation of Arabidopsis with Dicamba Decarboxylase Genes and Evaluation of Herbicide Response

Arabidopsis (Arabidopsis thaliana) expressing dicamba decarboxylase genes were produced using the floral dip method of Agrobacterium mediated transformation (Clough S J and Bent A F, 1998, Plant 16:735-43; Chung M. H., Chen M. K., Pan S. M. 2000. Transgenic Res. 9: 471-476; Weigel D. and Glazebrook J. 2006. In Planta Transformation of Arabidopsis. Cold Spring Harb. Protoc. 4668 3). Briefly, Arabidopsis (Col-O) plants were grown in soil in pots. The first inflorescence shoots were removed as soon as they emerged. Plants were ready for transformation when the secondary inflorescence shoots were about 3 inches tall. Agrobacterium carrying a suitable binary vector were cultured in 5 ml LB medium at 28° C. with shaking at 200 rpm for two days. 1 ml of the culture was then inoculated into 200 ml fresh LB media and incubated again with vigorous agitation for an additional 20-24 hours at 28° C. The Agrobacterium culture was then subjected to centrifugation at 6000 rpm in a GSA rotor (or equivalent) for 10 minutes. The pellet was resuspended in 20-100 ml of spraying medium containing 5% sucrose and 0.01-0.2% (v/v) Silwet L-77. The Agrobacterium suspension was transferred into a hand-held sprayer for spraying onto inflorescences of the transformation-ready Arabidopsis plants. The sprayed plants were covered with a humidity dome for 24 hours before the cover was removed for growth under normal growing conditions. Seeds were harvested. Screening of transformants was performed under sterile conditions. Surface sterilized seeds were placed onto MS-Agar plates (Phyto Technology labs Prod. No. M519) containing appropriate selective antibiotics (kanamycin 50 mg/L, hygromycin 20 mg/L, or bialaphos 10 mg/L). Anti-Agrobacteirum antibiotic timentin was also included in the media. Plates were cultured at 21° C. at 16 hr light for 7-14 days. Transgenic events harboring dicamba decarboxylase genes were germinated and transferred to soil pots in the greenhouse for evaluation of herbicide tolerance.

A selectable marker gene used to facilitate Arabidopsis transformation is a chimeric gene composed of the 35S promoter from Cauliflower Mosaic Virus (Odell et al. 1985. Nature 313:810-812), the bar gene from Streptomyces hygroscopicus (Thompson et al. (1987) EMBO J. 6:2519-2523) and the 3′UBQ14 terminator region from Arabidopsis (Callis et al., 1995. Genetics 139 (2), 921-939). Another visual selectable marker gene used to facilitate Arabidopsis transformation is a chimeric gene composed of the UBQ promoter from soybean (Xing et al., 2010. Plant Biotechnology Journal 8:772-782), the YFP coding sequence, and the 3′ region of the nopaline synthase gene from the T-DNA of the Ti plasmid of Agrobacterium tumefaciens. Bialophos was used as the selection agent during the transformation process. Dicamba decarboxylase genes were expressed with a constitutive promoter, for example, the Arabidopsis UBQ10 promoter (Norris et al., 1993. Plant Mol Biol 21:895-906) or UBQ3 promoter (Norris et al., 1993. Plant Mol Biol 21:895-906) for strong or moderate expression and the 3′ terminator region of the French bean phaseolin gene (Sun et al., 1981. Nature 289:37-41; Slightom et al., 1983. Proc. Natl. Acad. Sci. U.S.A. 80 (7), 1897-1901).

Seeds of Arabidopsis ecotype Columbia (Col-0) and dicamba decarboxylase transgenic events were surface sterilized with 70% (v/v) ethanol for 5 minutes and 10% (v/v) bleach for 15 minutes. After being washed three times with distilled water, the seeds were incubated at 4° C. for 4 days. The seeds were then germinated on 1× Murashige and Skoog (MS) medium with a pH of 5.7, 3% (w/v) sucrose, and 0.8% (w/v) agar. After incubation for 3.5 days, the seedlings were transferred to basal medium containing B5 vitamin, 3% (w/v) sucrose, 2.5 mm MES (pH 5.7), 1.2% (w/v) agar, and filter sterilized dicamba was added to the media at 60° C. The concentrations of dicamba were 0 μM, 5.0 μM, 7.0 μM, and 10 μM. The basal medium contained 1/10×MS macronutrients (2.05 mm NH₄NO₃, 1.8 mm KNO₃, 0.3 mm CaCl₂, and 0.156 mm MgSO₄) and 1×MS micronutrients (100 μm H₃BO₃, 100 μm MnSO₄, 30 μm ZnSO₄, 5 μm KI, 1 μm Na₂MoO₄, 0.1 μm CuSO₄, 0.1 μm CoCl₂, 0.1 mm FeSO₄, and 0.1 mm Na₂EDTA). The seedlings were placed vertically, and the temperature maintained at 23° C. to allow root growth along the surface of the agar, with a photoperiod of 16 h of light and 8 h of dark.

After 8 days on media with various concentrations of dicamba, the length of the primary root is measured. In wild type Arabidopsis, root growth inhibition is expected from auxin herbicide treatment. The length of the primary root in wild type plants is reduced with dicamba treatment. The more dicamba, the shorter the primary root. The difference in root growth inhibition between wild type and dicamba decarboxylase transgenic events is compared. Alleviation of root growth inhibition on dicamba is an indication of auxin herbicide detoxification due to dicamba decarboxylase activity.

### Example 11

### Transformation of Soybean with Dicamba Decarboxylase Genes

Soybean plants expressing dicamba decarboxylase transgenes are produced using the method of particle gun bombardment (Klein et al. (1987) Nature 327:70-73, U.S. Pat. No. 4,945,050) using a DuPont Biolistic PDS 1000/He instrument. Transgenes include coding sequences of active dicamba decarboxylases. A selectable marker gene used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. Another selectable marker used to facilitate soybean transformation is a chimeric gene composed of the S-adenosylmethionine synthase (SAMS) promoter (U.S. Pat. No. 7,741,537) from soybean, a highly resistant allele of ALS (U.S. Pat. Nos. 5,605,011, 5,378,824, 5,141,870, and 5,013,659), and the native soybean ALS terminator region. The selection agent used during the transformation process is either hygromycin or chlorsulfuron depending on the marker gene present. Dicamba decarboxylase genes are expressed with a constitutive promoter, for example, the Arabidopsis UBQ10 promoter (Norris et al. (1993) Plant Mol Biol 21:895-906), and the phaseolin gene terminator (Sun S M et al. (1981) Nature 289:37-41 and Slightom et al. (1983) Proc. Natl. Acad. Sci. U.S.A. 80 (7), 1897-1901). Bombardments are carried out with linear DNA fragments purified away from any bacterial vector DNA. The selectable marker gene cassette is in the same DNA fragment as the dicamba decarboxylase expression cassette. Bombarded soybean embryogenic suspension tissue is cultured for one week in the absence of selection agent, then placed in liquid selection medium for 6 weeks. Putative transgenic suspension tissue is sampled for PCR analysis to determine the presence of the dicamba decarboxylase gene. Putative transgenic suspension culture tissue is maintained in selection medium for 3 weeks to obtain enough tissue for plant regeneration. Suspension tissue is matured for 4 weeks using standard procedures; matured somatic embryos are desiccated for 4-7 days and then placed on germination induction medium for 2-4 weeks. Germinated plantlets are transferred to soil in cell pack trays for 3 weeks for acclimatization. Plantlets are potted to 10-inch pots in the greenhouse for evaluation of herbicide resistance. Transgenic soybean, Arabidopsis and other species of plants could also be produced using Agrobacterium transformation using a variety of ex-plants.

### Example 12

### Herbicide Tolerance Evaluation of Dicamba Decarboxylase Transgenic Soybean Plants

T0, T1 or homozygous T2 and later plants expressing dicamba decarboxylase transgenes are grown in a controlled environment (for example, 25° C., 70% humidity, 16 hr light) to either V2 or V8 growth stage and then sprayed with commercial dicamba herbicide formulations at a rate up to 450 g/ha. Herbicide applications may be made with added 0.25% nonionic surfactant and 1% ammonium sulfate in a spray volume of 374 L/ha. Individual plants are compared to untreated plants of similar genetic background, evaluated for herbicide response at seven to twenty-one days after treatment and assigned a visual response score from 0 to 100% injury (0=no effect to 100=dead plant). Expression of the dicamba decarboxylase gene varies due to the genomic location in the unique TO plants. Plants that do not express the transgenic dicamba decarboxylase gene are severely injured by dicamba herbicide. Plants expressing introduced dicamba decarboxylase genes may show tolerance to the dicamba herbicide due to activity of the dicamba decarboxylase.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

TABLE 1
Abbreviated version of HRAC Herbicide Classification

| I. | ALS Inhibitors (WSSA Group 2) | | | |
|---|---|---|---|---|
| | A. | Sulfonylureas | | |
| | | 1. | Azimsulfuron | |
| | | 2. | Chlorimuron-ethyl | |
| | | 3. | Metsulfuron-methyl | |
| | | 4. | Nicosulfuron | |
| | | 5. | Rimsulfuron | |
| | | 6. | Sulfometuron-methyl | |
| | | 7. | Thifensulfuron-methyl | |
| | | 8. | Tribenuron-methyl | |
| | | 9. | Amidosulfuron | |
| | | 10. | Bensulfuron-methyl | |
| | | 11. | Chlorsulfuron | |
| | | 12. | Cinosulfuron | |
| | | 13. | Cyclosulfamuron | |
| | | 14. | Ethametsulfuron-methyl | |
| | | 15. | Ethoxysulfuron | |
| | | 16. | Flazasulfuron | |
| | | 17. | Flupyrsulfuron-methyl | |
| | | 18. | Foramsulfuron | |
| | | 19. | Imazosulfuron | |
| | | 20. | Iodosulfuron-methyl | |
| | | 21. | Mesosulfuron-methyl | |
| | | 22. | Oxasulfuron | |
| | | 23. | Primisulfuron-methyl | |
| | | 24. | Prosulfuron | |
| | | 25. | Pyrazosulfuron-ethyl | |
| | | 26. | Sulfosulfuron | |
| | | 27. | Triasulfuron | |
| | | 28. | Trifloxysulfuron | |
| | | 29. | Triflusulfuron-methyl | |
| | | 30. | Tritosulfuron | |
| | | 31. | Halosulfuron-methyl | |
| | | 32. | Flucetosulfuron | |
| | B. | Sulfonylaminocarbonyltriazolinones | | |
| | | 1. | Flucarbazone | |
| | | 2. | Procarbazone | |
| | C. | Triazolopyrimidines | | |
| | | 1. | Cloransulam-methyl | |
| | | 2. | Flumetsulam | |
| | | 3. | Diclosulam | |
| | | 4. | Florasulam | |
| | | 5. | Metosulam | |
| | | 6. | Penoxsulam | |
| | | 7. | Pyroxsulam | |
| | D. | Pyrimidinyloxy(thio)benzoates | | |
| | | 1. | Bispyribac | |
| | | 2. | Pyriftalid | |
| | | 3. | Pyribenzoxim | |
| | | 4. | Pyrithiobac | |
| | | 5. | Pyriminobac-methyl | |
| | E. | Imidazolinones | | |
| | | 1. | Imazapyr | |
| | | 2. | Imazethapyr | |
| | | 3. | Imazaquin | |
| | | 4. | Imazapic | |
| | | 5. | Imazamethabenz-methyl | |
| | | 6. | Imazamox | |
| II. | Other Herbicides--Active Ingredients/ | | | |
| | Additional Modes of Action | | | |
| | A. | Inhibitors of Acetyl CoA carboxylase | | |
| | | (ACCase) (WSSA Group 1) | | |
| | | 1. | Aryloxyphenoxypropionates (‘FOPs’) | |
| | | | a. | Quizalofop-P-ethyl |
| | | | b. | Diclofop-methyl |
| | | | c. | Clodinafop-propargyl |
| | | | d. | Fenoxaprop-P-ethyl |
| | | | e. | Fluazifop-P-butyl |
| | | | f. | Propaquizafop |
| | | | g. | Haloxyfop-P-methyl |
| | | | h. | Cyhalofop-butyl |
| | | | i. | Quizalofop-P-ethyl |
| | | 2. | Cyclohexanediones (‘DIMs’) | |
| | | | a. | Alloxydim |
| | | | b. | Butroxydim |
| | | | c. | Clethodim |
| | | | d. | Cycloxydim |
| | | | e. | Sethoxydim |
| | | | f. | Tepraloxydim |
| | | | g. | Tralkoxydim |
| | B. | Inhibitors of Photosystem II-HRAC | | |
| | | Group C1/WSSA Group 5 | | |
| | | 1. | Triazines | |
| | | | a. | Ametryne |
| | | | b. | Atrazine |
| | | | c. | Cyanazine |
| | | | d. | Desmetryne |
| | | | e. | Dimethametryne |
| | | | f. | Prometon |
| | | | g. | Prometryne |
| | | | h. | Propazine |
| | | | i. | Simazine |
| | | | j. | Simetryne |
| | | | k. | Terbumeton |
| | | | l. | Terbuthylazine |
| | | | m. | Terbutryne |
| | | | n. | Trietazine |
| | | 2. | Triazinones | |
| | | | a. | Hexazinone |
| | | | b. | Metribuzin |
| | | | c. | Metamitron |
| | | 3. | Triazolinone | |
| | | | a. | Amicarbazone |
| | | 4. | Uracils | |
| | | | a. | Bromacil |
| | | | b. | Lenacil |
| | | | c. | Terbacil |
| | | 5. | Pyridazinones | |
| | | | a. | Pyrazon |
| | | 6. | Phenyl carbamates | |
| | | | a. | Desmedipham |
| | | | b. | Phenmedipham |
| | C. | Inhibitors of Photosystem II--HRAC | | |
| | | Group C2/WSSA Group 7 | | |
| | | 1. | Ureas | |
| | | | a. | Fluometuron |
| | | | b. | Linuron |
| | | | c. | Chlorobromuron |
| | | | d. | Chlorotoluron |
| | | | e. | Chloroxuron |
| | | | f. | Dimefuron |
| | | | g. | Diuron |
| | | | h. | Ethidimuron |
| | | | i. | Fenuron |
| | | | j. | Isoproturon |
| | | | k. | Isouron |
| | | | l. | Methabenzthiazuron |
| | | | m. | Metobromuron |
| | | | n. | Metoxuron |
| | | | o. | Monolinuron |
| | | | p. | Neburon |
| | | | q. | Siduron |
| | | | r. | Tebuthiuron |
| | | 2. | Amides | |
| | | | a. | Propanil |
| | | | b. | Pentanochlor |
| | D. | Inhibitors of Photosystem II--HRAC | | |
| | | Group C3/WSSA Group 6 | | |
| | | 1. | Nitriles | |
| | | | a. | Bromofenoxim |
| | | | b. | Bromoxynil |
| | | | c. | Ioxynil |
| | | 2. | Benzothiadiazinone (Bentazon) | |
| | | | a. | Bentazon |
| | | 3. | Phenylpyridazines | |
| | | | a. | Pyridate |
| | | | b. | Pyridafol |
| | E. | Photosystem-I-electron diversion | | |
| | | (Bipyridyliums) (WSSA Group 22) | | |
| | | 1. | Diquat | |
| | | 2. | Paraquat | |
| | F. | Inhibitors of PPO (protoporphyrinogen | | |
| | | oxidase) (WSSA Group 14) | | |
| | | 1. | Diphenylethers | |
| | | | a. | Acifluorfen-Na |
| | | | b. | Bifenox |
| | | | c. | Chlomethoxyfen |
| | | | d. | Fluoroglycofen-ethyl |
| | | | e. | Fomesafen |
| | | | f. | Halosafen |
| | | | g. | Lactofen |
| | | | h. | Oxyfluorfen |
| | | 2. | Phenylpyrazoles | |
| | | | a. | Fluazolate |
| | | | b. | Pyraflufen-ethyl |
| | | 3. | N-phenylphthalimides | |
| | | | a. | Cinidon-ethyl |
| | | | b. | Flumioxazin |
| | | | c. | Flumiclorac-pentyl |
| | | 4. | Thiadiazoles | |
| | | | a. | Fluthiacet-methyl |
| | | | b. | Thidiazimin |
| | | 5. | Oxadiazoles | |
| | | | a. | Oxadiazon |
| | | | b. | Oxadiargyl |
| | | 6. | Triazolinones | |
| | | | a. | Carfentrazone-ethyl |
| | | | b. | Sulfentrazone |
| | | 7. | Oxazolidinediones | |
| | | | a. | Pentoxazone |
| | | 8. | Pyrimidindiones | |
| | | | a. | Benzfendizone |
| | | | b. | Butafenicil |
| | | 9. | Others | |
| | | | a. | Pyrazogyl |
| | | | b. | Profluazol |
| | G. | Bleaching: Inhibition of carotenoid | | |
| | | biosynthesis at the phytoene desaturase step | | |
| | | (PDS) (WSSA Group 12) | | |
| | | 1. | Pyridazinones | |
| | | | a. | Norflurazon |
| | | 2. | Pyridinecarboxamides | |
| | | | a. | Diflufenican |
| | | | b. | Picolinafen |
| | | 3. | Others | |
| | | | a. | Beflubutamid |
| | | | b. | Fluridone |
| | | | c. | Flurochloridone |
| | | | d. | Flurtamone |
| | H. | Bleaching: Inhibition of 4- | | |
| | | hydroxyphenyl-pyruvate-dioxygenase (4-HPPD) | | |
| | | (WSSA Group 28) | | |
| | | 1. | Triketones | |
| | | | a. | Mesotrione |
| | | | b. | Sulcotrione |
| | | | c. | topramezone |
| | | | d. | tembotrione |
| | | 2. | Isoxazoles | |
| | | | a. | Pyrasulfotole |
| | | | b. | Isoxaflutole |
| | | 3. | Pyrazoles | |
| | | | a. | Benzofenap |
| | | | b. | Pyrazoxyfen |
| | | | c. | Pyrazolynate |
| | | 4. | Others | |
| | | | a. | Benzobicyclon |
| | I. | Bleaching: Inhibition of carotenoid | | |
| | | biosynthesis (unknown target) (WSSA Group 11 | | |
| | | and 13) | | |
| | | 1. | Triazoles (WSSA Group 11) | |
| | | | a. | Amitrole |
| | | 2. | Isoxazolidinones (WSSA Group 13) | |
| | | | a. | Clomazone |
| | | 3. | Ureas | |
| | | | a. | Fluometuron |
| | | 3. | Diphenylether | |
| | | | a. | Aclonifen |
| | J. | Inhibition of EPSP Synthase | | |
| | | 1. | Glycines (WSSA Group 9) | |
| | | | a. | Glyphosate |
| | | | b. | Sulfosate |
| | K. | Inhibition of glutamine synthetase | | |
| | | 1. | Phosphinic Acids | |
| | | | a. | Glufosinate-ammonium |
| | | | b. | Bialaphos |
| | L. | Inhibition of DHP (dihydropteroate) | | |
| | | synthase (WSSA Group 18) | | |
| | | 1 | Carbamates | |
| | | | a. | Asulam |
| | M. | Microtubule Assembly Inhibition | | |
| | | (WSSA Group 3) | | |
| | | 1. | Dinitroanilines | |
| | | | a. | Benfluralin |
| | | | b. | Butralin |
| | | | c. | Dinitramine |
| | | | d. | Ethalfluralin |
| | | | e. | Oryzalin |
| | | | f. | Pendimethalin |
| | | | g. | Trifluralin |
| | | 2. | Phosphoroamidates | |
| | | | a. | Amiprophos-methyl |
| | | | b. | Butamiphos |
| | | 3. | Pyridines | |
| | | | a. | Dithiopyr |
| | | | b. | Thiazopyr |
| | | 4. | Benzamides | |
| | | | a. | Pronamide |
| | | | b. | Tebutam |
| | | 5. | Benzenedicarboxylic acids | |
| | | | a. | Chlorthal-dimethyl |
| | N. | Inhibition of mitosis/microtubule | | |
| | | organization WSSA Group 23) | | |
| | | 1. | Carbamates | |
| | | | a. | Chlorpropham |
| | | | b. | Propham |
| | | | c. | Carbetamide |
| | O. | Inhibition of cell division (Inhibition of | | |
| | | very long chain fatty acids as proposed | | |
| | | mechanism; WSSA Group 15) | | |
| | | 1. | Chloroacetamides | |
| | | | a. | Acetochlor |
| | | | b. | Alachlor |
| | | | c. | Butachlor |
| | | | d. | Dimethachlor |
| | | | e. | Dimethanamid |
| | | | f. | Metazachlor |
| | | | g. | Metolachlor |
| | | | h. | Pethoxamid |
| | | | i. | Pretilachlor |
| | | | j. | Propachlor |
| | | | k. | Propisochlor |
| | | | l. | Thenylchlor |
| | | 2. | Acetamides | |
| | | | a. | Diphenamid |
| | | | b. | Napropamide |
| | | | c. | Naproanilide |
| | | 3. | Oxyacetamides | |
| | | | a. | Flufenacet |
| | | | b. | Mefenacet |
| | | 4. | Tetrazolinones | |
| | | | a. | Fentrazamide |
| | | 5. | Others | |
| | | | a. | Anilofos |
| | | | b. | Cafenstrole |
| | | | c. | Indanofan |
| | | | d. | Piperophos |
| | P. | Inhibition of cell wall (cellulose) | | |
| | | synthesis | | |
| | | 1. | Nitriles (WSSA Group 20) | |
| | | | a. | Dichlobenil |
| | | | b. | Chlorthiamid |
| | | 2. | Benzamides (isoxaben (WSSA | |
| | | | Group 21)) | |
| | | | a. | Isoxaben |
| | | 3. | Triazolocarboxamides (flupoxam) | |
| | | | a. | Flupoxam |
| | Q. | Uncoupling (membrane disruption): | | |
| | | (WSSA Group 24) | | |
| | | 1. | Dinitrophenols | |
| | | | a. | DNOC |
| | | | b. | Dinoseb |
| | | | c. | Dinoterb |
| | R. | Inhibition of Lipid Synthesis by other | | |
| | | than ACC inhibition | | |
| | | 1. | Thiocarbamates (WSSA Group 8) | |
| | | | a. | Butylate |
| | | | b. | Cycloate |
| | | | c. | Dimepiperate |
| | | | d. | EPTC |
| | | | e. | Esprocarb |
| | | | f. | Molinate |
| | | | g. | Orbencarb |
| | | | h. | Pebulate |
| | | | i. | Prosulfocarb |
| | | | j. | Benthiocarb |
| | | | k. | Tiocarbazil |
| | | | l. | Triallate |
| | | | m. | Vernolate |
| | | 2. | Phosphorodithioates | |
| | | | a. | Bensulide |
| | | 3. | Benzofurans | |
| | | | a. | Benfuresate |
| | | | b. | Ethofumesate |
| | | 4. | Halogenated alkanoic acids | |
| | | | (WSSA Group 26) | |
| | | | a. | TCA |
| | | | b. | Dalapon |
| | | | c. | Flupropanate |
| | S. | Synthetic auxins (IAA-like) (WSSA | | |
| | | Group 4) | | |
| | | 1. | Phenoxycarboxylic acids | |
| | | | a. | Clomeprop |
| | | | b. | 2,4-D |
| | | | c. | Mecoprop |
| | | 2. | Benzoic acids | |
| | | | a. | Dicamba |
| | | | b. | Chloramben |
| | | | c. | TBA |
| | | 3. | Pyridine carboxylic acids | |
| | | | a. | Clopyralid |
| | | | b. | Fluroxypyr |
| | | | c. | Picloram |
| | | | d. | Tricyclopyr |
| | | 4. | Quinoline carboxylic acids | |
| | | | a. | Quinclorac |
| | | | b. | Quinmerac |
| | | 5. | Others (benazolin-ethyl) | |
| | | | a. | Benazolin-ethyl |
| | | 6. | aminocyclopyrachlor | |
| | T. | Inhibition of Auxin Transport | | |
| | | 1. | Phthalamates; semicarbazones | |
| | | | (WSSA Group 19) | |
| | | | a. | Naptalam |
| | | | b. | Diflufenzopyr-Na |
| | U. | Other Mechanism of Action | | |
| | | 1. | Arylaminopropionic acids | |
| | | | a. | Flamprop-M-methyl/- |
| | | | | isopropyl |
| | | 2. | Pyrazolium | |
| | | | a. | Difenzoquat |
| | | 3. | Organoarsenicals | |
| | | | a. | DSMA |
| | | | b. | MSMA |
| | | 4. | Others | |
| | | | a. | Bromobutide |
| | | | b. | Cinmethylin |
| | | | c. | Cumyluron |
| | | | d. | Dazomet |
| | | | e. | Daimuron-methyl |
| | | | f. | Dimuron |
| | | | g. | Etobenzanid |
| | | | h. | Fosamine |
| | | | i. | Metam |
| | | | j. | Oxaziclomefone |
| | | | k. | Oleic acid |
| | | | l. | Pelargonic acid |
| | | | m. | Pyributicarb |

### Megatable Legends

Megatable 1. The definitions of the column headings are as follows: “MUT ID,” a unique identifier for each substitutions; “Backbone,” the SEQ ID corresponding to the polypeptide backbone in which the substitution was made; “Position,” amino acid position according to the numbering convention of SEQ ID NO: 109, “Ref. A.A.,” the standard single letter code for the amino acid present in the backbone sequence at the indicated position; “Substitution,” the standard single letter code for the amino acid present in the mutant sequence at the indicated position; and “Fold Activity,” refers to the decarboxylation activity of the mutant protein when compared with that of the unmutated backbone protein (SEQ ID NO: 109). Decarboxylation activity of the respective protein samples is determined by measuring the amount of carbon dioxide released from the enzymatic reaction as described herein above.
Megatable 2. The definitions of the column headings are as follows: “SEQ ID NO:”, a unique identifier for each mutated DNA or amino acid sequence; “Trivial Name”, a trivial but unique name for each DNA or protein sequence; “Backbone,” the SEQ ID corresponding to the polypeptide backbone in which the substitution was made; “Fold Activity,” refers to the decarboxylation activity of the mutant or mutant combination protein when compared with that of the unmutated backbone protein (SEQ ID NO: 126, 380, or 509, as appropriate). Decarboxylation activity of the respective protein samples is determined by measuring the amount of carbon dioxide released from the enzymatic reaction as described herein above.

MEGATABLE 1

| MUT | Back- | Posi- | Ref | Substi- | Fold |
|---|---|---|---|---|---|
| ID NO: | bone | tion | A.A. | tution | Activity |
| 1 | 109 | 3 | Q | G | 1.2 |
| 2 | 109 | 3 | Q | M | 1.1 |
| 3 | 109 | 5 | K | E | 0.9 |
| 4 | 109 | 5 | K | I | 1 |
| 5 | 109 | 5 | K | L | 0.8 |
| 6 | 109 | 5 | K | W | 0.9 |
| 7 | 109 | 7 | A | C | 1.3 |
| 8 | 109 | 12 | F | M | 1.3 |
| 9 | 109 | 12 | F | V | 1.2 |
| 10 | 109 | 12 | F | W | 1.2 |
| 11 | 109 | 13 | A | C | 1 |
| 12 | 109 | 15 | P | A | 0.9 |
| 13 | 109 | 15 | P | D | 1 |
| 14 | 109 | 15 | P | E | 1 |
| 15 | 109 | 15 | P | Q | 1 |
| 16 | 109 | 15 | P | T | 1.1 |
| 17 | 109 | 16 | E | A | 1.8 |
| 18 | 109 | 19 | Q | E | 1.2 |
| 19 | 109 | 19 | Q | N | 1.6 |
| 20 | 109 | 20 | D | C | 1.8 |
| 21 | 109 | 20 | D | F | 1.9 |
| 22 | 109 | 20 | D | M | 1.6 |
| 23 | 109 | 20 | D | W | 1.5 |
| 24 | 109 | 21 | S | A | 1.6 |
| 25 | 109 | 21 | S | C | 1 |
| 26 | 109 | 21 | S | G | 1.2 |
| 27 | 109 | 21 | S | L | 1 |
| 28 | 109 | 21 | S | V | 1.2 |
| 29 | 109 | 23 | G | D | 1.5 |
| 30 | 109 | 27 | G | A | 2 |
| 31 | 109 | 27 | G | D | 1.7 |
| 32 | 109 | 27 | G | E | 1.5 |
| 33 | 109 | 27 | G | P | 1.6 |
| 34 | 109 | 27 | G | R | 1.6 |
| 35 | 109 | 27 | G | S | 2.2 |
| 36 | 109 | 27 | G | T | 2 |
| 37 | 109 | 27 | G | Y | 1.6 |
| 38 | 109 | 28 | D | C | 1.8 |
| 39 | 109 | 28 | D | E | 1.6 |
| 40 | 109 | 28 | D | F | 1.4 |
| 41 | 109 | 28 | D | G | 1.5 |
| 42 | 109 | 30 | W | L | 1.7 |
| 43 | 109 | 30 | W | Q | 1 |
| 44 | 109 | 30 | W | S | 0.7 |
| 45 | 109 | 30 | W | V | 1.7 |
| 46 | 109 | 32 | E | V | 1.1 |
| 47 | 109 | 34 | Q | A | 1.2 |
| 48 | 109 | 34 | Q | W | 1.5 |
| 49 | 109 | 38 | L | I | 2 |
| 50 | 109 | 38 | L | M | 1.7 |
| 51 | 109 | 38 | L | R | 1.7 |
| 52 | 109 | 38 | L | T | 1.9 |
| 53 | 109 | 38 | L | V | 1.6 |
| 54 | 109 | 40 | I | M | 1.4 |
| 55 | 109 | 40 | I | S | 1.5 |
| 56 | 109 | 40 | I | V | 1.3 |
| 57 | 109 | 42 | D | A | 2 |
| 58 | 109 | 42 | D | G | 1.5 |
| 59 | 109 | 42 | D | H | 0.9 |
| 60 | 109 | 42 | D | K | 1.6 |
| 61 | 109 | 42 | D | M | 2.4 |
| 62 | 109 | 42 | D | R | 1 |
| 63 | 109 | 42 | D | S | 2 |
| 64 | 109 | 42 | D | T | 1.8 |
| 65 | 109 | 43 | T | C | 1.7 |
| 66 | 109 | 43 | T | D | 1.6 |
| 67 | 109 | 43 | T | E | 1.3 |
| 68 | 109 | 43 | T | G | 1.3 |
| 69 | 109 | 43 | T | M | 1.3 |
| 70 | 109 | 43 | T | Q | 1.7 |
| 71 | 109 | 43 | T | R | 1.5 |
| 72 | 109 | 43 | T | Y | 1.2 |
| 73 | 109 | 46 | K | G | 1.2 |
| 74 | 109 | 46 | K | N | 1.4 |
| 75 | 109 | 46 | K | R | 1.7 |
| 76 | 109 | 47 | L | C | 1.1 |
| 77 | 109 | 47 | L | E | 1.3 |
| 78 | 109 | 47 | L | K | 1.1 |
| 79 | 109 | 47 | L | N | 0.9 |
| 80 | 109 | 47 | L | R | 0.8 |
| 81 | 109 | 47 | L | S | 1.2 |
| 82 | 109 | 50 | A | I | 0.9 |
| 83 | 109 | 50 | A | K | 1.9 |
| 84 | 109 | 50 | A | L | 1 |
| 85 | 109 | 50 | A | R | 1.4 |
| 86 | 109 | 50 | A | S | 1.4 |
| 87 | 109 | 50 | A | T | 1.4 |
| 88 | 109 | 50 | A | V | 1.3 |
| 89 | 109 | 52 | G | E | 3.1 |
| 90 | 109 | 52 | G | L | 1.7 |
| 91 | 109 | 52 | G | N | 1.6 |
| 92 | 109 | 52 | G | Q | 1.7 |
| 93 | 109 | 54 | E | G | 1.6 |
| 94 | 109 | 55 | T | L | 1.5 |
| 95 | 109 | 57 | I | A | 1.4 |
| 96 | 109 | 57 | I | V | 1.1 |
| 97 | 109 | 61 | N | A | 2.9 |
| 98 | 109 | 61 | N | G | 2.3 |
| 99 | 109 | 61 | N | L | 1.7 |
| 100 | 109 | 61 | N | S | 2.5 |
| 101 | 109 | 63 | P | V | 1.8 |
| 102 | 109 | 64 | A | G | 2.6 |
| 103 | 109 | 64 | A | H | 1.7 |
| 104 | 109 | 64 | A | S | 2.1 |
| 105 | 109 | 67 | A | E | 0.9 |
| 106 | 109 | 67 | A | G | 0.8 |
| 107 | 109 | 67 | A | S | 1.7 |
| 108 | 109 | 68 | I | Q | 1.6 |
| 109 | 109 | 69 | P | G | 1.6 |
| 110 | 109 | 69 | P | R | 1.1 |
| 111 | 109 | 69 | P | S | 1.2 |
| 112 | 109 | 69 | P | V | 1.2 |
| 113 | 109 | 70 | D | H | 1.4 |
| 114 | 109 | 72 | R | K | 1.6 |
| 115 | 109 | 72 | R | V | 1.6 |
| 116 | 109 | 73 | K | E | 1.5 |
| 117 | 109 | 73 | K | Q | 1.8 |
| 118 | 109 | 73 | K | R | 1.4 |
| 119 | 109 | 75 | I | R | 1.6 |
| 120 | 109 | 76 | E | G | 1.3 |
| 121 | 109 | 77 | I | C | 1 |
| 122 | 109 | 77 | I | L | 0.9 |
| 123 | 109 | 77 | I | M | 1.3 |
| 124 | 109 | 77 | I | R | 1.4 |
| 125 | 109 | 77 | I | S | 1.5 |
| 126 | 109 | 77 | I | V | 1.2 |
| 127 | 109 | 79 | R | K | 0.7 |
| 128 | 109 | 79 | R | Q | 1.2 |
| 129 | 109 | 81 | A | S | 1.4 |
| 130 | 109 | 84 | V | C | 1.2 |
| 131 | 109 | 84 | V | F | 1.6 |
| 132 | 109 | 84 | V | M | 1.6 |
| 133 | 109 | 88 | E | K | 1.3 |
| 134 | 109 | 89 | C | I | 1.5 |
| 135 | 109 | 89 | C | V | 1.5 |
| 136 | 109 | 91 | K | R | 1.2 |
| 137 | 109 | 93 | P | A | 1.1 |
| 138 | 109 | 93 | P | K | 0.7 |
| 139 | 109 | 93 | P | R | 1.4 |
| 140 | 109 | 94 | D | C | 1.1 |
| 141 | 109 | 94 | D | G | 1.1 |
| 142 | 109 | 94 | D | N | 1 |
| 143 | 109 | 94 | D | Q | 1.2 |
| 144 | 109 | 94 | D | S | 1.2 |
| 145 | 109 | 97 | L | K | 1.2 |
| 146 | 109 | 97 | L | R | 1.3 |
| 147 | 109 | 100 | A | G | 1.3 |
| 148 | 109 | 100 | A | S | 1.5 |
| 149 | 109 | 101 | A | G | 1.6 |
| 150 | 109 | 102 | L | V | 1.4 |
| 151 | 109 | 104 | L | M | 1.9 |
| 152 | 109 | 107 | P | V | 1.8 |
| 153 | 109 | 108 | D | E | 1.7 |
| 154 | 109 | 109 | A | G | 1.3 |
| 155 | 109 | 109 | A | M | 1.5 |
| 156 | 109 | 109 | A | V | 1.5 |
| 157 | 109 | 111 | T | A | 1.4 |
| 158 | 109 | 111 | T | C | 1.6 |
| 159 | 109 | 111 | T | G | 1.5 |
| 160 | 109 | 111 | T | S | 1.7 |
| 161 | 109 | 111 | T | V | 1.5 |
| 162 | 109 | 112 | E | G | 1.4 |
| 163 | 109 | 112 | E | R | 1.5 |
| 164 | 109 | 112 | E | S | 1.5 |
| 165 | 109 | 117 | C | A | 1.7 |
| 166 | 109 | 117 | C | T | 1.8 |
| 167 | 109 | 119 | N | A | 1.4 |
| 168 | 109 | 119 | N | C | 1.3 |
| 169 | 109 | 119 | N | R | 1.5 |
| 170 | 109 | 119 | N | S | 1.3 |
| 171 | 109 | 120 | D | T | 1.7 |
| 172 | 109 | 123 | F | L | 1.3 |
| 173 | 109 | 127 | L | M | 2.4 |
| 174 | 109 | 133 | Q | V | 1.6 |
| 175 | 109 | 134 | E | G | 0.8 |
| 176 | 109 | 137 | G | A | 1.2 |
| 177 | 109 | 137 | G | E | 1.2 |
| 178 | 109 | 138 | Q | G | 1.1 |
| 179 | 109 | 138 | Q | L | 0.9 |
| 180 | 109 | 139 | T | E | 0.7 |
| 181 | 109 | 147 | Q | I | 1.1 |
| 182 | 109 | 150 | P | G | 0.9 |
| 183 | 109 | 153 | G | K | 1.6 |
| 184 | 109 | 167 | R | E | 1.6 |
| 185 | 109 | 174 | S | A | 1.2 |
| 186 | 109 | 178 | D | E | 1.2 |
| 187 | 109 | 181 | P | E | 0.9 |
| 188 | 109 | 195 | A | G | 1.2 |
| 189 | 109 | 212 | R | G | 1.6 |
| 190 | 109 | 212 | R | Q | 1.7 |
| 191 | 109 | 214 | N | Q | 1.8 |
| 192 | 109 | 215 | I | V | 0.8 |
| 193 | 109 | 220 | M | L | 1.7 |
| 194 | 109 | 228 | M | L | 1.4 |
| 195 | 109 | 229 | W | Y | 1.7 |
| 196 | 109 | 231 | I | M | 0.8 |
| 197 | 109 | 234 | R | H | 0.9 |
| 198 | 109 | 234 | R | K | 1 |
| 199 | 109 | 235 | V | I | 1.8 |
| 200 | 109 | 236 | A | G | 1.6 |
| 201 | 109 | 236 | A | Q | 1.2 |
| 202 | 109 | 236 | A | W | 1.4 |
| 203 | 109 | 237 | W | L | 1.1 |
| 204 | 109 | 238 | V | G | 2 |
| 205 | 109 | 238 | V | P | 1.3 |
| 206 | 109 | 239 | K | A | 1.7 |
| 207 | 109 | 239 | K | D | 1.3 |
| 208 | 109 | 239 | K | E | 1.5 |
| 209 | 109 | 239 | K | G | 1.6 |
| 210 | 109 | 239 | K | H | 1.8 |
| 211 | 109 | 240 | L | A | 2.3 |
| 212 | 109 | 240 | L | D | 2.2 |
| 213 | 109 | 240 | L | E | 2.1 |
| 214 | 109 | 240 | L | G | 1.5 |
| 215 | 109 | 240 | L | V | 1.6 |
| 216 | 109 | 243 | R | A | 1.8 |
| 217 | 109 | 243 | R | D | 1.6 |
| 218 | 109 | 243 | R | K | 1.5 |
| 219 | 109 | 243 | R | S | 1.4 |
| 220 | 109 | 243 | R | V | 1.4 |
| 221 | 109 | 245 | P | A | 1.5 |
| 222 | 109 | 248 | R | K | 1.1 |
| 223 | 109 | 249 | R | P | 1.1 |
| 224 | 109 | 251 | M | G | 0.9 |
| 225 | 109 | 251 | M | V | 1.3 |
| 226 | 109 | 252 | D | E | 1 |
| 227 | 109 | 255 | N | A | 1.3 |
| 228 | 109 | 255 | N | L | 1.6 |
| 229 | 109 | 255 | N | M | 1.2 |
| 230 | 109 | 255 | N | Q | 1.1 |
| 231 | 109 | 255 | N | R | 1.3 |
| 232 | 109 | 255 | N | S | 1.3 |
| 233 | 109 | 256 | E | A | 0.9 |
| 234 | 109 | 259 | H | W | 1.1 |
| 235 | 109 | 260 | I | L | 1.1 |
| 236 | 109 | 260 | I | V | 1 |
| 237 | 109 | 267 | R | C | 1 |
| 238 | 109 | 272 | I | V | 0.8 |
| 239 | 109 | 276 | L | G | 0.8 |
| 240 | 109 | 278 | I | L | 1.1 |
| 241 | 109 | 286 | S | A | 0.9 |
| 242 | 109 | 298 | S | A | 2.1 |
| 243 | 109 | 298 | S | T | 2.3 |
| 244 | 109 | 299 | D | A | 2 |
| 245 | 109 | 302 | N | A | 1.9 |
| 246 | 109 | 303 | A | C | 2 |
| 247 | 109 | 303 | A | D | 1.5 |
| 248 | 109 | 303 | A | E | 2.3 |
| 249 | 109 | 303 | A | S | 2.6 |
| 250 | 109 | 304 | T | A | 0.7 |
| 251 | 109 | 305 | S | A | 1 |
| 252 | 109 | 305 | S | G | 0.7 |
| 253 | 109 | 307 | A | S | 0.9 |
| 254 | 109 | 312 | V | L | 1.9 |
| 255 | 109 | 320 | R | L | 1.1 |
| 256 | 109 | 321 | R | N | 1.7 |
| 257 | 109 | 327 | G | L | 2.4 |
| 258 | 109 | 327 | G | Q | 2.8 |
| 259 | 109 | 327 | G | V | 2.4 |
| 260 | 109 | 328 | A | C | 1.7 |
| 261 | 109 | 328 | A | D | 2.3 |
| 262 | 109 | 328 | A | R | 3 |
| 263 | 109 | 328 | A | S | 2.2 |
| 264 | 109 | 328 | A | T | 1.6 |
| 265 | 109 | 328 | A | V | 1.8 |
| 266 | 509 | 3 | Q | P | 1.2 |
| 267 | 509 | 75 | I | R | 1.0 |
| 268 | 509 | 85 | L | A | 1.1 |
| 269 | 509 | 92 | R | K | 1.1 |
| 270 | 509 | 105 | Q | G | 1.1 |
| 271 | 509 | 316 | R | S | 1.3 |
| 272 | 509 | 304 | T | V | 1.0 |
| 273 | 509 | 65 | V | C | 1.0 |

MEGATABLE 2

| SEQ | Trivial | Back- | Fold |
|---|---|---|---|
| ID NO: | Name | Bone | Activity |
| 133 | DDEC0201 | Self | 1.0 |
| 134 | S04087550 | 133 | 1.1 |
| 135 | S04087651 | 133 | 1.3 |
| 136 | S04087682 | 133 | 1.4 |
| 137 | S04087724 | 133 | 1.4 |
| 138 | S04087726 | 133 | 1.1 |
| 139 | S04087758 | 133 | 1.1 |
| 140 | S04087816 | 133 | 1.1 |
| 141 | S04087817 | 133 | 0.9 |
| 142 | S04087867 | 133 | 1.4 |
| 143 | S04087869 | 133 | 1.3 |
| 144 | S04087874 | 133 | 1.2 |
| 145 | S04087904 | 133 | 1.1 |
| 146 | S04087906 | 133 | 1.2 |
| 147 | S04087910 | 133 | 0.8 |
| 148 | S04087922 | 133 | 0.8 |
| 149 | S04087951 | 133 | 1.1 |
| 150 | S04087955 | 133 | 1.1 |
| 151 | S04087989 | 133 | 1.0 |
| 152 | S04088002 | 133 | 1.1 |
| 153 | S04088006 | 133 | 1.8 |
| 154 | S04088059 | 133 | 1.3 |
| 155 | S04088062 | 133 | 1.2 |
| 156 | S04088065 | 133 | 1.5 |
| 157 | S04088073 | 133 | 1.2 |
| 158 | S04088096 | 133 | 1.0 |
| 159 | S04088099 | 133 | 1.0 |
| 160 | S04088106 | 133 | 1.1 |
| 161 | S04088161 | 133 | 1.1 |
| 162 | S04088163 | 133 | 1.0 |
| 163 | S04088168 | 133 | 1.3 |
| 164 | S04088173 | 133 | 0.9 |
| 165 | S04088185 | 133 | 1.1 |
| 166 | S04088201 | 133 | 1.0 |
| 167 | S04088213 | 133 | 1.1 |
| 168 | S04088238 | 133 | 1.1 |
| 169 | S04088328 | 133 | 1.0 |
| 170 | S04088406 | 133 | 1.1 |
| 171 | S04088438 | 133 | 1.1 |
| 172 | S04088440 | 133 | 1.1 |
| 173 | S04088448 | 133 | 1.4 |
| 174 | S04088458 | 133 | 1.1 |
| 175 | S04088522 | 133 | 1.3 |
| 176 | S04088555 | 133 | 1.0 |
| 177 | S04088647 | 133 | 1.0 |
| 178 | S04088672 | 133 | 1.2 |
| 179 | S04088678 | 133 | 0.9 |
| 180 | S04088695 | 133 | 1.2 |
| 181 | S04088702 | 133 | 1.0 |
| 182 | S04088703 | 133 | 1.1 |
| 183 | S04088710 | 133 | 1.0 |
| 184 | S04088744 | 133 | 0.8 |
| 185 | S04088787 | 133 | 1.2 |
| 186 | S04088838 | 133 | 1.2 |
| 187 | S04088881 | 133 | 1.1 |
| 188 | S04088909 | 133 | 1.1 |
| 189 | S04088926 | 133 | 0.9 |
| 190 | S04088929 | 133 | 1.0 |
| 191 | S04088935 | 133 | 1.4 |
| 192 | S04088938 | 133 | 1.0 |
| 193 | S04088987 | 133 | 1.9 |
| 194 | S04089008 | 133 | 2.2 |
| 195 | S04089015 | 133 | 3.0 |
| 196 | S04089044 | 133 | 1.1 |
| 197 | S04089049 | 133 | 1.1 |
| 198 | S04089092 | 133 | 2.0 |
| 199 | S04089093 | 133 | 1.2 |
| 200 | S04089106 | 133 | 1.0 |
| 201 | S04089113 | 133 | 1.5 |
| 202 | S04089148 | 133 | 2.2 |
| 203 | S04089157 | 133 | 2.3 |
| 204 | S04089193 | 133 | 1.0 |
| 205 | S04089275 | 133 | 1.0 |
| 206 | S04089289 | 133 | 1.3 |
| 207 | S04089300 | 133 | 1.4 |
| 208 | S04089344 | 133 | 2.2 |
| 209 | S04089354 | 133 | 1.3 |
| 210 | S04089375 | 133 | 1.3 |
| 211 | S04089378 | 133 | 1.2 |
| 212 | S04089379 | 133 | 1.3 |
| 213 | S04089387 | 133 | 1.5 |
| 214 | S04089392 | 133 | 1.5 |
| 215 | S04089394 | 133 | 1.1 |
| 216 | S04089406 | 133 | 2.1 |
| 217 | S04089407 | 133 | 1.8 |
| 218 | S04089411 | 133 | 2.1 |
| 219 | S04089429 | 133 | 1.4 |
| 220 | S04089431 | 133 | 2.1 |
| 221 | S04089436 | 133 | 1.1 |
| 222 | S04089449 | 133 | 1.1 |
| 223 | S04089460 | 133 | 1.7 |
| 224 | S04089461 | 133 | 1.6 |
| 225 | S04089466 | 133 | 0.9 |
| 226 | S04089471 | 133 | 1.0 |
| 227 | S04089493 | 133 | 2.1 |
| 228 | S04089512 | 133 | 1.6 |
| 229 | S04089536 | 133 | 1.0 |
| 230 | S04089558 | 133 | 1.2 |
| 231 | S04089560 | 133 | 0.9 |
| 232 | S04089564 | 133 | 1.3 |
| 233 | S04089565 | 133 | 1.0 |
| 234 | S04089576 | 133 | 0.9 |
| 235 | S04089589 | 133 | 1.5 |
| 236 | S04089597 | 133 | 0.9 |
| 237 | S04089598 | 133 | 1.0 |
| 238 | S04089614 | 133 | 0.8 |
| 239 | S04089621 | 133 | 1.2 |
| 240 | S04089627 | 133 | 0.9 |
| 241 | S04089630 | 133 | 0.9 |
| 242 | S04089654 | 133 | 1.0 |
| 243 | S04089656 | 133 | 1.6 |
| 244 | S04089681 | 133 | 1.0 |
| 245 | S04089686 | 133 | 1.0 |
| 246 | S04089707 | 133 | 0.8 |
| 247 | S04089714 | 133 | 1.0 |
| 248 | S04089716 | 133 | 1.5 |
| 249 | S04089729 | 133 | 0.9 |
| 250 | S04089733 | 133 | 0.8 |
| 251 | S04089736 | 133 | 1.2 |
| 252 | S04089737 | 133 | 0.9 |
| 253 | S04089738 | 133 | 1.7 |
| 254 | S04089739 | 133 | 1.2 |
| 255 | S04089752 | 133 | 1.0 |
| 256 | S04089758 | 133 | 1.0 |
| 257 | S04089780 | 133 | 1.6 |
| 258 | S04089781 | 133 | 1.2 |
| 259 | S04089795 | 133 | 1.8 |
| 260 | S04089797 | 133 | 1.5 |
| 261 | S04090008 | 133 | 1.2 |
| 262 | S04090070 | 133 | 1.2 |
| 263 | S04090112 | 133 | 0.9 |
| 264 | S04090217 | 133 | 1.1 |
| 265 | S04090480 | 133 | 1.0 |
| 266 | S04090496 | 133 | 1.3 |
| 267 | S04090497 | 133 | 2.2 |
| 268 | S04090502 | 133 | 1.3 |
| 269 | S04090508 | 133 | 1.1 |
| 270 | S04090509 | 133 | 1.0 |
| 271 | S04090557 | 133 | 1.2 |
| 272 | S04090558 | 133 | 1.0 |
| 273 | S04090566 | 133 | 1.0 |
| 274 | S04090625 | 133 | 1.0 |
| 275 | S04090637 | 133 | 1.0 |
| 276 | S04090649 | 133 | 1.0 |
| 277 | S04090657 | 133 | 0.9 |
| 278 | S04090658 | 133 | 1.2 |
| 279 | S04090659 | 133 | 0.9 |
| 280 | S04090677 | 133 | 1.0 |
| 281 | S04090685 | 133 | 1.2 |
| 282 | S04090702 | 133 | 1.0 |
| 283 | S04090705 | 133 | 1.1 |
| 284 | S04090737 | 133 | 0.9 |
| 285 | S04090748 | 133 | 0.9 |
| 286 | S04090752 | 133 | 0.9 |
| 287 | S04090761 | 133 | 0.9 |
| 288 | S04090777 | 133 | 0.9 |
| 289 | S04090785 | 133 | 1.1 |
| 290 | S04090800 | 133 | 1.0 |
| 291 | S04090803 | 133 | 1.2 |
| 292 | S04090816 | 133 | 1.0 |
| 293 | S04090932 | 133 | 1.1 |
| 294 | S04090952 | 133 | 1.4 |
| 295 | S04091022 | 133 | 1.1 |
| 296 | S04091074 | 133 | 1.0 |
| 297 | S04091079 | 133 | 0.9 |
| 298 | S04091121 | 133 | 1.1 |
| 299 | S04091138 | 133 | 1.4 |
| 300 | S04091140 | 133 | 1.4 |
| 301 | S04091164 | 133 | 1.2 |
| 302 | S04091202 | 133 | 0.9 |
| 303 | S04091206 | 133 | 1.0 |
| 304 | S04091207 | 133 | 1.2 |
| 305 | S04091218 | 133 | 0.9 |
| 306 | S04091219 | 133 | 1.3 |
| 307 | S04091234 | 133 | 0.8 |
| 308 | S04091246 | 133 | 1.0 |
| 309 | S04091278 | 133 | 1.0 |
| 310 | S04091288 | 133 | 1.1 |
| 311 | S04091316 | 133 | 1.1 |
| 312 | S04091320 | 133 | 1.0 |
| 313 | S04091339 | 133 | 0.9 |
| 314 | S04091345 | 133 | 1.0 |
| 315 | S04091373 | 133 | 1.0 |
| 316 | S04091375 | 133 | 1.4 |
| 317 | S04091402 | 133 | 1.1 |
| 318 | S04091404 | 133 | 1.3 |
| 319 | S04091407 | 133 | 1.3 |
| 320 | S04091409 | 133 | 1.8 |
| 321 | S04091411 | 133 | 1.6 |
| 322 | S04091416 | 133 | 1.2 |
| 323 | S04091433 | 133 | 1.3 |
| 324 | S04091442 | 133 | 1.0 |
| 325 | S04091461 | 133 | 1.2 |
| 326 | S04091471 | 133 | 1.3 |
| 327 | S04091490 | 133 | 1.1 |
| 328 | S04091495 | 133 | 1.1 |
| 329 | S04091499 | 133 | 0.9 |
| 330 | S04091501 | 133 | 0.9 |
| 331 | S04091502 | 133 | 0.9 |
| 332 | S04091507 | 133 | 1.1 |
| 333 | S04091519 | 133 | 1.1 |
| 334 | S04091526 | 133 | 1.2 |
| 335 | S04091544 | 133 | 1.2 |
| 336 | S04091546 | 133 | 0.8 |
| 337 | S04091566 | 133 | 1.2 |
| 338 | S04091572 | 133 | 1.1 |
| 339 | S04091587 | 133 | 1.0 |
| 340 | S04091590 | 133 | 1.1 |
| 341 | S04091600 | 133 | 1.0 |
| 342 | S04091609 | 133 | 0.9 |
| 343 | S04091611 | 133 | 1.1 |
| 344 | S04091614 | 133 | 1.1 |
| 345 | S04091618 | 133 | 1.0 |
| 346 | S04091621 | 133 | 1.0 |
| 347 | S04091622 | 133 | 1.7 |
| 348 | S04091639 | 133 | 1.1 |
| 349 | S04091640 | 133 | 0.9 |
| 350 | S04091647 | 133 | 0.9 |
| 351 | S04091650 | 133 | 1.0 |
| 352 | S04091655 | 133 | 0.9 |
| 353 | S04091677 | 133 | 1.7 |
| 354 | S04091687 | 133 | 0.9 |
| 355 | S04091721 | 133 | 1.0 |
| 356 | S04091727 | 133 | 1.0 |
| 357 | S04091733 | 133 | 1.4 |
| 358 | S04091736 | 133 | 0.9 |
| 359 | S04091737 | 133 | 1.3 |
| 360 | S04091750 | 133 | 1.1 |
| 361 | S04091757 | 133 | 1.0 |
| 362 | S04091765 | 133 | 0.9 |
| 363 | S04091776 | 133 | 0.9 |
| 364 | S04091784 | 133 | 1.0 |
| 365 | S04091791 | 133 | 1.6 |
| 366 | S04091795 | 133 | 0.9 |
| 367 | S04091812 | 133 | 0.9 |
| 368 | S04091844 | 133 | 0.9 |
| 369 | S04091847 | 133 | 1.1 |
| 370 | S04091869 | 133 | 0.9 |
| 371 | S04091876 | 133 | 0.9 |
| 372 | S04091882 | 133 | 1.1 |
| 373 | S04091909 | 133 | 1.2 |
| 374 | S04091918 | 133 | 1.3 |
| 375 | S04091929 | 133 | 0.9 |
| 376 | S04091931 | 133 | 1.3 |
| 377 | S04091943 | 133 | 1.0 |
| 378 | S04091946 | 133 | 1.1 |
| 379 | S04091948 | 133 | 1.1 |
| 380 | DDEC0301 | Self | 1.0 |
| 381 | S04248889 | 380 | 1.3 |
| 382 | S04248953 | 380 | 1.3 |
| 383 | S04249228 | 380 | 1.6 |
| 384 | S04249439 | 380 | 1.3 |
| 385 | S04249604 | 380 | 1.3 |
| 386 | S04250094 | 380 | 1.1 |
| 387 | S04250281 | 380 | 0.9 |
| 388 | S04250412 | 380 | 1.2 |
| 389 | S04250467 | 380 | 1.3 |
| 390 | S04250942 | 380 | 1.2 |
| 391 | S04251253 | 380 | 1.5 |
| 392 | S04251277 | 380 | 1.4 |
| 393 | S04251419 | 380 | 1.1 |
| 394 | S04251446 | 380 | 1.2 |
| 395 | S04251900 | 380 | 1.0 |
| 396 | S04251964 | 380 | 1.9 |
| 397 | S04251967 | 380 | 1.8 |
| 398 | S04252089 | 380 | 1.0 |
| 399 | S04252092 | 380 | 1.5 |
| 400 | S04252179 | 380 | 1.6 |
| 401 | S04252265 | 380 | 1.2 |
| 402 | S04252918 | 380 | 1.0 |
| 403 | S04253146 | 380 | 1.6 |
| 404 | S04253214 | 380 | 2.0 |
| 405 | S04253311 | 380 | 1.6 |
| 406 | S04253359 | 380 | 1.4 |
| 407 | S04253596 | 380 | 1.8 |
| 408 | S04253796 | 380 | 0.8 |
| 409 | S04254138 | 380 | 1.5 |
| 410 | S04254247 | 380 | 1.3 |
| 411 | S04254262 | 380 | 1.6 |
| 412 | S04254326 | 380 | 1.2 |
| 413 | S04254781 | 380 | 1.4 |
| 414 | S04254783 | 380 | 1.1 |
| 415 | S04254977 | 380 | 1.1 |
| 416 | S04254985 | 380 | 1.1 |
| 417 | S04257584 | 380 | 1.9 |
| 418 | S04257591 | 380 | 1.8 |
| 419 | S04257645 | 380 | 2.2 |
| 420 | S04257663 | 380 | 1.5 |
| 421 | S04257674 | 380 | 2.4 |
| 422 | S04257682 | 380 | 2.2 |
| 423 | S04257687 | 380 | 2.1 |
| 424 | S04257715 | 380 | 1.8 |
| 425 | S04257721 | 380 | 1.8 |
| 426 | S04257735 | 380 | 1.6 |
| 427 | S04257745 | 380 | 2.4 |
| 428 | S04257771 | 380 | 1.1 |
| 429 | S04257772 | 380 | 1.0 |
| 430 | S04257783 | 380 | 2.1 |
| 431 | S04257791 | 380 | 2.1 |
| 432 | S04257822 | 380 | 2.1 |
| 433 | S04257844 | 380 | 1.9 |
| 434 | S04257916 | 380 | 0.8 |
| 435 | S04257946 | 380 | 1.2 |
| 436 | S04257952 | 380 | 1.8 |
| 437 | S04257961 | 380 | 1.2 |
| 438 | S04257968 | 380 | 1.5 |
| 439 | S04257972 | 380 | 1.9 |
| 440 | S04258020 | 380 | 1.3 |
| 441 | S04258197 | 380 | 1.8 |
| 442 | S04258198 | 380 | 1.1 |
| 443 | S04258282 | 380 | 1.6 |
| 444 | S04258336 | 380 | 2.3 |
| 445 | S04258378 | 380 | 1.5 |
| 446 | S04258401 | 380 | 1.0 |
| 447 | S04258456 | 380 | 1.2 |
| 448 | S04258536 | 380 | 1.8 |
| 449 | S04258558 | 380 | 1.3 |
| 450 | S04258572 | 380 | 0.9 |
| 451 | S04259135 | 380 | 1.4 |
| 452 | S04259209 | 380 | 2.0 |
| 453 | S04270153 | 380 | 1.7 |
| 454 | S04270223 | 380 | 1.8 |
| 455 | S04270322 | 380 | 2.1 |
| 456 | S04270340 | 380 | 1.7 |
| 457 | S04270824 | 380 | 1.7 |
| 458 | S04272119 | 380 | 1.2 |
| 459 | S04272152 | 380 | 1.1 |
| 460 | S04272230 | 380 | 1.9 |
| 461 | S04272235 | 380 | 1.7 |
| 462 | S04272236 | 380 | 1.1 |
| 463 | S04272266 | 380 | 1.6 |
| 464 | S04272282 | 380 | 1.0 |
| 465 | S04272335 | 380 | 1.6 |
| 466 | S04272449 | 380 | 1.8 |
| 467 | S04272458 | 380 | 1.7 |
| 468 | S04272506 | 380 | 2.1 |
| 469 | S04272550 | 380 | 1.8 |
| 470 | S04272603 | 380 | 1.8 |
| 471 | S04272623 | 380 | 1.3 |
| 472 | S04272639 | 380 | 1.4 |
| 473 | S04272708 | 380 | 1.9 |
| 474 | S04272711 | 380 | 1.6 |
| 475 | S04273140 | 380 | 1.2 |
| 476 | S04273437 | 380 | 1.8 |
| 477 | S04276453 | 380 | 2.1 |
| 478 | S04276487 | 380 | 1.9 |
| 479 | S04276519 | 380 | 1.4 |
| 480 | S04276690 | 380 | 1.1 |
| 481 | S04276719 | 380 | 1.1 |
| 482 | S04276738 | 380 | 0.9 |
| 483 | S04276757 | 380 | 1.4 |
| 484 | S04276825 | 380 | 0.9 |
| 485 | S04276881 | 380 | 0.9 |
| 486 | S04276959 | 380 | 0.8 |
| 487 | S04277132 | 380 | 1.1 |
| 488 | S04277140 | 380 | 1.4 |
| 489 | S04277170 | 380 | 1.4 |
| 490 | S04278562 | 380 | 2.2 |
| 491 | S04278670 | 380 | 2.1 |
| 492 | S04278687 | 380 | 2.3 |
| 493 | S04278724 | 380 | 2.2 |
| 494 | S04278750 | 380 | 1.9 |
| 495 | S04278814 | 380 | 2.2 |
| 496 | S04278816 | 380 | 2.2 |
| 497 | S04279302 | 380 | 1.0 |
| 498 | S04279398 | 380 | 1.3 |
| 499 | S04279437 | 380 | 0.9 |
| 500 | S04279453 | 380 | 0.9 |
| 501 | S04279471 | 380 | 1.5 |
| 502 | S04279484 | 380 | 1.0 |
| 503 | S04280774 | 380 | 2.1 |
| 504 | S04280791 | 380 | 2.3 |
| 505 | S04280865 | 380 | 2.0 |
| 506 | S04280944 | 380 | 1.1 |
| 507 | S04280958 | 380 | 1.8 |
| 508 | S04280989 | 380 | 1.0 |
| 509 | DDEC0810 | Self | 1.0 |
| 510 | S04319768 | 509 | 1.0 |
| 511 | S04319801 | 509 | 1.3 |
| 512 | S04319804 | 509 | 1.2 |
| 513 | S04319806 | 509 | 1.2 |
| 514 | S04319891 | 509 | 1.1 |
| 515 | S04319906 | 509 | 1.0 |
| 516 | S04319916 | 509 | 1.1 |
| 517 | S04319947 | 509 | 1.2 |
| 518 | S04319952 | 509 | 1.5 |
| 519 | S04319968 | 509 | 1.1 |
| 520 | S04320007 | 509 | 0.8 |
| 521 | S04320019 | 509 | 1.5 |
| 522 | S04320046 | 509 | 1.1 |
| 523 | S04320063 | 509 | 1.2 |
| 524 | S04320064 | 509 | 1.1 |
| 525 | S04320066 | 509 | 1.0 |
| 526 | S04320091 | 509 | 1.0 |
| 527 | S04320184 | 509 | 1.1 |
| 528 | S04320223 | 509 | 1.3 |
| 529 | S04320224 | 509 | 1.2 |
| 530 | S04320274 | 509 | 1.1 |
| 531 | S04320366 | 509 | 1.3 |
| 532 | S04320431 | 509 | 1.3 |
| 533 | S04320434 | 509 | 0.9 |
| 534 | S04320440 | 509 | 1.1 |
| 535 | S04320519 | 509 | 1.1 |
| 536 | S04320520 | 509 | 1.3 |
| 537 | S04320545 | 509 | 1.3 |
| 538 | S04320597 | 509 | 1.0 |
| 539 | S04320606 | 509 | 0.9 |
| 540 | S04320610 | 509 | 1.0 |
| 541 | S04320629 | 509 | 0.9 |
| 542 | S04320636 | 509 | 1.0 |
| 543 | S04320673 | 509 | 0.9 |
| 544 | S04320735 | 509 | 1.2 |
| 545 | S04320744 | 509 | 1.1 |
| 546 | S04320751 | 509 | 1.3 |
| 547 | S04320771 | 509 | 1.6 |
| 548 | S04320802 | 509 | 0.8 |
| 549 | S04320808 | 509 | 1.1 |
| 550 | S04320859 | 509 | 1.1 |
| 551 | S04320860 | 509 | 1.0 |
| 552 | S04320875 | 509 | 1.7 |
| 553 | S04320879 | 509 | 0.9 |
| 554 | S04320889 | 509 | 0.9 |
| 555 | S04320899 | 509 | 0.8 |
| 556 | S04320957 | 509 | 1.3 |
| 557 | S04321009 | 509 | 1.0 |
| 558 | S04321096 | 509 | 1.0 |
| 559 | S04321111 | 509 | 1.0 |
| 560 | S04321170 | 509 | 0.9 |
| 561 | S04321275 | 509 | 1.1 |
| 562 | S04321300 | 509 | 1.7 |
| 563 | S04321304 | 509 | 0.9 |
| 564 | S04321440 | 509 | 0.9 |
| 565 | S04321451 | 509 | 1.1 |
| 566 | S04321468 | 509 | 0.9 |
| 567 | S04321471 | 509 | 1.1 |
| 568 | S04321475 | 509 | 1.6 |
| 569 | S04321512 | 509 | 1.3 |
| 570 | S04321514 | 509 | 1.3 |
| 571 | S04321522 | 509 | 0.9 |
| 572 | S04321531 | 509 | 1.0 |
| 573 | S04321545 | 509 | 0.8 |
| 574 | S04321555 | 509 | 1.1 |
| 575 | S04321608 | 509 | 1.3 |
| 576 | S04321610 | 509 | 1.2 |
| 577 | S04321613 | 509 | 1.2 |
| 578 | S04321667 | 509 | 0.9 |
| 579 | S04321761 | 509 | 1.0 |
| 580 | S04321771 | 509 | 1.3 |
| 581 | S04321781 | 509 | 1.1 |
| 582 | S04321814 | 509 | 1.4 |
| 583 | S04321817 | 509 | 1.0 |
| 584 | S04321906 | 509 | 0.9 |
| 585 | S04321944 | 509 | 1.8 |
| 586 | S04321952 | 509 | 1.2 |

1. A recombinant polypeptide having dicamba decarboxylase activity comprising:
(a) an amino acid sequence having a similarity score of at least 548 for any one of SEQ ID NO: 51, 89, 79, 81, 95, or 100, wherein the similarity score is generated using the BLAST alignment program, with the BLOSUM62 substitution matrix, a gap existence penalty of 11, and a gap extension penalty of 1;
(b) an amino acid sequence having at least 60%, 70%, 75%, 80% 90%, 95% or 100% sequence identity to any one of SEQ ID NOS: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129; or,
(c) an amino acid sequence having at least 60%, 70%, 75%, 80% 90%, or 95% sequence identity to SEQ ID NO: 1, 2, 4, 5, 16, 19, 21, 22, 26, 28, 30, 21, 32, 33, 34, 35, 36, 41, 43, 44, 46, 47, 48, 49, 50, 51, 52, 53, 54, 55, 56, 57, 58, 79, 81, 87, 88, 89, 91, 108, 109, 111, 112, 113, 114, 115, 116, 117, 118, 119, 120, 121, 122, 123, 124, 125, 126, 127, 128, or 129;
wherein the recombinant polypeptide comprises an active site having a catalytic residue geometry as set forth in Table 3 or having a substantially similar catalytic residue geometry;
wherein (a), (b), or (c) comprise the following amino acids:
(i) the amino acid residue in the encoded polypeptide that corresponds to amino acid position 27 of SEQ ID NO: 109 comprises alanine, serine, or threonine;
(ii) the amino acid residue in the encoded polypeptide that corresponds to amino acid position 38 of SEQ ID NO: 109 comprises isoleucine;
(iii) the amino acid residue in the encoded polypeptide that corresponds to amino acid position 42 of SEQ ID NO: 109 comprises alanine, methionine, or serine;
(iv) the amino acid residue in the encoded polypeptide that corresponds to amino acid position 52 of SEQ ID NO: 109 comprises glutamic acid;
(v) the amino acid residue in the encoded polypeptide that corresponds to amino acid position 61 of SEQ ID NO: 109 comprises alanine or serine;
(vi) the amino acid residue in the encoded polypeptide that corresponds to amino acid position 64 of SEQ ID NO: 109 comprises glycine, or serine;
(vii) the amino acid residue in the encoded polypeptide that corresponds to amino acid position 127 of SEQ ID NO: 109 comprises methionine;
(iix) the amino acid residue in the encoded polypeptide that corresponds to amino acid position 238 of SEQ ID NO: 109 comprises glycine;
(ix) the amino acid residue in the encoded polypeptide that corresponds to amino acid position 240 of SEQ ID NO: 109 comprises alanine, aspartic acid, or glutamic acid;
(x) the amino acid residue in the encoded polypeptide that corresponds to amino acid position 298 of SEQ ID NO: 109 comprises alanine or threonine,
(xi) the amino acid residue in the encoded polypeptide that corresponds to amino acid position 299 of SEQ ID NO: 109 comprises alanine;
(xii) the amino acid residue in the encoded polypeptide that corresponds to amino acid position 303 of SEQ ID NO: 109 comprises cysteine, glutamic acid, or serine;
(xiii) the amino acid residue in the encoded polypeptide that corresponds to amino acid position 327 of SEQ ID NO: 109 comprises leucine, glutamine, or valine;
(ixv) the amino acid residue in the encoded polypeptide that corresponds to amino acid position 328 of SEQ ID NO: 109 comprises aspartic acid, arginine, or serine; and/or,
(xv) the amino acid residue in the encoded protein that corresponds to the amino acid position of SEQ ID NO: 109 as set forth in Table 7 and corresponds to the specific amino acid substitution also set forth in Table 7 or any combination of residues denoted in Table 7.

2. A recombinant polypeptide having dicamba decarboxylase activity comprising an amino acid sequence of the formula:
```
(SEQ ID NO: 1041)
                                5 10
Met Ala Xaa Gly Lys Val Xaa Leu Glu Glu His Xaa
                15 20
Ala Ile Xaa Xaa Thr Leu Xaa Xaa Xaa Ala Xaa Phe
25 30 35
Val Pro Xaa Xaa Tyr Xaa Lys Xaa Leu Xaa His Arg
                        40 45
Leu Xaa Asp Xaa Gln Xaa Xaa Arg Leu Xaa Xaa Met
        50 55 60
Asp Xaa His Xaa Ile Xaa Xaa Met Xaa Leu Ser Leu
                                65 70
Xaa Ala Xaa Xaa Xaa Gln Xaa Xaa Xaa Xaa Arg Xaa
                75 80
Xaa Ala Xaa Xaa Xaa Ala Xaa Arg Xaa Asn Asp Xaa
85 90 95
Xaa Ala Glu Xaa Xaa Ala Xaa Xaa Xaa Xaa Arg Phe
                        100 105
Xaa Ala Phe Xaa Xaa Xaa Pro Xaa Xaa Asp Xaa Xaa
        110 115 120
Xaa Ala Xaa Xaa Glu Leu Gln Arg Xaa Val Xaa Xaa
                                125 130
Leu Gly Xaa Val Gly Ala Xaa Val Asn Gly Phe Ser
                135 140
Xaa Glu Gly Asp Xaa Xaa Thr Pro Leu Tyr Tyr Asp
145 150 155
Leu Pro Xaa Tyr Arg Pro Phe Trp Xaa Glu Val Glu
                        160 165
Lys Leu Asp Val Pro Phe Tyr Leu His Pro Xaa Asn
        170 175 180
Pro Leu Pro Gln Asp Xaa Arg Ile Tyr Xaa Gly His
                                185 190
Pro Trp Leu Leu Gly Pro Thr Trp Ala Phe Ala Gln
                195 200
Glu Thr Xaa Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Xaa
        230 235 240
Xaa Arg Ile Asp His Arg Xaa Xaa Xaa Xaa Xaa Xaa
                                245 250
Pro Pro Xaa Tyr Xaa Ala Lys Xaa Xaa Phe Xaa Asp
                255 260
Tyr Phe Xaa Glu Asn Phe Xaa Xaa Thr Thr Ser Gly
265 270 275
Asn Phe Arg Thr Gln Thr Leu Ile Asp Ala Ile Leu
                        280 285
Glu Xaa Gly Ala Asp Arg Ile Leu Phe Ser Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Xaa Thr Asn Ala Xaa Xaa Leu Phe Lys
325
Leu Asp Xaa Xaa,
```
wherein
xaa at position 3 is Gln, Gly, Met or Pro;
xaa at position 7 is Ala or Gys;
xaa at position 12 is Phe, Met, Val or Trp;
xaa at position 15 is Pro or Thr;
xaa at position 16 is Glu or Ala;
xaa at position 19 is Gln, Glu or Asn;
xaa at position 20 is Asp, Cys, Phe, Met or Trp;
xaa at position 21 is Ser, Ala, Gly or Val;
xaa at position 23 is Gly, or Asp;
xaa at position 27 is Gly, Ala, Asp, Flu, Pro, Arg, Ser, Thr or Tyr;
xaa at position 28 is Asp, Cys, Glu, Phe or Gly;
xaa at position 30 is Trp, Leu or Val;
xaa at position 32 is Glu or Val;
xaa at position 34 is Gln, Ala or Trp;
xaa at position 38 is Leu, Ile, Met, Arg, Thr or Val;
xaa at position 40 is Ile, Met, Ser or Val;
xaa at position 42 is Asp, Ala, Gly, Lys, Met, Ser or Thr;
xaa at position 43 is Thr, Cys, Asp, Glu, Gly, Met, Gln, Arg or Tyr;
xaa at position 46 is Lys, Gly, Asn or Arg;
xaa at position 47 is Leu, Cys, Glu, Lys or Ser;
xaa at position 50 is Ala, Lys, Arg, Ser, Thr or Val;
xaa at position 52 is Gly, Glu, Leu, Asn or Gln;
xaa at position 54 is Glu or Gly;
xaa at position 55 is Thr or Leu;
xaa at position 57 is Ile, Ala or Val;
xaa at position 61 is Asn, Ala, Gly, Leu or Ser;
xaa at position 63 is Pro or Val;
xaa at position 64 is Ala, Gly, His or Ser;
xaa at position 65 is Val or Cys;
xaa at position 67 is Ala or Ser;
xaa at position 68 is Ile or Gln;
xaa at position 69 is Pro, Gly, Arg, Ser or Val;
xaa at position 70 is Asp or His;
xaa at position 72 is Arg, Lys or Val;
xaa at position 73 is Lys, Glu, Gln or Arg;
xaa at position 75 is Ile or Arg;
xaa at position 76 is Glu or Gly;
xaa at position 77 is Ile, Met, Arg, Ser or Val;
xaa at position 79 is Arg or Gln;
xaa at position 81 is Ala or Ser;
xaa at position 84 is Val, Cys, Phe or Met;
xaa at position 85 is Leu or Ala;
xaa at position 88 is Glu or Lys;
xaa at position 89 is Cys, Ile or Val;
xaa at position 91 is Lys or Arg;
xaa at position 92 is Arg or Lys;
xaa at position 93 is Pro, Ala or Arg;
xaa at position 94 is Asp, Cys, Gly, Gln or Ser;
xaa at position 97 is Leu, Lys or Arg;
xaa at position 100 is Ala, Gly or Ser;
xaa at position 101 is Ala or Gly;
xaa at position 102 is Leu or Val;
xaa at position 104 is Leu or Met;
xaa at position 105 is Gln or Gly;
xaa at position 107 is Pro or Val;
xaa at position 108 is Asp or Glu;
xaa at position 109 is Ala, Gly, Met or Val;
xaa at position 111 is Thr, Ala, Cys, Gly, Ser or Val;
xaa at position 112 is Glu, Gly, Arg or Ser;
xaa at position 117 is Cys, Ala or Thr;
xaa at position 119 is Asn, Ala, Cys, Arg or Ser;
xaa at position 120 is Asp or Thr;
xaa at position 123 is Phe or Leu;
xaa at position 127 is Leu or Met;
xaa at position 133 is Gln or Val;
xaa at position 137 is Gly, Ala or Glu;
xaa at position 138 is Gln or Gly;
xaa at position 147 is Gln or Ile;
xaa at position 153 is Gly or Lys;
xaa at position 167 is Arg or Glu;
xaa at position 174 is Ser or Ala;
xaa at position 178 is Asp or Glu;
xaa at position 195 is Ala or Gly;
xaa at position 212 is Arg, Gly or Gln;
xaa at position 214 is Asn or Gln;
xaa at position 220 is Met or Leu;
xaa at position 228 is Met or Leu;
xaa at position 229 is Trp or Tyr;
xaa at position 235 is Val or Ile;
xaa at position 236 is Ala, Gly, Gln or Trp;
xaa at position 237 is Trp or Leu;
xaa at position 238 is Val, Gly or Pro;
xaa at position 239 is Lys, Ala, Asp, Glu, Gly or His;
xaa at position 240 is Leu, Ala, Asp, Glu, Gly or Val;
xaa at position 243 is Arg, Ala, Asp, Lys, Ser or Val;
xaa at position 248 is Arg or Lys;
xaa at position 249 is Arg or Pro;
xaa at position 251 is Met or Val;
xaa at position 255 is Asn, Ala, Leu, Met, Gln, Arg or Ser;
xaa at position 259 is His or Trp;
xaa at position 260 is Ile or Leu;
xaa at position 278 is Ile or Leu;
xaa at position 298 is Ser, Ala or Thr;
xaa at position 299 is Asp or Ala;
xaa at position 302 is Asn or Ala;
xaa at position 303 is Ala, Cys, Asp, Glu or Ser;
xaa at position 304 is Thr or Val;
xaa at position 312 is Val or Leu;
xaa at position 316 is Arg or Ser;
xaa at position 320 is Arg or Leu;
xaa at position 321 is Arg or Asn;
xaa at position 327 is Glv, Leu, Gln or Val;
xaa at position 328 is Ala, Cys, Asp, Arg, Ser, Thr or Val;
wherein one or more amino acid(s) designated by Xaa in SEQ ID NO: 1041 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

3. A recombinant polypeptide having dicamba decarboxylase activity comprising an amino acid sequence of the formula:
```
(SEQ ID NO: 1042)
                                5 10
Met Ala Gln Gly Xaa Val Ala Leu Glu Glu His Phe
                15 20
Ala Ile Pro Xaa Thr Leu Xaa Asp Xaa Ala Xaa Phe
25 30 35
Val Pro Xaa Xaa Tyr Xaa Lys Glu Leu Gln His Arg
                        40 45
Leu Xaa Asp Xaa Gln Asp Xaa Arg Leu Xaa Xaa Met
        50 55 60
Asp Xaa His Xaa Ile Xaa Thr Met Xaa Leu Ser Leu
                                65 70
Xaa Ala Xaa Xaa Val Gln Xaa Ile Xaa Asp Arg Xaa
                75 80
Xaa Ala Ile Glu Xaa Ala Xaa Arg Ala Asn Asp Xaa
85 90 95
Leu Ala Glu Glu Xaa Ala Lys Arg Pro Xaa Arg Phe
                        100 105
Leu Ala Phe Ala Ala Leu Pro Xaa Gln Asp Xaa Xaa
        110 115 120
Ala Ala Xaa Xaa Glu Leu Gln Arg Xaa Val Xaa Xaa
                                125 130
Leu Gly Phe Val Gly Ala Xaa Val Asn Gly Phe Ser
                135 140
Xaa Glu Gly Asp Gly Gln Thr Pro Leu Tyr Tyr Asp
145 150 155
Leu Pro Gln Tyr Arg Pro Phe Trp Xaa Glu Val Glu
                        160 165
Lys Leu Asp Val Pro Phe Tyr Leu His Pro Arg Asn
        170 175 180
Pro Leu Pro Gln Asp Xaa Arg Ile Tyr Asp Gly His
                                185 190
Pro Trp Leu Leu Gly Pro Thr Trp Ala Phe Ala Gln
                195 200
Glu Thr Ala Val His Ala Leu Arg Leu Met Ala Ser
205 210 215
Gly Leu Phe Asp Glu His Pro Xaa Leu Xaa Ile Ile
                        220 225
Leu Gly His Xaa Gly Glu Gly Leu Pro Tyr Met Met
        230 235 240
Xaa Arg Ile Asp His Arg Xaa Xaa Trp Val Xaa Xaa
                                245 250
Pro Pro Xaa Tyr Xaa Ala Lys Arg Arg Phe Met Asp
                255 260
Tyr Phe Xaa Glu Asn Phe Xaa Ile Thr Thr Ser Gly
265 270 275
Asn Phe Arg Thr Gln Thr Leu Ile Asp Ala Ile Leu
                        280 285
Glu Ile Gly Ala Asp Arg Ile Leu Phe Xaa Thr Asp
        290 295 300
Trp Pro Phe Glu Asn Ile Asp His Ala Xaa Xaa Trp
                                305 310
Phe Xaa Xaa Xaa Ser Ile Ala Glu Ala Asp Arg Xaa
                315 320
Lys Ile Gly Arg Thr Asn Ala Xaa Xaa Leu Phe Lys
325
Leu Asp Xaa Xaa
```
wherein one or more amino acid(s) designated by Xaa in SEQ ID NO: 1042 is an amino acid different from the corresponding amino acid of SEQ ID NO: 109; and wherein the polypeptide having dicamba decarboxylase activity has increased dicamba decarboxylase activity compared to the polypeptide of SEQ ID NO: 109.

4. The recombinant polypeptide of claim 2, wherein the amino acid position at 21 is Ser or Ala; the amino acid at position 27 is Gly or Ser; the amino acid at position 50 is Ala or Lys; the amino acid at position 52 is Gly or Glu; the amino acid at position 54 is Glu or Gly; the amino acid at position 61 is Asn or Ala; the amino acid at position 84 is Val or Phe; the amino acid at position 127 is Leu or Met; the amino acid at position 235 is Asn or Val or Ile; the amino acid at position 240 is Leu or Ala or Glu; the amino acid at position 298 is Ser or Ala or Thr; the amino acid at position 327 is Gly or Leu or Val; or the amino acid at position 328 is Ala or Arg or Asp or Ser; or combinations thereof.

5. The recombinant polypeptide of claim 2, further comprising substitution of one or more conservative amino acids, insertion of one or more amino acids, deletion of one or more amino acids, and combinations thereof.

6. The recombinant polypeptide of claim 2, wherein the dicamba decarboxylase activity is increased about 1.2 fold or greater compared to SEQ ID NO: 109.

7. The recombinant polypeptide of claim 2, wherein the dicamba decarboxylase activity is increased about 1.4 fold or greater compared to SEQ ID NO: 109.

8. The recombinant polypeptide of claim 2, wherein the dicamba decarboxylase activity is increased about 1.6 fold or greater compared to SEQ ID NO: 109.

9. The recombinant polypeptide of claim 2, wherein the dicamba decarboxylase activity is increased about 1.8 fold or greater compared to SEQ ID NO: 109.

10. The recombinant polypeptide of claim 2, wherein the dicamba decarboxylase activity is increased about 2.0 fold or greater compared to SEQ ID NO: 109.

11. The recombinant polypeptide of claim 2, wherein the dicamba decarboxylase activity is increased about 2.2 fold or greater compared to SEQ ID NO: 109.

12. The recombinant polypeptide of any of claim 1, wherein the polypeptide having dicamba decarboxylase activity has a k_{cat}/Kₘ of at least 0.0001 mM⁻¹ min⁻¹ for dicamba.

13. A polynucleotide construct comprising a nucleotide sequence encoding the polypeptide of claim 1.

14. The polynucleotide construct of claim 14, further comprising a promoter operably linked to the polynucleotide construct.

15. A cell comprising the polynucleotide construct of claim 13.

16. The cell of claim 15, wherein the cell comprises a microbial cell.

17. A method of producing a host cell comprising a heterologous polynucleotide encoding a polypeptide having dicamba decarboxylase activity comprising transforming a host cell with the polynucleotide construct of claim 13.

18. The method of claim 17, wherein the host cell comprises a microbial cell.

19. A method to decarboxylate dicamba, a dicamba derivative or a dicamba metabolite comprising contacting the dicamba, the dicamba derivative or the dicamba metabolite with a composition comprising an effective amount of the polypeptide of any of claims 1-12 or an effective amount of the host cell of claim 17, wherein the effective amount is sufficient to decarboxylate the dicamba, the dicamba derivative or the dicamba metabolite.

20. The method of claim 19, wherein the composition is contacted with dicamba.

21. A method for detecting a polypeptide comprising using an antibody or antibodies that specifically recognize a polypeptide having dicamba decarboxylase activity in an immunoassay; wherein the polypeptide recognized in the immunoassay comprises the polypeptide of claim 1.

22. A method for detecting the presence of a polynucleotide encoding a polypeptide having dicamba decarboxylase activity comprising detecting a polynucleotide encoding the polypeptide of claim 1 in a PCR amplification reaction.
